(19) United States
(12) Patent Application Publication
(10) Pub. No.: US 2021/0038646 A1
(43) Pub. Date: Feb. 11, 2021

(54) CHIMERIC ANTIGEN RECEPTORS TARGETING THE TUMOR MICROENVIRONMENT

(71) Applicant: The General Hospital Corporation, Boston, MA (US)

(72) Inventors: Marcela V. Maus, Lexington, MA (US); Bryan Choi, Boston, MA (US)

(73) Assignee: The General Hospital Corporation, Boston, MA (US)

(21) Appl. No.: 16/969,098

(22) PCT Filed: Feb. 12, 2019
(86) PCT No.: PCT/US2019/017727

§ 371 Date: Aug. 11, 2020

Related U.S. Application Data
(63) Continuation of application No. PCT/US2018/027783, filed on Apr. 16, 2018.
(60) Provisional application No. 62/746,895, filed on Oct. 17, 2018.
(60) Provisional application No. 62/658,307, filed on Apr. 16, 2018.
(60) Provisional application No. 62/629,593, filed on Feb. 12, 2018.

(51) Int. Cl. A61K 35/17; C12N 15/86; A61P 35/00; C07K 14/705; C07K 16/28

(52) U.S. Cl. CPC A61K 35/17; C12N 15/86; A61P 35/00; C07K 16/2809; C07K 14/70517; C07K 14/70578; C07K 14/70521

(57) ABSTRACT

The invention provides methods and compositions for use in treating cancer, which advantageously may be achieved by targeting of a tumor microenvironment. The invention provides chimeric antigen receptors (CARs) that target a tumor microenvironment. In one aspect, the invention features an immune cell engineered to express: (a) a chimeric antigen receptor (CAR) polypeptide including an extracellular domain including a first antigen binding domain that binds to a first antigen and a second antigen-binding domain that binds to a second antigen; and (b) a bispecific T cell engager (BiTE), wherein the BiTE binds to a target antigen and a T cell antigen. In another aspect, the invention features a pharmaceutical composition including the immune cell. In another aspect, the invention features a method of treating a cancer in a subject in need thereof, the method comprising administering the immune cell.

### CROSS-REFERENCE TO RELATED APPLICATIONS

This application claims benefit of U.S. Provisional Application No. 62/629,593, filed Feb. 12, 2018; U.S. Provisional Application No. 62/658,307, filed Apr. 16, 2018; International Patent Application No. PCT/US2018/027783, filed Apr. 16, 2018; and U.S. Provisional Application No. 62/746,895, filed Oct. 17, 2018; the contents of which are incorporated herein by reference in their entirety.

### TECHNICAL FIELD

The technology described herein relates to immunotherapy.

### SEQUENCE LISTING

The instant application contains a Sequence Listing which has been submitted electronically in ASCII format and is hereby incorporated by reference in its entirety. The ASCII copy, created on Feb. 12, 2019, is named 51295-013WO2_Sequence_Listing_2.12.19_ST25 and is 190,819 bytes in size.

### BACKGROUND OF THE INVENTION

Chimeric antigen receptor (CARs) provide a way to direct a cytotoxic T cell response to target cells expressing a selected target antigen, most often a tumor antigen or tumor-associated antigen. CARs are an adaptation of the T cell receptor, where the antigen binding domain is replaced with the antigen binding domain of an antibody that specifically binds the derived target antigen. Engagement of the target antigen on the surface of a target cell by a CAR expressed on, e.g., a T cell (“CART cell” or “CAR-T”) promotes killing of the target cell.

### SUMMARY OF THE INVENTION

The invention provides chimeric antigen receptors (CARs) that target the tumor microenvironment.

In one aspect, the invention, in general, features an immune cell engineered to express: (a) a chimeric antigen receptor (CAR) polypeptide including an extracellular domain including a first antigen-binding domain that binds to a first antigen and a second antigen-binding domain that binds to a second antigen; and (b) a bispecific T cell engager (BiTE), wherein the BiTE binds to a target antigen and a T cell antigen.

In some embodiments, the CAR polypeptide includes a transmembrane domain and an intracellular signaling domain. In some embodiments, the CAR polypeptide further includes one or more co-stimulatory domains. In some embodiments, the CAR includes a transmembrane domain, an intracellular signaling domain, and one or more co-stimulatory domains.

In some embodiments, the first and second antigens are glioblastoma antigens. In further embodiments, the first and second antigens are independently selected from epidermal growth factor receptor (EGFR), epidermal growth factor receptor variant III (EGFRvIII), CD19, CD79b, CD37, prostate-specific membrane antigen (PSMA), prostate stem cell antigen (PSCA), interleukin-13 receptor alpha 2 (IL-13Rα2), ephrin type-A receptor 1 (EphA1), human epidermal growth factor receptor 2 (HER2), mesothelin, mucin 1, cell surface associated (MUC1), or mucin 16, cell surface associated (MUC16).

In some embodiments, the first antigen-binding domain and/or the second antigen-binding domain includes an antigen-binding fragment of an antibody, e.g., a single domain antibody or a single chain variable fragment (scFv). In other embodiments, the first antigen-binding domain and/or the second antigen-binding domain includes a ligand of the first and/or second antigen.

In further embodiments, the extracellular domain does not include a linker between the first antigen-binding domain and the second antigen-binding domain. In other embodiments, the first antigen-binding domain is connected to the second antigen-binding domain by a linker, e.g., wherein the linker includes the amino acid sequence of SEQ ID NO: 102, 107, 108, 109, or 110, or includes an amino acid having at least 90% sequence identity (e.g., 90%, 91%, 92%, 93%, 94%, 95%, 96%, 97%, 98%, or 99% sequence identity) to the linker of SEQ ID NO: 102, 107, 108, 109, or 110.

In some embodiments, the transmembrane domain includes a hinge/transmembrane domain. In some embodiments, the hinge/transmembrane domain includes the hinge/transmembrane domain of an immunoglobulin-like protein (e.g., IgA, IgD, IgE, IgG, or IgM), CD28, CD8, or 4-1 BB. In particular embodiments, the transmembrane domain includes the hinge/transmembrane domain of CD8, optionally including the amino acid sequence of SEQ ID NO: 4, 10, 16, 22, 28, 37, 46, 58, 66, 72, 78, or 104, or an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 4, 10, 16, 22, 28, 37, 46, 58, 66, 72, 78, or 104.

In some embodiments, the intracellular signaling domain includes the intracellular signaling domain of TCRζ, FcRγ, FcRβ, CD3γ, CD3θ, CD3ε, CD3η, CD3ζ, CD22, CD79a, CD79b, or CD66d. In some embodiments, the intracellular signaling domain includes the intracellular signaling domain of CD3, optionally including the amino acid sequence of SEQ ID NO: 6, 12, 18, 24, 30, 39, 48, 60, 68, 74, 80, or 106, or an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 6, 12, 18, 24, 30, 39, 48, 60, 68, 74, 80, or 106.

In further embodiments, the co-stimulatory domain includes the co-stimulatory domain of 4-1 BB, CD27, CD28, or OX-40. In some embodiments, the co-stimulatory domain includes the co-stimulatory domain of 4-1 BB, optionally including the amino acid sequence of SEQ ID NO: 5, 11, 17, 23, 29, 38, 47, 59, 67, 73, 79, or 105, or an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 5, 11, 17, 23, 29, 38, 47, 59, 67, 73, 79, or 105.

In some embodiments, the first antigen-binding domain includes an IL-13Rα2-binding domain. In some embodiments, the second antigen-binding domain includes an EGFRvIII-binding domain.

In some embodiments, the IL-13Rα2-binding domain includes an anti-IL-13Rα2 scFv or a ligand of IL-13Rα2. In some embodiments, the ligand of IL-13Rα2 includes IL-13 or IL-13 zetakine, or an antigen-binding fragment thereof. In further embodiments, the IL-13Rα2-binding domain includes the amino acid sequence of SEQ ID NO: 101, or includes an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 101.

In further embodiments, the EGFRvIII-binding domain includes an antigen-binding fragment of an antibody, e.g., wherein the EGFRvIII-binding domain includes an anti-EGFRvIII scFv. In some embodiments, the anti-EGFRvIII scFv includes a heavy chain variable domain (VH) including the amino acid sequence of SEQ ID NO: 111 or 113, or a VH including an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 111 or 113 and/or a light chain variable domain (VL) including the amino acid sequence of SEQ ID NO: 112 or 114, or a VL including an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 112 or 114. In particular embodiments, the EGFRvIII-binding domain includes the amino acid sequence of SEQ ID NO: 103, or includes an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 103.

In some embodiments, the CAR polypeptide includes the amino acid sequence of SEQ ID NO: 100, or includes an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 100.

In another aspect, the invention features an immune cell engineered to express: (i) a CAR polypeptide including an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 100; and (ii) a BiTE, wherein the BiTE binds to a target antigen and a T cell antigen.

In another aspect, the invention features an immune cell engineered to express: (i) a CAR polypeptide including the amino acid sequence of SEQ ID NO: 100; and (ii) a BiTE, wherein the BiTE binds to a target antigen and a T cell antigen.

In some embodiments of any of the preceding aspects, the target antigen is a glioblastoma-associated antigen selected from one of EGFR, EGFRvIII, CD19, CD79b, CD37, PSMA, PSCA, IL-13Rα2, EphA1, HER2, mesothelin, MUC1, or MUC16. In some embodiments, the T cell antigen is CD3. In particular embodiments, the target antigen is EGFR and the T cell antigen is CD3.

In some embodiments of any of the preceding aspects, the BiTE includes the amino acid sequence of SEQ ID NO: 98 or 99, or includes an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 98 or 99.

In some embodiments of any of the preceding aspects, the immune cell is a T or natural killer (NK) cell. In some embodiments, the immune cell is a human cell.

In another aspect, the invention features, in general, a polynucleotide encoding the CAR polypeptide and the BiTE of any one of the preceding aspects.

In some embodiments, the polynucleotide includes a CAR polypeptide encoding sequence and a BiTE encoding sequence, and wherein the CAR polypeptide encoding sequence and the BiTE encoding sequence are separated by a ribosome skipping moiety. In some embodiments, the CAR polypeptide and/or the BiTE is expressed under a constitutive promoter, e.g., an elongation factor-1 alpha (EF1α) promoter. In other embodiments, the CAR polypeptide and/or the BiTE is expressed under an inducible promoter, e.g., wherein the inducible promoter is inducible by T cell receptor (TCR) or CAR signaling, e.g., a nuclear factor of activated T cells (NFAT) response element. In certain embodiments, the CAR polypeptide and the BiTE are each expressed under a constitutive promoter. In other embodiments, the CAR polypeptide is expressed under a constitutive promoter and the BiTE is expressed under an inducible promoter. In further embodiments, the polynucleotide further includes a suicide gene. In still further embodiments, the polynucleotide includes a sequence encoding one or more signal sequences.

In another aspect, the invention features, in general, a vector including the polynucleotide of the preceding aspect. In some embodiments, the vector is a lentiviral vector.

In another aspect, the invention features, in general, a pharmaceutical composition including the immune cell, the polynucleotide, or the vector of any one of the preceding aspects.

In another aspect, the invention features, in general, a method of treating a cancer in a subject in need thereof, the method including administering the immune cell, the polynucleotide, the vector, or the pharmaceutical composition of any one of the preceding aspects to the subject. In some embodiments, the cancer is glioblastoma, lung cancer, pancreatic cancer, lymphoma, or myeloma, optionally wherein the cancer includes expressing one or more of the group consisting of EGFR, EGFRvIII, CD19, CD79b, CD37, PSMA, PSCA, IL-13Rα2, EphA1, HER2, mesothelin, MUC1, and MUC16. In some embodiments, the glioblastoma includes cells expressing one or more of the group consisting of IL-13Rα2, EGFRvIII, EGFR, HER2, mesothelin, and EphA1. In further embodiments, the glioblastoma includes cells with reduced EGFRvIII expression.

In another aspect, the invention features an immune cell engineered to express: (i) a CAR polypeptide including an EGFR-binding domain, wherein the CAR polypeptide includes the amino acid sequence of SEQ ID NO: 117, or an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 117; and (ii) an anti-GARP camelid including the amino acid sequence of SEQ ID NO: 25, or an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 25.

In another aspect, the invention features an immune cell engineered to express: (i) a CAR polypeptide including an EGFRvIII-binding domain, wherein the CAR polypeptide includes the amino acid sequence of SEQ ID NO: 115 or 116, or an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 115 or 116; and (ii) a BiTE, wherein the BiTE binds to EGFR and CD3, including the amino acid sequence of SEQ ID NO: 98 or 99, or an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 98 or 99.

In another aspect, the invention features a polynucleotide encoding the CAR polypeptide and the anti-GARP camelid of the preceding aspect.

In another aspect, the invention features the CAR polypeptide and the BiTE of the preceding aspect.

In some embodiments of the preceding polynucleotides, the polynucleotide further includes a suicide gene. In some embodiments, the polynucleotide further includes a sequence encoding one or more signal sequences.

In another aspect, the invention features, in general, a vector including the polynucleotide of any one of the preceding aspects. In some embodiments, the vector is a lentiviral vector.

In another aspect, the invention features, in general, a pharmaceutical composition including the immune cell, the polynucleotide, or the vector of any one of the preceding aspects.

In another aspect, the invention features a method of treating glioblastoma having reduced EGFRvIII expression in a subject including administering to the subject an immune cell engineered to express: (i) a CAR polypeptide including an extracellular EGFRvIII-binding domain; and (ii) a BiTE, wherein the immune cell is optionally selected from the immune cell of any one of the preceding aspects. In some embodiments, the CAR includes a transmembrane domain, an intracellular signaling domain, and one or more co-stimulatory domains.

In another aspect, the invention features a method of preventing or reducing immunosuppression in the tumor microenvironment in a subject including administering to the subject an immune cell including (i) a CAR including an extracellular target binding domain; and (ii) a BiTE, wherein the immune cell is optionally selected from the immune cell of any one of the preceding aspects. In some embodiments, the CAR includes a transmembrane domain, an intracellular signaling domain, and one or more co-stimulatory domains.

In another aspect, the invention features a method of preventing or reducing T cell exhaustion in the tumor microenvironment in a subject, the method including administering to the subject an immune cell including (i) a CAR including an extracellular target binding domain; and (ii) a BiTE, wherein the immune cell is optionally selected from the immune cell of any one of the preceding aspects. In some embodiments, the CAR includes a transmembrane domain, an intracellular signaling domain, and one or more co-stimulatory domains.

In another aspect, the invention features a method of treating a cancer in a subject, the method including administering to the subject an immune cell including (i) a CAR including an extracellular target binding domain; and (ii) a BiTE, wherein the immune cell is optionally selected from the immune cell of any one of the preceding aspects. In some embodiments, the CAR includes a transmembrane domain, an intracellular signaling domain, and one or more co-stimulatory domains. In some embodiments, the cancer is glioblastoma, prostate cancer, lung cancer, pancreatic cancer, lymphoma, or myeloma. In some embodiments, the cancer includes cells expressing one or more of the group consisting of EGFR, EGFRvIII, CD19, PSMA, PSCA, IL-13Rα2, EphA1, Her2, mesothelin, MUC1, and MUC16. In some embodiments, the cancer expresses a heterogeneous antigen. Example of such cancers are glioblastoma (which expresses, e.g., EGFR, EGFRvIII, IL-13Rα2, HER2, and/or EphA1).

In another aspect, the invention features, in general, a CAR T cell including a heterologous nucleic acid molecule, wherein the heterologous nucleic acid molecule includes: (a) a first polynucleotide encoding a CAR including an extracellular antigen-binding domain, a transmembrane domain, and an intracellular signaling domain; and (b) a second polynucleotide encoding a therapeutic agent.

In some embodiments, the therapeutic agent includes an antibody reagent, e.g., a single chain antibody or a single domain antibody (e.g., a camelid antibody). In further embodiments, the antibody reagent includes a bispecific antibody reagent, e.g., a BiTE. In still other embodiments, the therapeutic agent includes a cytokine.

In some embodiments, the CAR and the therapeutic agent are produced as separate CAR and therapeutic agent molecules. In some embodiments, the CAR T cell includes a ribosome skipping moiety between the first polynucleotide encoding the CAR and the second polynucleotide encoding the therapeutic agent. In some embodiments, the ribosome skipping moiety includes a 2A peptide, e.g., P2A or T2A.

In further embodiments, the CAR and the therapeutic agent are each constitutively expressed. In some embodiments, expression of the CAR and the therapeutic agent is driven by an EF1α promoter. In other embodiments, the therapeutic agent is expressed under the control of an inducible promoter, which is optionally inducible by T cell receptor or CAR signaling, e.g., wherein the inducible promoter includes the NFAT promoter. In still further embodiments, the CAR is expressed under the control of a constitutive promoter and the therapeutic agent is expressed under the control of an inducible promoter, which is optionally inducible by T cell receptor or CAR signaling.

In some embodiments, the CAR further includes one or more co-stimulatory domains. In some embodiments, the antigen-binding domain of the CAR includes an antibody, a single chain antibody, a single domain antibody, or a ligand.

In some embodiments, the transmembrane domain includes a hinge/transmembrane domain, e.g., the hinge/transmembrane domain of an immunoglobulin-like protein (e.g., IgA, IgD, IgE, IgG, or IgM), CD28, CD8, or 4-1 BB. In some embodiments, the transmembrane domain of the CAR includes a CD8 hinge/transmembrane domain, which optionally includes the sequence of any one of SEQ ID NOs: 4, 10, 16, 22, 28, 37, 46, 58, 66, 72, 78, and 104, or a variant thereof, or a sequence having at least 90% sequence identity (e.g., 90%, 91%, 92%, 93%, 94%, 95%, 96%, 97%, 98%, or 99% sequence identity) to any one of SEQ ID NOs: 4, 10, 16, 22, 28, 37, 46, 58, 66, 72, 78, and 104.

In further embodiments, the intracellular signaling domain includes the intracellular signaling domain of TCRζ, FcRγ, FcRβ, CD3γ, CD3θ, CD3ε, CD3η, CD3ζ, CD22, CD79a, CD79b, or CD66d. In some embodiments, the intracellular signaling domain includes a CD3ζ intracellular signaling domain, which optionally includes the sequence of any one of SEQ ID NOs: 6, 12, 18, 24, 30, 39, 48, 60, 68, 74, 80, and 106, or a variant thereof, or a sequence having at least 90% sequence identity (e.g., 90%, 91%, 92%, 93%, 94%, 95%, 96%, 97%, 98%, or 99% sequence identity) to any one of SEQ ID NOs: 6, 12, 18, 24, 30, 39, 48, 60, 68, 74, 80, and 106.

In still further embodiments, the co-stimulatory domain includes the co-stimulatory domain of 4-1BB, CD27, CD28, or OX-40. In particular embodiments, the co-stimulatory domain includes a 4-1 BB co-stimulatory domain, which optionally includes the sequence of any one of SEQ ID NOs: 5, 11, 17, 23, 29, 38, 47, 59, 67, 73, 79, and 105, or a variant thereof, or a sequence having at least 90% sequence identity (e.g., 90%, 91%, 92%, 93%, 94%, 95%, 96%, 97%, 98%, or 99% sequence identity) to any one of SEQ ID NOs: 5, 11, 17, 23, 29, 38, 47, 59, 67, 73, 79, and 105.

In some embodiments, the CAR antigen-binding domain binds to a tumor-associated antigen or a Treg-associated antigen. In some embodiments, the camelid antibody binds to a tumor-associated antigen or a Treg-associated antigen. In some embodiments, the BiTE binds to (i) a tumor-associated antigen or a Treg-associated antigen, and (ii) a T cell antigen.

In certain embodiments, the tumor-associated antigen is a solid tumor-associated antigen, e.g., EGFRvIII, EGFR, CD19, PSMA, PSCA, IL-13Rα2, EphA1, Her2, mesothelin, MUC1, or MUC16. Optionally, the CAR antigen-binding domain or the therapeutic agent includes a sequence selected from the group consisting of SEQ ID NO: 21, 27, 33, 36, 42, 45, 51, 55, 57, 63, 65, 103, and variants thereof, or a sequence having at least 90% sequence identity (e.g., 90%, 91%, 92%, 93%, 94%, 95%, 96%, 97%, 98%, or 99% sequence identity) to the amino acid sequence of SEQ ID NO: 21, 27, 33, 36, 42, 45, 51, 55, 57, 63, 65, or 103.

In further embodiments, the Treg-associated antigen is selected from the group consisting of glycoprotein A repetitions predominant (GARP), latency-associated peptide (LAP), CD25, and cytotoxic T lymphocyte-associated antigen-4 (CTLA-4). Optionally, the CAR antigen-binding domain or the therapeutic agent includes a sequence selected from the group consisting of SEQ ID NO: 3, 9, 15, 25, 71, 77, and variants thereof, or a sequence having at least 90% sequence identity (e.g., 90%, 91%, 92%, 93%, 94%, 95%, 96%, 97%, 98%, or 99% sequence identity) to the amino acid sequence of SEQ ID NO: 3, 9, 15, 25, 71, or 77.

In another aspect, the invention features a CAR polypeptide including an extracellular antigen-binding domain, a transmembrane domain, and an intracellular signaling domain; and the antigen-binding domain binds to a Treg-associated antigen. In some embodiments, the Treg-associated antigen is selected from the group consisting of GARP, LAP, CD25, and CTLA-4.

In some embodiments, the CAR further includes one or more co-stimulatory domains.

In certain embodiments, the Treg-associated antigen is GARP or LAP.

In some embodiments, the antigen-binding domain of the CAR includes: (a) a heavy chain variable domain (VH) including three complementarity determining regions CDR-H1, CDR-H2, and CDR-H3, wherein the CDR-H1 includes an amino acid sequence of SEQ ID NO: 81, or an amino acid sequence with no more than 1, 2, or 3 amino acid substitutions of SEQ ID NO: 81; the CDR-H2 includes an amino acid sequence of SEQ ID NO: 82, or an amino acid sequence with no more than 1, 2, or 3 amino acid substitutions of SEQ ID NO: 82; and the CDR-H3 includes an amino acid sequence of SEQ ID NO: 83, or an amino acid sequence with no more than 1, 2, or 3 amino acid substitutions of SEQ ID NO: 83, and/or (b) a light chain variable domain (VL) including three complementarity determining regions CDR-L1, CDR-L2, and CDR-L3, wherein the CDR-L1 includes an amino acid sequence of SEQ ID NO: 84, or an amino acid sequence with no more than 1, 2, or 3 amino acid substitutions of SEQ ID NO: 84; the CDR-L2 includes an amino acid sequence of SEQ ID NO: 85, or an amino acid sequence with no more than 1, 2, or 3 amino acid substitutions of SEQ ID NO: 85; and the CDR-L3 includes an amino acid sequence of SEQ ID NO: 86, or an amino acid sequence with no more than 1, 2, or 3 amino acid substitutions of SEQ ID NO: 86. In some embodiments, the VH includes an amino acid sequence of SEQ ID NO: 87, or an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 87, and/or the VL includes an amino acid sequence of SEQ ID NO: 88, or an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 88.

In other embodiments, the antigen-binding domain of the CAR includes: (a) a heavy chain variable domain (VH) including three complementarity determining regions CDR-H1, CDR-H2, and CDR-H3, wherein the CDR-H1 includes an amino acid sequence of SEQ ID NO: 89, or an amino acid sequence with no more than 1, 2, or 3 amino acid substitutions of SEQ ID NO: 89; the CDR-H2 includes an amino acid sequence of SEQ ID NO: 90, or an amino acid sequence with no more than 1, 2, or 3 amino acid substitutions of SEQ ID NO: 90; and the CDR-H3 includes an amino acid sequence of SEQ ID NO: 91, or an amino acid sequence with no more than 1, 2, or 3 amino acid substitutions of SEQ ID NO: 91, and/or (b) a light chain variable domain (VL) including three complementarity determining regions CDR-L1, CDR-L2, and CDR-L3, wherein the CDR-L1 includes an amino acid sequence of SEQ ID NO: 92, or an amino acid sequence with no more than 1, 2, or 3 amino acid substitutions of SEQ ID NO: 92; the CDR-L2 includes an amino acid sequence of SEQ ID NO: 93, or an amino acid sequence with no more than 1, 2, or 3 amino acid substitutions of SEQ ID NO: 93; and the CDR-L3 includes an amino acid sequence of SEQ ID NO: 94, or an amino acid sequence with no more than 1, 2, or 3 amino acid substitutions of SEQ ID NO: 94. In some embodiments, the VH includes an amino acid sequence of SEQ ID NO: 95, or an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 95, and/or the VL includes an amino acid sequence of SEQ ID NO: 96, or an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 96.

In some embodiments, the VH is N-terminal to the VL. In other embodiments, the VL is N-terminal to the VH.

In further embodiments, the antigen-binding domain of the CAR includes a scFv or a single domain antibody, which optionally includes a sequence selected from the group consisting of SEQ ID NO: 3, 9, 15, 25, 71, 77, and variants thereof, or a sequence having at least 90% sequence identity (e.g., 90%, 91%, 92%, 93%, 94%, 95%, 96%, 97%, 98%, or 99% sequence identity) to the amino acid sequence of any one of SEQ ID NO: 3, 9, 15, 25, 71, and 77.

In some embodiments, the transmembrane domain includes a hinge/transmembrane domain, e.g., the hinge/transmembrane domain of an immunoglobulin-like protein (e.g., IgA, IgD, IgE, IgG, or IgM), CD28, CD8, or 4-1 BB. In some embodiments, transmembrane domain of the CAR includes a CD8 hinge/transmembrane domain, which optionally includes the sequence of any one of SEQ ID NOs: 4, 10, 16, 22, 28, 37, 46, 58, 66, 72, 78, and 104, or a variant thereof, or a sequence having at least 90% sequence identity (e.g., 90%, 91%, 92%, 93%, 94%, 95%, 96%, 97%, 98%, or 99% sequence identity) to the amino acid sequence of any one of SEQ ID NOs: 4, 10, 16, 22, 28, 37, 46, 58, 66, 72, 78, and 104.

In still further embodiments, the intracellular signaling domain includes the intracellular signaling domain of TCRζ, FcRγ, FcRβ, CD3γ, CD3θ, CD3ε, CD3η, CD3ζ, CD22, CD79a, CD79b, or CD66d. In certain embodiments, the intracellular signaling domain includes a CD3ζ intracellular signaling domain, which optionally includes the sequence of any one of SEQ ID NOs: 6, 12, 18, 24, 30, 39, 48, 60, 68, 74, 80, and 106, or a variant thereof, or a sequence having at least 90% sequence identity (e.g., 90%, 91%, 92%, 93%, 94%, 95%, 96%, 97%, 98%, or 99% sequence identity) to the amino acid sequence of any one of SEQ ID NOs: 6, 12, 18, 24, 30, 39, 48, 60, 68, 74, 80, and 106.

In some embodiments, the co-stimulatory domain includes the co-stimulatory domain of 4-1 BB, CD27, CD28, or OX-40. In certain embodiments, the co-stimulatory domain includes a 4-1 BB co-stimulatory domain, which optionally includes the sequence of any one of SEQ ID NOs: 5, 11, 17, 23, 29, 38, 47, 59, 67, 73, 79, and 105, or a variant thereof, or a sequence having at least 90% sequence identity (e.g., 90%, 91%, 92%, 93%, 94%, 95%, 96%, 97%, 98%, or 99% sequence identity) to the amino acid sequence of any one of SEQ ID NOs: 5, 11, 17, 23, 29, 38, 47, 59, 67, 73, 79, and 105.

In another aspect, the invention features a CAR polypeptide including the amino acid sequence of any one of SEQ ID NO: 26, SEQ ID NO: 35, SEQ ID NO: 44, SEQ ID NO: 53, SEQ ID NO: 61, SEQ ID NO: 19, SEQ ID NO: 1, SEQ ID NO: 7, SEQ ID NO: 13, SEQ ID NO: 69, SEQ ID NO: 75, and SEQ ID NO: 100, or including an amino acid sequence having at least 90% sequence identity (e.g., 90%, 91%, 92%, 93%, 94%, 95%, 96%, 97%, 98%, or 99% sequence identity) to the amino acid sequence of any one of SEQ ID NO: 26, SEQ ID NO: 35, SEQ ID NO: 44, SEQ ID NO: 53, SEQ ID NO: 61, SEQ ID NO: 19, SEQ ID NO: 1, SEQ ID NO: 7, SEQ ID NO: 13, SEQ ID NO: 69, SEQ ID NO: 75, and SEQ ID NO: 100.

In another aspect, the invention, in general, features a nucleic acid molecule encoding (i) the CAR polypeptide, or (ii) a polyprotein including the CAR polypeptide and the therapeutic agent, of any one of the preceding aspects. In some embodiments, the nucleic acid molecule further a suicide gene. In some embodiments, the nucleic acid molecule further includes a sequence encoding a signal sequence.

In another aspect, the invention, in general, features a vector including the nucleic acid molecule of any one of the preceding aspects. In some embodiments, the vector is a lentiviral vector.

In yet another aspect, the invention, in general, features a polypeptide including the CAR polypeptide, or a polyprotein including the CAR polypeptide and the therapeutic agent, of any one of the preceding aspects.

In still another aspect, the invention features, in general, an immune cell including the CAR polypeptide, the nucleic acid molecule, the vector, and/or the polypeptide of any one of the preceding aspects. In some embodiments, the immune cell is a T or NK cell. In some embodiments, the immune cell is a human cell.

In another aspect, the invention features, in general, a pharmaceutical composition including one or more CAR T cells, nucleic acid molecules, CAR polypeptides, polyproteins, or immune cells of any one of the preceding aspects.

In still another aspect, the invention features, in general, method of treating a patient having cancer, the method including administering to the patient the pharmaceutical composition any one of the preceding aspects.

In some embodiments, systemic toxicity is reduced by targeting the tumor microenvironment. In some embodiments, the cancer is characterized by the presence of one or more solid tumors. In further embodiments, the cancer is characterized by tumor-infiltrating Tregs. In certain embodiments, the cancer is a glioblastoma.

In another aspect, the invention features a method of treating a patient having cancer, the method including administering to the patient a CAR T cell product, genetically modified to secrete a tumor-toxic antibody or cytokine, wherein by directing the cancer toxicity locally to the tumor microenvironment, systemic toxicity is reduced.

In some embodiments, the CAR T cell is genetically modified to deliver an antibody against CTLA4, CD25, GARP, LAP, IL-15, CSF1R, or EGFR, EGFRvIII, CD19, CD79b, CD37, PSMA, PSCA, IL-13Rα2, EphA1, Her2, mesothelin, MUC1, or MUC16, or a bispecific antibody to the tumor microenvironment. In certain embodiments, the bispecific antibody is a BiTE directed against EGFR and CD3.

In another aspect, the invention features a method of delivering a therapeutic agent to a tissue or organ in a patient to treat a disease or pathology, the method including administering to said patient a CAR T cell, genetically modified to secrete a therapeutic antibody, toxin, or agent, wherein the therapeutic antibody, toxin, or agent would, by itself, be unable to enter or penetrate the tissue or organ.

In some embodiments, the tissue or organ is in the nervous system, e.g., the central nervous system, e.g., the brain. In some embodiments, the disease or pathology is a cancer, e.g., glioblastoma, prostate cancer, lung cancer, pancreatic cancer, lymphoma, or myeloma. In some embodiments, the therapeutic antibody is anti-EGFR or anti-EGFRvIII.

In another aspect, the invention features a method of treating glioblastoma having reduced EGFRvIII expression in a subject including administering to the subject a CAR T cell engineered to express: (i) a CAR polypeptide including an extracellular EGFRvIII-binding domain; and (ii) a BiTE, wherein the CAR T cell is optionally selected from the CAR T cell of any one of the preceding aspects. In some embodiments, the CAR includes a transmembrane domain, an intracellular signaling domain, and one or more co-stimulatory domains.

In another aspect, the invention features a method of preventing or reducing immunosuppression in the tumor microenvironment in a subject including administering to the subject a CAR T cell engineered to express: (i) a CAR polypeptide including an extracellular target binding domain; and (ii) a BiTE, wherein the CAR T cell is optionally selected from the CAR T cell of any one of the preceding aspects. In some embodiments, the CAR includes a transmembrane domain, an intracellular signaling domain, and one or more co-stimulatory domains.

In a further aspect, the invention features a method of preventing or reducing T cell exhaustion in the tumor microenvironment in a subject, the method including administering to the subject a CAR T cell engineered to express: (i) a CAR polypeptide including an extracellular target binding domain; and (ii) a BiTE, wherein the CAR T cell is optionally selected from the CAR T cell of any one of the preceding aspects. In some embodiments, the CAR includes a transmembrane domain, an intracellular signaling domain, and one or more co-stimulatory domains.

In still another aspect, the invention features a method of treating a cancer in a subject, the method including administering to the subject a CAR T cell engineered to express: (i) a CAR polypeptide including an extracellular target binding domain; and (ii) a BiTE, wherein the CAR T cell is optionally selected from the CAR T cell of any one of the preceding aspects. In some embodiments, the CAR includes a transmembrane domain, an intracellular signaling domain, and one or more co-stimulatory domains. In some embodiments, the cancer is glioblastoma, prostate cancer, lung cancer, pancreatic cancer, lymphoma, or myeloma. In some embodiments, the cancer includes cells expressing one or more of EGFR, EGFRvIII, CD19, PSMA, PSCA, IL-13Rα2, EphA1, Her2, mesothelin, MUC1, and MUC16. In some embodiments, the cancer expresses a heterogeneous antigen. Example of such cancers are glioblastoma (which expresses, e.g., EGFR, EGFRvIII, IL-13Rα2, HER2, and/or EphA1).

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in immunology and molecular biology can be found in The Merck Manual of Diagnosis and Therapy, 19^{th} Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of each of which are all incorporated by reference herein in their entireties.

The terms “decrease,” “reduced,” “reduction,” or “inhibit” are all used herein to mean a decrease by a statistically significant amount. In some embodiments, “reduce,” “reduction,” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. Where applicable, a decrease can be preferably down to a level accepted as within the range of normal for an individual without a given disorder.

The terms “increased,” “increase,” “enhance,” or “activate” are all used herein to mean an increase by a statically significant amount. In some embodiments, the terms “increased,” “increase,” “enhance,” or “activate” can mean an increase of at least 10% as compared to a reference level, for example, an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include, for example, chimpanzees, cynomolgus monkeys, spider monkeys, and macaques, e.g., rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient,” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of disease, e.g., cancer. A subject can be male or female.

A subject can be one who has been previously diagnosed with or identified as suffering from or having a condition in need of treatment (e.g., glioblastoma, glioma, leukemia, or another type of cancer, among others) or one or more complications related to such a condition, and optionally, have already undergone treatment for the condition or the one or more complications related to the condition. Alternatively, a subject can also be one who has not been previously diagnosed as having such condition or related complications. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

A “disease” is a state of health of an animal, for example, a human, wherein the animal cannot maintain homeostasis, and wherein if the disease is not ameliorated, then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “tumor antigen” and “cancer antigen” are used interchangeably to refer to antigens that are differentially expressed by cancer cells and can thereby be exploited in order to target cancer cells. Cancer antigens are antigens that can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), and fusion proteins resulting from internal deletions or chromosomal translocations. Still other cancer antigens can be encoded by viral genes such as those carried on RNA and DNA tumor viruses. Many tumor antigens have been defined in terms of multiple solid tumors: MAGE 1, 2, & 3, defined by immunity; MART-1/Melan-A, gp100, carcinoembryonic antigen (CEA), human epidermal growth factor receptor (HER2), mucins (i.e., MUC-1), prostate-specific antigen (PSA), and prostatic acid phosphatase (PAP). In addition, viral proteins such as some encoded by hepatitis B (HBV), Epstein-Barr (EBV), and human papilloma (HPV) have been shown to be important in the development of hepatocellular carcinoma, lymphoma, and cervical cancer, respectively. Examples of tumor antigens are provided below and include, e.g., EGFR, EGFRvIII, CD19, PSMA, B cell maturation antigen (BCMA), interleukin-13 receptor subunit alpha-2 (IL13Rα2), etc.

As used herein, “Treg antigen” or “Treg-associated antigen” is used interchangeably to refer to antigens that are expressed by T regulatory (Treg) cells. These antigens may optionally be targeted by the cells and methods of the invention. Examples of Treg antigens are provided below and include, e.g., GARP, LAP, CD25, and CTLA-4.

As used herein, the term “chimeric” refers to the product of the fusion of portions of at least two or more different polynucleotide molecules. In one embodiment, the term “chimeric” refers to a gene expression element produced through the manipulation of known elements or other polynucleotide molecules.

By “bispecific T cell engagers,” “BiTE antibody constructs,” or BiTEs” is meant polypeptides that each include tandemly linked single-chain variable fragments (scFvs). Optionally, the scFvs are linked by a linker (e.g., a glycine-rich linker). One scFv of the BiTE binds to the T cell receptor (TCR) (e.g., to the CD3c subunit) and the other binds to a target antigen (e.g., a tumor-associated antigen).

In some embodiments, “activation” can refer to the state of a T cell that has been sufficiently stimulated to induce detectable cellular proliferation. In some embodiments, activation can refer to induced cytokine production. In other embodiments, activation can refer to detectable effector functions. At a minimum, an “activated T cell” as used herein is a proliferative T cell.

As used herein, the terms “specific binding” and “specifically binds” refer to a physical interaction between two molecules, compounds, cells and/or particles wherein the first entity binds to the second, target, entity with greater specificity and affinity than it binds to a third entity which is a non-target. In some embodiments, specific binding can refer to an affinity of the first entity for the second target, entity, which is at least 10 times, at least 50 times, at least 100 times, at least 500 times, at least 1000 times or more greater than the affinity for the third non-target entity under the same conditions. A reagent specific for a given target is one that exhibits specific binding for that target under the conditions of the assay being utilized. A non-limiting example includes an antibody, or a ligand, which recognizes and binds with a cognate binding partner (for example, a stimulatory and/or costimulatory molecule present on a T cell) protein.

A “stimulatory ligand,” as used herein, refers to a ligand that when present on an antigen presenting cell (APC) (e.g., a macrophage, a dendritic cell, a B-cell, an artificial APC, and the like) can specifically bind with a cognate binding partner (referred to herein as a “stimulatory molecule” or “co-stimulatory molecule”) on a T cell, thereby mediating a primary response by the T cell, including, but not limited to, proliferation, activation, initiation of an immune response, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

A “stimulatory molecule,” as the term is used herein, means a molecule on a T cell that specifically binds with a cognate stimulatory ligand present on an antigen presenting cell.

“Co-stimulatory ligand,” as the term is used herein, includes a molecule on an APC that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, 4-1 BBL, OX40L, CD7, B7-1 (CD80), B7-2 (CD86), PD-L1, PD-L2, inducible COStimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll-like receptor and a ligand that specifically binds with B7-H3. A co-stimulatory ligand also can include, but is not limited to, an antibody that specifically binds with a co-stimulatory molecule present on a T cell, such as, but not limited to, CD27, CD28, 4-1 BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA, a Toll-like receptor, CD27, CD28, 4-1 BB, OX40, CD30, CD40, PD-1, ICOS, lymphocyte function-associated antigen-1 (LFA-1), CD2, CD7, LIGHT, NKG2C, B7-H3, and CD83.

In one embodiment, the term “engineered” and its grammatical equivalents as used herein can refer to one or more human-designed alterations of a nucleic acid, e.g., the nucleic acid within an organism's genome. In another embodiment, engineered can refer to alterations, additions, and/or deletion of genes. An “engineered cell” can refer to a cell with an added, deleted and/or altered gene. The term “cell” or “engineered cell” and their grammatical equivalents as used herein can refer to a cell of human or non-human animal origin.

As used herein, the term “operably linked” refers to a first polynucleotide molecule, such as a promoter, connected with a second transcribable polynucleotide molecule, such as a gene of interest, where the polynucleotide molecules are so arranged that the first polynucleotide molecule affects the function of the second polynucleotide molecule. The two polynucleotide molecules may or may not be part of a single contiguous polynucleotide molecule and may or may not be adjacent. For example, a promoter is operably linked to a gene of interest if the promoter regulates or mediates transcription of the gene of interest in a cell.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of ordinary skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g., ligand-mediated receptor activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, a polypeptide described herein (or a nucleic acid encoding such a polypeptide) can be a functional fragment of one of the amino acid sequences described herein. As used herein, a “functional fragment” is a fragment or segment of a peptide that retains at least 50% of the wildtype reference polypeptide's activity according to an assay known in the art or described below herein. A functional fragment can comprise conservative substitutions of the sequences disclosed herein.

In some embodiments, a polypeptide described herein can be a variant of a polypeptide or molecule as described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions, or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity of the non-variant polypeptide. A wide variety of PCR-based site-specific mutagenesis approaches are known in the art and can be applied by the ordinarily skilled artisan.

A variant amino acid or DNA sequence can be at least 80%, at least 85%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g., BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of a polypeptide also can be substituted, generally with serine, to improve the oxidative stability of the molecule and prevent aberrant crosslinking. Conversely, cysteine bond(s) can be added to a polypeptide to improve its stability or facilitate oligomerization.

As used herein, the term “DNA” is defined as deoxyribonucleic acid. The term “polynucleotide” is used herein interchangeably with “nucleic acid” to indicate a polymer of nucleosides. Typically a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically characterizes a specific nucleic acid. A polynucleotide sequence presented herein is presented in a 5′ to 3′ direction unless otherwise indicated.

The term “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide.” Exemplary modifications include glycosylation and palmitoylation. Polypeptides can be purified from natural sources, produced using recombinant DNA technology or synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

In some embodiments, a nucleic acid encoding a polypeptide as described herein (e.g., a CAR polypeptide) is comprised by a vector. In some of the aspects described herein, a nucleic acid sequence encoding a given polypeptide as described herein, or any module thereof, is operably linked to a vector. The term “vector,” as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example, in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′ UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

By “recombinant vector” is meant a vector that includes a heterologous nucleic acid sequence or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra-chromosomal DNA thereby eliminating potential effects of chromosomal integration.

As used herein, a “signal peptide” or “signal sequence” refers to a peptide at the N-terminus of a newly synthesized protein that serves to direct a nascent protein into the endoplasmic reticulum. In some embodiments, the signal peptide is a CD8 or Igκ signal peptide.

As used herein, the terms “treat,” “treatment,” “treating,” or “amelioration” refer to therapeutic treatments, wherein the object is to reverse, alleviate, ameliorate, inhibit, slow down, or stop the progression or severity of a condition associated with a disease or disorder, e.g., glioblastoma, glioma, acute lymphoblastic leukemia or other cancer, disease, or disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier in which the active ingredient would not be found to occur in nature.

As used herein, the term “administering,” refers to the placement of a therapeutic or pharmaceutical composition as disclosed herein into a subject by a method or route that results in at least partial delivery of the agent at a desired site. Pharmaceutical compositions comprising agents as disclosed herein can be administered by any appropriate route that results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the technology.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined within the description of the various aspects and embodiments of the technology, as set forth below.

The invention provide several advantages. For example, the CAR T cells of the invention can be used to deliver therapeutic agents for cancer treatment. In one example, the CAR T cells of the invention can be used to deliver otherwise toxic antibodies (e.g., anti-CTLA4 or anti-CD25 (e.g., daclizumab)) or other molecules (e.g., cytokines) to the tumor microenvironment, where they can advantageously enable activation of surrounding tumor infiltrating lymphocytes, provide checkpoint blockade, and deplete regulatory T cells (Tregs). The CAR T cells of the invention can further be directed against Treg antigens to facilitate targeting of Treg cells. Furthermore, certain CAR T cells of the invention can be used to deliver genetically encoded molecules (e.g., antibodies or cytokines) to regions of the body (e.g., the central nervous system, including the brain) that these molecules otherwise cannot reach. In one example, CART cells targeting EGFRvIII can be used to target brain tumors, and can deliver antibodies (e.g., antibodies against EGFR, such as cetuximab; also see below) to the tumors. The invention thus provides genetically-encoded Treg targeting in the tumor microenvironment. In addition, the invention provides genetically-encoded delivery of antibodies that cannot reach certain tissues, and can enhance the potency of T cell therapies by broadening the specificity of the anti-tumor target. The invention accordingly provides for gene-modified T cell therapy for cancer.

Other features and advantages of the invention will be apparent from the following detailed description, the drawings, and the claims.

### BRIEF DESCRIPTION OF THE DRAWINGS

FIG. 1 is a graph showing killing of human glioma target cell line U87vIII by CART-EGFRvIII cells as a function of CART-EGFRvIII:U87vIII target cell ratio. Untransduced cells were incubated with target cells as a negative control.

FIGS. 2A and 2B are a series of bioluminescence images showing the location of EGFRvIII expressing tumor (U87vIII) in a subcutaneous model of human glioma. FIG. 2A shows mice treated with untransduced cells as a negative control. FIG. 2B shows mice treated with CART-EGFRvIII on day 4 after implantation (top row), with successful treatment by day 21 (bottom row).

FIGS. 3A and 3B are a series of X-ray overlays showing the location of EGFRvIII expressing tumor (U87vIII) in an intracranial model of human glioma. FIG. 3A shows mice treated with untransduced (UTD) cells as a negative control at day 5 (D5; top row) and D11 (bottom row). FIG. 3B shows mice treated with CART-EGFRvIII on day 2 after implantation at D5 (top row) and at D11 (bottom row).

FIGS. 4A and 4B are photomicrographs showing immunohistochemistry of tumor tissue in one patient five days following infusion of CART-EGFRvIII. FIG. 4A shows T cells stained for CD3. FIG. 4B shows CD25+ cells. CD25 is the IL-2 receptor alpha chain, a marker of activated or regulatory T cells.

FIGS. 5A-5C are fluorescence micrographs qualitatively demonstrating Treg suppression of CAR T cell antitumor activity after 18 hours of coincubation with human glioma cells in vitro. FIG. 5A shows relative concentration of CART-nonspecific cells to glioma cells. FIG. 5B shows relative concentration of CART-EGFRvIII cells to glioma cells with no Tregs in the culture. FIG. 5C shows relative concentration of CART-EGFRvIII cells to glioma cells with Tregs included in the culture.

FIG. 5D is a graph showing quantitative readouts of green object confluence as a measure of glioma cell viability as a function of time (up to 48 hours). The top line represents the results shown in FIG. 5A (glioma cell growth), the bottom line represents the results shown in FIG. 5B (glioma cell killing), and the middle line represents the results shown in FIG. 5C (glioma cell resistance to CART-killing).

FIGS. 6A-6C are flow cytometry plots showing expression of LAP (x-axis) and GARP (y-axis) on control T cells (FIG. 6A), unactivated Tregs (FIG. 6B), and activated Tregs (FIG. 6C). Tregs were sorted from leukopak on CD4+CD25+CD127- and expanded with CD3/CD28 beads for seven days in the presence of IL-2. On day 1, they were transduced to express GFP. After debeading on day 7, expanded Tregs were rested for four days before freezing. After thawing, Tregs were stained for LAP and GARP expression after overnight rest (non-activated) or overnight activation with anti-CD3 and anti-CD28. Untransduced T cells (CD4+ and CD8+) from the same donor were used as controls for expression (FIG. 6A).

FIGS. 7A and 7B are flow cytometry histograms corresponding to the results shown in FIGS. 6A-6C showing expression of LAP (FIG. 7A) and GARP (FIG. 7B).

FIGS. 8A-8D are schematic drawings of CAR constructs for targeting Treg-associated antigens. FIG. 8A shows a LAP-targeting CAR construct having an anti-LAP scFv with its light chain (L) and heavy chain (H) arranged in a 5′-to-3′ direction, respectively (CART-LAP-L-H). FIG. 8B shows a LAP-targeting CAR construct having an anti-LAP scFv with its heavy chain (H) and light chain (L) arranged in a 5′-to-3′ direction, respectively (CART-LAP-H-L). FIG. 8C shows a GARP-targeting CAR construct having an anti-GARP camelid antibody binding domain (CART-GARP). FIG. 8D shows an EGFR-targeted CAR construct having an anti-GARP camelid antibody.

FIGS. 9A and 9B are graphs showing target Treg killing as a function of CAR T cell-to-target Treg cell ratio. Tregs were transduced with GFP, and cytotoxicity was quantified by monitoring GFP expression. FIG. 9A shows killing of activated Tregs, and FIG. 9B shows killing of non-activated Tregs. CART-LAP-H-L was more effective at killing non-activated Tregs in comparison to CART-LAP-L-H.

FIGS. 10A and 10B are graphs showing target Treg killing by various anti-Treg CAR T cells (i.e., CART-GARP, CART-LAP-H-L, CART-LAP-L-H, or untransduced control cells) at a 1:1 ratio of CAR T cells to Tregs for four days. FIGS. 10A and 10B show results from the same experiment conducted in two different donors.

FIGS. 11A-11D are graphs showing target Treg killing as a function of CAR T cell-to-target Treg cell ratio by LAP-targeted CAR T cells after three days of coculture. FIGS. 11A and 11B show number of target cells remaining in coculture as measured by flow cytometry. A dashed line indicates the number of target cells in a control sample containing no CAR cells. FIG. 11A shows non-activated Tregs as target cells, whereas FIG. 11B shows activated Tregs as target cells. FIGS. 11C and 11D show percent cytotoxicity as measured by luciferase expression by target cells. FIG. 11C shows non-activated Tregs as target cells, whereas FIG. 11D shows activated Tregs as target cells. In each of FIGS. 11A-11D, circles represent CART-LAP-H-L, squares represent CART-LAP-L-H, and triangles represent untransduced CAR cells.

FIGS. 12A and 12B are flow cytometry histograms showing the expression of GARP (FIG. 12A) and LAP (FIG. 12B) by HUT78 cells.

FIGS. 13A and 13B are graphs showing killing of target HUT78 cells as a function of CAR T cell-to-target cell ratio by LAP-targeted CART cells after three days of coculture. FIG. 13A shows the number of target cells remaining in culture after three days, as measured by flow cytometry. A dashed line indicates the number of target cells in a control sample containing no CAR cells. FIG. 13B shows percent cytotoxicity as measured by luciferase expression by target cells. Circles represent CART-LAP-H-L, squares represent CART-LAP-L-H, and triangles represent untransduced CAR cells.

FIGS. 14A and 14B are flow cytometry histograms showing the expression of GARP (FIG. 14A) and LAP (FIG. 14B) by SeAx cells.

FIGS. 15A and 15B are graphs showing killing of target SeAx cells as a function of CAR T cell-to-target cell ratio by GARP and LAP-targeted CAR T cells after 24 (FIG. 15A) hours and 48 hours (FIG. 15B) of coculture, as measured by luciferase expression by target cells. Squares represent CART-GARP, upward-facing triangles represent CART-LAP-H-L, downward-facing triangles represent CART-LAP-H-L cells, and diamonds represent untransduced CAR cells.

FIGS. 16A-16C are photographs of western blots showing the presence of protein components of supernatants obtained from cultures of CART-EGFR-GARP T cells. FIGS. 16A and 16B show the full gel, including molecular weight reference ladders. FIG. 16C is a longer exposure of the bottom region of the gel shown in FIG. 16B, in which a band between 10 and 15 kD is identified with an arrow, indicating the presence of a camelid antibody.

FIG. 17 is a schematic drawing of CAR-EGFR-BiTE-(EGFR-CD3), an exemplary nucleic acid molecule encoding a CAR and a BiTE.

FIG. 18 is a schematic drawing of a BiTE having an anti-EGFR domain derived from cetuximab and an anti-CD3 domain derived from blinatumomab.

FIG. 19 is a set of photographs showing a western blot experiment verifying the presence of BiTE in lane 2.

FIGS. 20A and 20B are a set of flow cytometry graphs showing binding of BiTE expressed by HEK293 cells transduced with CAR-EGFR-BiTE-(EGFR-CD3) to EGFR expressed by K562 cells (FIG. 20A) and CD3 expressed by Jurkat cells (FIG. 20B).

FIGS. 21A and 21B are a set of flow cytometry graphs showing binding of BiTE expressed by SupT1 cells transduced with CAR-EGFR-BiTE-(EGFR-CD3) to EGFR expressed by K562 cells (FIG. 21A) and CD3 expressed by CAR-EGFR-BiTE-(EGFR-CD3)-expressing SupT1 cells (FIG. 21B).

FIGS. 22A and 22B are a set of flow cytometry graphs showing binding of BiTE expressed by ND4 cells transduced with CAR-EGFR-BiTE-(EGFR-CD3) to EGFR expressed by K562 cells (FIG. 22A) and CD3 expressed by CAR-EGFR-BiTE-(EGFR-CD3)-expressing ND4 cells (FIG. 22B).

FIG. 23 is a graph showing killing of U87vIII cells by ND4 cells incubated with BiTE secreted by HEK293T cells that were transduced with CAR-EGFR-BiTE-(EGFR-CD3), as a function of effector (untransduced ND4) to target (U87vIII) cell ratio. Squares represent the experimental group in which the supernatant contained BiTE, and circles represent a negative control containing no BiTE.

FIG. 24 is a drawing of an exemplary nucleic acid molecule encoding a CAR under control of an EF1α promoter and GFP under control of an NFAT promoter.

FIGS. 25A and 25B are a set of flow cytometry graphs showing GFP expression by cells transduced with the construct of FIG. 24. The red histogram shows GFP expression in unstimulated cells; the blue histogram shows GFP expression in cells stimulated with PMA and ionomycin; and the orange histogram shows GFP expression in cells coated with PEPvIII.

FIG. 26A is a schematic drawing of GFP-CAR-EGFR-BiTE-(EGFR-CD3), an exemplary nucleic acid molecule encoding a CAR and a constitutively expressed BiTE.

FIG. 26B is a schematic drawing of GFP-CAR-EGFR-BiTE-(CD19-CD3), an exemplary nucleic acid molecule encoding a CAR and a constitutively expressed BiTE.

FIG. 27A is a schematic drawing of BiTE-(CD19-CD3)-CAR-EGFR, an exemplary nucleic acid molecule encoding a CAR and an inducibly expressed BiTE.

FIG. 27B is a schematic drawing of BiTE-(CD19-CD3)-CAR-EGFR, an exemplary nucleic acid molecule encoding a CAR and an inducibly expressed BiTE.

FIG. 28 shows confocal microscopy of CAR-BiTE cells and binding of EGFR (biotin-streptavidin-FITC). Transduced cells are red (due to mCherry reporter gene).

FIGS. 29A and 29B are a series of graphs showing antitumor activity of CAR-BiTE. FIG. 29A shows IFN-γ and TNF-α were produced from CART-EGFRvIII.BiTE-EGFR in the presence of target U87 glioma cells. FIG. 29B shows CART-EGFRvIII.BiTE-EGFR mediated specific lysis against U87 cells, reaching near 100% lysis after 40 h co-culture.

FIG. 29C is a schematic diagram of ACEA Transwell (pore size: 1 micron) experiments where CAR.BiTE T cells were seeded in the top well with UTD and target tumor are seeded in the bottom.

FIG. 29D is a graph showing transwells containing CAR.BiTE led to selective lysis of U87, but not wells with inserts containing UTD or CAR.BiTE control.

FIG. 30A is a schematic diagram of in vivo evaluation of CART-EGFRvIII.BiTE-EGFR antitumor activity against intracranial U251. Tumors were implanted with stereotactic assistance at day −1 followed by adoptive transfer of 1×10⁶ CAR-transduced cells into the contralateral lateral ventricle.

FIG. 30B shows in vivo efficacy of CAR-BiTE in mice treated with CART-EGFRvIII.BiTE-EGFR. CART-EGFRvIII.BiTE-EGFR demonstrated near complete eradication of intracranial tumor by day 21.

FIG. 31 shows EGFR expression in glioblastoma and normal tissues of the central nervous system (CNS). Tissue microarray showing EGFR expression by immunohistochemistry across several normal healthy human CNS tissues (top) and glioblastoma specimens (bottom). Details regarding each specimen may be found in Table 2.

FIG. 32A shows the experimental design, where a heterogeneous population (30% EGFRvIII-positive, 70% wild-type) of U87 glioma cells (5×10⁴) is implanted in the flanks of NSG mice.

FIG. 32B shows bioluminescence analysis of EGFRvIII-expressing tumor growth over time.

FIG. 32C shows caliper measurements of overall tumor growth in mice treated with UTD alone versus CART-EGFRvIII, n=5 mice.

FIG. 32D shows hematoxylin and eosin (H&E) staining and immunohistochemistry (IHC) for EGFR and EGFRvIII on tumors harvested from mice treated with UTD cells or CART-EGFRvIII (scale bar=50 μm).

FIG. 32E shows heterogeneous EGFRvIII expression.

FIG. 33A shows a schematic representation of transgenes for two BiTE-secreting anti-EGFRvIII CAR constructs targeting EGFR and CD19.

FIG. 33B shows transduction efficiency. All constructs demonstrated efficient transduction of primary human T cells from 3 normal donors (mean±SEM).

FIG. 33C shows the overall scFv orientation for each BiTE, which is light-heavy-heavy-light bridged by flexible glycine-serine linkers.

FIG. 33D shows a schematic representation of BiTE-EGFR and BiTE-CD19.

FIG. 33E shows Western blot analysis for BiTEs in the supernatants of HEK298T cells transduced with CART-EGFRvIII.BiTE-CD19 or CART-EGFRvIII.BiTE-EGFR.

FIG. 33F shows flow cytometric histograms demonstrating secondary His-tag detection of BiTE binding to K562 cells expressing respective targets. Unconcentrated supernatant from CART-EGFRvIII, CART-EGFRvIII.BiTE-CD19, and CART-EGFRvIII.BiTE-EGFR cells 10 days post-transduction were incubated with K562 cells expressing CD19 or EGFR.

FIG. 33G shows flow cytometric histograms demonstrating BiTE binding to CD3 on primary human T cells. Data reflects cultures stained with anti-His-tag antibody corresponding to the following: UTD alone, UTDs cultured with CART-EGFRvIII.BiTE-CD19 cells, or CART-EGFRvIII.BiTE-EGFR cells. UTDs stained with concentrated supernatant (1000×) from respective cultures are depicted.

FIG. 33H shows BiTE concentration in supernatant increases over time. Untransduced T cells (UTD) or those transduced with CART-EGFRvIII.BiTE-EGFR were cultured with supernatant collected for His-tag ELISA analysis on day 0, 7, and 14. Assays were performed in triplicate (mean±SEM is depicted; unpaired t-test, *=p<0.05).

FIG. 34A shows expression of EGFR and EGFRvIII on U87 and U251 cell lines relative to unstained cells by flow cytometry.

FIG. 34B shows Jurkat reporter T cells either untransduced (UTD) or transduced with CART-EGFRvIII.BiTE-CD19 or CART-EGFRvIII.BiTE-EGFR and co-cultured with U87 or U251 glioma cell lines for 18 hours at an E:T of 1:1. Activation is reflected by relative luminescence.

FIG. 34C shows cytokine production by primary human UTD, CAR T, and CART.BiTE cells when cocultured overnight with U87 or U251 at an E:T of 1:1.

FIG. 35 shows antitumor-specific lysis of CART.BiTE against EGFR-expressing tumor. Cytotoxicity of UTD cells or CART-EGFRvIII.BiTE-EGFR cells against U87 by bioluminescence-based assay at indicated E:T ratios after 18 hours.

FIGS. 36A and 36B show impedance-based cytotoxicity assay of UTD and CAR T cells against U87 and U251 at an E:T of 3:1 (Hi) and 1:1 (Lo) (FIG. 36A), also represented as percent lysis normalized to UTD over time (FIG. 36B). Data was recorded with readings obtained every 15 minutes.

FIG. 36C shows correlation between EGFR expression on GBM cell lines and percent specific lysis by CAR T cells. Quantification of EGFR expression by U251 and U87 was determined by flow cytometry and plotted as mean fluorescence intensity (MFI). Percent specific lysis was measured by impedance-based killing assay. Effector cells were incubated with target cells at an E:T of 1:1 for 24 hours. Cytotoxicity was reflected by decreases in cell index relative to targets incubated with UTD controls.

FIG. 37A shows characterization of EGFR and EGFRvIII expression on the PDX neurosphere line, BT74, by flow cytometry. Positive events (gray) were gated relative to isotype staining (black).

FIG. 37B shows reporter T cells either UTD, transduced with CART-EGFRvIII.BiTE-CD19 or CART-EGFRvIII.BiTE-EGFR and cocultured with BT74 at an E:T of 1:1.

FIG. 37C shows cytotoxicity assessment against BT74 transduced with eGFP at an E:T of 3:1 in duplicate. Total green image area (pmt) was recorded as a proxy for BT74 viability.

FIG. 37D shows representative images of neurospheres from FIG. 37C over the course of 4 days (scale bar=100 μm).

FIG. 38A shows a schematic representation of experimental design in which 5×10³ U87vIII cells were implanted orthotopically into the brains of NSG mice and treated with either intravenous (IV) or intraventricular (IVT) CAR T cells (1×10⁶ transduced cells).

FIG. 38B shows the survival plot of mice treated by CART-EGFRvIII, grouped by route-of-delivery, compared to treatment with UTD cells; n=5 per group.

FIG. 39A shows a schematic representation of experimental design in which 5×10⁵ BT74 cells transduced with CBG-GFP were implanted into NSG mice intracranially (IC) and treated on day 7 post-implantation with intraventricular (IVT) infusion of UTD cells, CART-EGFRvIII.BiTE-CD19 cells, or CART-EGFRvIII.BiTE-EGFR cells (1×10⁶ transduced cells).

FIG. 39B shows tumor growth over time; data represents three consecutive mice treated with corresponding regimens.

FIG. 39C shows average bioluminescence values per group displayed over time (mean+SD is depicted).

FIG. 40A shows U251 cells (2×10⁴) implanted orthotopically into NSG mice and treated on day 5 post-implantation with intraventricular (IVT) untransduced T cells (UTD), CART-EGFRvIIIv.BiTE-CD19 cells, or CART-EGFRvIII.BiTE-EGFR cells.

FIG. 40B shows bioluminescence imaging of U251 tumor growth over time, n=5 mice.

FIG. 40C shows tumor growth for individual mice (left panel) and as average values (right panel) (mean±SD is depicted; unpaired t test, ***=p<0.001).

FIG. 40D shows the experimental design. Human skin was engrafted onto the dorsum of NSG mice and allowed to heal for six weeks. CART-EGFR, CART-EGFRvIII.BiTE-CD19, or CART-EGFRvIII.BiTE-EGFR cells were then administered intravenously (IV) by tail vein. Grafts were observed for up to two weeks prior to excision and histopathologic analysis.

FIG. 40E shows hematoxylin counter staining and immunohistochemistry (IHC) for CD3 (T cells) and apoptotic cells identified by terminal deoxynucleotidyl transferase dUTP nick end labeling (TUNEL) in formalin-fixed, paraffin-embedded skin specimens from mice treated with intravenous CAR T cells or CART.BiTE cells (scale bar=100 μm).

FIGS. 40F and 40G show quantification of infiltrating CD3+ cells (FIG. 40F) and TUNEL⁺ cells (FIG. 40G) in skin grafts of mice treated with CART-EGFR, CART-EGFRvIII.BiTE-CD19, or CART-EGFRvIII.BiTE-EGFR. Cells counts were recorded in 10 consecutive high power fields (HPF) at 40× magnification. The experiment was repeated. Bars represent mean values, n=10 (unpaired t-test, **=P<0.01, ***=p<0.001).

FIG. 41A shows confocal microscopy depicting BiTEs binding to T cells. CAR transduction is depicted as mCherry-positive cells. EGFR-specificity is determined by the ability to bind biotinylated EGFR and areas of overlap are also present (scale bar=10 μm).

FIG. 41B shows a schematic representation of panels shown in FIG. 41A; CART-EGFR (top), CART-EGFRvIII.BiTE-CD19 (middle), and CART-EGFRvIII.BiTE-EGFR (bottom).

FIG. 41C shows CD25 and CD69 expression on CAR T cells and CART.BiTE cells (mCherry-positive) as well as bystander T cells (mCherry-negative) after coculture with EGFR-expressing tumor, U87.

FIG. 41D shows bystander reporter T-cell activation. UTDs, CAR T cells, and CART.BiTE cells were co-cultured overnight with reporter T cells and EGFR-expressing tumor cells, with bystander activation subsequently measured by relative luminescence.

FIG. 41E shows CAR T cell and CART.BiTE cell culture proliferation against U87. CAR T cells and CART.BiTE cells were cocultured with target cells, revealing transduced cells, untransduced bystander cells, and U87.

FIG. 41F shows flow cytometric quantification of bystander cells from cultures shown in FIG. 41E by counting beads.

FIG. 41G shows a schematic representation of the transwell system used to assess bystander cytokine secretion and cytotoxicity against U87. Jurkat T cells untransduced or transduced with CART.BiTE constructs were cultured in top wells while primary human UTD cells and U87 targets were placed in bottom wells.

FIG. 41H shows cytokine production by bystander UTD cells when cocultured with targets and exposed to supernatant from top wells.

FIG. 41I shows impedance-based cytotoxicity assay measuring activity of bystander cells against U87 and U87-CD19, using the transwell system depicted in FIG. 41G.

FIG. 42 shows bioluminescence-based cytotoxicity assay measuring activity of bystander Tregs against U87 using a transwell system. T cells transduced with either CART-EGFRvIII.BiTE-CD19 or CART-EGFRvIII.BiTE-EGFR were cultured in top wells while sorted primary human Tregs (CD4⁰25⁺CD127^{dim/-}) and U87 targets were placed in bottom wells.

FIG. 43A shows a schematic representation of experimental design in which a heterogeneous population (10% EGFRvIII-positive, 90% wild-type) of U87 glioma cells (5×10³) was implanted orthotopically into the brains of NSG mice. Both U87 and U87vIII cells were modified with CBG-luc so that total intracranial tumor burden could be visualized by bioluminescent imaging. Mice were treated intraventricularly on day 2 post-implantation with untransduced T cells (UTD), CART-EGFRvIII.BiTE-CD19 cells, or CART-EGFRvIII.BiTE-EGFR cells.

FIG. 43B shows bioluminescence analysis of mixed tumor growth over time, n=5.

FIG. 43C shows tumor growth shown as average values (mean±SD is depicted; unpaired t test, ***=p<0.001).

FIG. 43D shows sorted CAR T cell and CART.BiTE cell purity. Shown are representative flow cytometry data before and after cell sorting.

FIGS. 43E and 43F show bioluminescence-based cytotoxicity assay of UTDs, sorted CART-EGFRvIII cells, or sorted CART.BiTE cells against U87, U87-CD19 (FIG. 41E), or U87vIII (FIG. 41F) at indicated E:T ratios over 18 h.

FIG. 43G shows proliferation assays of sorted transduced cells. Effectors cells were stimulated (arrows) using irradiated U87, U87vIII, or U87-CD19. UTD cells, sorted CART-EGFRvIII cells and sorted CART.BiTE cells were then stimulated through CAR alone (CART-EGFRvIII.BiTE-CD19 with U87vIII), BiTE alone (CART-EGFRvIII.BiTE-CD19 with U87-CD19), or CAR and BiTE (CART-EGFRvIII.BiTE-EGFR and U87vIII). Assay was performed in triplicate (mean±SEM is depicted; unpaired t test, ***=p<0.001).

FIG. 43H shows phenotype of T cells as outlined in FIG. 41G after 3 weeks of stimulation. Cells were grouped by flow cytometry according to T-cell phenotype as follows: naïve (T_{N}) CCR7⁺CD45RO⁻, central memory (T_{CM}) CCR7³⁰ CD45RO⁺, effector memory (T_{EM}) CCR7⁻CD45RO⁺, and effector (TE) CCR7⁻CD45RO⁻. Pie graphs demonstrate phenotype of CAR T cells stimulated through BiTE alone, CAR alone, or CAR and BiTE.

FIG. 43I shows exhaustion markers (PD-1, TIM-3, and LAG-3) after 12 days of stimulation through BiTE alone, CAR alone, or CAR and BiTE.

FIGS. 44A-44C are a series of schematic diagrams showing exemplary chimeric antigen receptors (CARs), including tandem CARs that target two distinct antigens. FIG. 44A shows a schematic diagram of an exemplary anti-IL-13Rα2 CAR construct, which includes an EF1α promoter, an IL-13 receptor alpha 2 ligand (such as IL-13 zetakine, an anti-IL-13Rα2 single chain variable fragment or single domain antibody), a 4-1 BB transmembrane domain, a 4-1 BB co-stimulatory domain, a CD3ζ domain, a T2A peptide sequence, and a reporter gene (mCherry). FIG. 44B shows a schematic diagram of an exemplary anti-EGFRvIII CAR construct, which includes an EF1α promoter, an anti-EGFRvIII scFv, a CD8 transmembrane domain, a 4-1 BB co-stimulatory domain, a CD3 domain, a T2A peptide sequence, and a reporter gene (mCherry). FIG. 44C shows a schematic diagram of an exemplary tandem anti-IL-13Rα2/anti-EGFRvI11 CAR construct, which includes an EF1α promoter, an IL-13 ligand (IL-13 zetakine), an anti-EGFRvIII scFv, a CD8 transmembrane domain, a 4-1 BB co-stimulatory domain, a CD3ζ domain, a T2A peptide sequence, and a reporter gene (mCherry).

FIG. 44D shows schematic diagrams of the constructs of FIGS. 44A-44C without mCherry.

FIG. 45A is a series of graphs showing the results of flow cytometry analysis to assess expression of IL-13Rα2 in U87 human glioblastoma cells and U87 cells transduced to express EGFRvIII (U87vIII).

FIG. 45B is a graph showing the results of a cytotoxicity assay in which a heterogeneous population of glioblastoma cells (a 1:1 ratio of U87 cells:U87vIII cells) were incubated with control untransduced T cells (UTD) or T cells transduced with the indicated CAR constructs from FIGS. 44A-44C. The y-axis shows percent specific lysis, and the x-axis shows the effector to target (E:T) ratios.

### DETAILED DESCRIPTION

The invention provides improved approaches to chimeric antigen receptor T cell (“CAR T cell”)-based therapy. In general, the improvements relate to different aspects of targeting in antitumor therapy, for example, targeting of the tumor microenvironment.

For example, described herein are immune cells, e.g., T cells engineered to express a CAR as well as to secrete a therapeutic agent, such as a bispecific T cell engager (BiTE). CAR T cells engineered to secrete BiTEs are referred to herein as CART.BiTE. The CART.BiTE strategy allows for locoregional delivery of therapeutics for tumors in, e.g., the central nervous system (CNS) while reducing the risk of undesired activity in systemic tissues. Such CART.BiTE constructs are useful for treating cancers such as glioblastoma, prostate cancer, lung cancer, pancreatic cancer, lymphoma, or myeloma, among others as described herein.

Additionally, as is explained further below, we have demonstrated that regulatory T cells (also referred to herein as “Tregs”), which play a role in the suppression of a subject's immune response against tumors (e.g., in the tumor microenvironment), can be targeted with CAR T cells. The invention thus provides CAR T cells, in which the CAR is directed against a Treg antigen or marker (e.g., GARP, LAP, CTLA4, or CD25; also see below). In other examples, the invention provides CAR T cells that secrete antibodies (e.g., single chain antibodies, single domain antibodies (e.g., camelid antibodies), or bispecific antibodies (e.g., bispecific T cell engagers)) against one or more Treg antigens or markers (e.g., GARP, LAP, CTLA4 and CD25; also see below). In addition to targeting Tregs, the invention provides CAR T cells and related methods for delivering other therapeutic agents (e.g., antibodies and related molecules) to tumors. In one example, a CAR T cell having a CAR specific for EGFRvIII is used to target brain tumors (e.g., glioblastomas). Such CAR T cells may also be used to deliver therapeutic agents, such as antibody reagents (e.g., single chain antibodies, single domain antibodies (e.g., camelid antibodies), or bispecific antibodies (e.g., bispecific T cell engagers)) to these tumors. These methods are particularly advantageous, as they, in effect, facilitate antibody administration to the brain, despite the blood brain barrier through which antibodies do not normally pass. These approaches, as well as related methods and compositions, are described further, as follows.

### Chimeric Antigen Receptors (CARs)

The technology described herein provides improved chimeric antigen receptors (CARs) for use in immunotherapy. The following discusses CARs and the various improvements.

The terms “chimeric antigen receptor” or “CAR” or “CARs” as used herein refer to engineered T cell receptors, which graft a ligand or antigen specificity onto T cells (for example, naïve T cells, central memory T cells, effector memory T cells or combinations thereof). CARs are also known as artificial T-cell receptors, chimeric T-cell receptors or chimeric immunoreceptors.

A CAR places a chimeric extracellular target-binding domain that specifically binds a target, e.g., a polypeptide, expressed on the surface of a cell to be targeted for a T cell response onto a construct including a transmembrane domain and intracellular domain(s) of a T cell receptor molecule. In one embodiment, the chimeric extracellular target-binding domain includes the antigen-binding domain(s) of an antibody that specifically binds an antigen expressed on a cell to be targeted for a T cell response. The properties of the intracellular signaling domain(s) of the CAR can vary as known in the art and as disclosed herein, but the chimeric target/antigen-binding domains(s) render the receptor sensitive to signaling activation when the chimeric target/antigen binding domain binds the target/antigen on the surface of a targeted cell.

With respect to intracellular signaling domains, so-called “first-generation” CARs include those that solely provide CD3zeta (CD3) signals upon antigen binding. So-called “second-generation” CARs include those that provide both co-stimulation (e.g., CD28 or CD137) and activation (CD3) domains, and so-called “third-generation” CARs include those that provide multiple costimulatory (e.g., CD28 and CD137) domains and activation domains (e.g., CD3). In various embodiments, the CAR is selected to have high affinity or avidity for the target/antigen—for example, antibody-derived target or antigen binding domains will generally have higher affinity and/or avidity for the target antigen than would a naturally-occurring T cell receptor. This property, combined with the high specificity one can select for an antibody provides highly specific T cell targeting by CAR T cells.

As used herein, a “CAR T cell” or “CAR-T” refers to a T cell that expresses a CAR. When expressed in a T cell, CARs have the ability to redirect T-cell specificity and reactivity toward a selected target in a non-MHC-restricted manner, exploiting the antigen-binding properties of monoclonal antibodies. The non-MHC-restricted antigen recognition gives T-cells expressing CARs the ability to recognize an antigen independent of antigen processing, thus bypassing a major mechanism of tumor escape.

As used herein, the term “extracellular target binding domain” refers to a polypeptide found on the outside of the cell that is sufficient to facilitate binding to a target. The extracellular target binding domain will specifically bind to its binding partner, i.e., the target. As non-limiting examples, the extracellular target-binding domain can include an antigen-binding domain of an antibody or antibody reagent, or a ligand, which recognizes and binds with a cognate binding partner protein. In this context, a ligand is a molecule that binds specifically to a portion of a protein and/or receptor. The cognate binding partner of a ligand useful in the methods and compositions described herein can generally be found on the surface of a cell. Ligand:cognate partner binding can result in the alteration of the ligand-bearing receptor, or activate a physiological response, for example, the activation of a signaling pathway. In one embodiment, the ligand can be non-native to the genome. Optionally, the ligand has a conserved function across at least two species.

Antibody Reagents

In various embodiments, the CARs described herein include an antibody reagent or an antigen-binding domain thereof as an extracellular target-binding domain.

As used herein, the term “antibody reagent” refers to a polypeptide that includes at least one immunoglobulin variable domain or immunoglobulin variable domain sequence and which specifically binds a given antigen. An antibody reagent can include an antibody or a polypeptide including an antigen-binding domain of an antibody. In some embodiments of any of the aspects, an antibody reagent can include a monoclonal antibody or a polypeptide including an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g., de Wildt et al., Eur. J. Immunol. 26(3):629-639, 1996; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

Fully human antibody binding domains can be selected, for example, from phage display libraries using methods known to those of ordinary skill in the art. Furthermore, antibody reagents include single domain antibodies, such as camelid antibodies.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A. et al. (1991) Sequences of Proteins of Immunological Interest, Fifth Edition, U.S. Department of Health and Human Services, NIH Publication No. 91-3242, and Chothia et al., J. Mol. Biol. 196:901-917, 1987; each of which is incorporated by reference herein in its entirety). Each VH and VL is typically composed of three CDRs and four FRs, arranged from amino-terminus to carboxy-terminus in the following order: FR1, CDR1, FR2, CDR2, FR3, CDR3, FR4.

In one embodiment, the antibody or antibody reagent is not a human antibody or antibody reagent (i.e., the antibody or antibody reagent is mouse), but has been humanized. A “humanized antibody or antibody reagent” refers to a non-human antibody or antibody reagent that has been modified at the protein sequence level to increase its similarity to antibody or antibody reagent variants produced naturally in humans. One approach to humanizing antibodies employs the grafting of murine or other non-human CDRs onto human antibody frameworks.

In one embodiment, the extracellular target binding domain of a CAR includes or consists essentially of a single-chain Fv (scFv) fragment created by fusing the VH and VL domains of an antibody, generally a monoclonal antibody, via a flexible linker peptide. In various embodiments, the scFv is fused to a transmembrane domain and to a T cell receptor intracellular signaling domain, e.g., an engineered intracellular signaling domain as described herein. In another embodiment, the extracellular target binding domain of a CAR includes a camelid antibody.

Antibody binding domains and ways to select and clone them are well-known to those of ordinary skill in the art. In some embodiments, the antibody reagent is an anti-GARP antibody reagent and includes the sequence of SEQ ID NO: 3 or 25, or includes a sequence with at least 75%, at least 80%, at least 85%, at least 90%, at least 95%, at least 96%, at least 97%, at least 98%, at least 99%, or greater sequence identity to the sequence of SEQ ID NO: 3 or 25. In further embodiments, the antibody reagent is an anti-GARP antibody reagent and includes the complementarity determining regions (CDRs) of SEQ ID NOs: 81, 82, 83, 84, 85, and/or 86, or includes CDR sequences with at least 1, 2, or 3 amino acid substitutions of SEQ ID NOs: 81, 82, 83, 84, 85, and/or 86. In further embodiments, the anti-GARP antibody reagent includes the variable heavy (VH) and/or variable light (VL) of SEQ ID NOs: 87 and 88, or includes VH and/or VL sequences with at least 75%, at least 80%, at least 85%, at least 90%, at least 95%, at least 96%, at least 97%, at least 98%, at least 99%, or greater sequence identity to the sequences of SEQ ID NOs: 87 and 88. The VH may be positioned N-terminal to the VL, or the VL may be positioned N-terminal to the VH. In further embodiments, the anti-GARP antibody reagent includes the sequence of SEQ ID NO: 71 or 77, or includes a sequence with at least 75%, at least 80%, at least 85%, at least 90%, at least 95%, at least 96%, at least 97%, at least 98%, at least 99%, or greater sequence identity to the sequence of SEQ ID NO: 71 or 77.

In other embodiments, the antibody reagent is an anti-LAP antibody reagent and includes the complementarity determining regions (CDRs) of SEQ ID NOs: 89, 90, 91, 92, 93, and/or 94, or includes CDR sequences with at least 1, 2, or 3 amino acid substitutions of SEQ ID NOs: 89, 90, 91, 92, 93, and/or 94. In further embodiments, the anti-LAP antibody reagent includes the VH and/or VL of SEQ ID NOs: 95 and 96, or includes VH and/or VL sequences with at least 75%, at least 80%, at least 85%, at least 90%, at least 95%, at least 96%, at least 97%, at least 98%, at least 99%, or greater sequence identity to the sequences of SEQ ID NOs: 87 and 88. The VH may be positioned N-terminal to the VL, or the VL may be positioned N-terminal to the VH. In further embodiments, the antibody reagent is an anti-LAP antibody reagent and includes the sequence of SEQ ID NO: 9 or 15, or includes a sequence with at least 75%, at least 80%, at least 85%, at least 90%, at least 95%, at least 96%, at least 97%, at least 98%, at least 99%, or greater sequence identity to the sequence of SEQ ID NO: 9 or 15. In other embodiments, the antibody reagent is an anti-EGFR or anti-EGFRvIII antibody reagent and includes the sequence of SEQ ID NO: 21, 27, 33, 36, 42, 45, 55, 57, 65, or 103, or includes a sequence with at least 75%, at least 80%, at least 85%, at least 90%, at least 95%, at least 96%, at least 97%, at least 98%, at least 99%, or greater sequence identity to the sequence of SEQ ID NO: 21, 27, 33, 36, 42, 45, 55, 57, 65, or 103.

In particular embodiments, the antibody reagent is an anti-EGFRvIII scFv. For example, the anti-EGFRvIII scFv includes a VH corresponding to the amino acid sequence of SEQ ID NO: 111 or 113; including the amino acid sequence of SEQ ID NO: 111 or 113; or including an amino acid sequence having at least at least 75%, at least 80%, at least 85%, at least 90%, at least 95%, at least 96%, at least 97%, at least 98%, at least 99%, or greater sequence identity to the amino acid sequence of SEQ ID NO: 111 or 113. In further embodiments, the anti-EGFRvIII scFV includes a VL corresponding to the amino acid sequence of SEQ ID NO: 112 or 114; including the amino acid sequence of SEQ ID NO: 112 or 114; or including an amino acid sequence having at least 75%, at least 80%, at least 85%, at least 90%, at least 95%, at least 96%, at least 97%, at least 98%, at least 99%, or greater sequence identity to the amino acid sequence of SEQ ID NO: 112 or 114. In some embodiments, the anti-EGFRvIII scFv corresponds to the sequence of SEQ ID NO: 27, 36, 45, 57, 65, or 103; includes the sequence of SEQ ID NO: 27, 36, 45, 57, 65, or 103, or includes a sequence having at least 75%, at least 80%, at least 85%, at least 90%, at least 95%, at least 96%, at least 97%, at least 98%, at least 99%, or greater sequence identity to the sequence of SEQ ID NO: 27, 36, 45, 57, 65, or 103. An immune cell including a CAR polypeptide including an extracellular target binding domain including an anti-EGFRvIII scFv may secrete an anti-EGFR BiTE as described below.

In other embodiments, the antibody reagent is an anti-CD19 antibody reagent and includes the sequence of SEQ ID NO: 51 or 63, or includes a sequence with at least 75%, at least 80%, at least 85%, at least 90%, at least 95%, at least 96%, at least 97%, at least 98%, at least 99%, or greater sequence identity to the sequence of SEQ ID NO: 51 or 63.

In yet other embodiments, the antibody reagent is an anti-CD3 antibody reagent and includes the sequence of SEQ ID NO: 34, 43, 52, 56, or 64, or includes a sequence with at least 75%, at least 80%, at least 85%, at least 90%, at least 95%, at least 96%, at least 97%, at least 98%, at least 99%, or greater sequence identity to the sequence of SEQ ID NO: 34, 43, 52, 56, or 64. In various examples, the antibody reagent can be selected from C225, 3C10, Cetuximab, and 2173. Any antibody reagent described herein can be useful as an antigen-binding domain of a CAR, or as a therapeutic agent.

In one embodiment, the CARs useful in the technology described herein include at least two antigen-specific targeting regions, an extracellular domain, a transmembrane domain, and an intracellular signaling domain. In such embodiments, the two or more antigen-specific targeting regions target at least two different antigens and may be arranged in tandem and separated by linker sequences. In another embodiment, the CAR is a bispecific CAR. A bispecific CAR is specific to two different antigens.

For example, a bispecific CAR can be a tandem CAR that targets IL-13Rα2 and EGFRvIII. In some embodiments, the IL-13Rα2 binding sequence includes an anti-IL-13Rα2 antibody reagent, e.g., an scFv or a single domain antibody (e.g., a camelid). In some embodiments, the IL-13Rα2 binding sequence may include an IL-13Rα2 ligand or an antigen-binding fragment thereof, e.g., IL-13 or IL-13 zetakine. In some embodiments, the IL-13 zetakine corresponds to the sequence of SEQ ID NO: 101, or includes the sequence of SEQ ID NO: 101, or includes a sequence having at least 75%, at least 80%, at least 85%, at least 90%, at least 95%, at least 96%, at least 97%, at least 98%, at least 99%, or greater sequence identity to the sequence of SEQ ID NO: 101. In some embodiments, the EGFRvIII binding site may include an anti-EGFRvIII scFv. In some embodiments, the anti-EGFRvIII scFv includes a VH corresponding to the sequence of SEQ ID NO: 111 or 113, including the amino acid sequence of SEQ ID NO: 111 or 113, or including an amino acid sequence having at least 75%, at least 80%, at least 85%, at least 90%, at least 95%, at least 96%, at least 97%, at least 98%, at least 99%, or greater sequence identity to the amino acid sequence of SEQ ID NO: 111 or 113. In some embodiments, the anti-EGFRvIII scFv includes a VL corresponding to the amino acid sequence of SEQ ID NO: 112 or 114, including the amino acid sequence of SEQ ID NO: 112 or 114, or including an amino acid sequence having at least 75%, at least 80%, at least 85%, at least 90%, at least 95%, at least 96%, at least 97%, at least 98%, at least 99%, or greater sequence identity to the amino acid sequence of SEQ ID NO: 112 or 114. The VH may be positioned N-terminal to the VL, or the VL may be positioned N-terminal to the VH. In some embodiments, the anti-EGFRvIII scFv corresponds to the sequence of SEQ ID NO: 27, 36, 45, 57, 65, or 103, or includes the sequence of SEQ ID NO: 27, 36, 45, 57, 65, or 103, or includes a sequence having at least 75%, at least 80%, at least 85%, at least 90%, at least 95%, at least 96%, at least 97%, at least 98%, at least 99%, or greater sequence identity to the sequence of SEQ ID NO: 27, 36, 45, 57, 65, or 103. The IL-13Rα2 binding sequence may be positioned N-terminal to the EGFRvIII binding sequence, or the EGFRvIII binding sequence may be positioned N-terminal to the IL-13Rα2. The IL-13Rα2 binding sequence and EGFRvIII binding sequence may optionally be connected via a linker, e.g., of SEQ ID NO: 102, as well as any other linker described herein or known in the art.

Target/Antigen Any cell-surface moiety can be targeted by a CAR. Often, the target will be a cell-surface polypeptide that may be differentially or preferentially expressed on a cell that one wishes to target for a T cell response. To target Tregs, antibody reagents can be targeted against, e.g., Glycoprotein A Repetitions Predominant (GARP), latency-associated peptide (LAP), CD25, CTLA-4, ICOS, TNFR2, GITR, OX40, 4-1 BB, and LAG-3. To target tumors or cancer cells, antibody domains can be targeted against, e.g., EGFR or EGFRvIII, as described herein. Targeting tumor antigens or tumor-associated antigens that are specific to the tumors can provide a means to target tumor cells while avoiding or at least limiting collateral damage to non-tumor cells or tissues. Non-limiting examples of additional tumor antigens, tumor-associated antigens, or other antigen of interest include CD19, CD37, BCMA (tumor necrosis factor receptor superfamily member 17 (TNFRSF17); NCBI Gene ID: 608; NCBI Ref Seq: NP_001183.2 and mRNA (e.g., NCBI Ref Seq: NM_001192.2)), CEA, immature laminin receptor, TAG-72, HPV E6 and E7, BING-4, calcium-activated chloride channel 2, cyclin B1, 9D7, Ep-CAM, EphA3, her2/neu, telomerase, mesotheliun, SAP-1, survivin, BAGE family, CAGE family, GAGE family, MAGE family, SAGE family, XAGE family, NY-ESO-1/LAGE-1, PRAME, SSX-2, Melan-A/MART-1, gp100/pme117, tyrosinase, TRP-1/-2, MC1R, BRCA1/2, CDK4, MART-2, p53, Ras, MUC1, TGF-61:111, IL-15, IL13Rα2, and CSF1R.

In some embodiments, the target/antigen of the CAR is EGFR, EGFRvIII, CD19, CD79b, CD37, prostate-specific membrane antigen (PSMA), prostate stem cell antigen (PSCA), IL-13Rα2, EphA1, Her2, mesothelin, MUC1, or MUC16. In other embodiments, the target/antigen of the CAR is LAP or GARP. In further embodiments, the CAR is a bispecific CAR that binds to two of EGFR, EGFRvIII, CD19, CD79b, CD37, PSMA, PSCA, IL-13Rα2, EphA1, Her2, mesothelin, MUC1, and MUC16.

Hinge and Transmembrane Domains

Each CAR as described herein includes a transmembrane domain, e.g., a hinge/transmembrane domain, which joins the extracellular target-binding domain to the intracellular signaling domain.

The binding domain of the CAR is optionally followed by one or more “hinge domains,” which plays a role in positioning the antigen binding domain away from the effector cell surface to enable proper cell/cell contact, antigen binding and activation. A CAR optionally includes one or more hinge domains between the binding domain and the transmembrane domain (TM). The hinge domain may be derived either from a natural, synthetic, semi-synthetic, or recombinant source. The hinge domain can include the amino acid sequence of a naturally occurring immunoglobulin hinge region or an altered immunoglobulin hinge region. Illustrative hinge domains suitable for use in the CARs described herein include the hinge region derived from the extracellular regions of type 1 membrane proteins such as CD8 (e.g., CD8α), CD4, CD28, 4-1 BB, and CD7, which may be wild-type hinge regions from these molecules or may be altered. In some embodiments, the hinge region is derived from the hinge region of an immunoglobulin-like protein (e.g., IgA, IgD, IgE, IgG, or IgM), CD28, or CD8. In one embodiment, the hinge domain includes a CD8α hinge region.

As used herein, “transmembrane domain” (TM domain) refers to the portion of the CAR that fuses the extracellular binding portion, optionally via a hinge domain, to the intracellular portion (e.g., the co-stimulatory domain and intracellular signaling domain) and anchors the CAR to the plasma membrane of the immune effector cell. The transmembrane domain is a generally hydrophobic region of the CAR which crosses the plasma membrane of a cell. The TM domain can be the transmembrane region or fragment thereof of a transmembrane protein (for example a Type I transmembrane protein or other transmembrane protein), an artificial hydrophobic sequence, or a combination thereof. While specific examples are provided herein and used in the Examples, other transmembrane domains will be apparent to those of skill in the art and can be used in connection with alternate embodiments of the technology. A selected transmembrane region or fragment thereof would preferably not interfere with the intended function of the CAR. As used in relation to a transmembrane domain of a protein or polypeptide, “fragment thereof” refers to a portion of a transmembrane domain that is sufficient to anchor or attach a protein to a cell surface.

In some examples, the transmembrane domain or fragment thereof of the CAR described herein includes a transmembrane domain selected from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11 a, CD18), ICOS (CD278), 4-1BB (CD137), 4-1BBL, GITR, CD40, BAFFR, HVEM (LIGHTR), SLAMF7, NKp80 (KLRFI), CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11 a, LFA-1, ITGAM, CD11 b, ITGAX, CD11 c, ITGB1, CD29, ITGB2, CD18, LFA-1, ITGB7, TNFR2, DNAM1(CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Lyl08), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

As used herein, a “hinge/transmembrane domain” refers to a domain including both a hinge domain and a transmembrane domain. For example, a hinge/transmembrane domain can be derived from the hinge/transmembrane domain of CD8, CD28, CD7, or 4-1 BB. In one embodiment, the hinge/transmembrane domain of a CAR or fragment thereof is derived from or includes the hinge/transmembrane domain of CD8 (e.g., any one of SEQ ID NOs: 4, 10, 16, 22, 28, 37, 46, 58, 66, 72, 78, 104, or variants thereof).

CD8 is an antigen preferentially found on the cell surface of cytotoxic T lymphocytes. CD8 mediates cell-cell interactions within the immune system, and acts as a T cell co-receptor. CD8 consists of an alpha (CD8α or CD8a) and beta (CD8β or CD8b) chain. CD8a sequences are known for a number of species, e.g., human CD8a, (NCBI Gene ID: 925) polypeptide (e.g., NCBI Ref Seq NP_001139345.1) and mRNA (e.g., NCBI Ref Seq NM_000002.12). CD8 can refer to human CD8, including naturally occurring variants, molecules, and alleles thereof. In some embodiments of any of the aspects, e.g., in veterinary applications, CD8 can refer to the CD8 of, e.g., dog, cat, cow, horse, pig, and the like. Homologs and/or orthologs of human CD8 are readily identified for such species by one of skill in the art, e.g., using the NCBI ortholog search function or searching available sequence data for a given species for sequence similar to a reference CD8 sequence.

In some embodiments, the CD8 hinge and transmembrane sequence corresponds to the amino acid sequence of SEQ ID NO: 4, 10, 16, 22, 28, 37, 46, 58, 66, 72, 78, or 104; or includes the sequence of SEQ ID NO: 4, 10, 16, 22, 28, 37, 46, 58, 66, 72, 78, or 104; or includes a sequence with at least 75%, at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 4, 10, 16, 22, 28, 37, 46, 58, 66, 72, 78, or 104.

Co-Stimulatory Domains

Each CAR described herein optionally includes the intracellular domain of one or more co-stimulatory molecule or co-stimulatory domain. As used herein, the term “co-stimulatory domain” refers to an intracellular signaling domain of a co-stimulatory molecule. Co-stimulatory molecules are cell surface molecules other than antigen receptors or Fc receptors that provide a second signal required for efficient activation and function of T lymphocytes upon binding to antigen. The co-stimulatory domain can be, for example, the co-stimulatory domain of 4-1 BB, CD27, CD28, or OX40. In one example, a 4-1 BB intracellular domain (ICD) can be used (see, e.g., below and SEQ ID NOs: 5, 11, 17, 23, 29, 38, 47, 59, 67, 73, 79, 105, or variants thereof). Additional illustrative examples of such co-stimulatory molecules include CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAGS), CD270 (HVEM), CD273 (PD-L2), CD274 (PD-L1), CD278 (ICOS), DAP10, LAT, NKD2C SLP76, TRIM, and ZAP70. In one embodiment, the intracellular domain is the intracellular domain of 4-1 BB. 4-1 BB (CD137; TNFRS9) is an activation-induced costimulatory molecule, and is an important regulator of immune responses.

4-1 BB is a membrane receptor protein, also known as CD137, which is a member of the tumor necrosis factor (TNF) receptor superfamily. 4-1 BB is expressed on activated T lymphocytes. 4-1 BB sequences are known for a number of species, e.g., human 4-1 BB, also known as TNFRSF9 (NCBI Gene ID: 3604) and mRNA (NCBI Reference Sequence: NM_001561.5). 4-1 BB can refer to human 4-1 BB, including naturally occurring variants, molecules, and alleles thereof. In some embodiments of any of the aspects, e.g., in veterinary applications, 4-1 BB can refer to the 4-1 BB of, e.g., dog, cat, cow, horse, pig, and the like. Homologs and/or orthologs of human 4-1 BB are readily identified for such species by one of skill in the art, e.g., using the NCBI ortholog search function or searching available sequence data for a given species for sequence similar to a reference 4-1 BB sequence.

In some embodiments, the intracellular domain is the intracellular domain of a 4-1 BB. In one embodiment, the 4-1 BB intracellular domain corresponds to an amino acid sequence selected from SEQ ID NO: 5, 11, 17, 23, 29, 38, 47, 59, 67, 73, 79, or 105; or includes a sequence selected from SEQ ID NO: 5, 11, 17, 23, 29, 38, 47, 59, 67, 73, 79, or 105; or includes at least 75%, at least 80%, at least 85%, at least 90%, at least 95%, at least 96%, at least 97%, at least 98%, at least 99%, or at least 100% sequence identity to a sequence selected from SEQ ID NO: 5, 11, 17, 23, 29, 38, 47, 59, 67, 73, 79, or 105.

Intracellular Signaling Domains

CARs as described herein include an intracellular signaling domain. An “intracellular signaling domain,” refers to the part of a CAR polypeptide that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited following antigen binding to the extracellular CAR domain. In various examples, the intracellular signaling domain is from CD3ζ (see, e.g., below). Additional non-limiting examples of immunoreceptor tyrosine-based activation motif (ITAM)-containing intracellular signaling domains that are of particular use in the technology include those derived from TCRζ, FcRγ, FcRβ, CD3γ, CD3θ, CD3θ, CD3η, CD3ε, CD3ζ, CD22, CD79a, CD79b, and CD66d.

CD3 is a T cell co-receptor that facilitates T lymphocyte activation when simultaneously engaged with the appropriate co-stimulation (e.g., binding of a co-stimulatory molecule). A CD3 complex consists of 4 distinct chains; mammalian CD3 consists of a CD3γ chain, a CD3δchain, and two CD3ε chains. These chains associate with a molecule known as the T cell receptor (TCR) and the CD3ζ to generate an activation signal in T lymphocytes. A complete TCR complex includes a TCR, CD3ζ, and the complete CD3 complex.

In some embodiments of any aspect, a CAR polypeptide described herein includes an intracellular signaling domain that includes an Immunoreceptor Tyrosine-based Activation Motif or ITAM from CD3 zeta (CD3ζ), including variants of CD3ζ such as ITAM-mutated CD3ζ, CD3η, or CD3θ. In some embodiments of any aspect, the ITAM includes three motifs of ITAM of CD3ζ (ITAM3). In some embodiments of any aspect, the three motifs of ITAM of CD3ζ are not mutated and, therefore, include native or wild-type sequences. In some embodiments, the CD3ζ sequence includes the sequence of a CD3ζ as set forth in the sequences provided herein, e.g., a CD3ζ sequence of one of SEQ ID NOs: 6, 12, 18, 24, 30, 39, 48, 60, 68, 74, 80, 106, or variants thereof.

For example, a CAR polypeptide described herein includes the intracellular signaling domain of CD3ζ. In one embodiment, the CD3ζ intracellular signaling domain corresponds to an amino acid sequence selected from SEQ ID NO: 6, 12, 18, 24, 30, 39, 48, 60, 68, 74, 80, or 106; or includes a sequence selected from SEQ ID NO: 6, 12, 18, 24, 30, 39, 48, 60, 68, 74, 80, or 106; or includes a sequence with at least 75%, at least 80%, at least 85%, at least 90%, at least 91%, at least 92%, at least 93%, at least 94%, at least 95%, at least 96%, at least 97%, at least 98%, at least 99%, or at least 100% sequence identity to a sequence selected from SEQ ID NO: 6, 12, 18, 24, 30, 39, 48, 60, 68, 74, 80, or 106.

Individual CAR and other construct components as described herein can be used with one another and swapped in and out of various constructs described herein, as can be determined by those of skill in the art. Each of these components can include or consist of any of the corresponding sequences set forth herein, or variants thereof.

A more detailed description of CARs and CAR T cells can be found in Maus et al., Blood 123:2624-2635, 2014; Reardon et al., Neuro-Oncology 16:1441-1458, 2014; Hoyos et al., Haematologica 97:1622, 2012; Byrd et al., J. Clin. Oncol. 32:3039-3047, 2014; Maher et al., Cancer Res 69:4559-4562, 2009; and Tamada et al., Clin. Cancer Res. 18:6436-6445, 2012; each of which is incorporated by reference herein in its entirety.

In some embodiments, a CAR polypeptide as described herein includes a signal peptide. Signal peptides can be derived from any protein that has an extracellular domain or is secreted. A CAR polypeptide as described herein may include any signal peptides known in the art. In some embodiments, the CAR polypeptide includes a CD8 signal peptide, e.g., a CD8 signal peptide corresponding to the amino acid sequence of SEQ ID NO: 2, 8, 14, 20, 70, or 76, or including the amino acid sequence of SEQ ID NO: 2, 8, 14, 20, 70, or 76, or including an amino acid sequence having at least 75%, at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 2, 8, 14, 20, 70, or 76.

In further embodiments, a CAR polypeptide described herein may optionally exclude one of the signal peptides described herein, e.g., a CD8 signal peptide of SEQ ID NO: 2, 8, 14, 20, 70, or 76 or an Igκ signal peptide of SEQ ID NO: 32, 41, 50, 54, or 62.

In one embodiment, the CAR further includes a linker domain. As used herein, “linker domain” refers to an oligo- or polypeptide region from about 2 to 100 amino acids in length, which links together any of the domains/regions of the CAR as described herein. In some embodiment, linkers can include or be composed of flexible residues such as glycine and serine so that the adjacent protein domains are free to move relative to one another. Linker sequences useful for the invention can be from 2 to 100 amino acids, 5 to 50 amino acids, 10 to 15 amino acids, 15 to 20 amino acids, or 18 to 20 amino acids in length, and include any suitable linkers known in the art. For instance, linker sequences useful for the invention include, but are not limited to, glycine/serine linkers, e.g., GGGSGGGSGGGS (SEQ ID NO: 107) and Gly4Ser (G4S) linkers such as (G4S)3 (GGGGSGGGGSGGGGS (SEQ ID NO: 108)) and (G4S)4 (GGGGSGGGGSGGGGSGGGGS (SEQ ID NO: 102)); the linker sequence of GSTSGSGKPGSGEGSTKG (SEQ ID NO: 109) as described by Whitlow et al., Protein Eng. 6(8):989-95, 1993, the contents of which are incorporated herein by reference in its entirety; the linker sequence of GGSSRSSSSGGGGSGGGG (SEQ ID NO: 110) as described by Andris-Widhopf et al., Cold Spring Harb. Protoc. 2011(9), 2011, the contents of which are incorporated herein by reference in its entirety; as well as linker sequences with added functionalities, e.g., an epitope tag or an encoding sequence containing Cre-Lox recombination site as described by Sblattero et al., Nat. Biotechnol. 18(1):75-80, 2000, the contents of which are incorporated herein by reference in its entirety. Longer linkers may be used when it is desirable to ensure that two adjacent domains do not sterically interfere with one another.

Furthermore, linkers may be cleavable or non-cleavable. Examples of cleavable linkers include 2A linkers (e.g., P2A and T2A), 2A-like linkers or functional equivalents thereof and combinations thereof. For example, a P2A linker sequence can correspond to the amino acid sequence of SEQ ID NO: 31, 40, or 49. In various examples, linkers having sequences as set forth herein, or variants thereof, are used. It is to be understood that the indication of a particular linker in a construct in a particular location does not mean that only that linker can be used there. Rather, different linker sequences (e.g., P2A and T2A) can be swapped with one another (e.g., in the context of the constructs of the present invention), as can be determined by those of skill in the art. In one embodiment, the linker region is T2A derived from Thosea asigna virus. Non-limiting examples of linkers that can be used in this technology include T2A, P2A, E2A, BmCPV2A, and BmIFV2A. Linkers such as these can be used in the context of polyproteins, such as those described below. For example, they can be used to separate a CAR component of a polyprotein from a therapeutic agent (e.g., an antibody, such as a scFv, single domain antibody (e.g., a camelid antibody), or a bispecific antibody (e.g., a BiTE)) component of a polyprotein (see below).

In some embodiments, a CAR as described herein optionally further includes a reporter molecule, e.g., to permit for non-invasive imaging (e.g., positron-emission tomography PET scan). In a bispecific CAR that includes a reporter molecule, the first extracellular binding domain and the second extracellular binding domain can include different or the same reporter molecule. In a bispecific CAR T cell, the first CAR and the second CAR can express different or the same reporter molecule. In another embodiment, a CAR as described herein further includes a reporter molecule (for example hygromycin phosphotransferase (hph)) that can be imaged alone or in combination with a substrate or chemical (for example 9-[4-[¹⁸F]fluoro-3-(hydroxymethyl)butyl]guanine ([¹⁸F]FHBG)). In another embodiment, a CAR as described herein further includes nanoparticles at can be readily imaged using non-invasive techniques (e.g., gold nanoparticles (GNP) functionalized with ⁶⁴Cu²⁺). Labeling of CAR T cells for non-invasive imaging is reviewed, for example in Bhatnagar et al., Integr. Biol. (Camb). 5(1):231-238, 2013, and Keu et al., Sci. Transl. Med. 18; 9(373), 2017, which are incorporated herein by reference in their entireties.

GFP and mCherry are demonstrated herein as fluorescent tags useful for imaging a CAR expressed on a T cell (e.g., a CAR T cell). It is expected that essentially any fluorescent protein known in the art can be used as a fluorescent tag for this purpose. For clinical applications, the CAR need not include a fluorescent tag or fluorescent protein. In each instance of particular constructs provided herein, therefore, any markers present in the constructs can be removed. The invention includes the constructs with or without the markers. Accordingly, when a specific construct is referenced herein, it can be considered with or without any markers or tags (including, e.g., histidine tags, such as the histidine tag of HHHHHH (SEQ ID NO: 97)) as being included within the invention.

In some embodiments, the CAR polypeptide sequence corresponds to, includes, or includes a sequence with at least 75%, at least 80%, at least 85%, at least 90%, at least 91%, at least 92%, at least 93%, at least 94%, at least 95%, at least 96%, at least 97%, at least 98%, at least 99% or greater sequence identity of a sequence selected from SEQ ID NOs: 1, 7, 13, 69, 75, 100, 115, 116, or 117, optionally excluding a CD8 signal peptide as described herein, or the combination of SEQ ID NOs: 21-24, 27-30, 36-39, 45-48, 57-60, 65-68, 71-74, 77-80, or 101-106. As can be determined by those of skill in the art, various functionally similar or equivalent components of these CARs can be swapped or substituted with one another, as well as other similar or functionally equivalent components known in the art or listed herein.

### Therapeutic Agents Delivered by CAR T Cells

As noted above, the CAR T cells of the invention can optionally be used to deliver therapeutic agents, e.g., antibody reagents or other therapeutic molecules, such as cytokines, to tumors (i.e., to the tumor microenvironment). In various embodiments, the therapeutic agent is encoded by the same nucleic acid molecule as the CAR, thus facilitating transduction of cells (e.g., T cells) to express both a CAR and a therapeutic agent, e.g., an antibody reagent or cytokine. In such examples, the therapeutic agent (e.g., an antibody reagent or cytokine) can be expressed, e.g., such that it is separated from the CAR (and optionally other proteins, e.g., markers) by cleavable linker sequences (e.g., a 2A linker, such as, e.g., P2A or T2A; see above). The therapeutic agent (e.g., an antibody reagent or cytokine) can be expressed under the control of the same promoter as the CAR (e.g., by an EF1α promoter), and can be constitutively expressed. In other examples, the therapeutic agent (e.g., an antibody reagent or cytokine) is expressed under the control of an inducible promoter, e.g., a promoter that is expressed upon T cell activation (e.g., an NFAT promoter). Such an inducible promoter can be used, e.g., to ensure that the antibody is expressed only upon T cell activation, and thus only, e.g., when the CAR T cell is within the tumor microenvironment, to which locale it may be advantageous to have antibody production limited. As is understood in the art, the CAR coding sequences can be 5′ or 3′ to the therapeutic agent (e.g., an antibody reagent or cytokine) coding sequences in various vector designs within the invention. In some embodiments, the therapeutic agent includes an Igκ signal peptide, e.g., an Igκ signal peptide corresponding to the amino acid sequence of SEQ ID NO: 32, 41, 50, 54, or 62, or including the amino acid sequence of SEQ ID NO: 32, 41, 50, 54, or 62, or including an amino acid sequence having at least 75%, at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 32, 41, 50, 54, or 62.

In various examples, the therapeutic agent is an antibody reagent. The antibody reagent expressed within a CAR T cell (e.g., from the same nucleic acid molecule as the CAR) can be a single chain antibody (e.g., an scFv) or a single domain antibody (e.g., a camelid) as described herein. In the case of single chain antibodies, the light (L) and heavy (H) chains may be in the order (N-terminal to C-terminal) L-H or H-L, and optionally may be separated from one another by a linker (e.g., a glycine-based linker). In further examples, the antibody reagent is a bispecific antibody including, e.g., bispecific T cell engagers (BiTEs), described below.

Antibody reagents can be targeted against, e.g., tumor antigens, such as EGFR, EGFRvIII, CD19, IL-15, IL13Rα2, CSF1R. For example, the antibody reagent is an anti-EGFR or anti-EGFRvIII antibody reagent and includes the sequence of SEQ ID NO: 21, 27, 33, 36, 42, 45, 55, 57, or 65, or includes a sequence with at least 75%, at least 80%, at least 85%, at least 90%, at least 95%, at least 96%, at least 97%, at least 98%, at least 99%, or greater sequence identity to the sequence of SEQ ID NO: 21, 27, 33, 36, 42, 45, 55, 57, or 65. In another example, the antibody reagent is an anti-CD19 antibody reagent and includes the sequence of SEQ ID NO: 51 or 63, or includes a sequence with at least 75%, at least 80%, at least 85%, at least 90%, at least 95%, at least 96%, at least 97%, at least 98%, at least 99%, or greater sequence identity to the sequence of SEQ ID NO: 51 or 63. In another example, the antibody reagent is an anti-CD3 antibody reagent and includes the sequence of SEQ ID NO: 34, 43, 52, 56, or 64, or includes a sequence with at least 75%, at least 80%, at least 85%, at least 90%, at least 95%, at least 96%, at least 97%, at least 98%, at least 99%, or greater sequence identity to the sequence of SEQ ID NO: 34, 43, 52, 56, or 64. In various other examples, the antibody reagent can include C225, 3C10, Cetuximab, or 2173, or an antigen-binding fragment thereof.

In other examples, antibody reagents can be targeted against, e.g., Treg antigens, such as CTLA-4, CD25, GARP, LAP. For example, the antibody reagent is an anti-GARP antibody reagent and includes the sequence of SEQ ID NO: 3 or 25, or includes a sequence with at least 75%, at least 80%, at least 85%, at least 90%, at least 95%, at least 96%, at least 97%, at least 98%, at least 99%, or greater sequence identity to the sequence of SEQ ID NO: 3 or 25. In further embodiments, the antibody reagent is an anti-GARP antibody reagent and includes the complementarity determining regions (CDRs) of SEQ ID NOs: 81, 82, 83, 84, 85, and/or 86, or includes CDR sequences with at least 1, 2, or 3 amino acid substitutions of SEQ ID NOs: 81, 82, 83, 84, 85, and/or 86. In further embodiments, the anti-GARP antibody reagent includes the VH and/or VL of SEQ ID NOs: 87 and 88, or includes VH and/or VL sequences with at least 75%, at least 80%, at least 85%, at least 90%, at least 95%, at least 96%, at least 97%, at least 98%, at least 99%, or greater sequence identity to the sequences of SEQ ID NOs: 87 and 88. The VH may be positioned N-terminal to the VL, or the VL may be positioned N-terminal to the VH. In further embodiments, the anti-GARP antibody reagent includes the sequence of SEQ ID NO: 71 or 77, or includes a sequence with at least 75%, at least 80%, at least 85%, at least 90%, at least 95%, at least 96%, at least 97%, at least 98%, at least 99%, or greater sequence identity to the sequence of SEQ ID NO: 71 or 77. In another example, the antibody reagent is an anti-LAP antibody reagent and includes the complementarity determining regions (CDRs) of SEQ ID NOs: 89, 90, 91, 92, 93, and/or 94, or includes CDR sequences with at least 1, 2, or 3 amino acid substitutions of SEQ ID NOs: 89, 90, 91, 92, 93, and/or 94. In some embodiments, the anti-LAP antibody reagent includes the VH and/or VL of SEQ ID NOs: 95 and 96, or includes VH and/or VL sequences with at least 75%, at least 80%, at least 85%, at least 90%, at least 95%, at least 96%, at least 97%, at least 98%, at least 99%, or greater sequence identity to the sequences of SEQ ID NOs: 87 and 88. The VH may be positioned N-terminal to the VL, or the VL may be positioned N-terminal to the VH. In further embodiments, the antibody reagent is an anti-LAP antibody reagent and includes the sequence of SEQ ID NO: 9 or 15, or includes a sequence with at least 75%, at least 80%, at least 85%, at least 90%, at least 95%, at least 96%, at least 97%, at least 98%, at least 99%, or greater sequence identity to the sequence of SEQ ID NO: 9 or 15. In a further example, the antibody reagent can include daclizumab or an antigen-binding fragment thereof.

Antibody reagents can also be targeted against any other antigens described herein or known in the art. In addition to optionally delivering antibody reagents, as described herein, the CAR T cells of the invention can be used to deliver other therapeutic agents including, but not limited to, cytokines and toxins.

Bispecific T Cell Engagers (BiTEs)

In some embodiments, the therapeutic agent delivered by a CAR T cell as described herein is a bispecific T cell engager (BiTE). Such molecules can target T cells by binding to a T cell antigen (e.g., by binding CD3) as well as a target antigen, e.g., a tumor antigen. Exemplary tumor antigens include EGFR, EGFRvIII, CD19, CD79b, CD37, PSMA, PSCA, IL-13Rα2, EphA1, Her2, mesothelin, MUC1, or MUC16 (also see above). The BiTEs can be used to augment the T cell response in, e.g., the tumor microenvironment. The two components of a BiTE can optionally be separated from one another by a linker as described herein (e.g., a glycine-based linker), and may also be connected in either orientation, e.g., with the anti-CD3 component N-terminal to the anti-target antigen component, or vice versa. The anti-CD3 component or the anti-target antigen component of the BiTE may include any of the antibody reagents described herein.

The CAR T cell secreted BiTEs may, e.g., stimulate the CAR T cell itself, or operate in a paracrine fashion by redirecting nonspecific bystander T cells against tumors and therefore enhance the anti-tumor effects of CAR T cell immunotherapy. CAR T cell-mediated BiTE secretion may allow for the reduction of risk of undesired BiTE activity in systemic tissues by directing BiTE secretion to the tumor microenvironment. Exemplary BiTE constructs are provided below; however, BiTEs other than those described herein may also be useful for the invention.

An exemplary BiTE useful for the invention described herein includes, e.g., an anti-EGFR BiTE including an anti-EGFR scFv and an anti-CD3 scFv (also referred to herein as BiTE-EGFR). The anti-EGFR scFv may be arranged in the VH-VL orientation, or in the VL-VH orientation. In particular embodiments, the anti-EGFR scFv corresponds to the amino acid sequence of SEQ ID NO: 33, 42, or 55, or includes the amino acid sequence of SEQ ID NO: 33, 42, or 55, or includes an amino acid sequence having at least 75%, at least 80%, at least 85%, at least 90%, at least 95%, at least 96%, at least 97%, at least 98%, at least 99%, or greater sequence identity to the amino acid sequence of SEQ ID NO: 33, 42, or 55.

Another exemplary BiTE is an anti-CD19 BiTE including an anti-CD19 scFv and an anti-CD3 scFv (also referred to herein as BiTE-CD19). The anti-CD19 scFv may be arranged in the VH-VL orientation, or in the VL-VH orientation. In certain embodiments, the anti-CD19 scFv corresponds to the amino acid sequence of SEQ ID NO: 51 or 63, or includes the amino acid sequence of SEQ ID NO: 51 or 63, or includes an amino acid sequence having at least 75%, at least 80%, at least 85%, at least 90%, at least 95%, at least 96%, at least 97%, at least 98%, at least 99%, or greater sequence identity to the amino acid sequence of SEQ ID NO: 51 or 63.

In some embodiments, the anti-CD3 scFv of any of the BiTEs described herein may be arranged in the VH-VL orientation, or in the VL-VH orientation, and may optionally corresponds to the amino acid sequence of SEQ ID NO: 34, 43, 52, 56, or 64, or include the amino acid sequence of SEQ ID NO: 34, 43, 52, 56, or 64, or include an amino acid sequence having at least 75%, at least 80%, at least 85%, at least 90%, at least 95%, at least 96%, at least 97%, at least 98%, at least 99%, or greater sequence identity to the amino acid sequence of SEQ ID NO: 34, 43, 52, 56, or 64.

An anti-EGFR BiTE as described herein can correspond to the amino acid sequence of SEQ ID NO: 98, or include the amino acid sequence of SEQ ID NO: 98, or include an amino acid sequence having at least 75%, at least 80%, at least 85%, at least 90%, at least 95%, at least 96%, at least 97%, at least 98%, at least 99%, or greater sequence identity to the amino acid sequence of SEQ ID NO: 98. An anti-CD19 BiTE as described herein can correspond to the amino acid sequence of SEQ ID NO: 99, or include the amino acid sequence of SEQ ID NO: 99, or include an amino acid sequence having at least 75%, at least 80%, at least 85%, at least 90%, at least 95%, at least 96%, at least 97%, at least 98%, at least 99%, or greater sequence identity to the amino acid sequence of SEQ ID NO: 99.

Optionally, the BiTE may include a signal peptide described herein, such as an IgK signal peptide, e.g., an IgK signal peptide corresponding to the amino acid sequence of SEQ ID NO: 32, 41, 50, 54, or 62, or including the amino acid sequence of SEQ ID NO: 32, 41, 50, 54, or 62, or including an amino acid sequence having at least 75%, at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 32, 41, 50, 54, or 62.

In some embodiments, the CAR T cell includes a polyprotein including a CAR and a therapeutic agent and/or a nucleic acid encoding the polyprotein. In certain embodiments, the polyprotein sequence, including a CAR and a therapeutic agent, corresponds to, includes, or includes a sequence with at least 80%, at least 85%, at least 90%, at least 91%, at least 92%, at least 93%, at least 94%, at least 95%, at least 96%, at least 97%, at least 98%, at least 99% or greater sequence identity of a sequence selected from SEQ ID NOs: 19, 26, 35, 44, 53, and 61.

Other components of CARs and related constructs (or variants thereof), as described herein, such as an Igκ signal sequence (e.g., SEQ ID NO: 32, 41, 50, 54, 62, or variants thereof), a CD8 signal sequence (e.g., SEQ ID NO: 2, 8, 14, 20, 70, 76, or variants thereof), and related sequences, can be selected for use in making constructs of the invention, as will be apparent to those of skill in the art.

### Nucleic Acids Encoding CARs

Also provided are nucleic acid constructs and vectors encoding (i) a CAR polypeptide (e.g., of SEQ ID NO: 1, 7, 13, 69, 75, or 100) or (ii) a polyprotein including a CAR polypeptide and a therapeutic agent (e.g., of SEQ ID NO: 19, 26, 35, 44, 53, or 61) described herein for use in generating CAR T cells. In various examples, the invention provides constructs that each include separate coding sequences for multiple proteins to be expressed in a CAR T cell of the invention. These separate coding sequences can be separated from one another by a cleavable linker sequence as described herein. For example, sequences encoding viral 2A proteins (e.g., T2A and P2A) can be placed between the separate genes and, when transcribed, can direct cleavage of the generated polyprotein. As noted above, constructs and vectors of the invention can include any of a number of different combinations of sequences. For example, a construct or vector of the invention can include sequences encoding one a CAR as described herein, optionally in combination with a therapeutic agent (e.g., an antibody reagent (e.g., a single chain antibody, a single domain antibody (e.g., a camelid), or a bispecific antibody (e.g., a BiTE)) or a cytokine) as described herein.

Efficient expression of proteins in CAR T cells as described herein can be assessed using standard assays that detect the mRNA, DNA, or gene product of the nucleic acid encoding the proteins. For example, RT-PCR, FACS, northern blotting, western blotting, ELISA, or immunohistochemistry can be used. The proteins described herein can be constitutively expressed or inducibly expressed. In some examples, the proteins are encoded by a recombinant nucleic acid sequence. For example, the invention provides a vector that includes a first polynucleotide sequence encoding a CAR, wherein the CAR includes an extracellular domain including an antigen-binding sequence that binds to, e.g., a tumor antigen or a Treg-associated antigen, and, optionally, a second polynucleotide sequence encoding a therapeutic agent (e.g., an antibody reagent (e.g., a single chain antibody, a single domain antibody (e.g., a camelid), or a bispecific antibody (e.g., a BiTE)) or a cytokine).

In some embodiments, the first polynucleotide sequence and the second polynucleotide sequence are each operably linked to a promoter. In some embodiments, the first polynucleotide sequence is operably linked to a first promoter and the second polynucleotide sequence is operably linked to a second promoter. The promoter can be a constitutively expressed promoter (e.g., an EF1α promoter) or an inducibly expressed promoter (e.g., a NFAT promoter).

In some embodiments, expression of the CAR and therapeutic agent are driven by the same promoter, e.g., a constitutively expressed promoter (e.g., an EF1α promoter). In other embodiments, expression of the CAR and therapeutic agent are driven by different promoters. For instance, expression of the CAR can be driven by a constitutively expressed promoter (e.g., an EF1α promoter) while expression of the therapeutic agent can be driven by an inducibly expressed promoter (e.g., a NFAT promoter). The polynucleotide sequence encoding the CAR can be located upstream of the polynucleotide sequence encoding the therapeutic agent, or the polynucleotide sequence encoding the therapeutic agent can be located upstream the polynucleotide sequence encoding the CAR.

Furthermore, the polynucleotides of the invention can include the expression of a suicide gene. This can be done to facilitate external, drug-mediated control of administered cells. For example, by use of a suicide gene, modified cells can be depleted from the patient in case of, e.g., an adverse event. In one example, the FK506 binding domain is fused to the caspase9 pro-apoptotic molecule. T cells engineered in this manner are rendered sensitive to the immunosuppressive drug tacrolimus. Other examples of suicide genes are thymidine kinase (TK), CD20, thymidylate kinase, truncated prostate-specific membrane antigen (PSMA), truncated low affinity nerve growth factor receptor (LNGFR), truncated CD19, and modified Fas, which can be triggered for conditional ablation by the administration of specific molecules (e.g., ganciclovir to TK+ cells) or antibodies or antibody-drug conjugates.

Constructs including sequences encoding proteins for expression in the CAR T cells of the invention can be included within vectors. In various examples, the vectors are retroviral vectors. Retroviruses, such as lentiviruses, provide a convenient platform for delivery of nucleic acid sequences encoding a gene, or chimeric gene of interest. A selected nucleic acid sequence can be inserted into a vector and packaged in retroviral particles using techniques known in the art. The recombinant virus can then be isolated and delivered to cells, e.g., in vitro or ex vivo. Retroviral systems are well known in the art and are described in, for example, U.S. Pat. No. 5,219,740; Kurth and Bannert (2010) “Retroviruses: Molecular Biology, Genomics and Pathogenesis” Calster Academic Press (ISBN:978-1-90455-55-4); and Hu and Pathak Pharmacological Reviews 2000 52:493-512; which are incorporated by reference herein in their entirety. Lentiviral system for efficient DNA delivery can be purchased from OriGene; Rockville, Md. In various embodiments, the protein is expressed in the T cell by transfection or electroporation of an expression vector including nucleic acid encoding the protein using vectors and methods that are known in the art. In some embodiments, the vector is a viral vector or a non-viral vector. In some embodiments, the viral vector is a retroviral vector (e.g., a lentiviral vector), an adenovirus vector, or an adeno-associated virus vector.

The invention also provides a composition that includes a vector that includes a first polynucleotide sequence encoding a CAR, wherein the CAR includes an extracellular domain including a sequence that specifically binds to a tumor antigen or a Treg-associated antigen, and, optionally, a second polynucleotide sequence encoding a therapeutic agent. In certain embodiments, when the therapeutic agent is an antibody reagent (e.g., a single chain antibody, a single domain antibody (e.g., a camelid), or a bispecific antibody (e.g., a BiTE)), the antibody reagent specifically binds to a tumor antigen or a Treg-associated antigen.

### Cells and Therapy

One aspect of the technology described herein relates to a mammalian cell including any of the CAR polypeptides described herein (optionally together with another therapeutic agent (e.g., an antibody reagent (e.g., a scFv, a camelid antibody, or a BiTE) or a cytokine)); or a nucleic acid encoding any of the CAR polypeptides described herein (optionally together with another therapeutic agent (e.g., an antibody reagent (e.g., a scFv, a camelid antibody, or a cytokine)). In one embodiment, the mammalian cell includes an antibody, antibody reagent, antigen-binding portion thereof, any of the CARs described herein, or a cytokine, or a nucleic acid encoding such an antibody, antibody reagent, antigen-binding portion thereof, any of the CARs described herein, or a cytokine. The mammalian cell or tissue can be of human, primate, hamster, rabbit, rodent, cow, pig, sheep, horse, goat, dog or cat origin, but any other mammalian cell may be used. In a preferred embodiment of any aspect, the mammalian cell is human.

In some embodiments of any aspect, the mammalian cell is an immune cell. As used herein, “immune cell” refers to a cell that plays a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes. In some embodiments, the immune cell is a T cell; a NK cell; a NKT cell; lymphocytes, such as B cells and T cells; and myeloid cells, such as monocytes, macrophages, eosinophils, mast cells, basophils, and granulocytes. In one embodiment, the immune cell is a T cell.

In other embodiments, the immune cell is obtained from an individual having or diagnosed as having cancer, a plasma cell disorder, or autoimmune disease.

Cluster of differentiation (CD) molecules are cell surface markers present on leukocytes. As a leukocyte differentiates and matures its CD profile changes. In the case that a leukocytes turns into a cancer cell (i.e., a lymphoma), its CD profile is important in diagnosing the disease. The treatment and prognosis of certain types of cancers is reliant on determining the CD profile of the cancer cell. “CDX+”, wherein “X” is a CD marker, indicates the CD marker is present in the cancer cell, while “CDX−” indicates the marker is not present. One skilled in the art will be capable of assessing the CD molecules present on a cancer cell using standard techniques, for example, using immunofluorescence to detect commercially available antibodies bound to the CD molecules.

In some embodiments, the immune cells (e.g., T cells) including a CAR, such as a CART.BiTE described herein, can be used to treat cancer, e.g., lymphoma, myeloma, or a solid tumor, e.g., glioblastoma, prostate cancer, lung cancer, or pancreatic cancer. In some embodiments, the CART.BiTEs described herein, e.g., a CART-EGFRvIII.BiTE-EGFR, can be used to treat a glioblastoma having reduced EGFRvIII expression.

In further embodiments, the immune cells (e.g., T cells) including a CAR, such as a CART.BiTE described herein, can be used to prevent or reduce immunosuppression due to, e.g., Tregs, in the tumor microenvironment. Furthermore, such CART.BiTEs are useful for preventing or reducing T cell exhaustion in the tumor microenvironment.

The immune cells (e.g., T cells) including a CAR, such as a CART.BiTE described herein, can also be used to treat a cancer having heterogeneous antigen expression. For instance, the CAR component of the CART.BiTE construct can include an extracellular target binding domain that binds to one antigen expressed by the cancer, while the BiTE component of the CART.BiTE construct can bind a second antigen expressed by the cancer in addition to a T cell antigen (e.g., CD3).

“Cancer” as used herein can refer to a hyperproliferation of cells whose unique trait, loss of normal cellular control, results in unregulated growth, lack of differentiation, local tissue invasion, and metastasis. Exemplary cancers include, but are not limited to, glioblastoma, prostate cancer, glioma, leukemia, lymphoma, multiple myeloma, or a solid tumor, e.g., lung cancer and pancreatic cancer. Non-limiting examples of leukemia include acute myeloid leukemia (AML), chronic myeloid leukemia (CML), acute lymphocytic leukemia (ALL), and chronic lymphocytic leukemia (CLL). In one embodiment, the cancer is ALL or CLL. Non-limiting examples of lymphoma include diffuse large B-cell lymphoma (DLBCL), follicular lymphoma, small lymphocytic lymphoma (SLL), mantle cell lymphoma (MCL), marginal zone lymphomas, Burkitt's lymphoma, hairy cell leukemia (HCL), and T cell lymphoma (e.g., peripheral T cell lymphoma (PTCL), including cutaneous T cell lymphoma (CTCL) and anaplastic large cell lymphoma (ALCL)). In one embodiment, the cancer is DLBCL or follicular lymphoma. Non-limiting examples of solid tumors include adrenocortical tumor, alveolar soft part sarcoma, carcinoma, chondrosarcoma, colorectal carcinoma, desmoid tumors, desmoplastic small round cell tumor, endocrine tumors, endodermal sinus tumor, epithelioid hemangioendothelioma, Ewing sarcoma, germ cell tumors (solid tumor), giant cell tumor of bone and soft tissue, hepatoblastoma, hepatocellular carcinoma, melanoma, nephroma, neuroblastoma, non-rhabdomyosarcoma soft tissue sarcoma (NRSTS), osteosarcoma, paraspinal sarcoma, renal cell carcinoma, retinoblastoma, rhabdomyosarcoma, synovial sarcoma, and Wilms tumor. Solid tumors can be found in bones, muscles, or organs, and can be sarcomas or carcinomas. It is contemplated that any aspect of the technology described herein can be used to treat all types of cancers, including cancers not listed in the instant application. As used herein, the term “tumor” refers to an abnormal growth of cells or tissues, e.g., of malignant type or benign type.

As used herein, an “autoimmune disease or disorder” is characterized by the inability of one's immune system to distinguish between a foreign cell and a healthy cell. This results in one's immune system targeting one's healthy cells for programmed cell death. Non-limiting examples of an autoimmune disease or disorder include inflammatory arthritis, type 1 diabetes mellitus, multiples sclerosis, psoriasis, inflammatory bowel diseases, SLE, and vasculitis, allergic inflammation, such as allergic asthma, atopic dermatitis, and contact hypersensitivity. Other examples of auto-immune-related disease or disorder, but should not be construed to be limited to, include rheumatoid arthritis, multiple sclerosis (MS), systemic lupus erythematosus, Graves' disease (overactive thyroid), Hashimoto's thyroiditis (underactive thyroid), celiac disease, Crohn's disease and ulcerative colitis, Guillain-Barre syndrome, primary biliary sclerosis/cirrhosis, sclerosing cholangitis, autoimmune hepatitis, Raynaud's phenomenon, scleroderma, Sjogren's syndrome, Goodpasture's syndrome, Wegener's granulomatosis, polymyalgia rheumatica, temporal arteritis/giant cell arteritis, chronic fatigue syndrome CFS), psoriasis, autoimmune Addison's Disease, ankylosing spondylitis, acute disseminated encephalomyelitis, antiphospholipid antibody syndrome, aplastic anemia, idiopathic thrombocytopenic purpura, myasthenia gravis, opsoclonus myoclonus syndrome, optic neuritis, Ord's thyroiditis, pemphigus, pernicious anaemia, polyarthritis in dogs, Reiter's syndrome, Takayasu's arteritis, warm autoimmune hemolytic anemia, Wegener's granulomatosis and fibromyalgia (FM).

In one embodiment, the mammalian cell is obtained for a patient having an immune system disorder that results in abnormally low activity of the immune system, or immune deficiency disorders, which hinders one's ability to fight a foreign agent (e.g., a virus or bacterial cell).

A plasma cell is a white blood cell produces from B lymphocytes which function to generate and release antibodies needed to fight infections. As used herein, a “plasma cell disorder or disease” is characterized by abnormal multiplication of a plasma cell. Abnormal plasma cells are capable of “crowding out” healthy plasma cells, which results in a decreased capacity to fight a foreign object, such as a virus or bacterial cell. Non-limiting examples of plasma cell disorders include amyloidosis, Waldenstrom's macroglobulinemia, osteosclerotic myeloma (POEMS syndrome), monoclonal gammopathy of unknown significance (MGUS), and plasma cell myeloma.

A mammalian cell, e.g., a T cell, can be engineered to include any of the CAR polypeptides described herein (including CAR polypeptides that are cleavably linked to antibody reagents or cytokines, as described herein); or a nucleic acid encoding any of the CAR polypeptides (and optionally also a genetically encoded antibody reagent or cytokine) described herein. T cells can be obtained from a subject using standard techniques known in the field. For example, T cells can be isolated from peripheral blood taken from a donor or patient. T cells can be isolated from a mammal. Preferably, T cells are isolated from a human.

In some embodiments of any aspect, any of the CAR polypeptides (optionally together with an antibody reagent as described herein or a cytokine) described herein are expressed from a lentiviral vector. The lentiviral vector is used to express the CAR polypeptide (and optionally also the antibody reagent or cytokine) in a cell using infection standard techniques.

Retroviruses, such as lentiviruses, provide a convenient platform for delivery of nucleic acid sequences encoding a gene or chimeric gene of interest. A selected nucleic acid sequence can be inserted into a vector and packaged in retroviral particles using techniques known in the art. The recombinant virus can then be isolated and delivered to cells, e.g., in vitro or ex vivo. Retroviral systems are well known in the art and are described in, for example, U.S. Pat. No. 5,219,740; Kurth and Bannert (2010) “Retroviruses: Molecular Biology, Genomics and Pathogenesis” Calster Academic Press (ISBN:978-1-90455-55-4); and Hu et al., Pharmacological Reviews 52:493-512, 2000; which are each incorporated by reference herein in their entirety. Lentiviral system for efficient DNA delivery can be purchased from OriGene; Rockville, Md. In some embodiments, the CAR polypeptide (and optionally the antibody reagent or cytokine) of any of the CARs described herein is expressed in a mammalian cell via transfection or electroporation of an expression vector including a nucleic acid encoding the CAR. Transfection or electroporation methods are known in the art.

Efficient expression of the CAR polypeptide (and optionally the antibody reagent or cytokine) of any of the polypeptides described herein can be assessed using standard assays that detect the mRNA, DNA, or gene product of the nucleic acid encoding the CAR (and optional antibody reagent or cytokine), such as RT-PCR, FACS, northern blotting, western blotting, ELISA, or immunohistochemistry.

In some embodiments, the CAR polypeptide (and optional antibody reagent or cytokine) described herein is constitutively expressed. In other embodiments, the CAR polypeptide is constitutively expressed and the optional antibody reagent or cytokine is inducibly expressed. In some embodiments, the CAR polypeptide (and optional antibody reagent or cytokine) described herein is encoded by recombinant nucleic acid sequence.

One aspect of the technology described herein relates to a method of treating cancer, a plasma cell disorder, or an autoimmune disease in a subject in need thereof, the method including: engineering a T cell to include any of the CAR polypeptides (and optional antibody reagents or cytokines) described herein on the T cell surface; and administering the engineered T cell to the subject. In the case of cancer, the method can be for treating diagnosed cancer, preventing recurrence of cancer, or for use in an adjuvant or neoadjuvant setting.

One aspect of the technology described herein relates to a method of treating cancer, a plasma cell disorder, or an autoimmune disease in a subject in need thereof, the method including: administering the cell of any of the mammalian cells including the any of the CAR polypeptides (and optional antibody reagents or cytokines) described herein.

In some embodiments of any of aspect, the engineered CAR-T cell is stimulated and/or activated prior to administration to the subject.

### Administration

In some embodiments, the methods described herein relate to treating a subject having or diagnosed as having cancer, a plasma cell disease or disorder, or an autoimmune disease or disorder with a mammalian cell including any of the CAR polypeptides (and optional antibody reagents or cytokines) described herein, or a nucleic acid encoding any of the CAR polypeptides (and optional antibody reagents or cytokines) described herein. The CAR T cells described herein include mammalian cells including any of the CAR polypeptides (and optional antibody reagents or cytokines) described herein, or a nucleic acid encoding any of the CAR polypeptides (and optional antibody reagents or cytokines) described herein. As used herein, a “condition” refers to a cancer, a plasma cell disease or disorder, or an autoimmune disease or disorder. Subjects having a condition can be identified by a physician using current methods of diagnosing the condition. Symptoms and/or complications of the condition, which characterize these conditions and aid in diagnosis are well known in the art and include but are not limited to, fatigue, persistent infections, and persistent bleeding. Tests that may aid in a diagnosis of, e.g., the condition, but are not limited to, blood screening and bone marrow testing, and are known in the art for a given condition. A family history for a condition, or exposure to risk factors for a condition can also aid in determining if a subject is likely to have the condition or in making a diagnosis of the condition.

The compositions described herein can be administered to a subject having or diagnosed as having a condition. In some embodiments, the methods described herein include administering an effective amount of activated CAR T cells described herein to a subject in order to alleviate a symptom of the condition. As used herein, “alleviating a symptom of the condition” is ameliorating any condition or symptom associated with the condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. In one embodiment, the compositions described herein are administered systemically or locally. In a preferred embodiment, the compositions described herein are administered intravenously. In another embodiment, the compositions described herein are administered at the site of a tumor.

The term “effective amount” as used herein refers to the amount of activated CAR T cells needed to alleviate at least one or more symptom of the disease or disorder, and relates to a sufficient amount of the cell preparation or composition to provide the desired effect. The term “therapeutically effective amount” therefore refers to an amount of activated CAR T cells that is sufficient to provide a particular anti-condition effect when administered to a typical subject. An effective amount as used herein, in various contexts, would also include an amount sufficient to delay the development of a symptom of the disease, alter the course of a symptom disease (for example but not limited to, slowing the progression of a condition), or reverse a symptom of the condition. Thus, it is not generally practicable to specify an exact “effective amount.” However, for any given case, an appropriate “effective amount” can be determined by one of ordinary skill in the art using only routine experimentation.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of activated CAR T cells, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for bone marrow testing, among others. The dosage can be determined by a physician and adjusted, as necessary, to suit observed effects of the treatment.

In one aspect of the technology, the technology described herein relates to a pharmaceutical composition including activated CAR T cells as described herein, and optionally a pharmaceutically acceptable carrier. The active ingredients of the pharmaceutical composition at a minimum include activated CAR T cells as described herein. In some embodiments, the active ingredients of the pharmaceutical composition consist essentially of activated CAR T cells as described herein. In some embodiments, the active ingredients of the pharmaceutical composition consist of activated CAR T cells as described herein. Pharmaceutically acceptable carriers for cell-based therapeutic formulation include saline and aqueous buffer solutions, Ringer's solution, and serum component, such as serum albumin, HDL and LDL. The terms such as “excipient,” “carrier,” “pharmaceutically acceptable carrier” or the like are used interchangeably herein.

In some embodiments, the pharmaceutical composition including activated CAR T cells as described herein can be a parenteral dose form. Since administration of parenteral dosage forms typically bypasses the patient's natural defenses against contaminants, the components apart from the CAR T cells themselves are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. Any of these can be added to the activated CAR T cells preparation prior to administration.

Suitable vehicles that can be used to provide parenteral dosage forms of activated CAR T cells as disclosed within are well known to those skilled in the art. Examples include, without limitation: saline solution; glucose solution; aqueous vehicles including but not limited to, sodium chloride injection, Ringer's injection, dextrose injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

### Dosage

“Unit dosage form” as the term is used herein refers to a dosage for suitable one administration. By way of example, a unit dosage form can be an amount of therapeutic disposed in a delivery device, e.g., a syringe or intravenous drip bag. In one embodiment, a unit dosage form is administered in a single administration. In another, embodiment more than one unit dosage form can be administered simultaneously.

In some embodiments, the activated CAR T cells described herein are administered as a monotherapy, i.e., another treatment for the condition is not concurrently administered to the subject. A pharmaceutical composition including the T cells described herein can generally be administered at a dosage of 10⁴ to 10⁹ cells/kg body weight, in some instances 10⁵ to 10⁶ cells/kg body weight, including all integer values within those ranges. If necessary, T cell compositions can also be administered multiple times at these dosages. The cells can be administered by using infusion techniques that are commonly known in immunotherapy (see, e.g., Rosenberg et al., New Eng. J. Med. 319:1676, 1988).

In certain aspects, it may be desired to administer activated CAR T cells to a subject and then subsequently redraw blood (or have an apheresis performed), activate T cells therefrom as described herein, and reinfuse the patient with these activated and expanded T cells. This process can be carried out multiple times every few weeks. In certain aspects, T cells can be activated from blood draws of from 10 cc to 400 cc. In certain aspects, T cells are activated from blood draws of 20 cc, 30 cc, 40 cc, 50 cc, 60 cc, 70 cc, 80 cc, 90 cc, or 100 cc.

Modes of administration can include, for example intravenous (i.v.) injection or infusion. The compositions described herein can be administered to a patient transarterially, intratumorally, intranodally, or intramedullary. In some embodiments, the compositions of T cells may be injected directly into a tumor, lymph node, or site of infection. In one embodiment, the compositions described herein are administered into a body cavity or body fluid (e.g., ascites, pleural fluid, peritoneal fluid, or cerebrospinal fluid).

In a particular exemplary aspect, subjects may undergo leukapheresis, wherein leukocytes are collected, enriched, or depleted ex vivo to select and/or isolate the cells of interest, e.g., T cells. These T cell isolates can be expanded by contact with an artificial APC, e.g., an aAPC expressing anti-CD28 and anti-CD3 CDRs, and treated such that one or more CAR constructs of the technology may be introduced, thereby creating a CAR T cell. Subjects in need thereof can subsequently undergo standard treatment with high dose chemotherapy followed by peripheral blood stem cell transplantation. Following or concurrent with the transplant, subjects can receive an infusion of the expanded CAR T cells. In one embodiment, expanded cells are administered before or following surgery.

In some embodiments, lymphodepletion is performed on a subject prior to administering one or more CAR T cell as described herein. In such embodiments, the lymphodepletion can include administering one or more of melphalan, cytoxan, cyclophosphamide, and fludarabine.

The dosage of the above treatments to be administered to a patient will vary with the precise nature of the condition being treated and the recipient of the treatment. The scaling of dosages for human administration can be performed according to art-accepted practices.

In some embodiments, a single treatment regimen is required. In others, administration of one or more subsequent doses or treatment regimens can be performed. For example, after treatment biweekly for three months, treatment can be repeated once per month, for six months or a year or longer. In some embodiments, no additional treatments are administered following the initial treatment.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

### Combination Therapy

The activated CAR T cells described herein can optionally be used in combination with each other and with other known agents and therapies, as can determined to be appropriate by those of skill in the art. In one example, two or more CAR T cells targeting different Treg markers (e.g., GARP, LAP, etc.) can be administered in combination. In another example, two or more CAR T cells targeting different cancer antigens are administered in combination. In a further example, one or more CAR T cell targeting a Treg marker (e.g., GARP, LAP, etc.) and one or more CAR T cell targeting one or more tumor antigens are administered in combination.

Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The activated CAR T cells described herein and the at least one additional therapeutic agent can be administered simultaneously, in the same or in separate compositions, or sequentially. For sequential administration, the CAR-expressing cell described herein can be administered first, and the additional agent can be administered second, or the order of administration can be reversed. The CAR T therapy and/or other therapeutic agents, procedures or modalities can be administered during periods of active disorder, or during a period of remission or less active disease. The CAR T therapy can be administered before another treatment, concurrently with the treatment, post-treatment, or during remission of the disorder.

When administered in combination, the activated CAR T cells and the additional agent (e.g., second or third agent), or all, can be administered in an amount or dose that is higher, lower or the same as the amount or dosage of each agent used individually, e.g., as a monotherapy. In certain embodiments, the administered amount or dosage of the activated CAR T cells, the additional agent (e.g., second or third agent), or all, is lower (e.g., at least 20%, at least 30%, at least 40%, or at least 50%) than the amount or dosage of each agent used individually. In other embodiments, the amount or dosage of the activated CAR T cells, the additional agent (e.g., second or third agent), or all, that results in a desired effect (e.g., treatment of cancer) is lower (e.g., at least 20%, at least 30%, at least 40%, or at least 50% lower) than the amount or dosage of each agent individually required to achieve the same therapeutic effect. In further embodiments, the activated CAR T cells described herein can be used in a treatment regimen in combination with surgery, chemotherapy, radiation, an mTOR pathway inhibitor, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, rapamycin, mycophenolic acid, steroids, FR901228, cytokines, or a peptide vaccine, such as that described in Izumoto et al., J. Neurosurg. 108:963-971, 2008.

In one embodiment, the activated CAR T cells described herein can be used in combination with a checkpoint inhibitor. Exemplary checkpoint inhibitors include anti-PD-1 inhibitors (Nivolumab, MK-3475, Pembrolizumab, Pidilizumab, AMP-224, AMP-514), anti-CTLA4 inhibitors (Ipilimumab and Tremelimumab), anti-PDL1 inhibitors (Atezolizumab, Avelomab, MSB0010718C, MED14736, and MPDL3280A), and anti-TIM3 inhibitors.

In one embodiment, the activated CAR T cells described herein can be used in combination with a chemotherapeutic agent. Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide). General chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®). Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HC1 (Treanda®). Exemplary mTOR inhibitors include, e.g., temsirolimus; ridaforolimus (formally known as deferolimus, (IR,2R,45)-4-[(2R)-2 [1R,95,125,15R,16E,18R,19R,21R,235,24E,26E,28Z,305,325,35R)-I,18-dihydroxy-19,30-dimethoxy-15,17,21,23, 29,35-hexamethyl-2,3,10,14,20-pentaoxo-11,36-dioxa-4-azatricyclo[30.3.1.04′9] hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RADOOI); rapamycin (AY22989, Sirolimus®); simapimod (CAS 164301-51-3); emsirolimus, (5-{2,4-Bis[(35,)-3-methylmorpholin-4-yl]pyrido[2,3-(i]pyrimidin-7-yl}-2-methoxyphenyl)methanol (AZD8055); 2-Amino-8-[iraw5,-4-(2-hydroxyethoxy)cyclohexyl]-6-(6-methoxy-3-pyridinyl)-4-methyl-pyrido[2,3-JJpyrimidin-7(8H)-one (PF04691502, CAS 1013101-36-4); and N2-[1,4-dioxo-4-[[4-(4-oxo-8-phenyl-4H-I-benzopyran-2-yl)morpholinium-4-yl]methoxy]butyl]L-arginylglycyl-L-a-aspartylL-serine-, inner salt (SF1126, CAS 936487-67-1), and XL765. Exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon γ, CAS 951209-71-5, available from IRX Therapeutics). Exemplary anthracyclines include, e.g., doxorubicin (Adriamycin® and Rubex®); bleomycin (Ienoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin. Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®). Exemplary proteosome inhibitors include bortezomib (Velcade®); carfilzomib (PX-171-007, (5)-4-Methyl-N-((5)-1-(((5)-4-methyl-1-((R)-2-methyloxiran-2-yl)-1-oxopentan-2-yl)amino)-I-oxo-3-phenylpropan-2-yl)-2-((5,)-2-(2-morpholinoacetamido)-4-phenylbutanamido)-pentanamide); marizomib (NPT0052); ixazomib citrate (MLN-9708); delanzomib (CEP-18770); and O-Methyl-N-[(2-methyl-5-thiazolyl)carbonyl]-L-seryl-O-methyl-N-[(IIS′)-2-[(2R)-2-methyl-2-oxiranyl]-2-oxo-1-(phenylmethyl)ethyl]-L-serinamide (ONX-0912).

One of skill in the art can readily identify a chemotherapeutic agent of use (e.g., see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chapters 28-29 in Abeloff's Clinical Oncology, 2013 Elsevier; and Fischer D. S. (ed.): The Cancer Chemotherapy Handbook, 4th ed. St. Louis, Mosby-Year Book, 2003).

In an embodiment, activated CAR T cells described herein are administered to a subject in combination with a molecule that decreases the level and/or activity of a molecule targeting GITR and/or modulating GITR functions, a molecule that decreases the Treg cell population, an mTOR inhibitor, a GITR agonist, a kinase inhibitor, a non-receptor tyrosine kinase inhibitor, a CDK4 inhibitor, and/or a BTK inhibitor.

### Efficacy

The efficacy of activated CAR T cells in, e.g., the treatment of a condition described herein, or to induce a response as described herein (e.g., a reduction in cancer cells) can be determined by the skilled clinician. However, a treatment is considered “effective treatment,” as the term is used herein, if one or more of the signs or symptoms of a condition described herein is altered in a beneficial manner, other clinically accepted symptoms are improved, or even ameliorated, or a desired response is induced, e.g., by at least 10% following treatment according to the methods described herein. Efficacy can be assessed, for example, by measuring a marker, indicator, symptom, and/or the incidence of a condition treated according to the methods described herein or any other measurable parameter appropriate. Treatment according to the methods described herein can reduce levels of a marker or symptom of a condition, e.g. by at least 10%, at least 15%, at least 20%, at least 25%, at least 30%, at least 40%, at least 50%, at least 60%, at least 70%, at least 80% or at least 90% or more.

Efficacy can also be measured by a failure of an individual to worsen as assessed by hospitalization, or need for medical interventions (i.e., progression of the disease is halted). Methods of measuring these indicators are known to those of skill in the art and/or are described herein.
Treatment includes any treatment of a disease in an individual or an animal (some non-limiting examples include a human or an animal) and includes: (1) inhibiting the disease, e.g., preventing a worsening of symptoms (e.g., pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy of a given approach can be assessed in animal models of a condition described herein. When using an experimental animal model, efficacy of treatment is evidenced when a statistically significant change in a marker is observed.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior technology or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples, which in no way should be construed as being further limiting.

### EXAMPLES

The following are examples of the methods and compositions of the invention. It is understood that various other embodiments may be practiced, given the general description provided above.

### Example 1. CAR T Cell Mediated Secretion of Toxic Drugs to Modify the Tumor Microenvironment and Enhance CAR T Cell Potency

CAR-modified T cells can be used to deliver otherwise toxic antibodies to the tumor microenvironment. In this example, T cells are genetically modified to secrete an antibody or cytokine with the goal of modifying the inhibitory immune cell milieu of the tumor microenvironment.

Specifically, CAR T cells directed to an antigen that is heterogeneously expressed can have their potency enhanced by enabling activation of surrounding tumor infiltrating lymphocytes in the tumor microenvironment. Specific, non-limiting examples, include:

(1) genetically-encoded anti-CTLA4 CAR-T cell mediated secretion. Anti-CTLA4 checkpoint blockade can cause toxicity when delivered systemically. However, localized secretion of anti-CTLA4 is expected to provide checkpoint blockade and deplete regulatory T cells in the tumor microenvironment.

(2) genetically-encoded anti-CD25 (e.g., daclizumab) to deplete Tregs in the local tumor microenvironment. CAR T cells have been shown to traffic to tumors despite hostile environment and the blood brain barrier. New T cell immigrants also infiltrate tumors, but these are hypothesized to be inhibited by checkpoints and activation of Tregs. Localized secretion of anti-CD25 is expected to deplete Tregs. However, daclizumab given pharmacologically is toxic when administered systemically.

(3) genetically-encoded anti-EGFR (e.g., cetuximab). CAR T cells directed to a safe but heterogeneously expressed antigen (e.g., EGFRvIII) do not completely eliminate tumor if the antigen is heterogeneously expressed. However, other antigens may be expressed at high levels in the tumor microenvironment (e.g., EGFR). EGFR is expressed only in brain tumors within the brain, but it is expressed in many other epithelial tissues, which makes it an unsafe target for CAR T cells. However, CAR T cells directed to EGFRvIII could be engineered to secrete anti-EGFR such that only tissues in the tumor microenvironment where CAR T cells traffic to are exposed to high levels of anti-EGFR. In the case of anti-EGFR, the antibody is not expected to be severely toxic, given that it is used systemically in other cancers, such as head and neck cancer and colon cancer. However, it does not penetrate the CNS, and so is not efficacious in brain tumors. Genetically-encoded anti-EGFR in the form of a CAR T cell directed to the CNS has the capacity to use the T cells as the vehicle for localized delivery of anti-EGFR to the CNS and brain tumors, such as glioblastoma.

Thus, provided is genetically-encoded Treg depletion in the tumor microenvironment with two different formats. In addition, genetically-encoded delivery of antibodies that cannot get into certain tissues, and could enhance the potency of T cell therapies by broadening the specificity of the anti-tumor target. Accordingly, described is gene-modified T cell therapy for cancer.

### Example 2. Engineered CAR T Cells Overcome Tumor Heterogeneity and Immunosuppression in Glioblastoma

### Materials and Methods

T cells from leukapheresis products obtained from deidentified healthy donors were stimulated with Dynabeads Human T-Activator CD3/CD28 at a bead to cell ratio of 3:1 and cultured in complete RPMI 1640 medium. 10 days following stimulation and lentivirus transduction, cells were frozen and stored for use in functional assays. The ability of CAR T cells to kill target cells was tested in a 20-hour luciferase-based assay. Treg suppression was visualized by IncuCyte live cell analysis.

For in vivo experiments, tumor cells were collected in logarithmic growth phase, washed and loaded into a 50 microliter Hamilton syringe. The needle was positioned using a stereotactic frame at 2 mm to the right of the bregma and 4 mm below the surface of the skull at the coronal suture. For treatment, mice were infused once with CAR T cells (1×10⁶ CAR-transduced T cells per mouse) via tail vein.

### Results

EGFRvIII CAR T Cells Mediate Antitumor Activity In Vitro.

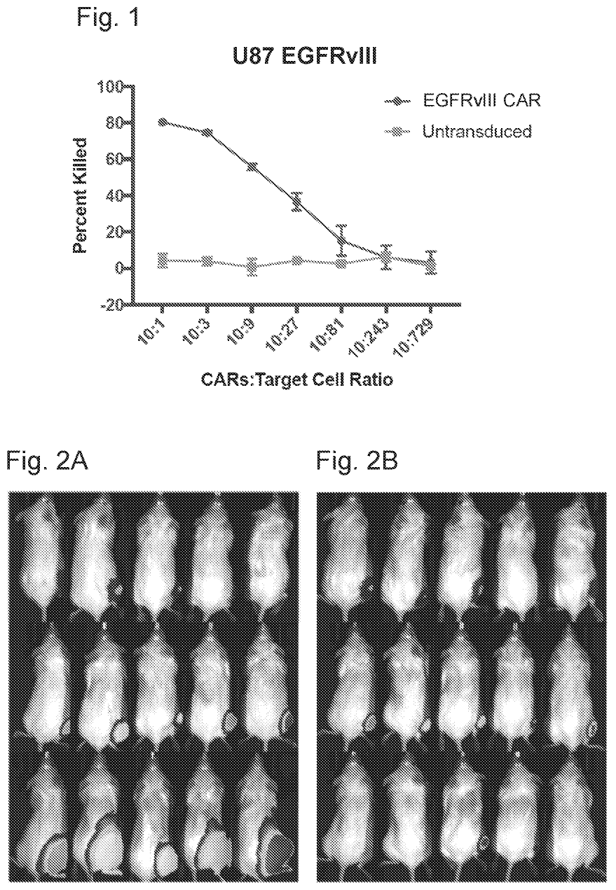

An EGFRvIII CAR was designed and synthesized, which was used with initial tests in vitro. In vitro characterization of this CAR demonstrates that the EGFRvIII CAR mediates significant and specific cytotoxicity against the human glioma U87vIII cell line (FIG. 1; EGFRvIII CAR transduced T cells potently and specifically mediate cytotoxicity against the U87vIII human glioma cell line). This effect was observed in a subcutaneous models of human GBM xenograft, where even established, bulky tumors responded to CART-EGFRvIII (FIGS. 2A and 2B; CART-EGFRvIII treats EGFRvIII expressing tumor (U87vIII) in a subcutaneous model of human glioma. Mice were treated with CART-EGFRvIII on day 4 after implantation (top row) with successful treatment by day 21 (bottom row). UTD, untransduced cells, serve as the negative control).

EGFRvIII CAR T cells mediate antitumor activity against EGFRvIII expressing tumors in the brain.

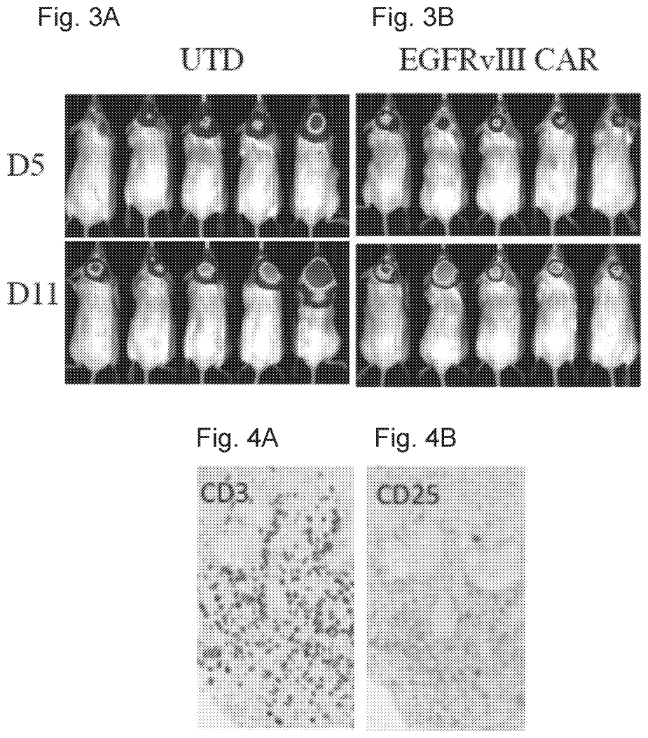

In a murine model of intracranial human glioma, EGFRvIII CART cells slowed the growth of tumors and led to prolonged survival (FIGS. 3A and 3B; CART-EGFRvIII slows growth of EGFRvIII expressing tumor (U87vIII) in an intracranial model of human glioma. Mice were treated with CART-EGFRvIII on day 2 after implantation). Although tumor growth was abrogated, the effects were not as pronounced as those observed against subcutaneous tumors. One critical barrier to translation of CAR T cells for patients with brain tumors has been the well-characterized infiltration of suppressive Tregs.

CAR T Cell Activity is Suppressed by Regulatory T Cells.

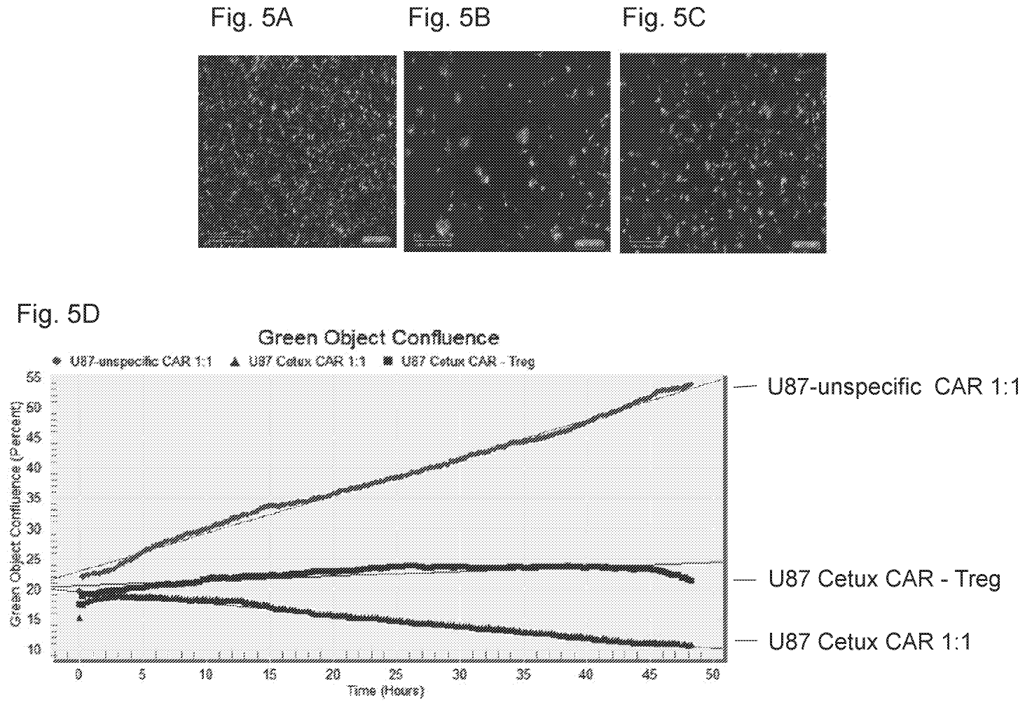

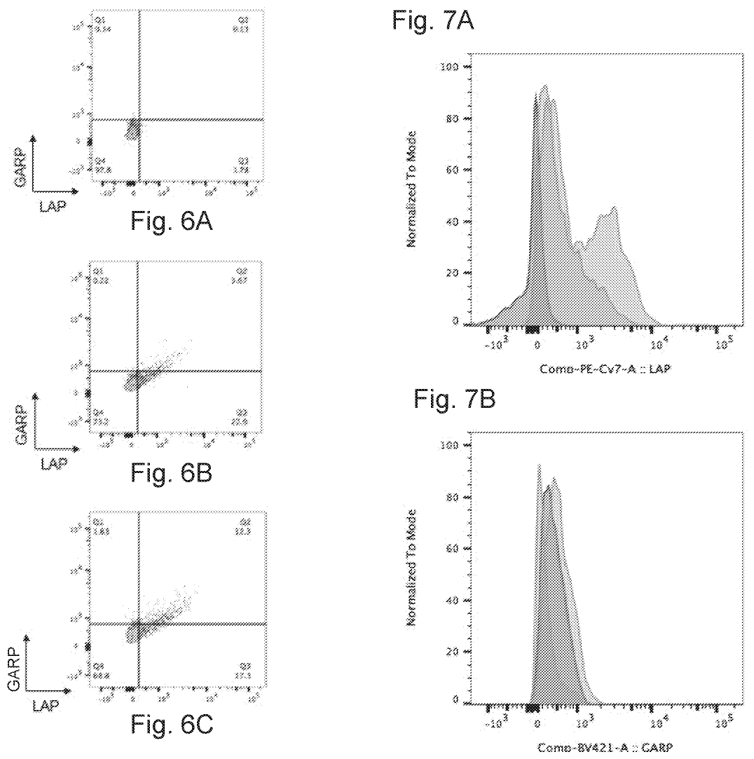

In coculture experiments with CAR T cells and target glioma cell lines, the presence of regulatory T cells was noted to abrogate antitumor activity of CAR T cells in vitro. FIGS. 5A-5D qualitatively (FIGS. 5A-5C) and quantitatively (FIG. 5D) demonstrate Treg suppression of CAR T cell antitumor activity after 18 hours of coincubation with human glioma cells in vitro. FIGS. 6A-6C show Tregs sorted from leukopak on CD4⁺CD25⁺CD127⁻ and expanded with CD3/CD28 beads for 7 days in the presence of IL-2. On Day 1, they were transduced to express GFP. After debeading on Day 7, expanded Tregs were rested for 4 days before freezing. After thaw, Tregs were stained for LAP and GARP expression after overnight rest (non-activated) or overnight activation with anti-CD3 and anti-CD28. Untransduced T cells (CD4+ and CD8+) from the same donor were used as controls for expression.

Anti-LAP CAR T Cells Kill Regulatory T Cells In Vitro.

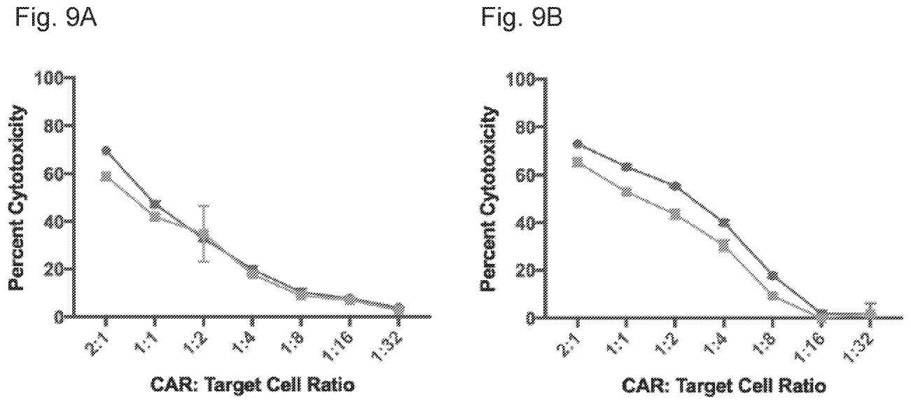

As shown in FIGS. 9A and 9B, CAR T cells co-cultured with isolated Tregs expanded from the same donor and transduced to express GFP. Tregs were activated overnight with anti-CD3 and anti-CD28 or rested overnight prior to the killing assay. 62,500 Tregs per well were plated. CARs were added at the ratio to Tregs labeled in the graph above. Cells were cultured for 3 days in the presence of 300 U/mL of IL-2. Flow ran on Day 3 with 30,000 events from each well. Percent cytotoxicity calculated as the percent of GFP+ cells missing compared to the untransduced T cell culture with Tregs.

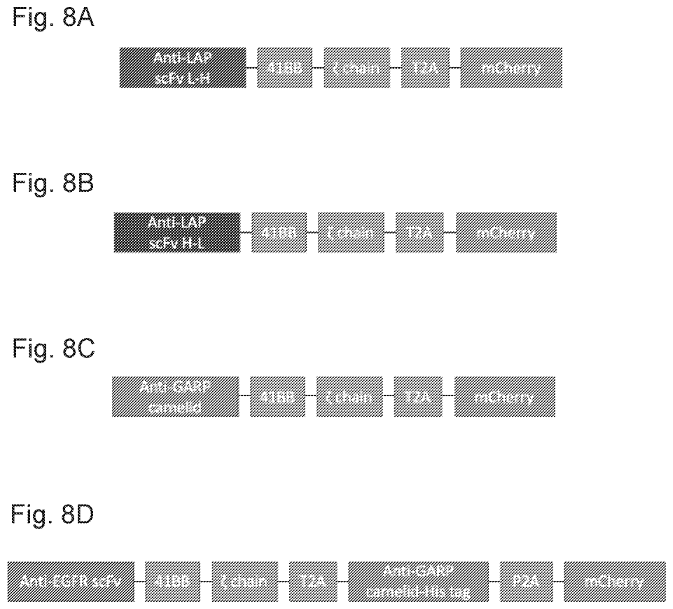

Based on these data, novel CAR constructs targeting surface markers found on Tregs were developed. The overall design of these CAR T cells is depicted in FIGS. 8A-8D.

Conclusion

The ultimate goal is to design, test, and improve CAR T cell therapy in preclinical murine models of human GBM. It has been demonstrated herein that CAR T cells can indeed mediate specific and potent effects against even bulky, established tumors in vivo. Additionally, it is shown that regulatory T cells may play a critical role in the suppression of these immune responses. New techniques that target Tregs may offer a way to modulate the local immune environment in order to enhance antitumor efficacy.

### Example 3. EGFRvIII-Targeted CAR T Cells

CAR T cells having an EGFRvIII antigen-binding moiety (e.g., CART-EGFRvIII cells) represent a promising cellular therapy for specific targeting of cytolytic cells to the tumor microenvironment, in part because EGFRvIII is specifically expressed on tumor tissue while generally absent from healthy tissue. In this example, CART-EGFRvIII cells were tested in vitro and in vivo in two animal models.

T cells from leukapheresis products obtained from deidentified healthy donors were stimulated with Dynabeads (Human T-Activator CD3/CD28) at a bead to cell ratio of 3:1 and cultured in complete RPMI 1640 medium. Ten days following stimulation and lentivirus transduction, cells were frozen and stored for use in functional assays.

Initial tests were performed in vitro to characterize the ability of CAR-EGFRvIII cells to preferentially kill tumor cells relative to untransduced control cells in a twenty-hour luciferase-based assay, shown in FIG. 1. U87vIII, a human glioma cell line, was used as target cells. In vitro characterization demonstrates that EGFRvIII CAR T cells mediate significant and specific cytotoxicity against U87vIII cells (FIG. 1).

For in vivo experiments, U87vIII tumor cells were collected in logarithmic growth phase, washed, and administered to mice subcutaneously in a xenograft model of human glioblastoma (FIGS. 2A and 2B) or intracranially in a model of human glioma (FIGS. 3A and 3B). For intracranial administrations, the needle of a 50 microliter Hamilton syringe was positioned using a stereotactic frame at 2 mm to the right of the bregma and 4 mm below the surface of the skull at the coronal suture. For treatment, mice were infused once with CAR T cells (1×10⁶ CAR-transduced T cells per mouse) via tail vein.

The potent antitumor effect observed in vitro was mirrored in the in vivo subcutaneous xenograft model of human glioblastoma (FIGS. 2A and 2B). In this model, established, bulky tumors (top rows) responded to CART-EGFRvIII (FIG. 2B), whereas untransduced cells did not prevent tumor growth (FIG. 2A). In the murine model of intracranial human glioma, EGFRvIII CAR T cells slowed the growth of tumors and led to prolonged survival (FIG. 3B) relative to untransduced cells (FIG. 3A). Although tumor growth was abrogated, the effects were not as pronounced as those observed against subcutaneous tumors.

The presence of regulatory T cells (Tregs) was observed in human patient tumor tissues after treatment with CART-EGFRvIII cells (FIGS. 4A and 4B). To determine if brain-infiltrating Tregs have a functional role in suppressing CART-EGFRvIII cells, an in vitro Treg suppression assay was performed in which CART-EGFRvIII cells and glioma cells were incubated in the presence of Tregs for 18 hours. Results were obtained by IncyCyte live cell analysis, as shown in FIGS. 5A-5C. While non-specific CAR cells permitted proliferation of glioma cells (FIGS. 5A and 5D, top line), CART-EGFRvIII cells killed glioma cells (FIGS. 5B and 5D, bottom line). However, addition of Tregs in the co-culture significantly reduced the ability of CART-EGFRvIII cells to kill target glioma cells (FIGS. 5C and 5D, middle line).

### Example 4. Design and Characterization of CAR T Cells Targeted to Treg-Associated Antigens

FIGS. 6A-6C, 7A, and 7B show results of an experiment in which LAP and GARP were identified as Treg-associated markers on human peripheral blood cells. In particular, among human Tregs that were not activated ex vivo, approximately 27% expressed LAP, approximately 4% were double positive for LAP and GARP (FIG. 6B). Once activated ex vivo using anti-CD3, anti-CD8, and IL-2, approximately 30% expressed LAP, and the number of LAP/GARP double positive Tregs increased to 12.3% (FIG. 6C).

Next, CAR constructs encoding CARs targeting LAP and GARP were designed. Schematic illustrations of these constructs are shown in FIGS. 8A-8D. Treg-targeting constructs include two LAP-targeting CARs (CAR-LAP-L-H (FIG. 8A) and CAR-LAP-H-L (FIG. 8B); in which each anti-LAP scFv contains a reversal in heavy (H) and light (L) chain arrangement), a GARP-targeting CAR construct (CAR-GARP; FIG. 8C), and an EGFR-targeting CAR construct further encoding an anti-GARP camelid antibody (CAR-EGFR-GARP; FIG. 8D). Transduction efficiencies of each construct were assessed using flow cytometry by measuring the percentage of mCherry-positive cells and are provided below.

TABLE 1
Transduction efficiencies of Treg-targeted CAR constructs

| CAR construct | ND47 | ND48 | ND50 |
|---|---|---|---|
| Construct 1 CAR-GARP | 68.0% | 81.0% | 72.8% |
| (SEQ ID NO: 1) | | | |
| Construct 2 CAR-LAP-H-L | 57.1% | 79.5% | 80.4% |
| (SEQ ID NO: 7) | | | |
| Construct 3 CAR-LAP-L-H | 72.2% | 88.2% | 90.1% |
| (SEQ ID NO: 13) | | | |
| Construct 4 CAR-EGFR-GARP | N/A | N/A | 51.2% |
| (SEQ ID NO: 19) | | | |

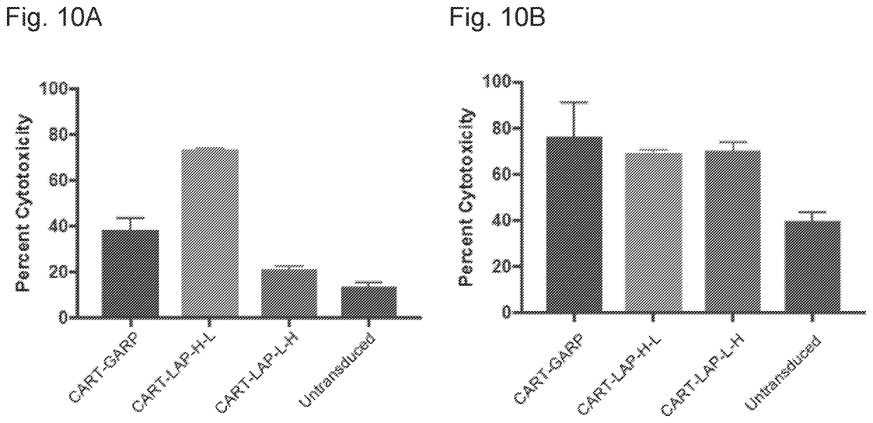

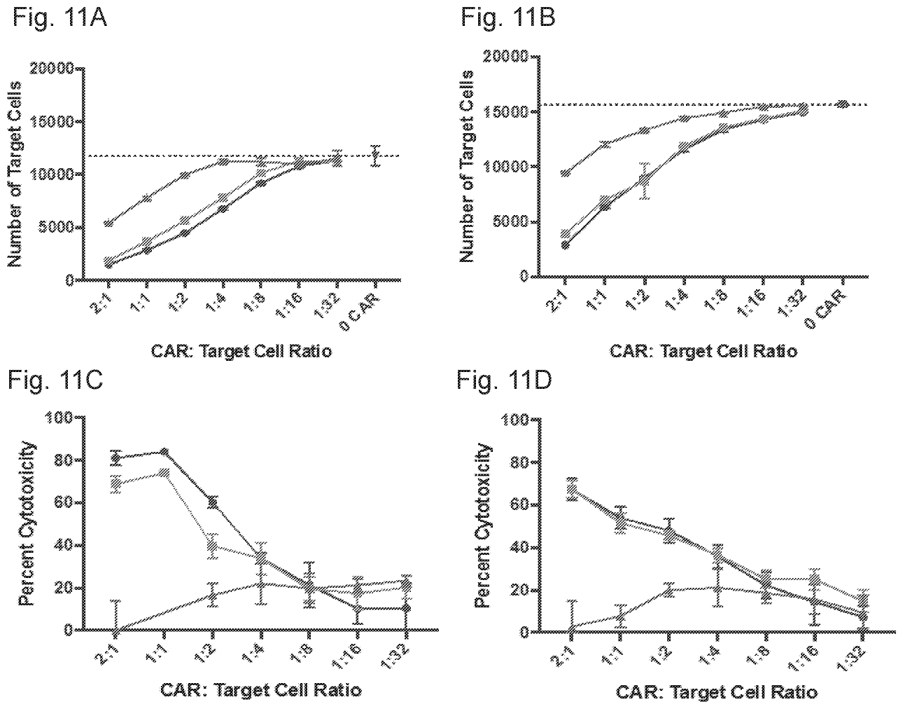

To test anti-LAP CART cells, CAR T cells were co-cultured with isolated Tregs expanded from the same donor and transduced to express GFP as a Treg marker. Tregs were activated overnight with anti-CD3 and anti-CD28 (FIG. 9B) or rested overnight (FIG. 9A) prior to the killing assay. 62,500 Tregs per well were plated. CARs were added at the indicated ratio to Tregs. Cultures were incubated for three days in the presence of 300 U/mL IL-2. Flow cytometry was performed on day 3 by collecting 30,000 events per well. Percent cytotoxicity was calculated as the percent reduction in GFP-positive cells compared to the untransduced T cell culture with Tregs. CART-LAP-H-L was more effective at killing non-activated Tregs in comparison to CART-LAP-L-H. LAP-targeted CAR T cells were then compared to GARP-targeted CART cells in an analogous Treg killing assay across two different donors at a CAR T cell-to-Treg ratio of 1:1 for four days (FIGS. 10A and 10B). FIGS. 11A and 11B characterize non-activated and activated Treg killing by LAP-targeted CAR T cells, relative to untransduced controls, by the number of target Tregs remaining at the end of a three-day coculture as a function of CAR T cell-to-Treg cell ratio. FIGS. 11C and 11D show analogous data from the same donor, in which cytotoxicity is measured by luciferase expression.

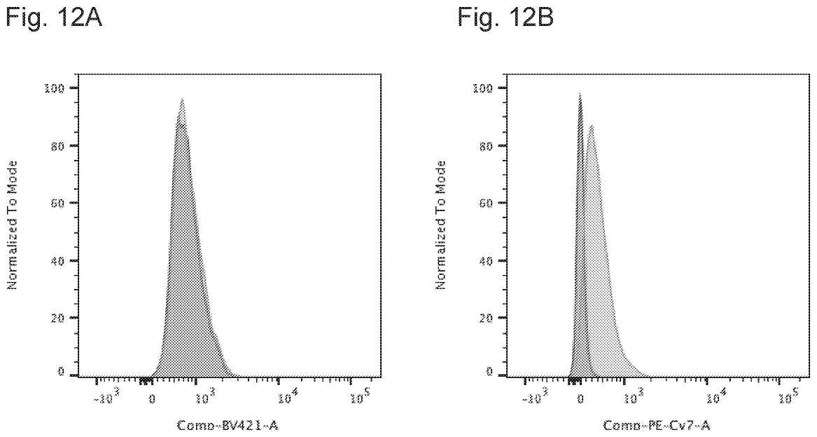

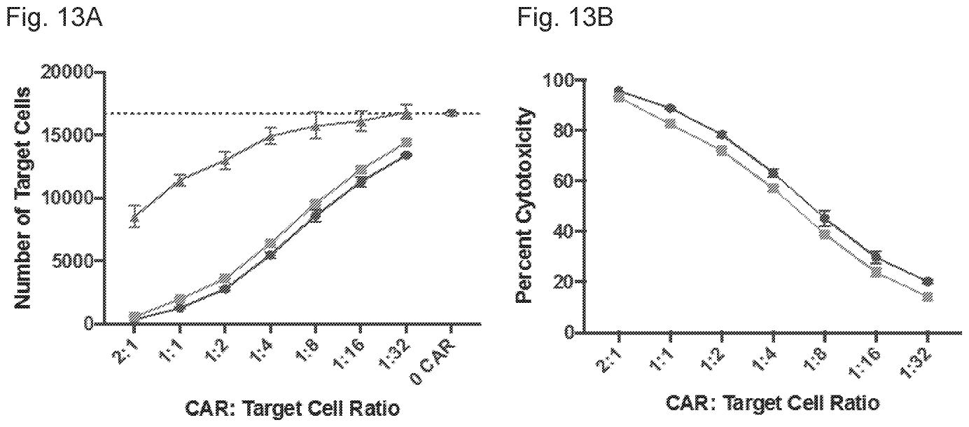

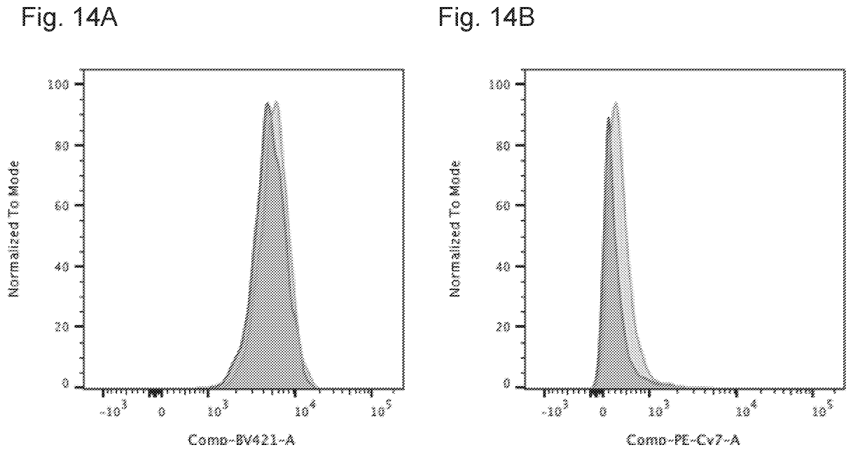

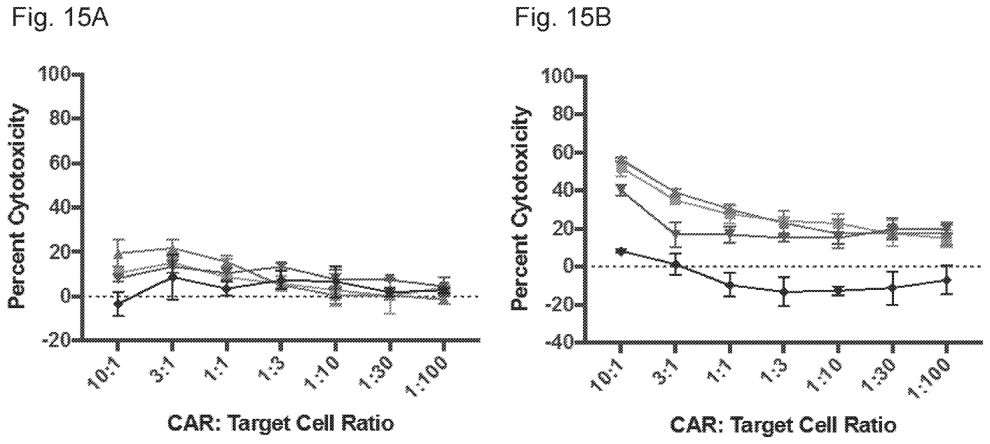

To further characterize the effect of antigen expression on function of LAP- and GARP-targeted CAR T cells, immortalized cell lines were screened for LAP and GARP antigen-expression, and the cytotoxic effect by each CAR T cell was assessed. First, HUT78 cells, a cutaneous human CD4 T cell lymphocyte-derived cell line that expresses IL-2, was stained for GARP and LAP (FIGS. 12A and 12B, respectively), and LAP expression by HUT78 cells was confirmed. Next, CART-LAP-H-L and CART-LAP-L-H cell-mediated cytotoxicity toward HUT78 cells was measured by cytotoxicity assays (FIGS. 13A and 13B). Next, SeAx, an IL-2 dependent human Sezary syndrome-derived cell, was stained for GARP and LAP (FIGS. 14A and 14B, respectively), and expression of both antigens was confirmed. SeAx cells were cocultured with CART-GARP cells, CART-LAP-H-L cells, CART-LAP-L-H cells, and untransduced cells to quantify CAR T cell-mediated killing at 24 hours (FIG. 15A) and 48 hours (FIG. 15B). Each CAR T exhibited superior SeAx target cell killing at 24 hours, with a more pronounced effect at 48 hours. CART-GARP and CART-LAP-H-L killed target SeAx cells with greater efficiency than CART-LAP-L-H cells by 48 hours.

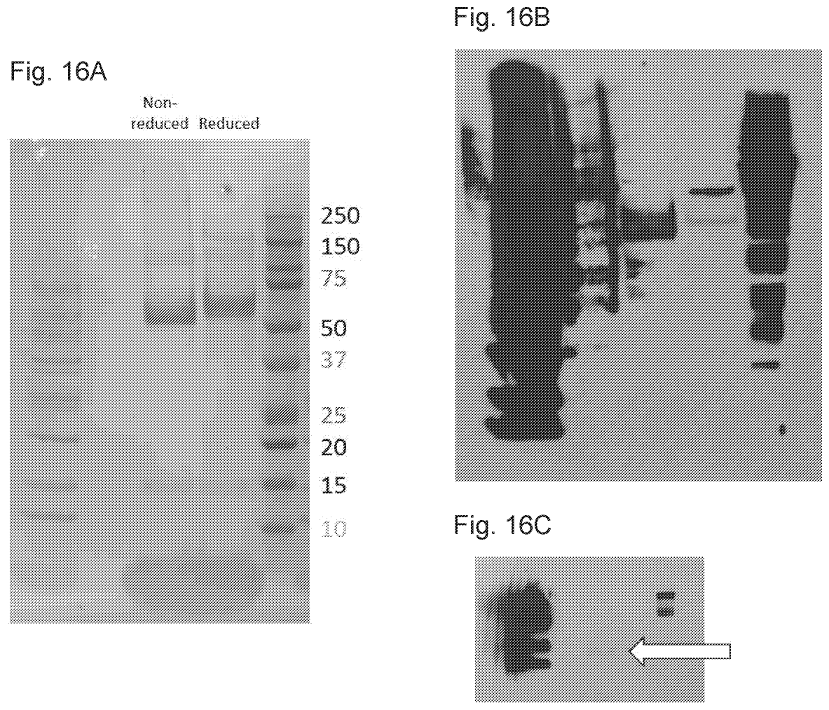

Next, secretion of anti-GARP camelid antibodies by CART-EGFR-GARP cells was characterized by western blot (FIGS. 16A-16C). Supernatant was collected from cultures containing CART-EGFR-GARP cells, treated in reducing and non-reducing conditions, and presence of a band between 10 and 15 kD was observed in the lane containing the non-reduced sample (FIG. 16C), confirming the presence of a camelid antibody.

### Example 5. Design and Characterization of BiTE-Secreting CAR T Cells

Another mechanism provided herein to enhance efficacy of CAR T cell activity within tumor microenvironments (e.g., to overcome immune regulation by Tregs) is through a CAR T cell that secretes an immune-modulating antibody, such as a BiTE. Without wishing to be bound by theory, the present inventors have discovered that expression of an immune-modulating antibody (e.g., a BiTE) from a construct that also encodes a CAR can further amplify antitumor effects.

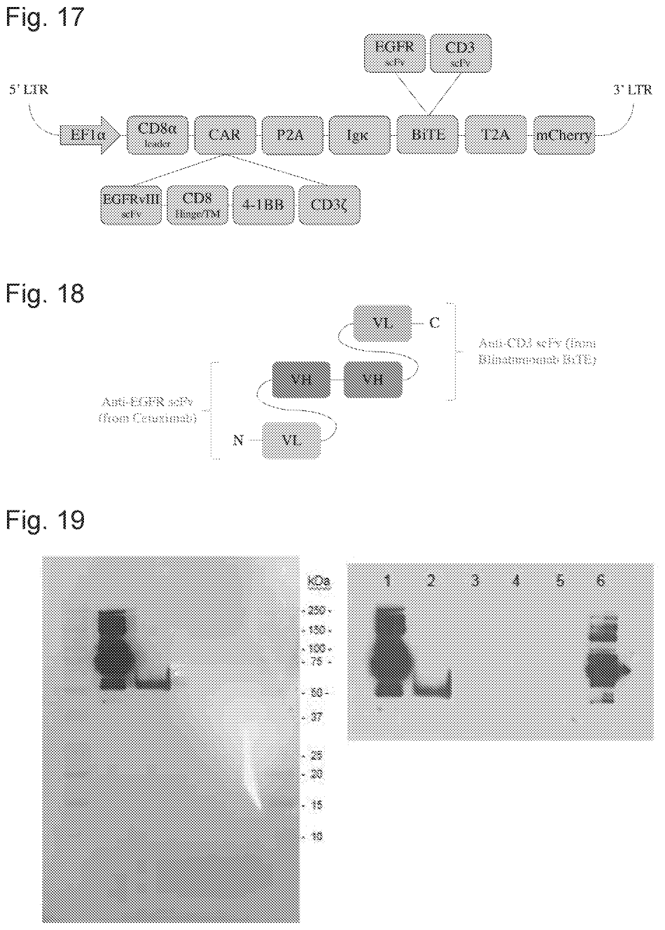

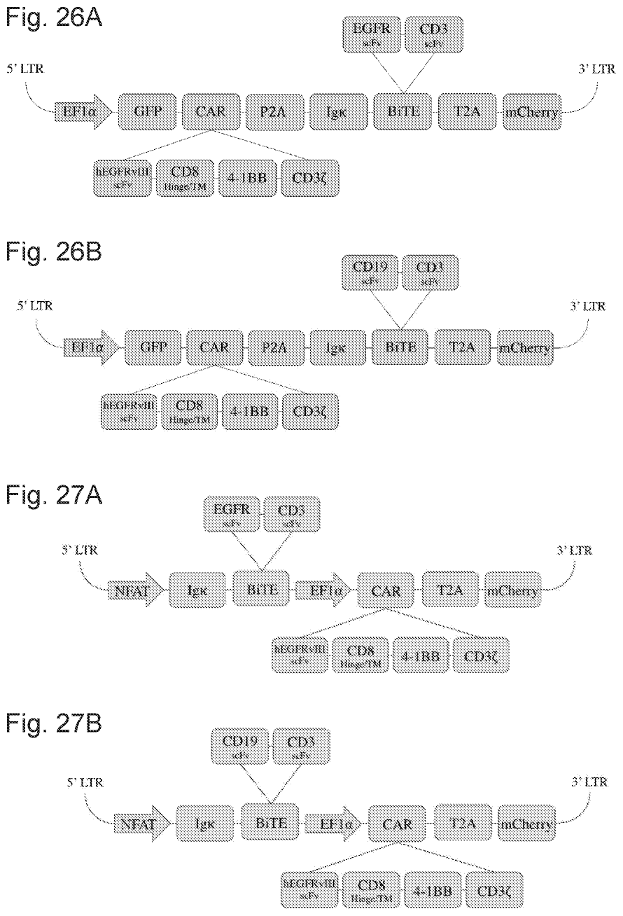

One exemplary nucleic acid construct, CAR-EGFR-BiTE-(EGFR-CD3), shown schematically in FIG. 17, includes a CAR-encoding polynucleotide operatively linked 5′ to a BiTE-encoding polynucleotide. The CAR features a tumor-antigen binding domain that binds to EGFRvIII, which directs the CAR T cell to the microenvironment of an EGFRvIII-positive tumor. The BiTE binds at one domain to EGFR and at the other domain to CD3, as shown in FIG. 18, which can (a) further enhance binding avidity of the host CAR T cell to the tumor cell or (b) arm neighboring (e.g., endogenous) T cells against the tumor. The BiTE is flanked by cleavable linkers P2A and T2A to enable separate secretion of the BiTE, while the CAR is targeted to the cell surface. Other exemplary BiTE-encoding CAR constructs (e.g., encoding a BiTE targeting CD19) are depicted in FIGS. 26A and 26B.

BiTE secretion by CART-EGFR-BiTE-(EGFR-CD3) cells was confirmed by isolating supernatant from cultures containing SupT1 cells transduced with CAR-EGFR-BiTE-(EGFR-CD3), calculating the concentration of BiTE in the supernatant based on OD450, and performing western blot analysis. The concentration of BiTE in the supernatant was 0.604 ng/mL. Results of a western blot experiment are shown in FIG. 19. A band in lane two at about 50-60 kD was observed, indicating the presence of BiTE molecules in the supernatant.

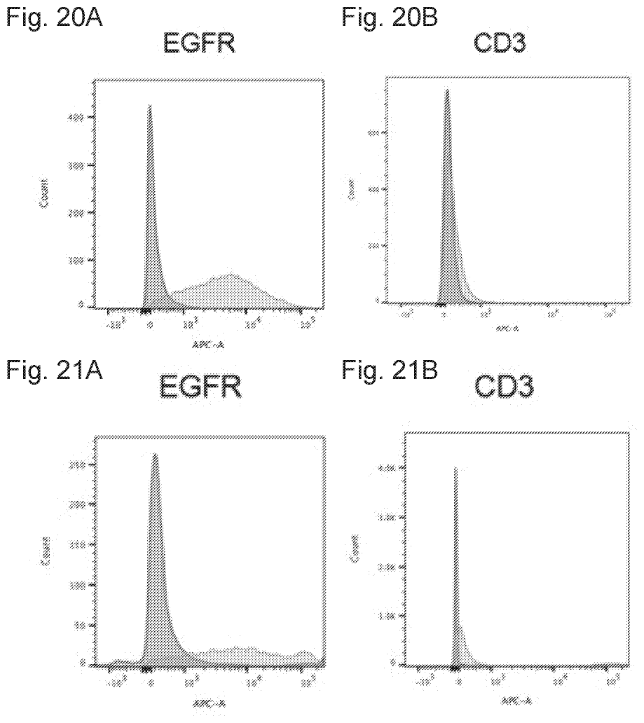

Next, binding of BiTE molecules was assessed by flow cytometry. HEK293T cells were transduced with CAR-EGFR-BiTE-(EGFR-CD3), and supernatants containing secreted BiTEs were collected and incubated with K562 cells (FIG. 20A) and Jurkat cells (FIG. 20B). As shown in FIG. 20A, BiTEs bound K562 cells expressing EGFR and did not bind K562 cells expressing CD19, confirming function of the EGFR-binding domain of the BiTE. As shown in FIG. 20B, CD3-expressing Jurkat cells showed stronger staining for BiTE after incubation with supernatant from CAR-EGFR-BiTE-(EGFR-CD3)-expressing HEK293T cells, compared to staining for BiTE after incubation with supernatant from untransduced HEK293T cells, indicating that BiTEs also functionally bind to CD3.

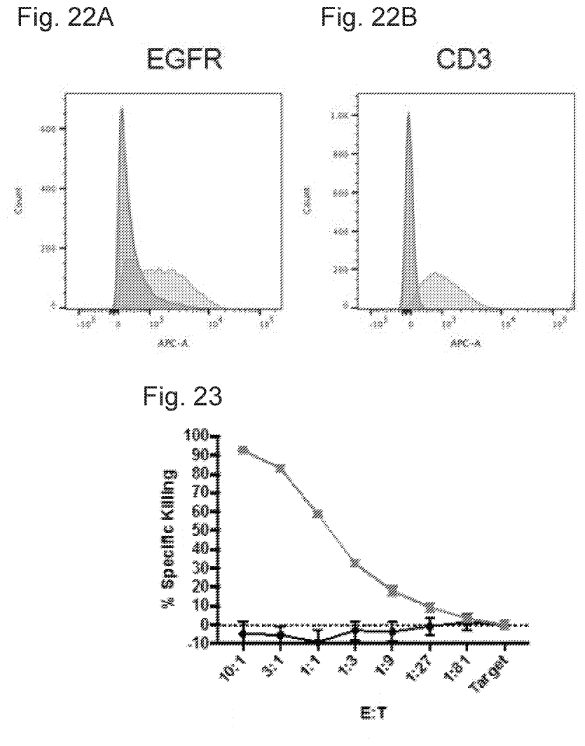

A similar experiment was conducted using SupT1 cells as transduction hosts for CAR-EGFR-BiTE-(EGFR-CD3). FIG. 21A shows BiTEs bound K562 cells expressing EGFR and did not bind K562 cells expressing CD19, confirming function of the EGFR-binding domain of the BiTE expressed by transduced SupT1 cells. To confirm that BiTEs bound to CD3 expressed on the surface of the host SupT1 cell, the transduced SupT1 cells were stained for BiTE. Results shown in FIG. 21B confirmed that transduced SupT1 cells stain positive for BiTEs. ND4 cells were also assessed for ability to secrete functional BiTEs upon transduction with CAR-EGFR-BiTE-(EGFR-CD3). FIG. 22A shows BiTEs secreted by transduced ND4 cells bound K562 cells expressing EGFR and did not bind K562 cells expressing CD19. As shown in FIG. 22B, BiTEs bound to CD3 expressed on the transduced ND4 cells from which they were secreted.

Next, the ability of BiTEs secreted from transduced CAR T cells was characterized in vitro. Supernatants containing BiTEs secreted from HEK293T cells transduced with CAR-EGFR-BiTE-(EGFR-CD3) were incubated with a coculture of untransduced ND4 cells and U87vIII target cells at varying ratios. As shown in FIG. 23, ND4 cells, when incubated with BiTE, in a dose-dependent manner, indicating that BiTEs were binding to both ND4-expressed CD3 and U87vIII-expressed EGFR to a degree sufficient to induce killing by ND4 cells.

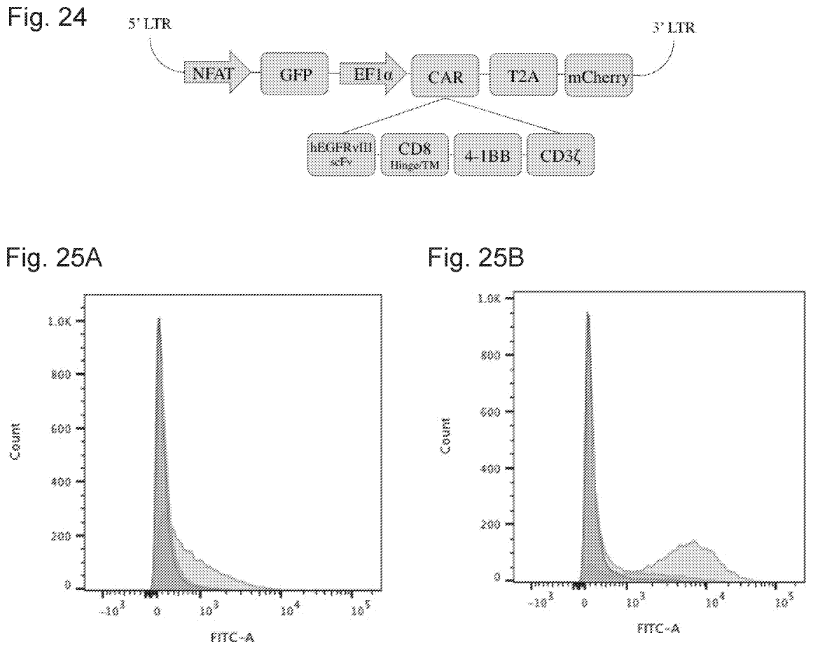

To enable inducible expression of BiTE upon T cell activation, a construct containing an NFAT promoter was designed and synthesized. As shown in FIG. 24, the NFAT promoter precedes a GFP-encoding polynucleotide, and the construct further includes a downstream CAR-encoding polynucleotide driven by EF1α, a constitutive promoter. To confirm the inducible expression of GFP, GFP expression was assessed by FACS in response to TCR stimulation by PMA/ionomycin. As shown in FIGS. 25A and 25B, stimulation triggered the expression of GFP. This inducible expression was inhibited by incubation with PEPvIII. Inducible BiTE constructs encoding CARs are designed by positioning the BiTE downstream of an inducible promoter, such as an NFAT promoter, as shown in FIGS. 27A and 27B.

### Example 6. CAR T Cells for Glioblastoma

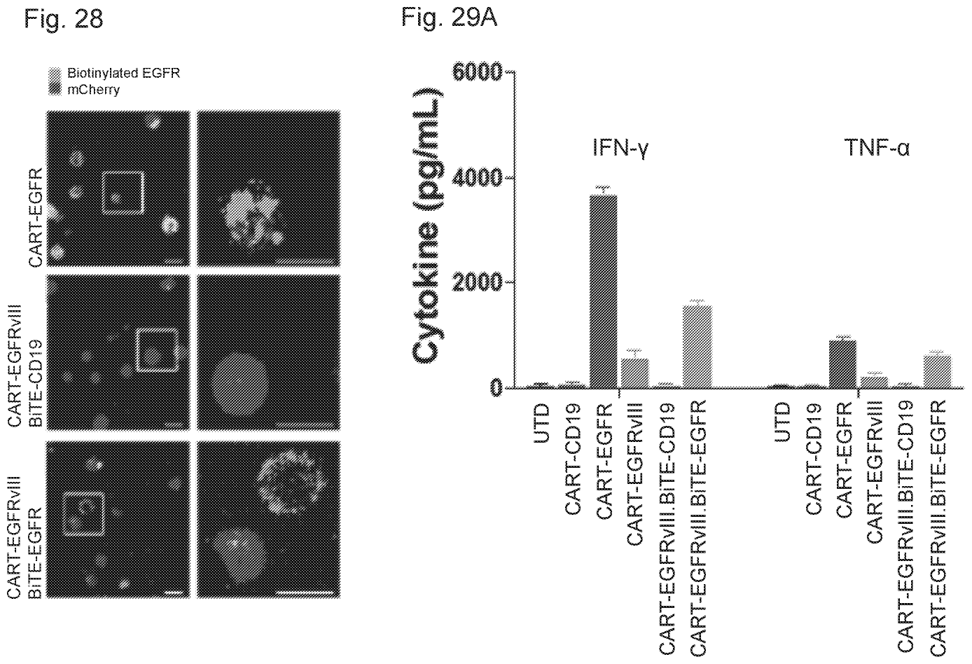

Using confocal microscopy, it was demonstrated that EGFR-targeted BiTEs are released into the supernatant and bind to both transduced (mcherry+) and bystander untransduced (mcherry−) T cells via CD3 (effector arm). In this experiment, CART-EGFR transduced cells (mcherry+) effectively bound biotinylated target antigen (green, FIG. 28, top); in contrast, CART-EGFRvIII secreting a non-specific BiTE did not bind (FIG. 28, middle). Cultures of CAR.BiTE had BiTEs bound in clusters (red/green colocalization), while bystander untransduced T cells in the culture (mcherry−) also bound biotinylated antigen (FIG. 28, bottom).

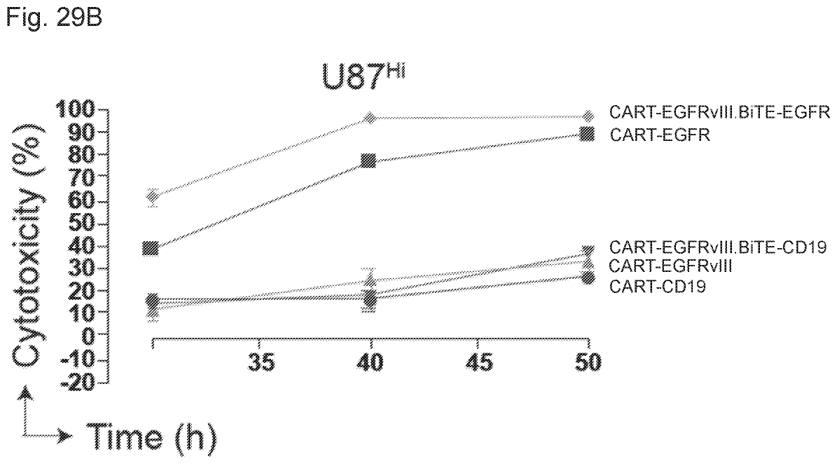

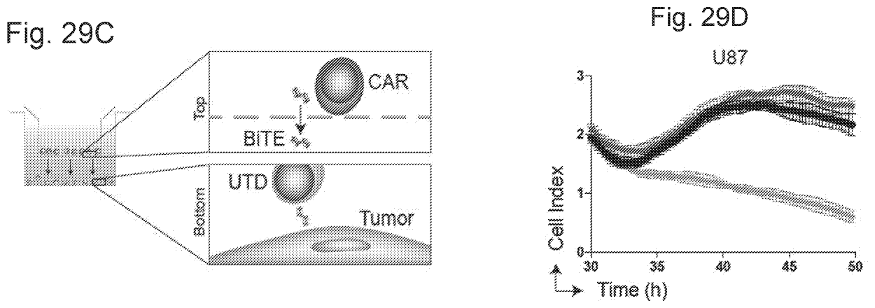

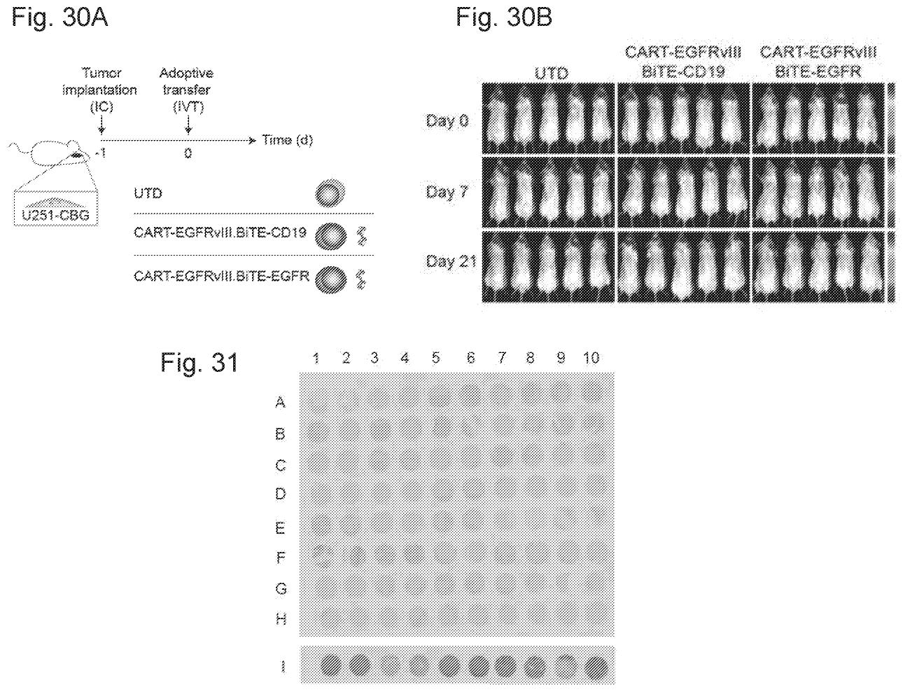

Next, cytokine production in response to antigen stimulation was analyzed. The pattern of IFN-γ and TNF-α production by different CAR constructs was compared after in vitro stimulation with U87, a human malignant glioma cell line that expresses EGFR but not EGFRvIII. This demonstrated EGFR-specific cytokine production mediated by BiTE-redirected T cells (FIG. 29). This finding was consistent with cytotoxicity assays that was performed on an ACEA instrument in which CAR.BiTE was able to mediate potent and specific antitumor efficacy against U87 in vitro (FIG. 29). In ACEA Transwell experiments, it was demonstrated that this was primarily due to redirection of bystander untransduced T cells (FIGS. 29C and 29D). Using an in vivo model of intracranial glioma (U251) that expresses EGFR but not EGFRvIII (FIG. 30A), intraventricular administration of CART-EGFRvIII.BiTE-EGFR was found to also be effective against tumors implanted in the brain of immune-compromised mice (FIG. 30B).

In this experiment, CAR T cells that secrete engineered BiTEs which have biological, antitumor effects were successfully generated. This is the first time to our knowledge that this has been demonstrated.

### Example 7. Materials and Methods

### Study Design

The overall purpose of this study was to provide proof-of-concept of a novel therapy seeking to combine both CAR and BiTE T-cell redirecting technologies. Both CAR designs and integrated CART.BiTE constructs were tested using several tumor models, techniques, and approaches. These employed five different xenogeneic models, including three orthotopic brain tumors as well as engrafted human skin to assist in toxicity assessments. Tumor growth was measured by calipers and bioluminescent imaging, and three different in vitro assays of cytotoxicity were used. Each experiment was performed multiple times with T cells derived from a variety of normal human donors.

### Mice and Cell Lines

NSG mice were purchased from Jackson Laboratory and bred under pathogen-free conditions at the MGH Center for Cancer Research. All experiments were performed according to protocols approved by the Institutional Animal Care and Use Committee. The human glioma cell lines U87 and U251, as well as wild-type parental K562 were obtained from American Type Culture Collection (ATCC) and maintained under conditions as outlined by the supplier. In some cases, cells were engineered to express EGFR, EGFRvIII, or CD19 by lentiviral transduction. Where indicated, cell lines were transduced to express click beetle green (CBG) luciferase or enhanced GFP (eGFP) and sorted on a BD FACSAria to obtain a clonal population of transduced cells. The patient-derived neurosphere culture, BT74, was a kind gift from Dr. Santosh Kesari, and was maintained in serum-free EF20 medium as previously described (Pandita et al., Genes Chromosomes Cancer. 39:29-36, 2004).

### Construction of CARs

Two anti-EGFRvIII CART.BiTE constructs and three additional CAR constructs (anti-EGFR, anti-EGFRvIII, and anti-CD19) were synthesized and cloned into a third-generation lentiviral plasmid backbone under the regulation of a human EF-1α promoter. All CAR and CART.BiTE constructs contained a CD8 transmembrane domain in tandem with an intracellular 4-1 BB costimulatory and CD3ζ signaling domain. BiTEs were designed against wild-type EGFR and CD19 with both sequences flanked by an Igκ signal peptide and a polyhistidine-tag (His-tag) element. Ribosomal skip sites were incorporated at appropriate locations. All constructs also contained a transgene coding for the fluorescent reporter, mCherry, to aid in the evaluation of transduction efficiency.

### CAR T-Cell Production

Human T cells were purified from anonymous human healthy donor leukapheresis product (Stem Cell Technologies) purchased from the MGH blood bank under an IND-exempt protocol. Cells were transduced with lentivirus corresponding to various second-generation CAR T-cell constructs. In brief, bulk human T cells were activated on day 0 using CD3/CD28 Dynabeads (Life Technologies) and cultured in RPMI 1640 medium with GlutaMAX and HEPES supplemented with 10% FBS and 20 IU/mL of recombinant human IL-2. Lentiviral transduction of cells was performed on day 1 and unless otherwise indicated, cells were permitted to expand until day 10 and subsequently transferred to storage in liquid nitrogen prior to functional assays. For all functional assays, CAR T cells and CART.BiTE cells were normalized for transduction efficiency using untransduced but cultured and activated T cells from the same donor and expansion. In certain experiments CAR T cells were sorted on a BD FACSAria to obtain a pure population of transduced, mCherry-positive T cells on day 10.

### T-Cell Activation and Functional Assays

Jurkat (NFAT-Luciferase) reporter cells (Signosis) were transduced with different CAR constructs prior to coculture with tumor targets at an E:T ratio of 1:1 for 24 hours. Bystander Jurkat activation was similarly assessed with coculture of untransduced Jurkat reporter cells (J) as well as accompanying primary human T cells and tumor targets at a J:E:T ratio of 1:1:1 for 24 hours. Luciferase activity was then assessed using a Synergy Neo2 luminescence microplate reader (Biotek). Cell-free supernatants from responder cells cocultured with tumor targets were also analyzed for cytokine expression using a Luminex array (Luminex Corp, FLEXMAP 3D) according to manufacturer instructions. In experiments where activation markers CD25 and CD69 were assessed, CAR T cells and CART.BiTE cells were incubated with irradiated U87 at an E:T of 1:1. Cells were cocultured for 72 hours and then subjected to flow cytometric analysis. For proliferation assays of sorted transduced cells, effectors were expanded for 10 days and then sorted on mCherry-positive events. Cells were then stimulated using irradiated U87, U87vIII, or U87-CD19. UTDs, sorted CART-EGFRvIII cells and sorted CART.BiTE cells were then stimulated through CAR alone (CART-EGFRvIII.BiTE-CD19 and U87vIII), BiTE alone (CART-EGFRvIII.BiTE-CD19 and U87-CD19), or CAR and BiTE (CART-EGFRvIII.BiTE-EGFR and U87vIII). Effector and target cells were plated at an E:T of 1:1. Cells were counted every 7 days and plated again with stimulation at 7 day intervals.

### Cytotoxicity Assays

For single time-point cytotoxicity assays, CAR T cells were incubated with luciferase-expressing tumor targets at indicated E:T ratios for 18 hours. Remaining luciferase activity was subsequently measured with a Synergy Neo2 luminescence microplate reader (Biotek). Percent specific lysis was calculated by the following equation: %=((target cells alone RLU−total RLU)/(target cells only RLU))×100. For real-time cytotoxicity assays against adherent cell lines, cell index was recorded as a measure of cell impedance using the xCELLigence RTCA SP instrument (ACEA Biosciences, Inc.). Percent specific lysis was calculated using the following equation: %=((cell index of UTDs−cell index of CAR T cells)/cell index of UTDs)×100. In transwell cytotoxicity assays using the ACEA instrument, Jurkat reporter cells transduced with CAR constructs were cultured in the top well of 0.4 μm transwell inserts (ACEA Biosciences). Untransduced T cells were cocultured in the bottom well with tumor targets at the indicated E:T ratios. Occasionally, spurious readings from certain wells due to ACEA machine malfunction were censored but did not affect interpretation of the data. In tests against neurospheres, cytotoxicity was measured by total average green area as recorded by IncuCyte Live Cell Analysis. Neurospheres were plated 3 days prior to adding effectors to encourage neurosphere formation. Effector cells were added at an E:T of 3:1 and monitored over time, with 4 images per well obtained every 10 minutes.

### BiTE Purification and Quantification

HEK293T cells were transduced with respective CAR constructs and cultured until confluence. Supernatants from cells were collected and incubated with HisPur Ni-NTA Resin (Thermo Fisher Scientific) for 24-48 hours at 4° C. under gentle agitation. The supernatant-resin mixture was then washed with Ni-NTA wash buffer (50 mM Tris pH 8.0, 500 mM NaCl, 5% glycerol, 25 mM imidazole). His-tag proteins were then eluted in Ni-NTA elution buffer (50 mM Tris pH 8.0, 500 mM NaCl, 5% glycerol, 250 mM imidazole). After elution, proteins were buffer exchanged into PBS using Slide-A-Lyzer Cassette Float Buoys (Thermo Fisher Scientific) according to manufacturer instructions. When indicated, further concentration of proteins was performed using Amicon Ultra-15 Centrifugal Filter Units (EMD Millipore). Protein concentrations of cell-free, BiTE-containing solutions were determined using the His Tag ELISA Detection Kit (GenScript). Briefly, BiTE-producing cells were seeded at 2×10⁵ cells/mL. Cells were allowed to grow for 2 weeks and supernatant was collected and analyzed intermittently. Where indicated samples were normalized to average values obtained from wells containing UTDs only.

### Western Blotting

Protein samples were separated by SDS-PAGE and transferred onto nitrocellulose membranes using Novex iBlot 2 Nitrocellulose Transfer Stacks (Invitrogen) and iBlot 2 Gel Transfer Device (Invitrogen) according to manufacturer protocols. Briefly, membranes were incubated in blocking buffer consisting of 5% nonfat dry milk (Bio-Rad) in TBST (Santa Cruz Biotechnology) for 1 hour. The membrane was washed once in TBST and probed with anti-His-tag antibody (1:2500, Clone 3D5, Invitrogen) overnight at 4° C. Membranes were washed three times for 5 minutes with TBST and incubated with horseradish peroxidase-conjugated sheep anti-mouse IgG antibody (1:5000, GE Healthcare) for 1 hour. Membranes were then washed three times for 5 minutes each with TBST and developed with Amersham ECL Prime Western Blotting Detection Reagent (GE Healthcare).

### Flow Cytometry and Immunohistochemistry

The following antibody clones targeting their respective antigens were used for flow cytometric analysis where indicated: EGFR (AY13, BioLegend), EGFRvIII (L8A4, Absolute Antibody), His-tag (4E3D10H2/E3, Thermo Fischer), CD25 (2A3, BD Biosciences), CD69 (FN50, BioLegend), CCR7 (3D12, BD Bioscience), CD45RO (UCHL1, BD Biosciences), PD-1 (EH12287, Biolegend), TIM-3 (F38-2E2, Biolegend), LAG-3 (3DS223H, Biolegend). In certain experiments purified BiTE, or supernatant with soluble BiTE was incubated with target cells prior to secondary staining with anti-His-tag antibody. Generally, cells were stained for 15 minutes in the dark at room temperature and washed twice in PBS with 2% FBS prior to analysis. DAPI was added to establish live versus dead separation. Antibody clones for immunohistochemistry included the following: EGFRvIII (D6T2Q, Cell Signaling) and EGFR (D38B1, Cell Signaling), diluted 1:200 and 1:50, respectively, following EDTA-based antigen retrieval. Formalin-fixed, paraffin-embedded specimens were either isolated from experiments or purchased in the form of commercially available tissue microarrays (GL805c, US Biomax; BNC17011a, US Biomax).

### Microscopic Imaging

Confocal microscopy of T cells was performed on a Zeiss LSM710 inverted confocal microscope in the MGH Cancer Center Molecular Pathology Confocal Core and analyzed on Fiji Is Just ImageJ software. In brief, transduced T cells that had been activated and expanded for 10 days were stained with biotinylated human EGFR (Acro Biosystems) at a concentration of 1 μg/mL for 40 minutes and then incubated with streptavidin (eBioscience) at 1 μg/mL for 15 minutes on ice prior to microscopic analysis. Otherwise, cell cultures were also visualized using an EVOS Cell Imaging System (Thermo Fisher Scientific). In experiments assessing proliferation, CAR T cells and CART.BiTE cells were cocultured for one week with irradiated U87 expressing eGFP at an E:T of 1:1.

### Animal Models

Tumor cells were harvested in logarithmic growth phase and washed twice with PBS prior to being loaded in a 50 μL syringe with an attached 25-gauge needle. With the assistance of a stereotactic frame, tumor cells were implanted at 2 mm to the right of bregma and a depth of 4 mm from the surface of the skull at the coronal suture. The number of tumor cells varied depending on the cell culture. In mouse models of flank tumor or human skin toxicity, effector cells were infused systemically by tail vein infusion in a volume of 100 μL. When delivered intraventricularly, cells were infused at 2 mm to the left of and 0.3 mm anterior to bregma at a depth of 3 mm. Effector cell populations were normalized to contain 1×10⁶ cells per infusion for all experiments. Tumor progression was then longitudinally evaluated by bioluminescence emission using an Ami HT optical imaging system (Spectral Instruments) following intraperitoneal substrate injection. For toxicity studies, deidentified, excess human skin was obtained from healthy donors during abdominoplasty surgeries under informed consent and approval by the Institutional Review Board. An approximately 1 cm×1 cm skin sample was sutured to the dorsa of NSG mice and allowed to heal for at least 6 weeks. Engrafted skin was monitored daily for up to 2 weeks prior to excision and histological analysis.

### Statistical Methods

All analyses were performed with GraphPad Prism 7.0c software. Data was presented as means±standard deviation (SD) or standard error of mean (SEM) with statistically significant differences determined by tests as indicated in figure legends.

### Example 8. CAR T Cells Against EGFRvIII for Glioblastoma (GBM) and Design of CART.BiTE

Glioblastoma (GBM) is the most common malignant brain tumor and is also the most deadly. Current treatment for GBM includes surgical resection, radiation and temozolomide chemotherapy, which provide only incremental benefit and are limited by systemic toxicity and damage to normal brain (Imperato et al., Annals of Neurology 28:818-822, 1990). In 2017, CART cells targeting CD19 were approved by the U.S. Food and Drug Administration (FDA) for B-cell malignancies and have since revolutionized the treatment of hematological cancers (Mullard et al., Nat. Rev. Drug. Discov. 16:699, 2017). Several different CARs have been described in recent clinical studies for GBM (O'Rourke et al., Sci. Transl. Med. 9, 2017; Ahmed et al., JAMA Oncol. 3:1094-1101, 2017; Brown et al., N. Engl. J. Med. 375:2561-2569, 2016), where peripherally injected CAR-transduced T cells have localized to brain tumors and, in at least one case, intracranially-administered CAR T cells mediated the regression of late-stage, multifocal, bulky disease (Brown et al., supra). However, clinical responses against GBM have not been consistent or durable, in large part due to heterogeneous antigen expression within these tumors and the emergence of antigen escape following treatment with CAR T cells directed at a single target. Approximately 30% of GBMs express EGFRvIII (Wikstrand et al., Cancer Res. 57:4130-4140, 1997), while 80% or more express EGFR (Verhaak et al., Cancer Cell 17:98-110, 2010). When the EGFRvIII mutation is lost in GBM, amplification of wild-type EGFR is maintained (O'Rourke et al., Sci. Transl. Med. 9, 2017; Felsberg et al., Clin. Cancer Res. 23:6846-6855, 2017). Although EGFR expression is also found in normal tissues such as the skin, lungs, and gut, EGFR was not detected in the analysis of 80 core samples from healthy human central nervous system (CNS) tissues (FIG. 31, Table 2), consistent with publicly available organ-specific data from The Human Protein Atlas (Uhlen et al. Mol. Cell Proteomics 4:1920-1932, 2005). This favorable expression pattern was exploited by creating EGFRvIII-specific CAR T cells that secrete BiTEs against wild-type EGFR (CART-EGFRvIII.BiTE-EGFR), with the hypothesis that this strategy could be used to safely enhance efficacy in GBM models of EGFRvIII antigen loss.

TABLE 2
Sample designations for normal CNS and GBM tissue microarrays

| Position | Age | Sex | Site | Location/Diagnosis |
|---|---|---|---|---|
| A1 | 15 | M | Cerebrum | Frontal lobe tissue |
| A2 | 15 | M | Cerebrum | Frontal lobe tissue |
| A3 | 18 | F | Cerebrum | Frontal lobe tissue |
| A4 | 18 | F | Cerebrum | Frontal lobe tissue |
| A5 | 38 | M | Cerebrum | Frontal lobe tissue |
| A6 | 38 | M | Cerebrum | Frontal lobe tissue |
| A7 | 45 | M | Cerebrum | Apical lobe tissue |
| A8 | 45 | M | Cerebrum | Apical lobe tissue |
| A9 | 15 | M | Cerebrum | Apical lobe tissue |
| A10 | 15 | M | Cerebrum | Apical lobe tissue |
| B1 | 18 | F | Cerebrum | Apical lobe tissue |
| B2 | 18 | F | Cerebrum | Apical lobe tissue |
| B3 | 19 | M | Cerebrum | Occipital lobe tissue |
| B4 | 19 | M | Cerebrum | Occipital lobe tissue |
| B5 | 18 | F | Cerebrum | Occipital lobe tissue |
| B6 | 18 | F | Cerebrum | Occipital lobe tissue |
| B7 | 2 | F | Cerebrum | Occipital lobe tissue |
| B8 | 2 | F | Cerebrum | Occipital lobe tissue |
| B9 | 18 | F | Cerebrum | Temporal lobe tissue |
| B10 | 18 | F | Cerebrum | Temporal lobe tissue |
| C1 | 45 | M | Cerebrum | Temporal lobe tissue |
| C2 | 45 | M | Cerebrum | Temporal lobe tissue |
| C3 | 42 | F | Cerebrum | Temporal lobe tissue |
| C4 | 42 | F | Cerebrum | Temporal lobe tissue |
| C5 | 21 | F | Cerebrum | Midbrain tissue |
| C6 | 21 | F | Cerebrum | Midbrain tissue |
| C7 | 38 | M | Cerebrum | Midbrain tissue |
| C8 | 38 | M | Cerebrum | Midbrain tissue |
| C9 | 21 | F | Cerebrum | Midbrain tissue |
| C10 | 21 | F | Cerebrum | Midbrain tissue |
| D1 | 45 | M | Cerebrum | Pons tissue |
| D2 | 45 | M | Cerebrum | Pons tissue |
| D3 | 47 | M | Cerebrum | Pons tissue |
| D4 | 47 | M | Cerebrum | Pons tissue |
| D5 | 35 | M | Cerebrum | Pons tissue |
| D6 | 35 | M | Cerebrum | Pons tissue |
| D7 | — | — | Cerebrum | Medulla oblongata tissue |
| D8 | — | — | Cerebrum | Medulla oblongata tissue |
| D9 | 27 | M | Cerebrum | Medulla oblongata tissue |
| D10 | 27 | M | Cerebrum | Medulla oblongata tissue |
| E1 | 50 | F | Cerebrum | Medulla oblongata tissue |
| E2 | 50 | F | Cerebrum | Medulla oblongata tissue |
| E3 | 43 | M | Cerebrum | Thalamus opticus tissue |
| E4 | 43 | M | Cerebrum | Thalamus opticus tissue |
| E5 | 15 | M | Cerebrum | Thalamus opticus tissue |
| E6 | 15 | M | Cerebrum | Thalamus opticus tissue |
| E7 | 2 | F | Cerebrum | Thalamus opticus tissue |
| E8 | 2 | F | Cerebrum | Thalamus opticus tissue |
| E9 | 24 | F | Cerebellum | Cerebellum tissue |
| E10 | 24 | F | Cerebellum | Cerebellum tissue |
| F1 | 35 | M | Cerebellum | Cerebellum tissue |
| F2 | 35 | M | Cerebellum | Cerebellum tissue |
| F3 | 35 | M | Cerebellum | Cerebellum tissue |
| F4 | 35 | M | Cerebellum | Cerebellum tissue |
| F5 | 38 | M | Cerebrum | Hippocampus tissue |
| F6 | 38 | M | Cerebrum | Hippocampus tissue |
| F7 | 50 | F | Cerebrum | Hippocampus tissue |
| F8 | 50 | F | Cerebrum | Hippocampus tissue |
| F9 | 48 | M | Cerebrum | Hippocampus tissue |
| F10 | 48 | M | Cerebrum | Hippocampus tissue |
| G1 | 19 | M | Cerebrum | Callositas tissue |
| G2 | 19 | M | Cerebrum | Callositas tissue |
| G3 | 45 | M | Cerebrum | Callositas tissue |
| G4 | 45 | M | Cerebrum | Callositas tissue |
| G5 | 21 | F | Cerebrum | Callositas tissue |
| G6 | 21 | F | Cerebrum | Callositas tissue |
| G7 | 33 | M | Cerebrum | Optic nerve tissue |
| G8 | 33 | M | Cerebrum | Optic nerve tissue |
| G9 | 30 | M | Cerebrum | Optic nerve tissue |
| G10 | 30 | M | Cerebrum | Optic nerve tissue |
| H1 | 45 | M | Cerebrum | Optic nerve tissue |
| H2 | 45 | M | Cerebrum | Optic nerve tissue |
| H3 | 40 | M | Cerebrum | Spinal cord tissue |
| H4 | 40 | M | Cerebrum | Spinal cord tissue (sparse) |
| H5 | 38 | M | Cerebrum | Spinal cord tissue |
| H6 | 38 | M | Cerebrum | Spinal cord tissue |
| H7 | 30 | M | Cerebrum | Spinal cord tissue |
| H8 | 30 | M | Cerebrum | Spinal cord tissue |
| H9 | 46 | M | Cerebrum | Brain tissue |
| H10 | 46 | M | Cerebrum | Caudate nucleus tissue |
| I1 | 59 | F | Cerebrum | GBM |
| I2 | 59 | F | Cerebrum | GBM |
| I3 | 80 | M | Cerebrum | GBM |
| I4 | 80 | M | Cerebrum | GBM |
| I5 | 59 | M | Cerebrum | GBM |
| I6 | 59 | M | Cerebrum | GBM |
| I7 | 48 | F | Cerebrum | GBM |
| I8 | 48 | F | Cerebrum | GBM |
| I9 | 63 | M | Cerebrum | GBM |
| I10 | 63 | M | Cerebrum | GBM |

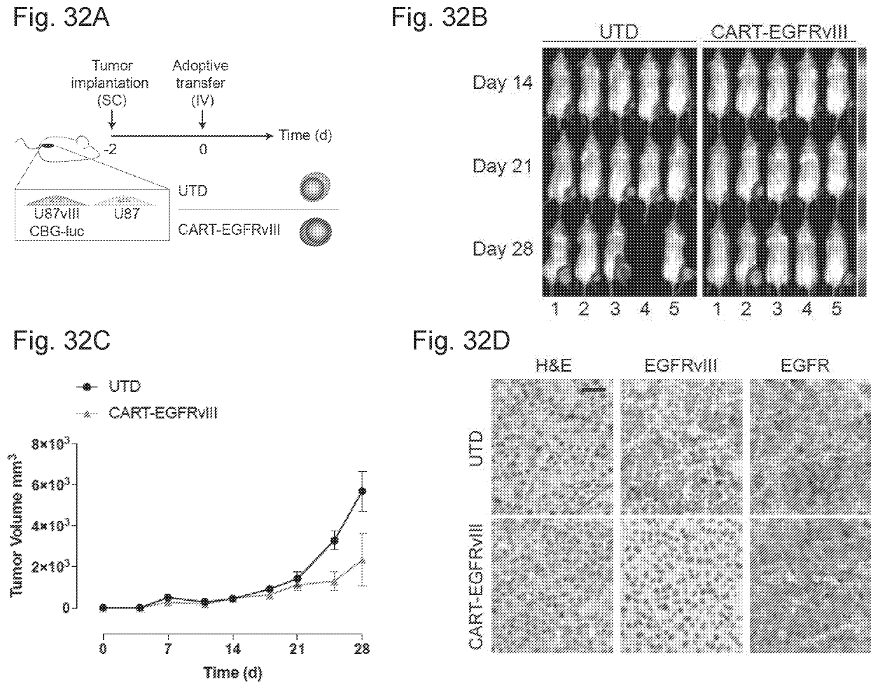

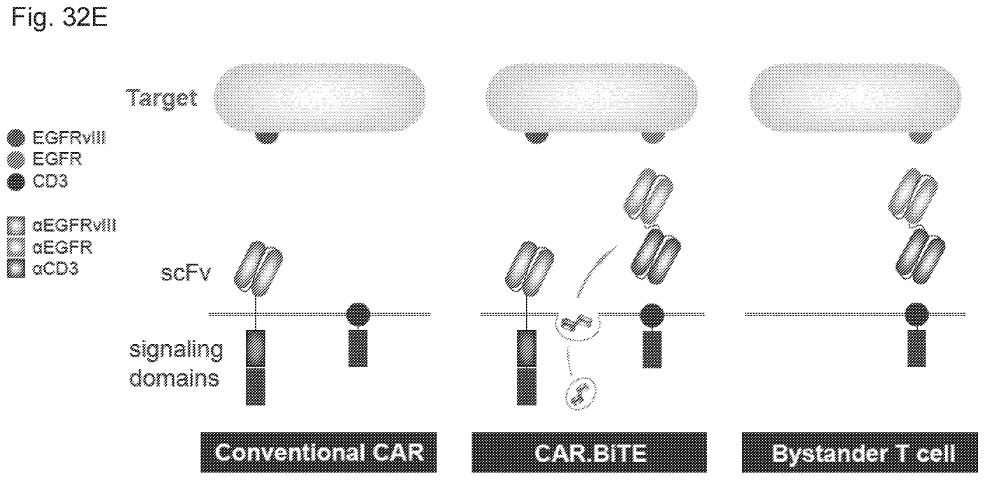

To recapitulate GBM heterogeneity and the emergence of antigen escape at recurrence in xenograft models, tumors with heterogeneous EGFRvIII expression were implanted into the flanks of NSG (NOD.Cg-Prkdc^{scid}Il2rg^{tm1Wjl}/SzJ) mice (FIG. 32A). Mice were treated intravenously (IV) by tail vein on day 2 post-implantation with untransduced T cells (UTD) or CART-EGFRvIII. EGFRvIII-positive cells were transduced with click beetle green luciferase (CBG-luc) to permit real-time assessment of tumor progression by bioluminescent imaging. Flank implantation allowed for concomitant caliper measurements of tumor growth once EGFRvIII-positive cells were eliminated. In this experiment, only EGFRvIII-expressing cells were transduced with luciferase, so that imaging signal would only be detected in this cell population. Whereas mice treated with intravenous (IV) untransduced (UTD) T cells demonstrated outgrowth of EGFRvIII-positive tumor, those treated with CART-EGFRvIII cells showed varying degrees of tumor growth, reflected by abrogated bioluminescent signal in some mice (FIG. 32B). Nevertheless, palpable, measurable tumors progressed in these mice (FIG. 32C). Immunohistochemical (IHC) analyses of harvested tumors were consistent with findings from the clinical trial (O'Rourke et al., supra); namely, recurrent viable tumor with simultaneous loss of EGFRvIII and maintenance of EGFR expression following treatment with CART-EGFRvIII (FIG. 32D). Thus, the concept of CART.BiTE was developed (FIG. 32E), which has the theoretical advantage of multi-antigen targeting, and also the ability to recruit and activate bystander T cells (Choi et al., Proc. Natl. Acad. Sci. USA. 110:270-275, 2013), which represent a major component of the cellular infiltrate observed in GBMs from patients treated with CART-EGFRvIII (O'Rourke et al., supra). Whereas conventional CART-EGFRvIII only targets EGFRvIII positive tumor, CART.BiTE cells have the added capacity of targeting EGFRvIII-negative tumor. In addition, secreted BiTEs may also redirect bystander T cells against residual tumor cells.

### Example 9. Generation of CART.BiTE for Heterogeneous Tumors

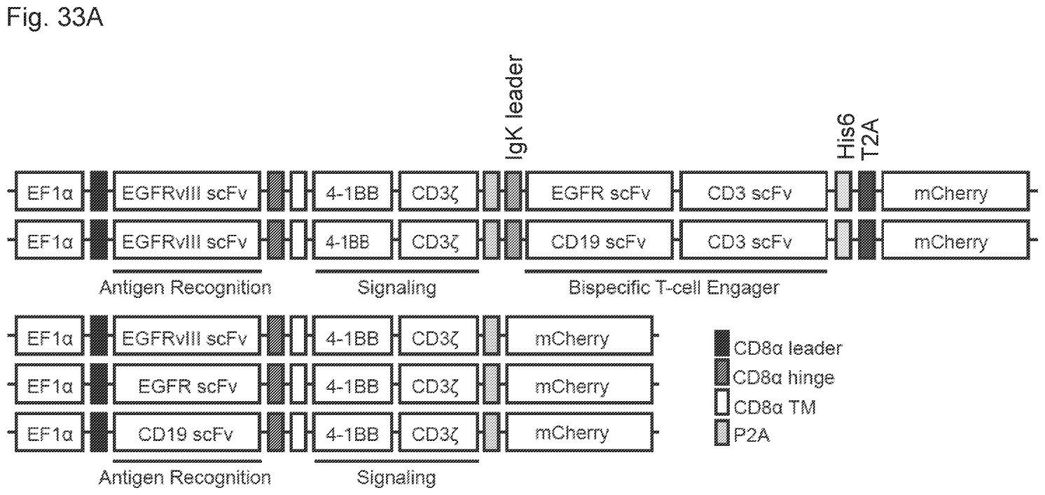

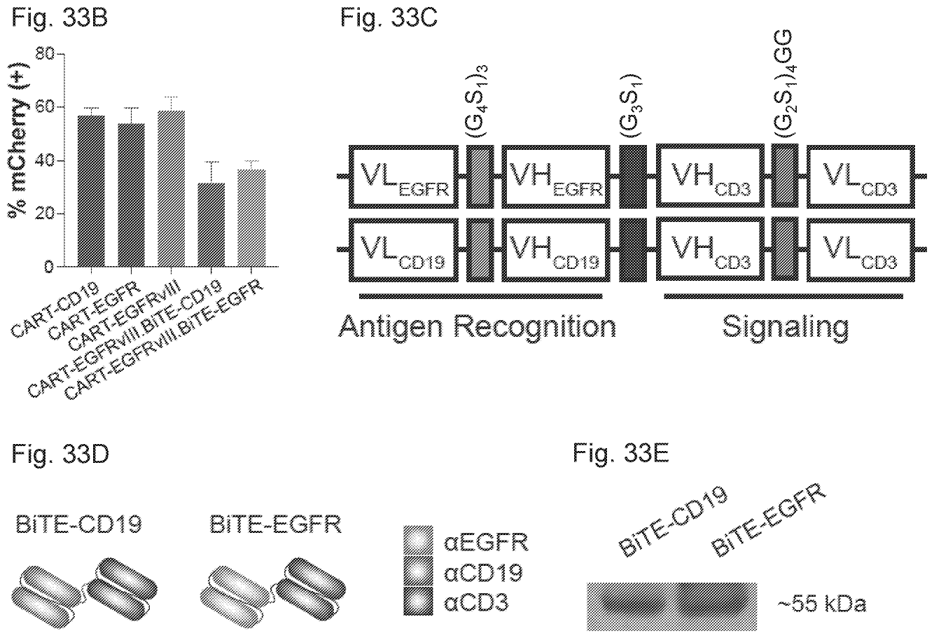

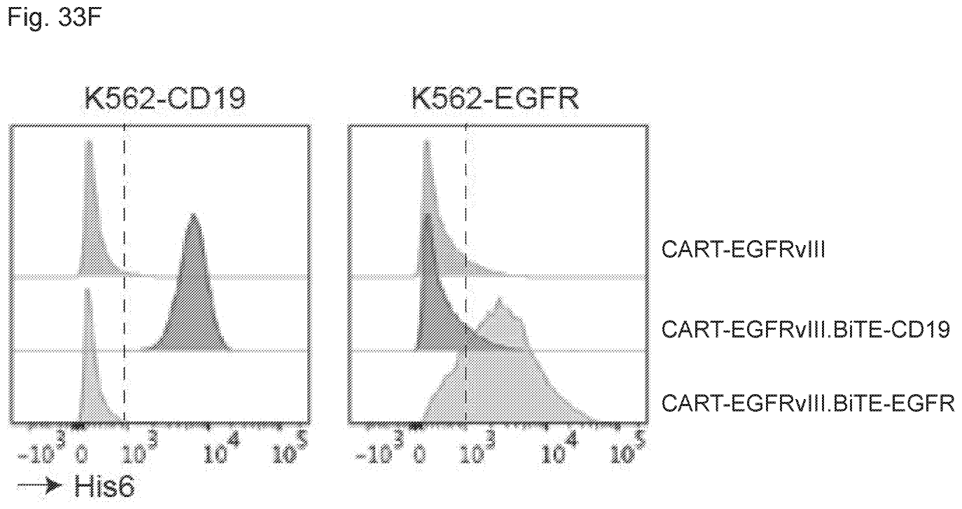

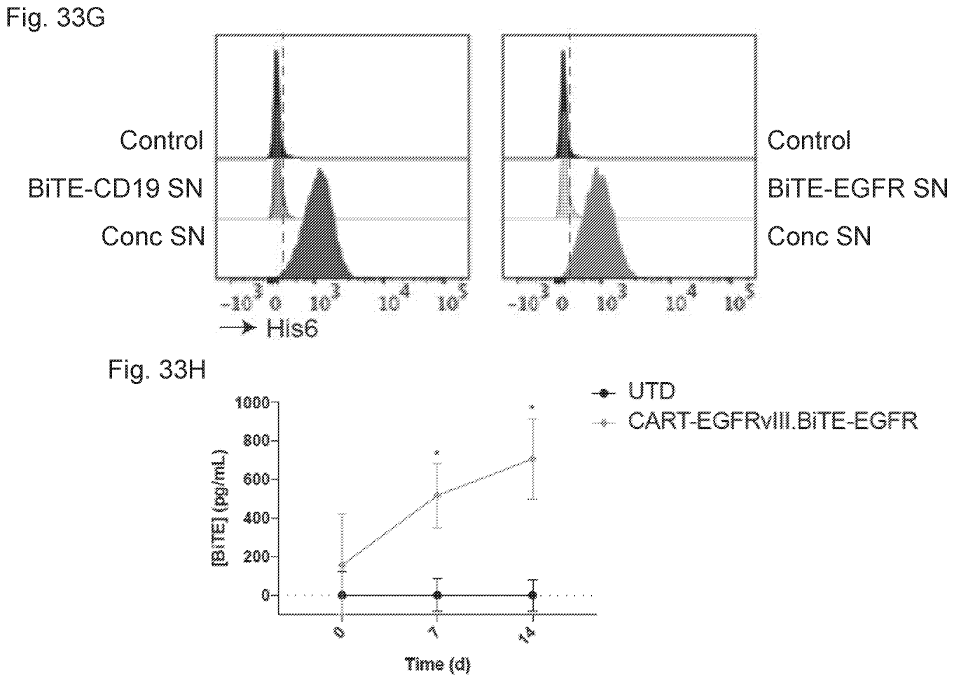

Two CART.BiTE constructs were generated, both based on the second-generation CART-EGFRvIII backbone containing 4-1 BB and CD3ζ intracellular signaling domains (FIG. 33A). The BiTEs were designed against wild-type EGFR or CD19, the latter serving as both a negative control and proof-of-concept for generalizing our findings across model antigens. Sequences for BiTEs were preceded by an Igκ signal peptide and followed by a polyhistidine-tag (His-tag) element to aid in detection and purification of the secreted product. Control CARs that did not secrete BiTEs consisted of the same 4-1BB and CD3ζ backbone as well as single chain variable fragments (scFvs) targeting EGFRvIII, EGFR, and CD19. An mCherry fluorescent reporter gene was included in all vectors to facilitate evaluation of transduction efficiency. Efficient gene transfer of CART.BiTE vectors into primary human T cells was achieved with lentiviral vectors (FIG. 33B).

BiTE cDNA was constructed following the general format previously described (Choi et al., Expert Opin. Biol. Ther. 11:843-853, 2011), incorporating two scFvs translated in tandem, bridged by a flexible glycine-serine linker (FIGS. 33C and 33D). Conventionally, one arm of a BiTE is designed to engage and activate T cells by binding CD3, while the opposing target-binding arm is directed against a tumor antigen. Supernatant from human embryonic kidney (HEK) cells transduced with each CART.BiTE vector demonstrated successful translation and secretion of both BiTE-EGFR and BiTE-CD19, as evidenced by western blot at a predicted molecular weight of approximately 55 kDa (FIG. 33E). Lanes were loaded with 10 μg of protein and subjected to SDS-PAGE and blotting with anti-His-tag antibody. Secreted BiTE product was also successfully isolated from transduced primary human T cells; supernatants from these cultures bound K562 target cells expressing the appropriate cognate antigen. This was confirmed to be antigen-specific since BiTEs isolated from CART-EGFRvIII.BiTE-CD19 and CART-EGFRvIII.BiTE-EGFR cells did not bind to K562s expressing EGFR and CD19, respectively (FIG. 33F). As anticipated, secreted BiTEs also demonstrated the ability to bind T cells via their anti-CD3 scFv domains (FIG. 33G). Detection was enhanced when supernatants from CART.BiTE cells were concentrated, a finding consistent with BiTEs employing the same anti-CD3 scFv clone (Fajardo et al., Cancer Res. 77:2052-2063), such as blinatumomab.

Next, the quantity of BiTE produced by human CART.BiTE cells was approximated using a competitive ELISA-based immunoassay. While UTD cells did not produce a detectable secretory His-tagged protein, soluble BiTE was readily measured in the supernatant of CART.BiTE cells (5×10⁵) and total BiTE concentration increased over time at an approximate rate of 10 pg/d (FIG. 33H). If scaled to an estimated target dose for clinical studies (e.g., approximately 5×10⁸ transduced cells) (O'Rourke et al., supra), this would yield BiTE secretion at an estimated rate of 10 ng/d. Importantly, BiTE-EGFR has been tested for toxicity in Cynomolgus monkeys and was safe at dose equivalents of approximately 800 μg/d for a 70 kg patient (Lutterbuese et al., Proc. Natl. Acad. Sci. USA 107:12065-12610, 2010); this is calculated to be 5 orders of magnitude greater than the projected BiTE secretion that would result from a systemic infusion of CART-EGFRvIII.BiTE-EGFR cells in humans.

Immune therapies with CAR T cells and BiTEs generate potent antitumor responses in patients with hematologic malignancies, but have had limited success in solid tumors like GBM. In the current study, an approach was developed that strategically combines CARs with BiTEs into a single gene-modified T-cell product. It was demonstrated this platform can be used to address critical barriers to effective immune therapy of solid tumors, including antigen escape, immune suppression, and T-cell exhaustion.

### Example 10. CART.BiTE Functions in the Setting of EGFRvIII Antigen Loss

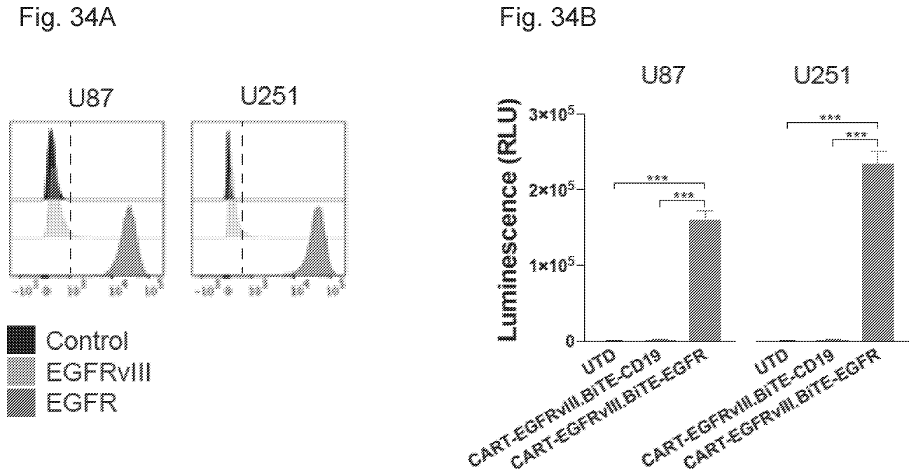

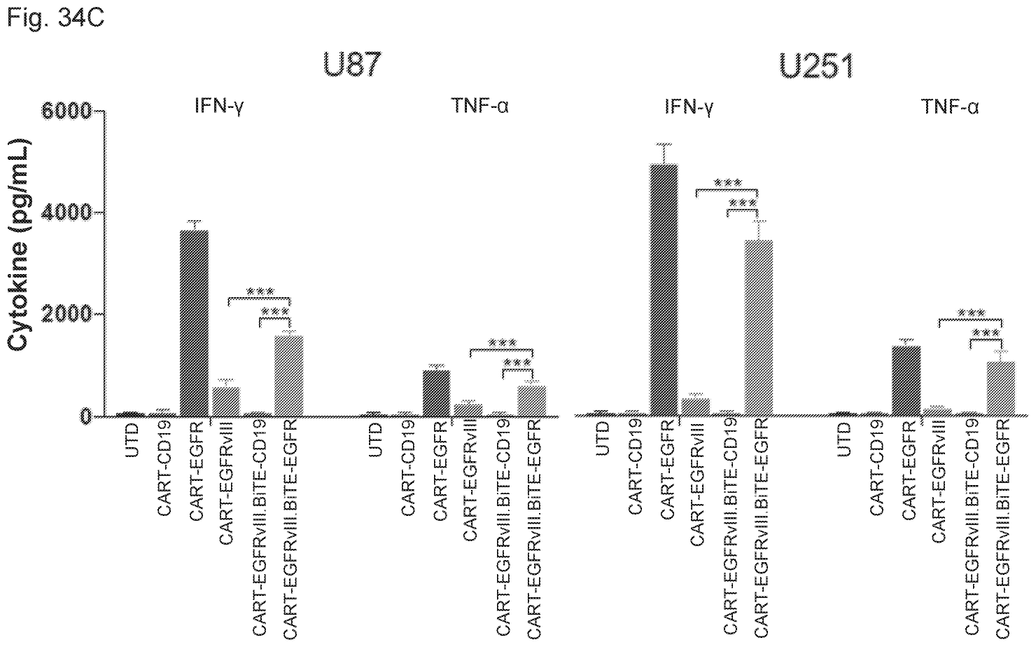

Having demonstrated that CAR-transduced T cells can be engineered to both translate and secrete BiTEs, the functional capacity of the CART.BiTE cells in mediating antitumor immune responses was next determined. First, Jurkat reporter T cells transduced with the candidate constructs were generated and assessed for selective activation against well-characterized EGFRvIII-negative, EGFR-positive glioma cell lines in vitro (FIG. 34A). Unless otherwise stated, all assays were performed in triplicate (mean±SEM is depicted; unpaired t test, ***=p<0.001). To control for off-target activation through the EGFRvIII-specific CAR or via the anti-CD3 scFv of the BiTE alone, we used cells transduced with CART-EGFRvIII.BiTE-CD19. Indeed, T-cell activation was not detected in wells containing either UTD cells or CAR T cells secreting BiTE-CD19. By contrast, T cells transduced with CART-EGFRvIII.BiTE-EGFR demonstrated significant, selective activation against GBM (FIG. 34B). In similar experiments using primary human T cells, CART-EGFRvIII.BiTE-EGFR cells were also found to produce Th1 proinflammatory cytokines IFN-γ and TNF-α when cultured with glioma cells in a BiTE-dependent, EGFR-specific fashion (FIG. 34C).

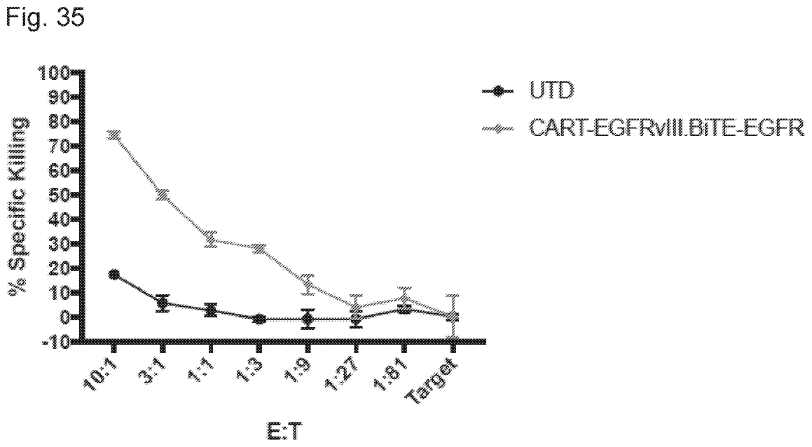

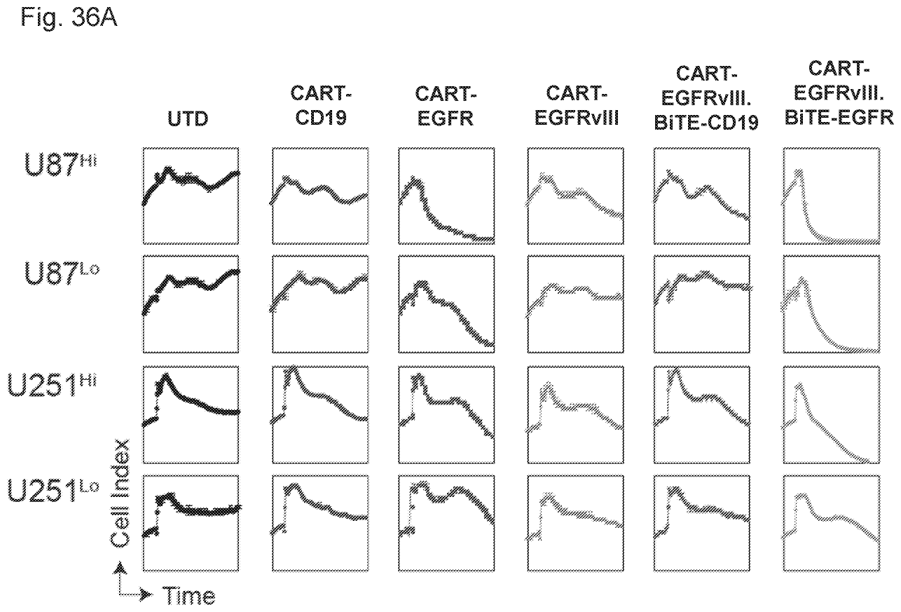

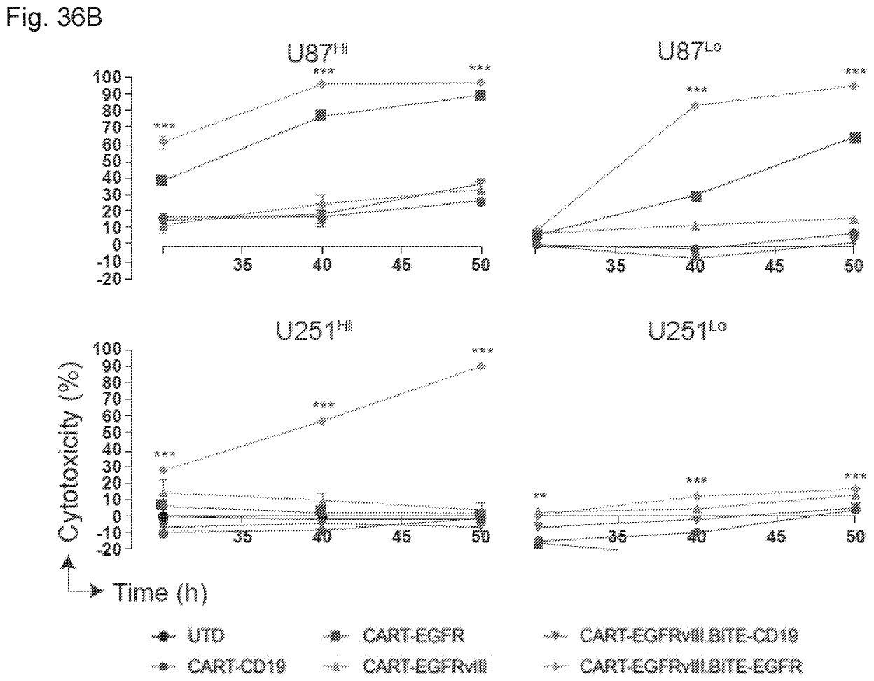

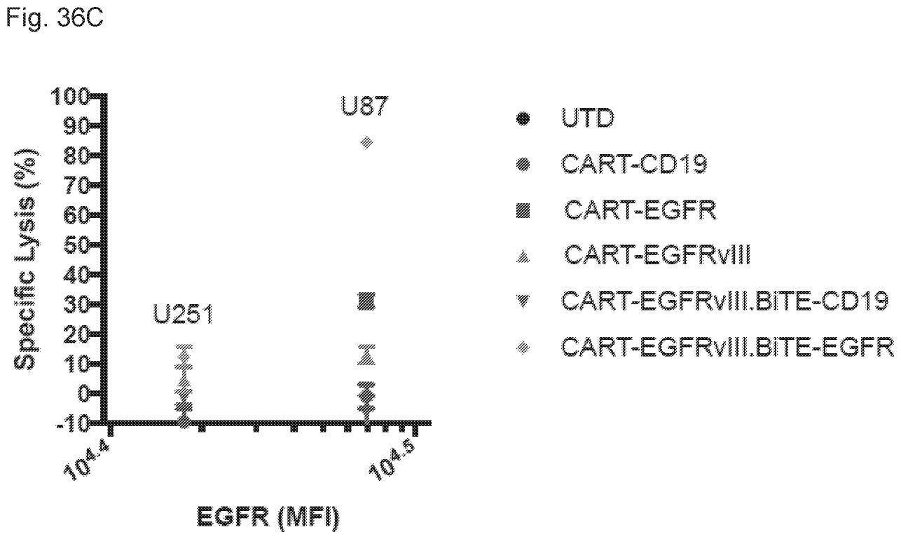

Next, the ability of CART.BiTE to elicit antigen-specific cytotoxic responses was tested. Using a standard bioluminescent cytotoxicity assay, we demonstrated that CART-EGFRvIII.BiTE-EGFR cells were highly cytotoxic and specific against EGFR-positive glioma (FIG. 35). These results were recapitulated using an impedance-based platform, which integrates microelectronics to capture real-time evaluation and kinetics of cell viability over time. In these assays, target EGFR-positive glioma cell lines were incubated with effector T cells and impedance, as represented by cell index (i.e., viability), was recorded longitudinally. Whereas wells cocultured with agnostic CART controls (e.g., CART-CD19, CART-EGFRvIII, and CART-EGFRvIII.BiTE-CD19) or UTD all displayed similar viability kinetics, CART-EGFRvIII.BiTE-EGFR cells were found to mediate rapid reduction in target cell viability against multiple glioma cell lines and at varied effector-to-target (E:T) ratios (FIG. 36A). When displayed as percent cytotoxicity at several time points, CART.BiTE cells were significantly more efficacious against GBM cells even when compared to positive control CART-EGFR (FIG. 36B). This effect correlated with the degree of EGFR expression on tumor cells, since targets with higher EGFR expression were lysed more efficiently by T cells transduced with either CART-EGFRvIII.BiTE-EGFR or CART-EGFR (FIG. 36C).

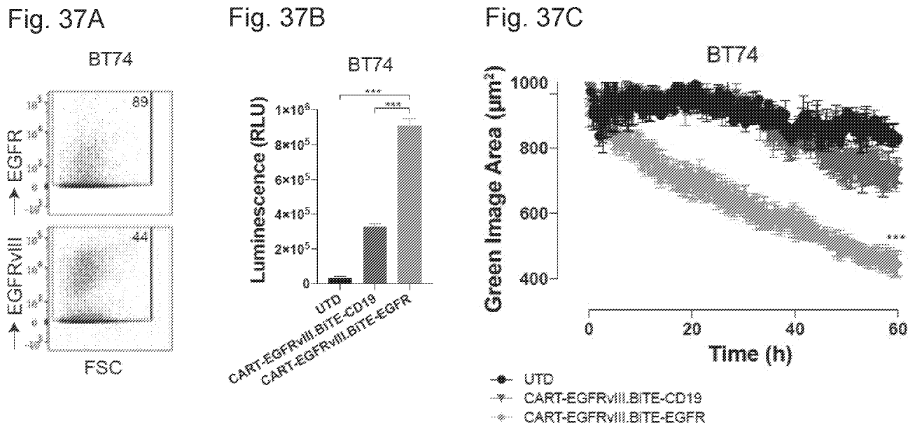

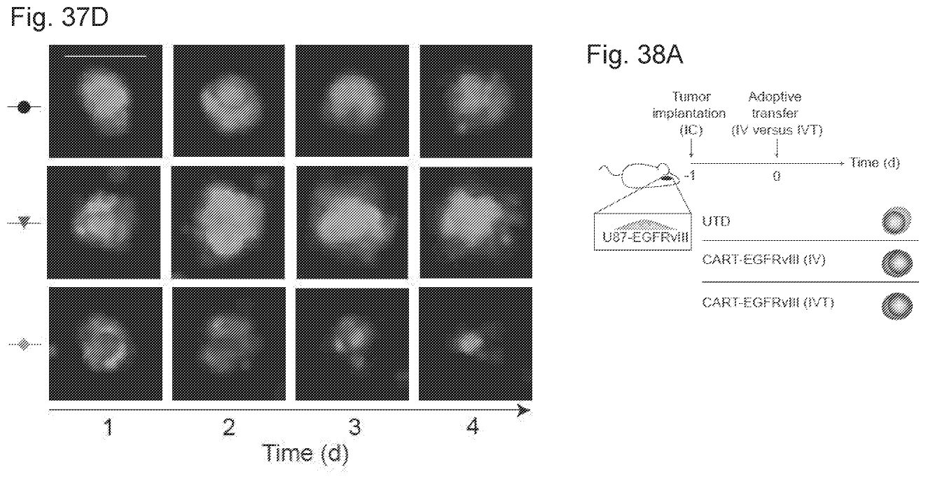

Patient-derived xenografts (PDXs) represent a recent focus of translational research in GBM and are thought to closely reproduce the genetic complexity and hallmark biological characteristics of brain tumors. Importantly, GBM PDXs have specifically been shown to maintain physiologically relevant EGFR copy number and amplification levels. In a study of more than 11 established GBM PDX neurospheres (Pandita et al., Genes Chromosomes Cancer 39:29-36, 2004), only one tumor contained both amplified EGFR and EGFRvIII. Given its natural dual antigen expression, it was reasoned that this model (i.e., BT74, formerly GBM6) would be an ideal platform for CART.BiTE evaluation. It was confirmed that BT74 reliably demonstrated heterogeneous expression of both EGFR and EGFRvIII (FIG. 37A). Highlighting this heterogeneity, Jurkat reporter T cells transduced with CART-EGFRvIII.BiTE-CD19 were activated in the presence of BT74—albeit to a significantly lesser degree than those transduced with CART-EGFRvIII.BiTE-EGFR—consistent with CAR-mediated recognition of EGFRvIII-expressing cells in culture (FIG. 37B).

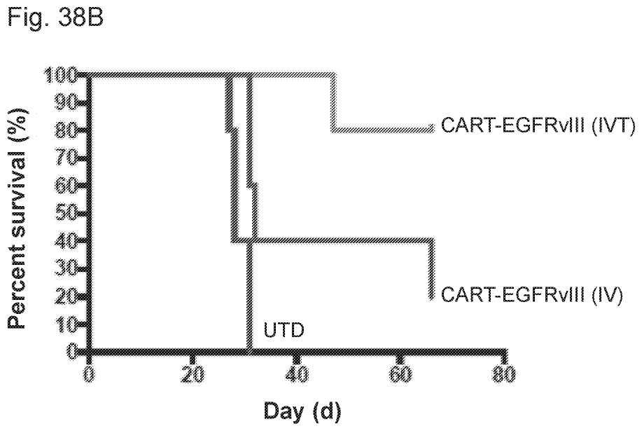

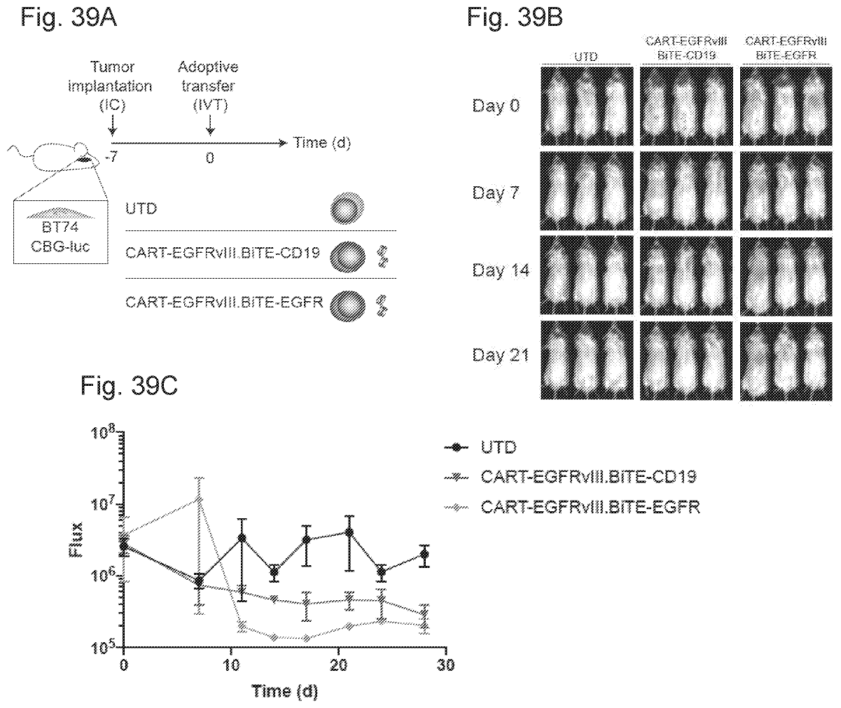

Because PDX neurospheres are nonadherent and therefore not amenable to viability measures based on impedance, antitumor cytotoxicity was assessed by live-cell, image-based analysis. Using this system, significant antitumor activity of CART-EGFRvIII.BiTE-EGFR cells against BT74 over time was demonstrated (FIG. 37C). This platform also enabled morphologic evaluation of the neurospheres themselves, which re-demonstrated selective antitumor efficacy in wells containing CAR T cells secreting BiTE-EGFR, compared to those secreting BiTE-CD19 or UTD controls (FIG. 37D). Given these observations, pilot experiments were designed to assess the activity of CART.BiTE against orthotopic PDX in immune compromised mice. It was found that regional, intraventricular delivery of CAR T cells was feasible, safe and superior to systemic delivery when treating tumors in the brain (FIGS. 38A and 38B), consistent with reported literature (Brown et al., N. Engl. J. Med. 375:2561-2569, 2016; Priceman et al., Clin. Cancer Res. 24:95-105, 2018; Choi et al., J. Clin. Neurosci. 21:189-190, 2014). When tested against BT74 in vivo, intraventricular administration of CART-EGFRvIII.BiTE-EGFR led to the durable regression of even 7-day established intracerebral PDX (FIGS. 39A-39C). Although mice treated with CART-EGFRvIII.BiTE-CD19 cells also eventually demonstrated treatment effect, this occurred late in the course of the experiment and, in retrospect, was consistent with reports that BT74 may have the ability to upregulate EGFRvIII when passaged in vivo over time (Pandita et al., Genes Chromosomes Cancer 39:29-36, 2004).

### Example 11. CART.BiTE is Efficacious and Safe Against EGFRvIII-Negative Tumors in Mice

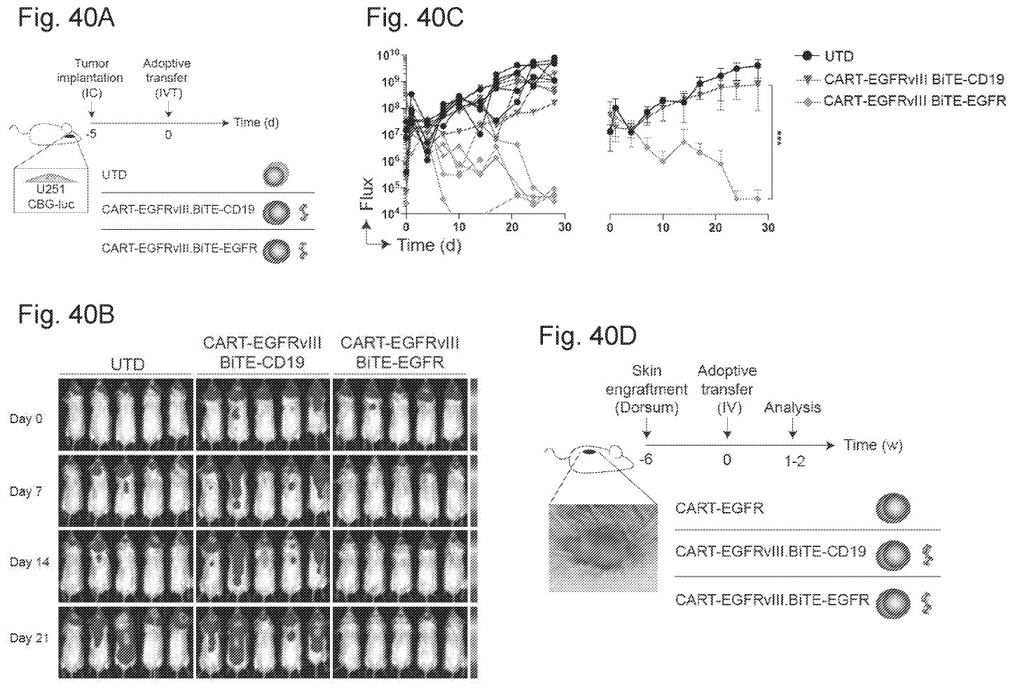

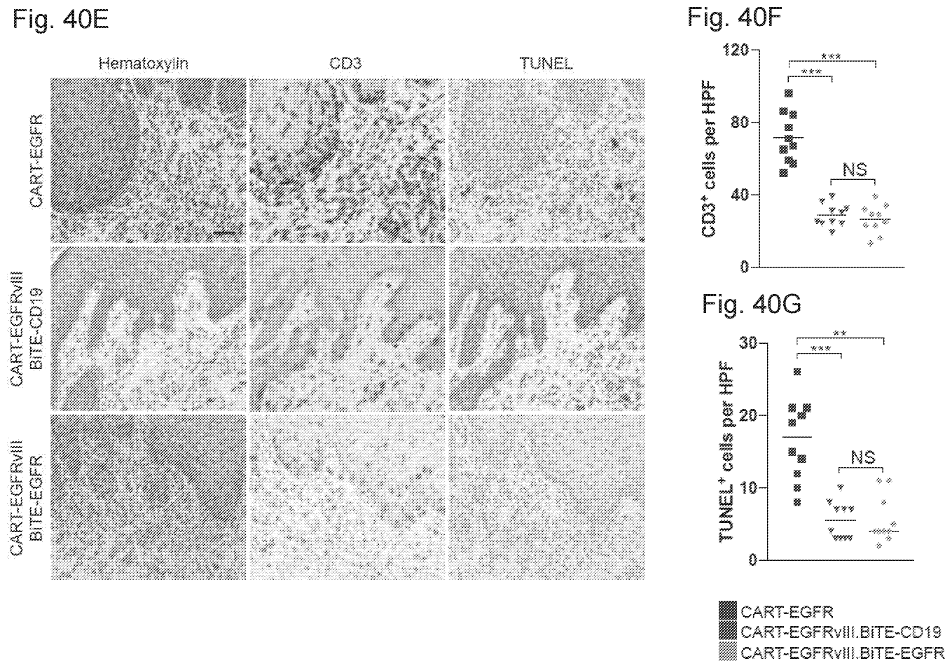

Given that the expression of EGFRvIII in BT74 proved variable in mice, it was next determined whether the in vivo efficacy of CART-EGFRvIII.BiTE-EGFR was dependent on CAR recognition of its cognate antigen, EGFRvIII, or if secreted BiTEs in the absence of CAR engagement were sufficient to detect measurable antitumor responses in vivo. To test this, human glioma cells (U251) were orthotopically implanted into NSG mice and proceeded with treatment (FIG. 40A). U251 is considered one of the most stringent glioma models in which to test efficacy, given its lack of EGFRvIII expression and relatively decreased surface expression of EGFR (FIG. 36C), as well as greater resistance to cell death from CART.BiTE cells in vitro (FIGS. 36A and 36B). Furthermore, compared to other cell lines, U251 has specifically been cited for its ability to most closely reflect the salient pathobiological features of human GBM when implanted in mice (Radaelli et al. Histol. Histopathol. 24:879-891, 2009). Using this xenograft model, we demonstrated durable regression of 5-day established glioma following injection with CART-EGFRvIII.BiTE-EGFR cells (FIGS. 40B and 40C). Conversely, mice treated with cells expressing CART-EGFRvIII and BiTE-CD19 demonstrated progressive tumor burden that was comparable to those receiving UTD control.

Because secreted BiTE-EGFR was necessary and sufficient to mediate antitumor efficacy against GBM, even in the absence of EGFRvIII, one potential concern could be that CART.BiTE might also result in significant on-target, off-tumor toxicity in normal human tissues that express wild-type EGFR. However, it was hypothesized that, given the very low levels of BiTE secretion from transduced T cells (FIG. 33H), local targeting of EGFR through secreted BiTE could result in an improved safety profile compared to alternative approaches such as direct immunotherapeutic targeting by EGFR-specific CAR T cells. To test this, the previously published skin graft toxicity model was used (Johnson et al., Sci. Transl. Med. 7:275ra222, 2015), which enables in vivo assessment of immune responses against human tissues expressing EGFR at endogenous levels. Skin grafting was selected for ease of visualization and harvest for analysis, and also because dermatologic reactions represent a major side effect of several FDA-approved therapies that target EGFR (Agero et al., J. Am. Acad. Dermatol. 55:657-670, 2006).

Human skin was transplanted onto the dorsa of NSG mice and allowed to fully heal prior to treatment with CAR T-cell therapy (FIG. 40D). CART-EGFR cells, based on the variable chains of cetuximab, served as positive controls for inducing skin toxicity, while CAR T cells against EGFRvIII—which have previously been shown to be safe in skin graft experiments (Johnson et al., supra) and clinical trials (O'Rourke et al., supra)—but modified to secrete CD19 BiTEs, were used as a negative control. All T cells were delivered intravenously, rather than intracranially, in order to increase the sensitivity for toxicity that might stem from pharmacokinetic distribution of CAR T cells and secreted BiTEs into systemic circulation. Skin samples were harvested up to two weeks after infusion and subjected to histologic examination. Mice treated with CART-EGFR demonstrated intense lymphocytic infiltration in the dermis and epidermis of their skin grafts. Analysis by IHC revealed a robust CD3⁺ T-cell infiltrate, as well as adjacent areas of keratinocyte apoptosis and TUNEL⁺ cells, consistent with cutaneous graft-versus-host disease (FIG. 40E). Conversely, these signs were absent in mice treated with CART-EGFRvIII.BiTE-EGFR cells, which, when quantified across 10 consecutive high-power fields, did not differ significantly from controls (FIGS. 40F and 40G). These results suggest that there is a therapeutic window for CARs designed to secrete low levels of BiTE, and that targeting an antigen on healthy tissues may be safe, even when CART.BiTE cells are administered systemically.

### Example 12. BiTEs Secreted by CAR T Cells Recruit Bystander Effector Activity

In patients, GBM tumors are variably infiltrated by endogenous T cells at baseline, and the presence of these cells has been shown to predict favorable clinical outcomes (Lohr et al., Clin. Cancer Res. 17:4296-4308, 2011). Likewise, the clinical study of patients receiving CART-EGFRvIII cells demonstrated robust bystander T-cell infiltrate within the tumor bed (O'Rourke et al., supra). However, it has also been shown that tumor-infiltrating lymphocytes (TILs) often have tumor-agonistic specificities and may recognize a wide range of epitopes completely unrelated to cancer (Simoni et al. Nature 557:575-579, 2018). Thus, these unmodified, endogenous T cells, may represent an untapped resource with the potential to be redirected into tumor-specific cytotoxic killers (FIG. 32E).

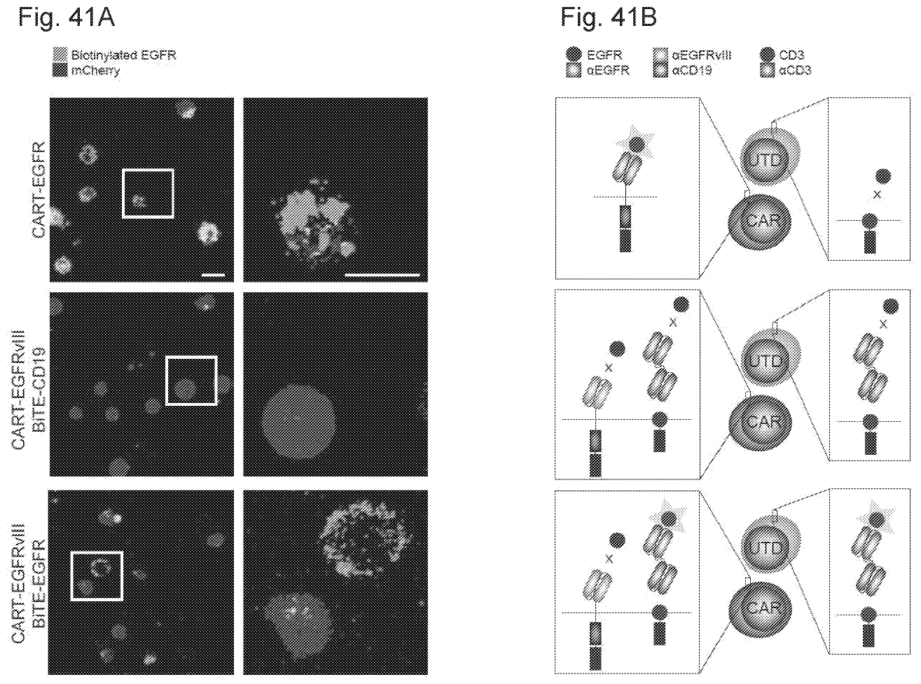

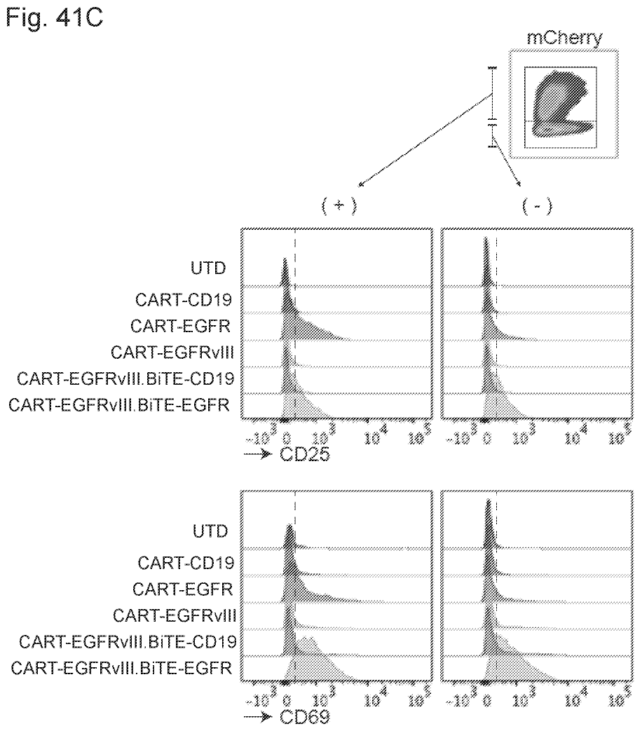

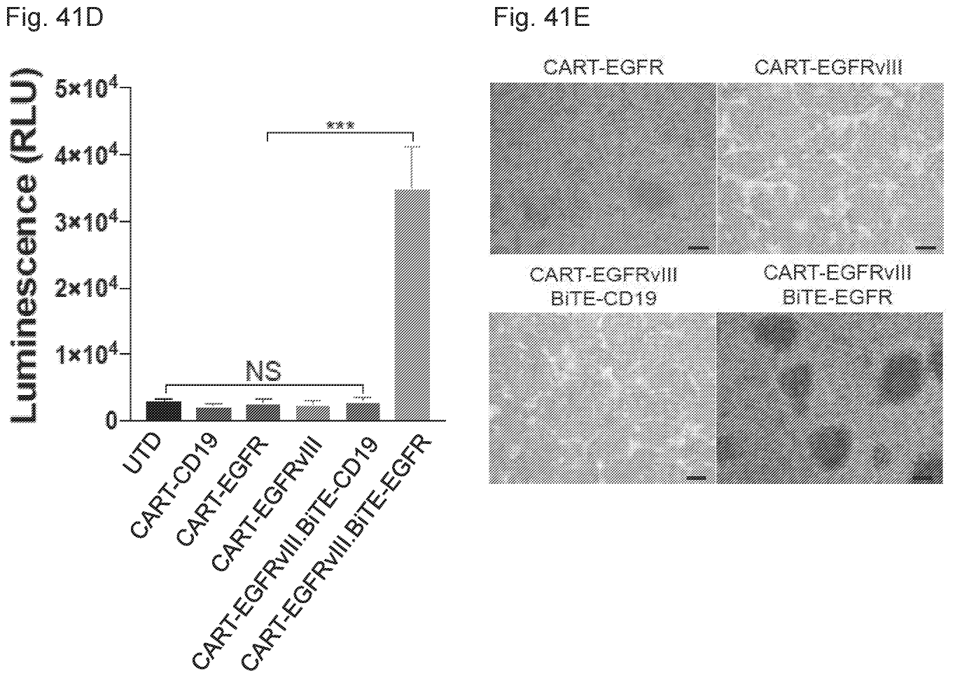

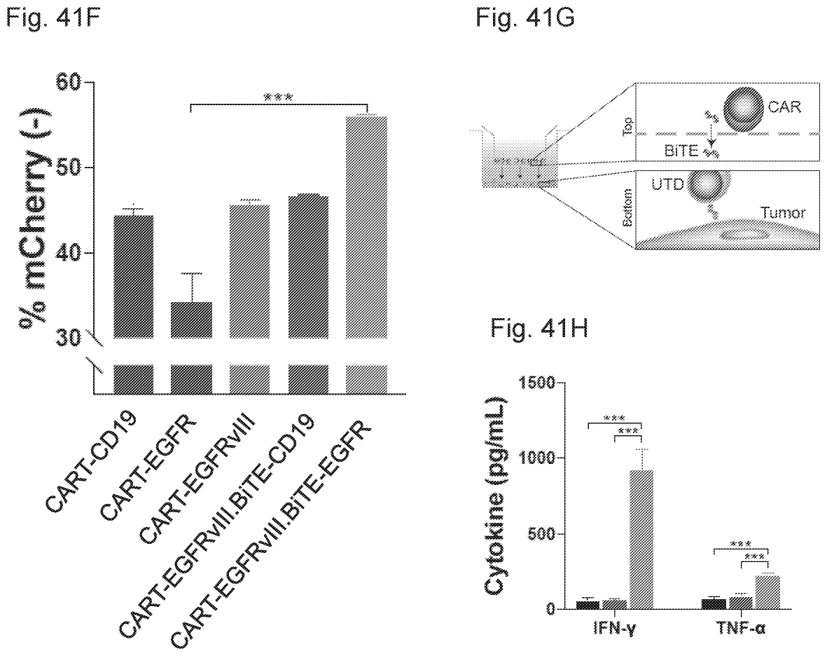

Mechanistically, it remained unclear whether secreted BiTEs were solely recruiting cells that had been modified to express the transgene, versus primarily redirecting the bystander T-cell compartment, which is incidentally present in all CAR T-cell preparations for both research and clinical use (FIG. 33B). In order to specifically characterize the interaction between secreted BiTE and bystander T cells, confocal microscopy was first used to visualize the distribution of EGFR-specific BiTEs on both CAR T cells and untransduced T cells in culture. As before, transduced cells were identified by expression of the mCherry fluorescent protein (FIG. 33A). In addition, biotinylated EGFR was used for sensitive detection of the anti-EGFR scFv, which in this case could be expressed either as a transmembrane protein in the form of a CAR, or as the unbound arm of a BiTE opposite to its binding site for CD3. Unless otherwise stated, all assays were performed in triplicate (mean±SEM is depicted; unpaired t test, ***=p<0.001). As anticipated, it was found that positive control CART-EGFR cells successfully bound free EGFR antigen (FIGS. 41A and 41B; top). Conversely, negative control T cells transduced with CART-EGFRvIII.BiTE-CD19 did not show signs of specificity for EGFR through either their CAR or secreted BiTE components (FIGS. 41A and 41B; middle). However, in cultures that had been transduced with CART-EGFRvIII.BiTE-EGFR, evidence of EGFR-specific BiTEs was found, bound in clusters not only bound to transduced mCherry-positive cells, but also on the surface of bystander, mCherry-negative, untransduced cells (FIGS. 41A and 41B; bottom).

Next, the ability of secreted BiTEs to potentiate paracrine immune responses against tumor cells was evaluated, specifically from the bystander compartment. Using flow cytometric analysis on cocultures of GBM and primary human T cells, it was found that only CART-EGFRvIII.BiTE-EGFR mediated activation of mCherry-negative cells, as indicated by early induction of CD25 and CD69 (FIG. 41C). As anticipated, activation was also observed in cocultures containing CART-EGFR cells, but this was confined to the mCherry-positive population, while bystander cells in these cultures remained unchanged. Additional experiments in which bystander T cells were replaced by untransduced Jurkat reporter T cells demonstrated antigen-specific activation, again only in the presence of human CAR T cells secreting BiTE-EGFR (FIG. 41D). Importantly, prior to the assay, these reporter cells had not been cultured under conditions during which BiTE molecules were being actively produced, as is typical during standard CAR T-cell expansion; therefore, bystander activation could be attributed specifically to BiTE secreted during the assay.

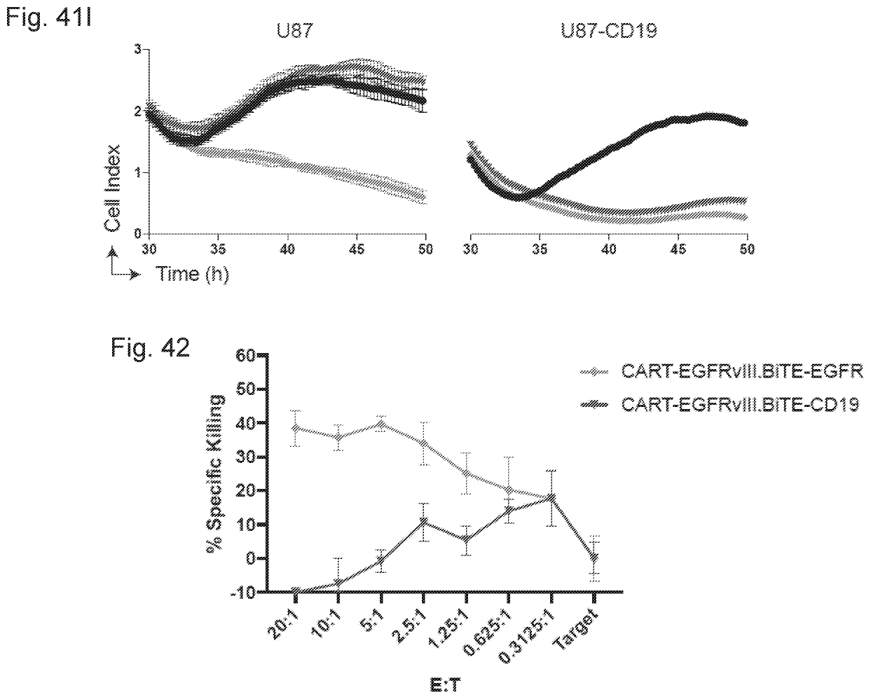

Also assessed was the degree to which CART.BiTE could elicit bystander T-cell functional activity, which was measured by parameters such as proliferation, cytokine secretion, and antitumor cytotoxicity. It was determined that, whereas mCherry-positive, CART-EGFR cells proliferated indiscriminately upon encountering their target antigen in culture, a significant proportion of proliferation in cultures transduced with CART-EGFRvIII.BiTE-EGFR was observed within the bystander T-cell compartment (FIGS. 41E and 41F). Finally, a 0.4 μm transwell system was used which provided a physical barrier between gene-modified cells and UTD effectors, while permitting soluble BiTE to freely pass between chambers (FIG. 41G). This strategy eliminated variables associated with direct cell-cell interaction or unexpected activity between nonspecific CAR T cells and tumors in culture. Using this system, it was found that BiTEs produced by CART.BiTE cells successfully translocated across the transwell membrane to mediate Th1 cytokine production and antigen specific cytotoxicity from unmodified T cells in the presence of GBM (FIGS. 41H and 41I). This effect was also generalizable in that results were recapitulated with CART-EGFRvIII.BiTE-CD19 in the setting of target cells expressing CD19. Notably, antitumor cytotoxicity was also observed when UTD effector cells were replaced by high-purity, flow cytometric cell-sorted regulatory T cells (T_{regs}) (FIG. 42). This finding is consistent with prior work demonstrating that BiTEs have the capacity to convert even T_{regs} into antitumor cytotoxic killers via the granzyme-perforin pathway (Choi et al., Cancer Immunol. Res. 1:163, 2013). T_{regs} represent a highly suppressive cell population in patients with GBM and were actually overrepresented in TILs from patients treated with CART-EGFRvIII (O'Rourke et al., supra). Thus, these findings highlight an additional mechanism by which CART.BiTE could enhance antitumor immunity and mitigate tumor escape from T-cell rejection.

### Example 13. Simultaneous Redirection Through CARs and BiTEs Results in Favorable T-Cell Differentiation and Phenotype

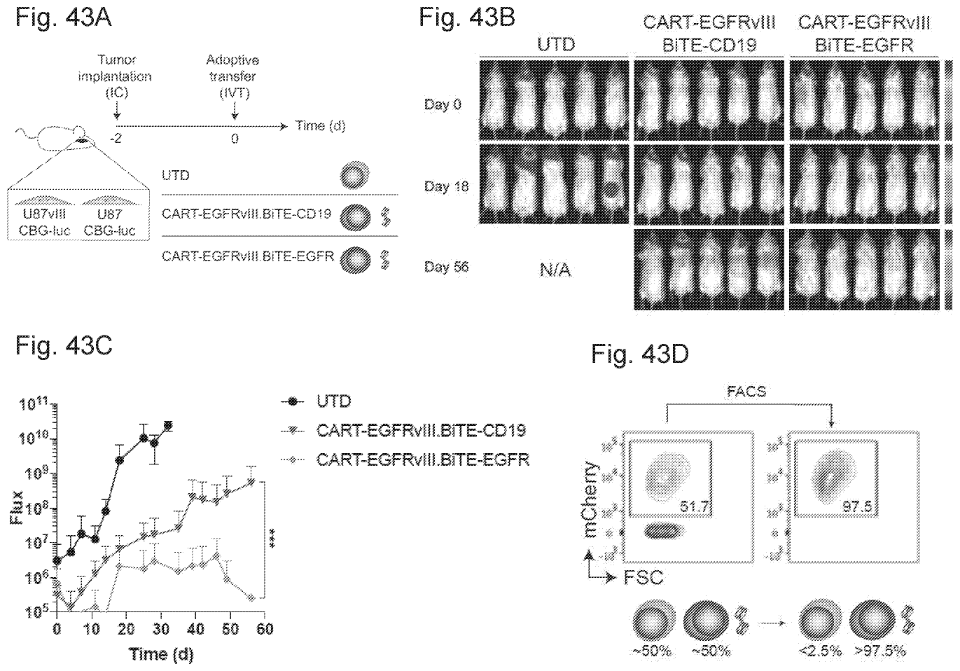

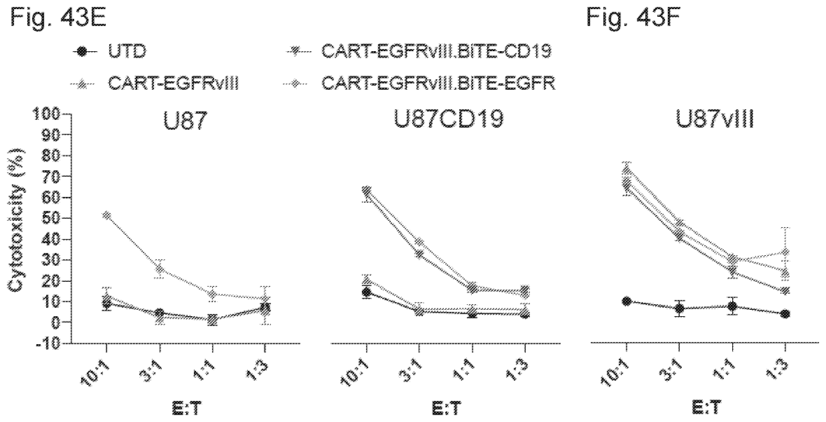

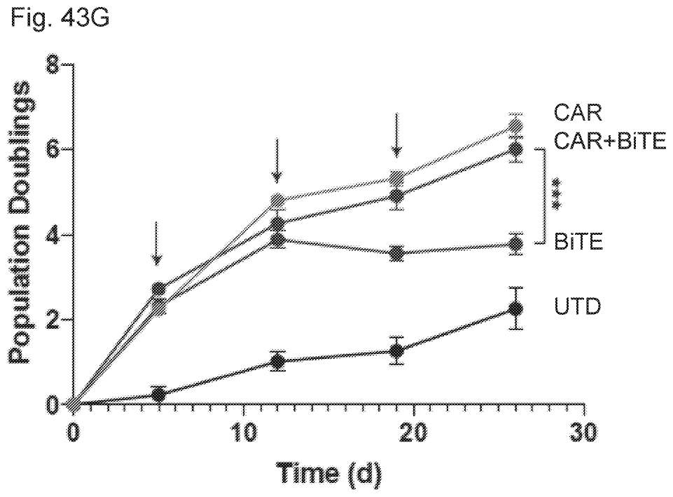

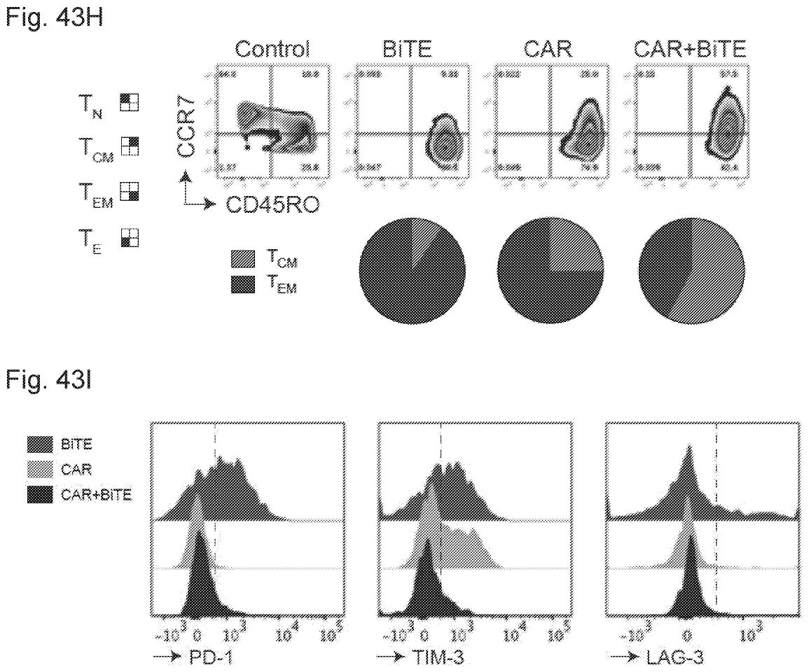

In a model of heterogeneous brain tumors expressing EGFRvIII in only 10% of cells, injection with CART-EGFRvIII.BiTE-EGFR cells resulted in complete and durable responses in all mice (FIGS. 43A-43C). In this setting, CART.BiTE cells would likely be activated both by their CAR and secreted, bound BiTEs. To understand the effects of simultaneous activation through CARs and BiTEs, fluorescence activated cell sorting (FACS) was used to isolate the transduced, mCherry-positive subpopulation of several CAR T-cell cultures at a purity of greater than 97.5% (FIG. 43D). This step was performed to largely exclude the contribution of CAR-negative bystander cells in subsequent analyses. Using this approach, it was demonstrated that T cells transduced to express CARs maintained the capacity to efficiently lyse target tumor cells through BiTE-mediated cytotoxicity (FIG. 43E). This was true for both BiTE-EGFR and BiTE-CD19 when tested against corresponding target tumor lines expressing EGFR and CD19, respectively. In addition, CAR T cells redirected through both CAR and BiTE (e.g., CART-EGFRvIII.BiTE-EGFR against U87vIII, which expresses both EGFRvIII and EGFR) yielded comparable cytotoxic activity when compared to CAR alone (FIG. 43F). These data provided early evidence that CARs and BiTEs might be able to signal together without necessarily generating conflicting, counterproductive effects, or immunodominance of one platform over the other.

To further characterize BiTE activity in the context of CAR-transduced cells, each mode of stimulation was also compared for its ability to initiate and maintain T-cell proliferation. Notably, while BiTEs are limited to activating T cells via CD3 stimulation, CAR T cells are engineered to express both intracellular CD3 as well as potent costimulatory domains such as 4-1 BB, in this case. Thus, by design, it was surmised that CARs might outperform BiTEs in assays measuring certain functional parameters when measured head-to-head. Indeed, following serial antigen stimulation with irradiated target cells, growth of sorted transduced cells undergoing BiTE stimulation plateaued after approximately 12 days, whereas repeated antigen stimulation through CARs maintained logarithmic growth for over one month (FIG. 43G). Interestingly, when activated simultaneously through CARs and BiTEs, the proliferation deficit observed with BiTEs alone was almost entirely abrogated.

T cells exist in various states of differentiation, each with unique functional capabilities. In clinical studies, BiTEs have been shown to selectively promote expansion of well-differentiated effector memory cells (T_{EM}) (Bargou et al., Science 321:974-977, 2008); however, superior outcomes for CARTs have been achieved using less differentiated stem cell memory (T_{SCM}) or central memory (T_{CM}) subtypes (Sadelain et al., Nature 545:423-431, 2017). These less differentiated phenotypes are associated with enhanced expansion and persistence, the capacity for self-renewal, and the ability to generate shorter-lived T_{EM}. Thus, it was hypothesized that the differences observed in proliferation during serial antigen stimulation through each modality (FIG. 43G) might also result in distinct T-cell differentiation patterns. Indeed, consistent with our data as well as with prior studies, CAR T cells undergoing prolonged stimulation with BiTEs alone preferentially enriched T_{EM} cells, while those activated through either CARs alone or CARs with BiTEs appeared to enrich for the less differentiated T_{CM} compartment (FIG. 43H). Finally, the surface markers indicative of T-cell exhaustion were characterized, which is a state characterized by general hypo-responsiveness, limited proliferative capacity and severely impaired effector function. It was found that stimulation through BiTEs alone upregulated the expression of several immune checkpoint inhibitors associated with exhausted T cells (e.g., PD-1, TIM-3 and LAG-3); however, when CARs and BiTEs were combined, the polarization toward T-cell exhaustion was reversed (FIG. 43I). These findings corroborate prior studies demonstrating the favorable effects of costimulation—especially through 4-1 BB—on mitigating exhaustion in CAR T cells (Long et al., Nat. Med. 21:581-590, 2015), and also suggest that these benefits might be extended to combination therapy with BiTEs against other antigens. Our findings reveal new insights into how CARs and BiTEs, which are typically thought to be competitive technologies, could instead be used to complement each other.

### Example 14. Immune Cells Genetically Modified to Target Multiple Antigens with Combinations of Tandem Chimeric Antigen Receptors (CARs) and Secreted BiTEs

Heterogeneous target antigen expression and outgrowth of tumors lacking the target antigen can limit responses of cancer to immunotherapy using immune cells (e.g., T cells) engineered to express CARs. For example, glioblastoma (GBM) is a cancer with extremely poor prognosis that is known to express surface antigens that may be targeted for effective antitumor immunity, including EGFRvIII, IL13Rα2, EGFR, HER2, and ephrins. However, to date, responses of GBM to CART cells directed against single antigens such as EGFRvIII or IL-13Rα2 have been limited, in part due to antigen escape.

To address this issue, a second generation CAR was designed comprised of two or more antigen-binding domains (e.g., two or more single chain fragment variable (scFv) regions, two or more ligands, or a combination of one or more scFvs and one or more ligands). Such a CAR has the capacity to be activated by engagement with two or more different antigens, for example, EGFRvIII and IL-13Rα2. To further increase the breadth of responses achievable through this approach and to protect against tumor progression via antigen escape, additional specificity or targets for the immune cells can be provided by engineering the immune cells to also secrete bispecific antibodies (e.g., BiTEs) targeting an additional antigen, for example, EGFR or HER2.

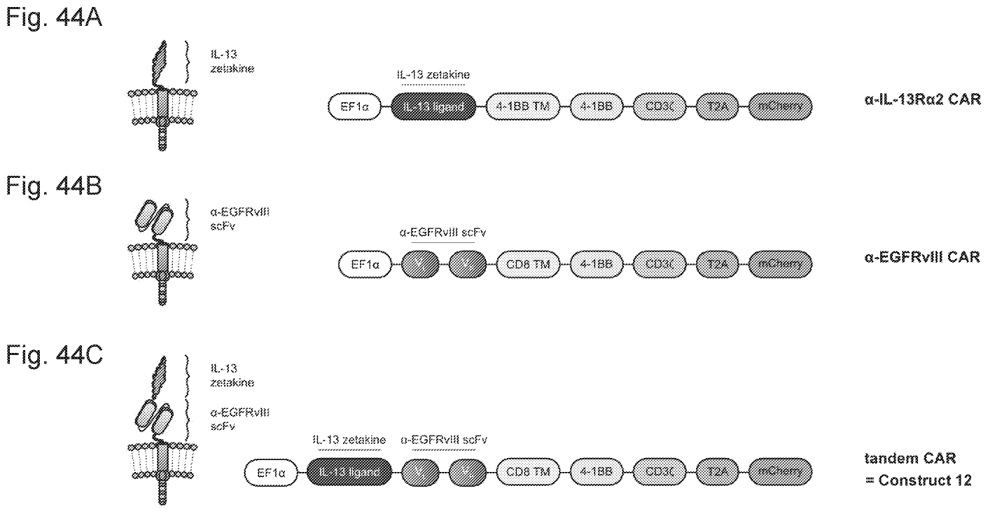

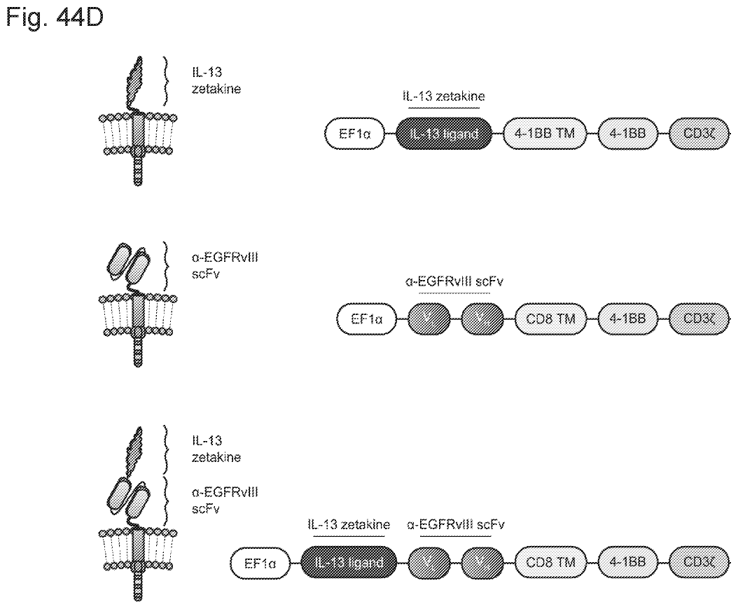

A tandem CAR construct directed against IL-13Rα2 and EGFRvIII was designed along with two control CARs directed against either single antigen (FIGS. 44A-44C). The tandem CAR (Construct 12) includes an EF1α promoter, an IL-13 ligand (IL-13 zetakine), an anti-EGFRvIII scFv, a CD8 transmembrane domain, a 4-1 BB co-stimulatory domain, a CD3ζ domain, a T2A peptide sequence, and a reporter gene (mCherry). The construct may be a polycistronic vector that further encodes a BiTE (e.g., a BiTE targeting EGFR or HER2), for example, using a T2A peptide or an internal ribosomal entry site (IRES). Such a polycistronic vector can be designed, for example, according to the methods described in Examples 8 and/or analogous the constructs described in Examples 5 and 10. In other examples, CAR-T cells transduced with a tandem CAR construct such as Construct 12 can be transduced with separate vectors for expression of a BiTE.

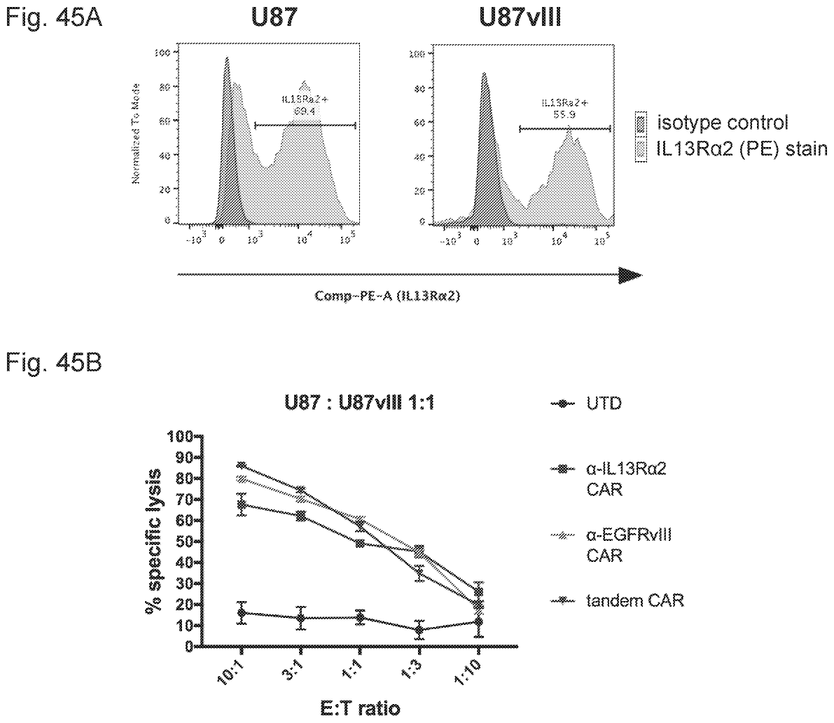

An in vitro model for heterogeneous glioblastoma was developed using U87 human glioblastoma cells and U87 cells transduced to express EGFRvIII (U87vIII). Expression of IL13Rα2 was confirmed in both U87 and U87vIII glioblastoma as assessed by flow cytometry (FIG. 45A). Next, a cytotoxicity assay of untransduced T cells (UTD), anti-IL-13Rα2 CAR T cells, anti-EGFRvIII CAR T cells, or tandem anti-IL-13Rα2/anti-EGFRvIII CAR T cells was performed. As shown in FIG. 45B, each CAR T cell population induced cytotoxicity of the target cell population (a 1:1 ratio of U87 and U87vIII glioblastoma cells), with the tandem anti-IL-13Rα2/anti-EGFRvIII CAR T cells showing the highest efficacy in specific lysis compared to the CAR T cells targeting the single antigens at the effector:target (E:T) ratios of 10:1 and 3:1.

In summary, we have developed tandem CARs targeting two or more distinct antigens, e.g., EGFRvIII and IL-13Rα2, which can be engineered to secrete bispecific antibodies (e.g., BiTEs) targeting an additional antigen, e.g., EGFR or HER2. This technique can be extended to other tandem CARs or BiTEs targeting other surface tumor antigens, e.g., EGFR, EGFRvIII, CD19, CD79b, CD37, PSMA, PSCA, IL-13Rα2, EphA1, Her2, mesothelin, MUC1, MUC16, or others. For example, a tandem CAR can be designed to target EGFR and EGFRvIII, PSMA and PSCA; CD19 and CD79b; CD79b and CD37; CD19 and CD37; EphA1 and Her2; EphA1 and mesothelin; Her2 and mesothelin, MUC1 and MUC16; as well as other combinations of the aforementioned tumor antigens.

### Example 15. Sequence Information

Anti-GARP CAR-Construct 1: CD8 signal sequence-anti-GARP-CD8 hinge+TM-4-1BB-CD3ζ (SEQ ID NO: 1) including CD8 signal sequence (amino acids 1-21 of SEQ ID NO: 1; SEQ ID NO: 2); anti-GARP camelid (amino acids 22-128 of SEQ ID NO: 1; SEQ ID NO: 3); CD8 hinge/TM domain (amino acids 129-197 of SEQ ID NO: 1; SEQ ID NO: 4); 4-1BB ICD (amino acids 198-239 of SEQ ID NO: 1; SEQ ID NO: 5); and CD3ζ (amino acids 240-351 of SEQ ID NO: 1; SEQ ID NO: 6).

(SEQ ID NO: 1)
MALPVTALLLPLALLLHAARPDIQMTQSPSSLSASLGDRVTITCQASQSI
SSYLAWYQQKPGQAPNILIYGASRLKTGVPSRFSGSGSGTSFTLTISGLE
AEDAGTYYCQQYASVPVTFGQGTKVELKTTTPAPRPPTPAPTIASQPLSL
RPEACRPAAGGAVHTRGLDFACDIYIWAPLAGTCGVLLLSLVITLYCKRG
RKKLLYIFKQPFMRPVQTTQEEDGCSCRFPEEEEGGCELRVKFSRSADAP
AYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPP
R

CD8 signal sequence (amino acids 1-21 of SEQ ID NO: 1)

(SEQ ID NO: 2)
MALPVTALLLPLALLLHAARP

Anti-GARP camelid (amino acids 22-128 of SEQ ID NO: 1)

(SEQ ID NO: 3)
DIQMTQSPSSLSASLGDRVTITCQASQSISSYLAWYQQKPGQAPNILIY
GASRLKTGVPSRFSGSGSGTSFTLTISGLEAEDAGTYYCQQYASVPVTF
GQGTKVELK

CD8 hinge/TM domain (amino acids 129-197 of SEQ ID NO: 1)

(SEQ ID NO: 4)
TTTPAPRPPTPAPTIASQPLSLRPEACRPAAGGAVHTRGLDFACDIYIW
APLAGTCGVLLLSLVITLYC

4-1 BB ICD (amino acids 198-239 of SEQ ID NO: 1)

(SEQ ID NO: 5)
KRGRKKLLYIFKQPFMRPVQTTQEEDGCSCRFPEEEEGGCEL

CD3ζ (amino acids 240-351 of SEQ ID NO: 1)

(SEQ ID NO: 6)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

Anti-LAP CAR (H-L)-Construct 2: CD8 signal sequence-anti-LAP-CD8 hinge+TM-4-1BB-CD3ζ (SEQ ID NO: 7) including CD8 signal sequence (amino acids 1-21 of SEQ ID NO: 7; SEQ ID NO: 8), anti-LAP scFv (H-L) (amino acids 22-307 of SEQ ID NO: 7; SEQ ID NO: 9), CD8 hinge/TM domain (amino acids 308-376 of SEQ ID NO: 7; SEQ ID NO: 10), 4-1 BB ICD (amino acids 377-418 of SEQ ID NO: 7; SEQ ID NO: 11), and CD3ζ (amino acids 419-530 of SEQ ID NO: 7; SEQ ID NO: 12).

(SEQ ID NO: 7)
MALPVTALLLPLALLLHAARPMKLWLNWIFLVTLLNDIQCEVKLVESGGG
LVQPGGSLSLSCAASGFTFTDYYMSWVRQPPGKALEWLGFIRNKPNGYTT
EYSASVKGRFTISRDNSQSILYLQMNVLRAEDSATYYCARYTGGGYFDYV
VGQGTTLTVSSGGGGSGGGGSGGGGSGGGGSMMSSAQFLGLLLLCFQGTR
CDIQMTQTTSSLSASLGDRLTISCRASQDISNYLNWYQQKPDGTVKLLIY
YTSRLHSGVPSRFSGSGSGTDYSLTISNLEQADIATYFCQQGDTLPWTFG
GGTKLEIKTTTPAPRPPTPAPTIASQPLSLRPEACRPAAGGAVHTRGLDF
ACDIYIWAPLAGTCGVLLLSLVITLYCKRGRKKLLYIFKQPFMRPVQTTQ
EEDGCSCRFPEEEEGGCELRVKFSRSADAPAYQQGQNQLYNELNLGRREE
YDVLDKRRGRDPEMGGKPRRKNPQEGLYNELQKDKMAEAYSEIGMKGERR
RGKGHDGLYQGLSTATKDTYDALHMQALPPR

CD8 signal sequence (amino acids 1-21 of SEQ ID NO: 7)

(SEQ ID NO: 8)
MALPVTALLLPLALLLHAARP

Anti-LAP scFv (H-L) (amino acids 22-307 of SEQ ID NO: 7)

(SEQ ID NO: 9)
MKLWLNWIFLVTLLNDIQCEVKLVESGGGLVQPGGSLSLSCAASGFTFTD
YYMSWVRQPPGKALEWLGFIRNKPNGYTTEYSASVKGRFTISRDNSQSIL
YLQMNVLRAEDSATYYCARYTGGGYFDYWGQGTTLTVSSGGGGSGGGGSG
GGGSGGGGSMMSSAQFLGLLLLCFQGTRCDIQMTQTTSSLSASLGDRLTI
SCRASQDISNYLNWYQQKPDGTVKLLIYYTSRLHSGVPSRFSGSGSGTDY
SLTISNLEQADIATYFCQQGDTLPWTFGGGTKLEIK

CD8 hinge/TM domain (amino acids 308-376 of SEQ ID NO: 7)

(SEQ ID NO: 10)
TTTPAPRPPTPAPTIASQPLSLRPEACRPAAGGAVHTRGLDFACDIYIWA
PLAGTCGVLLLSLVITLYC

4-1 BB ICD (amino acids 377-418 of SEQ ID NO: 7)

(SEQ ID NO: 11)
KRGRKKLLYIFKQPFMRPVQTTQEEDGCSCRFPEEEEGGCEL

CD3ζ (amino acids 419-530 of SEQ ID NO: 7).

(SEQ ID NO: 12)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

Anti-LAP CAR (L-H)-Construct 3: CD8 signal sequence-anti-LAP CD8 hinge+TM-4-1BB-CD3ζ (SEQ ID NO: 13) including CD8 signal (amino acids 1-21 of SEQ ID NO: 13; SEQ ID NO: 14), anti-LAP scFv (L-H) (amino acids 22-307 of SEQ ID NO: 13; SEQ ID NO: 15), CD8 hinge/TM (amino acids 308-376 of SEQ ID NO: 13; SEQ ID NO: 16), 4-1BB ICD (amino acids 377-418 of SEQ ID NO: 13; SEQ ID NO: 17), and CD3ζ (amino acids 419-530 of SEQ ID NO: 13; SEQ ID NO: 18).

(SEQ ID NO: 13)
MALPVTALLLPLALLLHAARPMMSSAQFLGLLLLCFQGTRCDIQMTQTTS
SLSASLGDRLTISCRASQDISNYLNWYQQKPDGTVKLLIYYTSRLHSGVP
SRFSGSGSGTDYSLTISNLEQADIATYFCQQGDTLPWTFGGGTKLEIKGG
GGSGGGGSGGGGSGGGGSMKLWLNWIFLVTLLNDIQCEVKLVESGGGLVQ
PGGSLSLSCAASGFTFTDYYMSWVRQPPGKALEWLGFIRNKPNGYTTEYS
ASVKGRFTISRDNSQSILYLQMNVLRAEDSATYYCARYTGGGYFDYVVGQ
GTTLTVSSTTTPAPRPPTPAPTIASQPLSLRPEACRPAAGGAVHTRGLDF
ACDIYIWAPLAGTCGVLLLSLVITLYCKRGRKKLLYIFKQPFMRPVQTTQ
EEDGCSCRFPEEEEGGCELRVKFSRSADAPAYQQGQNQLYNELNLGRREE
YDVLDKRRGRDPEMGGKPRRKNPQEGLYNELQKDKMAEAYSEIGMKGERR
RGKGHDGLYQGLSTATKDTYDALHMQALPPR

CD8 signal sequence (amino acids 1-21 of SEQ ID NO: 13)

(SEQ ID NO: 14)
MALPVTALLLPLALLLHAARP

Anti-LAP scFv (L-H) (amino acids 22-307 of SEQ ID NO: 13)

(SEQ ID NO: 15)
MMSSAQFLGLLLLCFQGTRCDIQMTQTTSSLSASLGDRLTISCRASQDIS
NYLNWYQQKPDGTVKLLIYYTSRLHSGVPSRFSGSGSGTDYSLTISNLEQ
ADIATYFCQQGDTLPWTFGGGTKLEIKGGGGSGGGGSGGGGSGGGGSMKL
WLNWIFLVTLLNDIQCEVKLVESGGGLVQPGGSLSLSCAASGFTFTDYYM
SWVRQPPGKALEWLGFIRNKPNGYTTEYSASVKGRFTISRDNSQSILYLQ
MNVLRAEDSATYYCARYTGGGYFDYVVGQGTTLTVSS

CD8 hinge/TM (amino acids 308-376 of SEQ ID NO: 13)

(SEQ ID NO: 16)
TTTPAPRPPTPAPTIASQPLSLRPEACRPAAGGAVHTRGLDFACDIYIWA
PLAGTCGVLLLSLVITLYC

4-1 BB ICD (amino acids 377-418 of SEQ ID NO: 13)

(SEQ ID NO: 17)
KRGRKKLLYIFKQPFMRPVQTTQEEDGCSCRFPEEEEGGCEL

CD3ζ (amino acids 419-530 of SEQ ID NO: 13)

(SEQ ID NO: 18)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

Anti-EGFR CAR secreting anti-GARP Camelid-Construct 4: CD8 signal sequence-anti-EGFR-CD8 hinge+TM-4-1 BB-CD3ζ-anti-GARP camelid (SEQ ID NO: 19) including CD8 signal sequence (amino acids 1-21 of SEQ ID NO: 19; SEQ ID NO: 20), anti-EGFR scFv (amino acids 22-267 of SEQ ID NO: 19; SEQ ID NO: 21), CD8 hinge/TM (amino acids 268-336 of SEQ ID NO: 19; SEQ ID NO: 22), 4-1 BB (amino acids 337-378 of SEQ ID NO: 19; SEQ ID NO: 23), CD3ζ (amino acids 379-490 of SEQ ID NO: 19; SEQ ID NO: 24), 2A cleavage sequence (amino acids 494-515 of SEQ ID NO: 19; SEQ ID NO: 31), Igκ leader (amino acids 519-539 of SEQ ID NO: 19; SEQ ID NO: 32), and anti-GARP camelid (amino acids 540-646 of SEQ ID NO: 19; SEQ ID NO: 25).

(SEQ ID NO: 19)
MALPVTALLLPLALLLHAARPQVQLKQSGPGLVQPSQSLSITCTVSGFSL
TNYGVHWVRQSPGKGLEWLGVIWSGGNTDYNTPFTSRLSINKDNSKSQVF
FKMNSLQSNDTAIYYCARALTYYDYEFAYWGQGTLVTVSAGGGGSGGGGS
GGGGSGGGGSDILLTQSPVILSVSPGERVSFSCRASQSIGTNIHWYQQRT
NGSPRLLIKYASESISGIPSRFSGSGSGTDFTLSINSVESEDIADYYCQQ
NNNWPTTFGAGTKLELKTTTPAPRPPTPAPTIASQPLSLRPEACRPAAGG
AVHTRGLDFACDIYIWAPLAGTCGVLLLSLVITLYCKRGRKKLLYIFKQP
FMRPVQTTQEEDGCSCRFPEEEEGGCELRVKFSRSADAPAYQQGQNQLYN
ELNLGRREEYDVLDKRRGRDPEMGGKPRRKNPQEGLYNELQKDKMAEAYS
EIGMKGERRRGKGHDGLYQGLSTATKDTYDALHMQALPPRPGSGSGATNF
SLLKQAGDVEENPGPRTAMETDTLLLWVLLLWVPGSTGDDIQMTQSPSSL
SASLGDRVTITCQASQSISSYLAWYQQKPGQAPNILIYGASRLKTGVPSR
FSGSGSGTSFTLTISGLEAEDAGTYYCQQYASVPVTFGQGTKVELKHHHH
HHSG

CD8 signal sequence (amino acids 1-21 of SEQ ID NO: 19)

(SEQ ID NO: 20)
MALPVTALLLPLALLLHAARP

Anti-EGFR scFv (amino acids 22-267 of SEQ ID NO: 19)

(SEQ ID NO: 1)
QVQLKQSGPGLVQPSQSLSITCTVSGFSLTNYGVHWVRQSPGKGLEWLGV
IWSGGNTDYNTPFTSRLSINKDNSKSQVFFKMNSLQSNDTAIYYCARALT
YYDYEFAYWGQGTLVTVSAGGGGSGGGGSGGGGSGGGGSDILLTQSPVIL
SVSPGERVSFSCRASQSIGTNIHWYQQRTNGSPRLLIKYASESISGIPSR
FSGSGSGTDFTLSINSVESEDIADYYCQQNNNWPTTFGAGTKLELK

CD8 hinge/TM (amino acids 268-336 of SEQ ID NO: 19)

(SEQ ID NO: 22)
TTTPAPRPPTPAPTIASQPLSLRPEACRPAAGGAVHTRGLDFACDIYIWA
PLAGTCGVLLLSLVITLYC

4-1BB (amino acids 337-378 of SEQ ID NO: 19)

(SEQ ID NO: 23)
KRGRKKLLYIFKQPFMRPVQTTQEEDGCSCRFPEEEEGGCEL

CD3ζ (amino acids 379-490 of SEQ ID NO: 19)

(SEQ ID NO: 24)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

2A cleavage sequence (amino acids 494-515 of SEQ ID NO: 19; SEQ ID NO: 31)

(SEQ ID NO: 31)
GSGATNFSLLKQAGDVEENPGP

Igκ signal sequence (amino acids 519-539 of SEQ ID NO: 19; SEQ ID NO: 32)

(SEQ ID NO: 32)
METDTLLLWVLLLWVPGSTGD

Anti-GARP camelid (amino acids 540-646 of SEQ ID NO: 19; SEQ ID NO: 25).

(SEQ ID NO: 25)
DIQMTQSPSSLSASLGDRVTITCQASQSISSYLAWYQQKPGQAPNILIYG
ASRLKTGVPSRFSGSGSGTSFTLTISGLEAEDAGTYYCQQYASVPVTFGQ
GTKVELK

Construct 5-3C10 (anti-EGFRvIII) scFv-CD8 Hinge/TM-4-1BB ICD-CD3ζ-P2A-Igκ signal sequence-Cetuximab (anti-EGFR) scFv-CD3scFv-His-tag (SEQ ID NO: 26) including 3C10 scFv (amino acids 1-243 of SEQ ID NO: 26; SEQ ID NO: 27), CD8 hinge/TM (amino acids 244-312 of SEQ ID NO: 26; SEQ ID NO: 28), 4-1 BB ICD (amino acids 313-354 of SEQ ID NO: 26; SEQ ID NO: 29), CD3ζ (amino acids 355-466 of SEQ ID NO: 26; SEQ ID NO: 30), P2A (amino acids 467-488 of SEQ ID NO: 26; SEQ ID NO: 31), Igκ signal sequence (amino acids 491-511 of SEQ ID NO: 26; SEQ ID NO: 32), Cetuximab scFv (amino acids 512-752 of SEQ ID NO: 26; SEQ ID NO: 33), CD3 scFv (amino acids 758-1000 of SEQ ID NO: 26; SEQ ID NO: 34).

(SEQ ID NO: 26)
EIQLQQSGAELVKPGASVKLSCTGSGFNIEDYYIHWVKQRTEQGLEWIGR
IDPENDETKYGPIFQGRATITADTSSNTVYLQLSSLTSEDTAVYYCAFRG
GVYWGPGTTLTVSSGGGGSGGGGSGGGGSHMDVVMTQSPLTLSVAIGQSA
SISCKSSQSLLDSDGKTYLNWLLQRPGQSPKRLISLVSKLDSGVPDRFTG
SGSGTDFTLRISRVEAEDLGIYYCWQGTHFPGTFGGGTKLEIKTTTPAPR
PPTPAPTIASQPLSLRPEACRPAAGGAVHTRGLDFACDIYIWAPLAGTCG
VLLLSLVITLYCKRGRKKLLYIFKQPFMRPVQTTQEEDGCSCRFPEEEEG
GCELRVKFSRSADAPAYQQGQNQLYNELNLGRREEYDVLDKRRGRDPEMG
GKPRRKNPQEGLYNELQKDKMAEAYSEIGMKGERRRGKGHDGLYQGLSTA
TKDTYDALHMQALPPRGSGATNFSLLKQAGDVEENPGPPRMETDTLLLWV
LLLWVPGSTGDDILLTQSPVILSVSPGERVSFSCRASQSIGTNIHWYQQR
TNGSPRLLIKYASESISGIPSRFSGSGSGTDFTLSINSVESEDIADYYCQ
QNNNWPTTFGAGTKLELKGGGGSGGGGSGGGGSQVQLKQSGPGLVQPSQS
LSITCTVSGFSLTNYGVHWVRQSPGKGLEWLGVIWSGGNTDYNTPFTSRL
SINKDNSKSQVFFKMNSLQSNDTAIYYCARALTYYDYEFAYWGQGTLVTV
SAGGGGSDIKLQQSGAELARPGASVKMSCKTSGYTFTRYTMHWVKQRPGQ
GLEWIGYINPSRGYTNYNQKFKDKATLTTDKSSSTAYMQLSSLTSEDSAV
YYCARYYDDHYCLDYWGQGTTLTVSSVEGGSGGSGGSGGSGGVDDIQLTQ
SPAIMSASPGEKVTMTCRASSSVSYMNWYQQKSGTSPKRWIYDTSKVASG
VPYRFSGSGSGTSYSLTISSMEAEDAATYYCQQWSSNPLTFGAGTKLELK
HHHHHH

3C10 (anti-EGFRvIII) scFv (amino acids 1-243 of SEQ ID NO: 26)

(SEQ ID NO: 27)
EIQLQQSGAELVKPGASVKLSCTGSGFNIEDYYIHWVKQRTEQGLEWIGR
IDPENDETKYGPIFQGRATITADTSSNTVYLQLSSLTSEDTAVYYCAFRG
GVYWGPGTTLTVSSGGGGSGGGGSGGGGSHMDVVMTQSPLTLSVAIGQSA
SISCKSSQSLLDSDGKTYLNWLLQRPGQSPKRLISLVSKLDSGVPDRFTG
SGSGTDFTLRISRVEAEDLGIYYCWQGTHFPGTFGGGTKLEIK

CD8 hinge/TM (amino acids 244-312 of SEQ ID NO: 26)

(SEQ ID NO: 28)
TTTPAPRPPTPAPTIASQPLSLRPEACRPAAGGAVHTRGLDFACDIYIWA
PLAGTCGVLLLSLVITLYC

4-1 BB ICD (amino acids 313-354 of SEQ ID NO: 26)

(SEQ ID NO: 29)
KRGRKKLLYIFKQPFMRPVQTTQEEDGCSCRFPEEEEGGCEL

CD3ζ ((amino acids 355-466 of SEQ ID NO: 26)

(SEQ ID NO: 30)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

P2A (amino acids 467-488 of SEQ ID NO: 26)

(SEQ ID NO: 31)
GSGATNFSLLKQAGDVEENPGP

Igκ signal sequence (amino acids 491-511 of SEQ ID NO: 26)

(SEQ ID NO: 32)
METDTLLLWVLLLWVPGSTGD

Cetuximab (anti-EGFR) scFv (amino acids 512-752 of SEQ ID NO: 26)

(SEQ ID NO: 33)
DILLTQSPVILSVSPGERVSFSCRASQSIGTNIHWYQQRTNGSPRLLIKY
ASESISGIPSRFSGSGSGTDFTLSINSVESEDIADYYCQQNNNWPTTFGA
GTKLELKGGGGSGGGGSGGGGSQVQLKQSGPGLVQPSQSLSITCTVSGFS
LTNYGVHWVRQSPGKGLEWLGVIWSGGNTDYNTPFTSRLSINKDNSKSQV
FFKMNSLQSNDTAIYYCARALTYYDYEFAYWGQGTLVTVSA

Anti-CD3 scFv (amino acids 758-1000 of SEQ ID NO: 26)

(SEQ ID NO: 34)
DIKLQQSGAELARPGASVKMSCKTSGYTFTRYTMHWVKQRPGQGLEWIGY
INPSRGYTNYNQKFKDKATLTTDKSSSTAYMQLSSLTSEDSAVYYCARYY
DDHYCLDYWGQGTTLTVSSVEGGSGGSGGSGGSGGVDDIQLTQSPAIMSA
SPGEKVTMTCRASSSVSYMNWYQQKSGTSPKRWIYDTSKVASGVPYRFSG
SGSGTSYSLTISSMEAEDAATYYCQQWSSNPLTFGAGTKLELK

Construct 6-2173 (anti-EGFRvIII) scFv-CD8 Hinge/TM-4-1BB ICD-CD3ζ-P2A-Igκ signal sequence-Cetuximab (anti-EGFR) scFv-CD3-scFv-His-tag (SEQ ID NO: 35) including 2173 scFv (amino acids 1-246 of SEQ ID NO: 35; SEQ ID NO: 36), CD8 hinge/TM (amino acids 247-315 of SEQ ID NO: 35; SEQ ID NO: 37), 4-1 BB ICD (amino acids 316-357 of SEQ ID NO: 36; SEQ ID NO: 38), CD3ζ(amino acids 358-469 of SEQ ID NO: 35; SEQ ID NO: 39), P2A (amino acids 470-491 of SEQ ID NO: 35; SEQ ID NO: 40), Igκ signal sequence (amino acids 494-514 of SEQ ID NO: 35; SEQ ID NO: 41), Cetuximab scFv (amino acids 515-755 of SEQ ID NO: 35; SEQ ID NO: 42), and CD3 scFv (amino acids 761-1003 of SEQ ID NO: 35; SEQ ID NO: 43).

(SEQ ID NO: 35)
EIQLVQSGAEVKKPGESLRISCKGSGFNIEDYYIHWVRQMPGKGLEWMGR
IDPENDETKYGPIFQGHVTISADTSINTVYLQWSSLKASDTAMYYCAFRG
GVYWGQGTTVTVSSGGGGSGGGGSGGGGSGGGGSDVVMTQSPDSLAVSLG
ERATINCKSSQSLLDSDGKTYLNWLQQKPGQPPKRLISLVSKLDSGVPDR
FSGSGSGTDFTLTISSLQAEDVAVYYCWQGTHFPGTFGGGTKVEIKTTTP
APRPPTPAPTIASQPLSLRPEACRPAAGGAVHTRGLDFACDIYIWAPLAG
TCGVLLLSLVITLYCKRGRKKLLYIFKQPFMRPVQTTQEEDGCSCRFPEE
EEGGCELRVKFSRSADAPAYQQGQNQLYNELNLGRREEYDVLDKRRGRDP
EMGGKPRRKNPQEGLYNELQKDKMAEAYSEIGMKGERRRGKGHDGLYQGL
STATKDTYDALHMQALPPRGSGATNFSLLKQAGDVEENPGPPRMETDTLL
LWVLLLWVPGSTGDDILLTQSPVILSVSPGERVSFSCRASQSIGTNIHWY
QQRTNGSPRLLIKYASESISGIPSRFSGSGSGTDFTLSINSVESEDIADY
YCQQNNNWPTTFGAGTKLELKGGGGSGGGGSGGGGSQVQLKQSGPGLVQP
SQSLSITCTVSGFSLTNYGVHWVRQSPGKGLEWLGVIWSGGNTDYNTPFT
SRLSINKDNSKSQVFFKMNSLQSNDTAIYYCARALTYYDYEFAYWGQGTL
VTVSAGGGGSDIKLQQSGAELARPGASVKMSCKTSGYTFTRYTMHWVKQR
PGQGLEWIGYINPSRGYTNYNQKFKDKATLTTDKSSSTAYMQLSSLTSED
SAVYYCARYYDDHYCLDYWGQGTTLTVSSVEGGSGGSGGSGGSGGVDDIQ
LTQSPAIMSASPGEKVTMTCRASSSVSYMNWYQQKSGTSPKRWIYDTSKV
ASGVPYRFSGSGSGTSYSLTISSMEAEDAATYYCQQWSSNPLTFGAGTKL
ELKHHHHHH

2173 (anti-EGFRvIII) scFv (amino acids 1-246 of SEQ ID NO: 35)

(SEQ ID NO: 36)
EIQLVQSGAEVKKPGESLRISCKGSGFNIEDYYIHWVRQMPGKGLEWMGR
IDPENDETKYGPIFQGHVTISADTSINTVYLQWSSLKASDTAMYYCAFRG
GVYWGQGTTVTVSSGGGGSGGGGSGGGGSGGGGSDVVMTQSPDSLAVSLG
ERATINCKSSQSLLDSDGKTYLNWLQQKPGQPPKRLISLVSKLDSGVPDR
FSGSGSGTDFTLTISSLQAEDVAVYYCWQGTHFPGTFGGGTKVEIK

CD8 hinge/TM (amino acids 247-315 of SEQ ID NO: 35)

(SEQ ID NO: 37)
TTTPAPRPPTPAPTIASQPLSLRPEACRPAAGGAVHTRGLDFACDIYIWA
PLAGTCGVLLLSLVITLYC

4-1 BB ICD (amino acids 316-357 of SEQ ID NO: 35)

(SEQ ID NO: 38)
KRGRKKLLYIFKQPFMRPVQTTQEEDGCSCRFPEEEEGGCEL

CD3ζ (amino acids 358-469 of SEQ ID NO: 35)

(SEQ ID NO: 39)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

P2A (amino acids 470-491 of SEQ ID NO: 35)

(SEQ ID NO: 40)
GSGATNFSLLKQAGDVEENPGP

Igκ signal sequence (amino acids 494-514 of SEQ ID NO: 35)

(SEQ ID NO: 41)
METDTLLLWVLLLWVPGSTGD

Cetuximab (anti-EGFR) scFv (amino acids 515-755 of SEQ ID NO: 35)

(SEQ ID NO: 42)
DILLTQSPVILSVSPGERVSFSCRASQSIGTNIHWYQQRTNGSPRLLIKY
ASESISGIPSRFSGSGSGTDFTLSINSVESEDIADYYCQQNNNWPTTFGA
GTKLELKGGGGSGGGGSGGGGSQVQLKQSGPGLVQPSQSLSITCTVSGFS
LTNYGVHWVRQSPGKGLEWLGVIWSGGNTDYNTPFTSRLSINKDNSKSQV
FFKMNSLQSNDTAIYYCARALTYYDYEFAYWGQGTLVTVSA

Anti-CD3 scFv (amino acids 761-1003 of SEQ ID NO: 35)

(SEQ ID NO: 43)
DIKLQQSGAELARPGASVKMSCKTSGYTFTRYTMHWVKQRPGQGLEWIGY
INPSRGYTNYNQKFKDKATLTTDKSSSTAYMQLSSLTSEDSAVYYCARYY
DDHYCLDYWGQGTTLTVSSVEGGSGGSGGSGGSGGVDDIQLTQSPAIMSA
SPGEKVTMTCRASSSVSYMNWYQQKSGTSPKRWIYDTSKVASGVPYRFSG
SGSGTSYSLTISSMEAEDAATYYCQQWSSNPLTFGAGTKLELK

Construct 7-2173 (anti-EGFRvIII) scFv-CD8 Hinge/TM-4-1BB ICD-CD3ζ-P2A-IgK signal sequence-CD19 scFvCD3-scFv-His-tag (SEQ ID NO: 44) including 2173 scFv (amino acids 1-246 of SEQ ID NO: 44; SEQ ID NO: 45), CD8 hinge/TM (amino acids 247-315 of SEQ ID NO: 44; SEQ ID NO: 46), 4-1 BB ICD (amino acids 316-357 of SEQ ID NO: 44; SEQ ID NO: 47), CD3 (amino acids 358-469 of SEQ ID NO: 44; SEQ ID NO: 48), P2A (amino acids 470-491 of SEQ ID NO: 44; SEQ ID NO: 49), Igκ signal sequence (amino acids 494-514 of SEQ ID NO: 44; SEQ ID NO: 50), CD19 scFv (amino acids 515-764 of SEQ ID NO: 44; SEQ ID NO: 51), CD3 scFv (amino acids 770-1012 of SEQ ID NO: 44; SEQ ID NO: 52).

(SEQ ID NO: 44)
EIQLVQSGAEVKKPGESLRISCKGSGFNIEDYYIHWVRQMPGKGLEWMGR
IDPENDETKYGPIFQGHVTISADTSINTVYLQWSSLKASDTAMYYCAFRG
GVYWGQGTTVTVSSGGGGSGGGGSGGGGSGGGGSDVVMTQSPDSLAVSLG
ERATINCKSSQSLLDSDGKTYLNWLQQKPGQPPKRLISLVSKLDSGVPDR
FSGSGSGTDFTLTISSLQAEDVAVYYCWQGTHFPGTFGGGTKVEIKTTTP
APRPPTPAPTIASQPLSLRPEACRPAAGGAVHTRGLDFACDIYIWAPLAG
TCGVLLLSLVITLYCKRGRKKLLYIFKQPFMRPVQTTQEEDGCSCRFPEE
EEGGCELRVKFSRSADAPAYQQGQNQLYNELNLGRREEYDVLDKRRGRDP
EMGGKPRRKNPQEGLYNELQKDKMAEAYSEIGMKGERRRGKGHDGLYQGL
STATKDTYDALHMQALPPRGSGATNFSLLKQAGDVEENPGPPRMETDTLL
LWVLLLWVPGSTGDDIQLTQSPASLAVSLGQRATISCKASQSVDYDGDSY
LNWYQQIPGQPPKLLIYDASNLVSGIPPRFSGSGSGTDFTLNIHPVEKVD
AATYHCQQSTEDPWTFGGGTKLEIKGGGGSGGGGSGGGGSQVQLQQSGAE
LVRPGSSVKISCKASGYAFSSYWMNWVKQRPGQGLEWIGQIWPGDGDTNY
NGKFKGKATLTADESSSTAYMQLSSLASEDSAVYFCARRETTTVGRYYYA
MDYWGQGTTVTVSSGGGGSDIKLQQSGAELARPGASVKMSCKTSGYTFTR
YTMHWVKQRPGQGLEWIGYINPSRGYTNYNQKFKDKATLTTDKSSSTAYM
QLSSLTSEDSAVYYCARYYDDHYCLDYWGQGTTLTVSSVEGGSGGSGGSG
GSGGVDDIQLTQSPAIMSASPGEKVTMTCRASSSVSYMNWYQQKSGTSPK
RWIYDTSKVASGVPYRFSGSGSGTSYSLTISSMEAEDAATYYCQQWSSNP
LTFGAGTKLELKHHHHHH

2173 (anti-EGFRvIII) scFv (amino acids 1-246 of SEQ ID NO: 44)

(SEQ ID NO: 45)
EIQLVQSGAEVKKPGESLRISCKGSGFNIEDYYIHWVRQMPGKGLEWMGR
IDPENDETKYGPIFQGHVTISADTSINTVYLQWSSLKASDTAMYYCAFRG
GVYWGQGTTVTVSSGGGGSGGGGSGGGGSGGGGSDVVMTQSPDSLAVSLG
ERATINCKSSQSLLDSDGKTYLNWLQQKPGQPPKRLISLVSKLDSGVPDR
FSGSGSGTDFTLTISSLQAEDVAVYYCWQGTHFPGTFGGGTKVEIK

CD8 hinge/TM (amino acids 247-315 of SEQ ID NO: 44)

(SEQ ID NO: 46)
TTTPAPRPPTPAPTIASQPLSLRPEACRPAAGGAVHTRGLDFACDIYIWA
PLAGTCGVLLLSLVITLYC

4-1 BB ICD (amino acids 316-357 of SEQ ID NO: 44)

(SEQ ID NO: 47)
KRGRKKLLYIFKQPFMRPVQTTQEEDGCSCRFPEEEEGGCEL

CD3ζ (amino acids 358-469 of SEQ ID NO: 44)

(SEQ ID NO: 48)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

P2A (amino acids 470-491 of SEQ ID NO: 44)

(SEQ ID NO: 49)
GSGATNFSLLKQAGDVEENPGP

Igκ signal sequence (amino acids 494-514 of SEQ ID NO: 44)

(SEQ ID NO: 50)
METDTLLLWVLLLWVPGSTGD

Anti-CD19 scFv (amino acids 515-764 of SEQ ID NO: 44)

(SEQ ID NO: 51)
DIQLTQSPASLAVSLGQRATISCKASQSVDYDGDSYLNWYQQIPGQPPKL
LIYDASNLVSGIPPRFSGSGSGTDFTLNIHPVEKVDAATYHCQQSTEDPW
TFGGGTKLEIKGGGGSGGGGSGGGGSQVQLQQSGAELVRPGSSVKISCKA
SGYAFSSYWMNWVKQRPGQGLEWIGQIWPGDGDTNYNGKFKGKATLTADE
SSSTAYMQLSSLASEDSAVYFCARRETTTVGRYYYAMDYWGQGTTVTVSS

Anti-CD3 scFv (amino acids 770-1012 of SEQ ID NO: 44)

(SEQ ID NO: 52)
DIKLQQSGAELARPGASVKMSCKTSGYTFTRYTMHWVKQRPGQGLEWIGY
INPSRGYTNYNQKFKDKATLTTDKSSSTAYMQLSSLTSEDSAVYYCARYY
DDHYCLDYWGQGTTLTVSSVEGGSGGSGGSGGSGGVDDIQLTQSPAIMSA
SPGEKVTMTCRASSSVSYMNWYQQKSGTSPKRWIYDTSKVASGVPYRFSG
SGSGTSYSLTISSMEAEDAATYYCQQWSSNPLTFGAGTKLELK

Construct 8-(NFAT response element)-Igκ signal sequence-Cetuximab (anti-EGFR) scFv-CD3-scFv-His-tag-(EF1α promoter)-2173 (anti-EGFRvIII) scFv-CD8 hinge/TM-4-1BB ICD-CD3ζ (SEQ ID NO: 53) including Igκ signal sequence (amino acids 1-21 of SEQ ID NO: 53; SEQ ID NO: 54), Cetuximab scFv (amino acids 22-262 of SEQ ID NO: 53; SEQ ID NO: 55), CD3 scFv (amino acids 268-510 of SEQ ID NO: 53; SEQ ID NO: 56), 2173 scFv (amino acids 517-762 of SEQ ID NO: 53; SEQ ID NO: 57), CD8 hinge/TM (amino 763-831 of SEQ ID NO: 53; SEQ ID NO: 58), 4-1 BB ICD (amino acids 832-873 of SEQ ID NO: 53; SEQ ID NO: 59), CD3ζ (amino acids 874-985 of SEQ ID NO: 53; SEQ ID NO: 60).
(NFAT response element)

METDTLLLWVLLLWVPGSTGDDILLTQSPVILSVSPGERVSFSCRASQSI
GTNIHWYQQRTNGSPRLLIKYASESISGIPSRFSGSGSGTDFTLSINSVE
SEDIADYYCQQNNNWPTTFGAGTKLELKGGGGSGGGGSGGGGSQVQLKQS
GPGLVQPSQSLSITCTVSGFSLTNYGVHWVRQSPGKGLEWLGVIWSGGNT
DYNTPFTSRLSINKDNSKSQVFFKMNSLQSNDTAIYYCARALTYYDYEFA
YWGQGTLVTVSAGGGGSDIKLQQSGAELARPGASVKMSCKTSGYTFTRYT
MHWVKQRPGQGLEWIGYINPSRGYTNYNQKFKDKATLTTDKSSSTAYMQL
SSLTSEDSAVYYCARYYDDHYCLDYWGQGTTLTVSSVEGGSGGSGGSGGS
GGVDDIQLTQSPAIMSASPGEKVTMTCRASSSVSYMNWYQQKSGTSPKRW
IYDTSKVASGVPYRFSGSGSGTSYSLTISSMEAEDAATYYCQQWSSNPLT
FGAGTKLELKHHHHHH

(EF1α Promoter)

(SEQ ID NO: 53)
EIQLVQSGAEVKKPGESLRISCKGSGFNIEDYYIHWVRQMPGKGLEWMGR
IDPENDETKYGPIFQGHVTISADTSINTVYLQWSSLKASDTAMYYCAFRG
GVYVVGQGTTVTVSSGGGGSGGGGSGGGGSGGGGSDVVMTQSPDSLAVSL
GERATINCKSSQSLLDSDGKTYLNWLQQKPGQPPKRLISLVSKLDSGVPD
RFSGSGSGTDFTLTISSLQAEDVAVYYCWQGTHFPGTFGGGTKVEIKTTT
PAPRPPTPAPTIASQPLSLRPEACRPAAGGAVHTRGLDFACDIYIWAPLA
GTCGVLLLSLVITLYCKRGRKKLLYIFKQPFMRPVQTTQEEDGCSCRFPE
EEEGGCELRVKFSRSADAPAYQQGQNQLYNELNLGRREEYDVLDKRRGRD
PEMGGKPRRKNPQEGLYNELQKDKMAEAYSEIGMKGERRRGKGHDGLYQG
LSTATKDTYDALHMQALPPR

(Note: the two polypeptides noted above are denoted with a single sequence identifier for convenience, but it should be understood that the CAR and BiTE components can be made separately, due to the two separate promoters; see above.)
   Igκ signal sequence (amino acids 1-21 of SEQ ID NO: 53)

(SEQ ID NO: 54)
METDTLLLWVLLLWVPGSTGD

Cetuximab (anti-EGFR) scFv (amino acids 22-262 of SEQ ID NO: 53)

(SEQ ID NO: 55)
DILLTQSPVILSVSPGERVSFSCRASQSIGTNIHWYQQRTNGSPRLLIK
YASESISGIPSRFSGSGSGTDFTLSINSVESEDIADYYCQQNNNWPTTF
GAGTKLELKGGGGSGGGGSGGGGSQVQLKQSGPGLVQPSQSLSITCTVS
GFSLTNYGVHWVRQSPGKGLEWLGVIWSGGNTDYNTPFTSRLSINKDNS
KSQVFFKMNSLQSNDTAIYYCARALTYYDYEFAYVVGQGTLVTVSA

Anti-CD3 scFv (amino acids 268-510 of SEQ ID NO: 53)

(SEQ ID NO: 56)
DIKLQQSGAELARPGASVKMSCKTSGYTFTRYTMHWVKQRPGQGLEWIG
YINPSRGYTNYNQKFKDKATLTTDKSSSTAYMQLSSLTSEDSAVYYCAR
YYDDHYCLDYWGQGTTLTVSSVEGGSGGSGGSGGSGGVDDIQLTQSPAI
MSASPGEKVTMTCRASSSVSYMNWYQQKSGTSPKRWIYDTSKVASGVPY
RFSGSGSGTSYSLTISSMEAEDAATYYCQQWSSNPLTFGAGTKLELK

2173 (anti-EGFRvIII) scFv (amino acids 517-762 of SEQ ID NO: 53)

(SEQ ID NO: 57)
EIQLVQSGAEVKKPGESLRISCKGSGFNIEDYYIHWVRQMPGKGLEWMG
RIDPENDETKYGPIFQGHVTISADTSINTVYLQWSSLKASDTAMYYCAF
RGGVYVVGQGTTVTVSSGGGGSGGGGSGGGGSGGGGSDVVMTQSPDSLA
VSLGERATINCKSSQSLLDSDGKTYLNWLQQKPGQPPKRLISLVSKLDS
GVPDRFSGSGSGTDFTLTISSLQAEDVAVYYCWQGTHFPGTFGGGTKVE
IK

CD8 hinge/TM (amino acids 763-831 of SEQ ID NO: 53)

(SEQ ID NO: 58)
TTTPAPRPPTPAPTIASQPLSLRPEACRPAAGGAVHTRGLDFACDIYIW
APLAGTCGVLLLSLVITLYC

4-1 BB ICD (amino acids 832-873 of SEQ ID NO: 53)

(SEQ ID NO: 59)
KRGRKKLLYIFKQPFMRPVQTTQEEDGCSCRFPEEEEGGCEL

CD3ζ (amino acids 874-985 of SEQ ID NO: 53)

(SEQ ID NO: 60)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

Construct 9-(NFAT response element)-IgK signal sequence-CD19 scFv-CD3-scFv-His-tag-(EF1α Promoter)-2173 (anti-EGFRvIII) scFv-CD8 Hinge/TM-4-1BB ICD-CD3ζ (SEQ ID NO: 61) including (NFAT response element), Igκ signal sequence (amino acids 1-21 of SEQ ID NO: 61; SEQ ID NO: 62), CD19 scFv (amino acids 22-271 of SEQ ID NO: 61; SEQ ID NO: 63), CD3 scFv (amino acids 277-519 of SEQ ID NO: 61; SEQ ID NO: 64), 2173 scFv (amino acids 526-771 of SEQ ID NO: 61; SEQ ID NO: 65), CD8 hinge/TM (amino acids 772-840 of SEQ ID NO: 61; SEQ ID NO: 66), 4-1 BB ICD (amino acids 841-882 of SEQ ID NO: 61; SEQ ID NO: 67), CD3ζ (amino acids 883-994 of SEQ ID NO: 61; SEQ ID NO: 68).
(NFAT response element)

METDTLLLWVLLLWVPGSTGDDIQLTQSPASLAVSLGQRATISCKASQS
VDYDGDSYLNWYQQIPGQPPKLLIYDASNLVSGIPPRFSGSGSGTDFTL
NIHPVEKVDAATYHCQQSTEDPWTFGGGTKLEIKGGGGSGGGGSGGGGS
QVQLQQSGAELVRPGSSVKISCKASGYAFSSYWMNWVKQRPGQGLEWIG
QIWPGDGDTNYNGKFKGKATLTADESSSTAYMQLSSLASEDSAVYFCAR
RETTTVGRYYYAMDYVVGQGTTVTVSSGGGGSDIKLQQSGAELARPGAS
VKMSCKTSGYTFTRYTMHWVKQRPGQGLEWIGYINPSRGYTNYNQKFKD
KATLTTDKSSSTAYMQLSSLTSEDSAVYYCARYYDDHYCLDYVVGQGTT
LTVSSVEGGSGGSGGSGGSGGVDDIQLTQSPAIMSASPGEKVTMTCRAS
SSVSYMNWYQQKSGTSPKRWIYDTSKVASGVPYRFSGSGSGTSYSLTIS
SMEAEDAATYYCQQWSSNPLTFGAGTKLELKHHHHHH

(EF1a Promoter)

(SEQ ID NO: 61)
EIQLVQSGAEVKKPGESLRISCKGSGFNIEDYYIHWVRQMPGKGLEWMGR
IDPENDETKYGPIFQGHVTISADTSINTVYLQWSSLKASDTAMYYCAFRG
GVYVVGQGTTVTVSSGGGGSGGGGSGGGGSGGGGSDVVMTQSPDSLAVSL
GERATINCKSSQSLLDSDGKTYLNWLQQKPGQPPKRLISLVSKLDSGVPD
RFSGSGSGTDFTLTISSLQAEDVAVYYCWQGTHFPGTFGGGTKVEIKTTT
PAPRPPTPAPTIASQPLSLRPEACRPAAGGAVHTRGLDFACDIYIWAPLA
GTCGVLLLSLVITLYCKRGRKKLLYIFKQPFMRPVQTTQEEDGCSCRFPE
EEEGGCELRVKFSRSADAPAYQQGQNQLYNELNLGRREEYDVLDKRRGRD
PEMGGKPRRKNPQEGLYNELQKDKMAEAYSEIGMKGERRRGKGHDGLYQG
LSTATKDTYDALHMQALPPR

(Note: the two polypeptides noted above are denoted with a single sequence identifier for convenience, but it should be understood that the CAR and BiTE components can be made separately, due to the two separate promoters; see above.)
   Igκ signal sequence (amino acids 1-21 of SEQ ID NO: 61)

(SEQ ID NO: 62)
METDTLLLWVLLLWVPGSTGD

Anti-CD19 scFv (amino acids 22-271 of SEQ ID NO: 61)

(SEQ ID NO: 63)
DIQLTQSPASLAVSLGQRATISCKASQSVDYDGDSYLNWYQQIPGQPPKL
LIYDASNLVSGIPPRFSGSGSGTDFTLNIHPVEKVDAATYHCQQSTEDPW
TFGGGTKLEIKGGGGSGGGGSGGGGSQVQLQQSGAELVRPGSSVKISCKA
SGYAFSSYWMNWVKQRPGQGLEWIGQIWPGDGDTNYNGKFKGKATLTADE
SSSTAYMQLSSLASEDSAVYFCARRETTTVGRYYYAMDYWGQGTTVTVSS

Anti-CD3 scFv (amino acids 277-519 of SEQ ID NO: 61)

(SEQ ID NO: 64)
DIKLQQSGAELARPGASVKMSCKTSGYTFTRYTMHWVKQRPGQGLEWIGY
INPSRGYTNYNQKFKDKATLTTDKSSSTAYMQLSSLTSEDSAVYYCARYY
DDHYCLDYWGQGTTLTVSSVEGGSGGSGGSGGSGGVDDIQLTQSPAIMSA
SPGEKVTMTCRASSSVSYMNWYQQKSGTSPKRWIYDTSKVASGVPYRFSG
SGSGTSYSLTISSMEAEDAATYYCQQWSSNPLTFGAGTKLELK

2173 (anti-EGFRvIII) scFv (amino acids 526-771 of SEQ ID NO: 61)

(SEQ ID NO: 65)
EIQLVQSGAEVKKPGESLRISCKGSGFNIEDYYIHWVRQMPGKGLEWMGR
IDPENDETKYGPIFQGHVTISADTSINTVYLQWSSLKASDTAMYYCAFRG
GVYVVGQGTTVTVSSGGGGSGGGGSGGGGSGGGGSDVVMTQSPDSLAVSL
GERATINCKSSQSLLDSDGKTYLNWLQQKPGQPPKRLISLVSKLDSGVPD
RFSGSGSGTDFTLTISSLQAEDVAVYYCWQGTHFPGTFGGGTKVEIK

CD8 hinge/TM (amino acids 772-840 of SEQ ID NO: 61)

(SEQ ID NO: 66)
TTTPAPRPPTPAPTIASQPLSLRPEACRPAAGGAVHTRGLDFACDIYIWA
PLAGTCGVLLLSLVITLYC

4-1 BB ICD (amino acids 841-882 of SEQ ID NO: 61)

(SEQ ID NO: 67)
KRGRKKLLYIFKQPFMRPVQTTQEEDGCSCRFPEEEEGGCEL

CD3 (amino acids 883-994 of SEQ ID NO: 61)

(SEQ ID NO: 68)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

Construct 10-CD8 signal sequence-Anti-GARP scFv (H-L)-CD8 hinge/TM-4-1BB ICD-CD3ζ (SEQ ID NO: 69) including CD8 signal sequence (amino acids 1-21 of SEQ ID NO: 69; SEQ ID NO: 70), anti-GARP scFv (H-L) (amino acids 22-274 of SEQ ID NO: 69; SEQ ID NO: 71), CD8 hinge/TM (amino acids 275-343 of SEQ ID NO: 69; SEQ ID NO: 72), 4-1 BB ICD (amino acids 344-385 of SEQ ID NO: 69; SEQ ID NO: 73), CD3ζ (amino acids 386-497 of SEQ ID NO: 69; SEQ ID NO: 74).

(SEQ ID NO: 69)
MALPVTALLLPLALLLHAARPEVQLVQPGAELRNSGASVKVSCKASGYRF
TSYYIDWVRQAPGQGLEWMGRIDPEDGGTKYAQKFQGRVTFTADTSTSTA
YVELSSLRSEDTAVYYCARNEWETVVVGDLMYEYEYWGQGTQVTVSSGGG
GSGGGGSGGGGSGGGGSDIQMTQSPSSLSASLGDRVTITCQASQSISSYL
AWYQQKPGQAPNILIYGASRLKTGVPSRFSGSGSGTSFTLTISGLEAEDA
GTYYCQQYASVPVTFGQGTKVELKTTTPAPRPPTPAPTIASQPLSLRPEA
CRPAAGGAVHTRGLDFACDIYIWAPLAGTCGVLLLSLVITLYCKRGRKKL
LYIFKQPFMRPVQTTQEEDGCSCRFPEEEEGGCELRVKFSRSADAPAYQQ
GQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR

CD8 signal sequence (amino acids 1-21 of SEQ ID NO: 69)

(SEQ ID NO: 70)
MALPVTALLLPLALLLHAARP

Anti-GARP scFv (H-L) (amino acids 22-274 of SEQ ID NO: 69)

(SEQ ID NO: 71)
EVQLVQPGAELRNSGASVKVSCKASGYRFTSYYIDWVRQAPGQGLEWMGR
IDPEDGGTKYAQKFQGRVTFTADTSTSTAYVELSSLRSEDTAVYYCARNE
WETVVVGDLMYEYEYWGQGTQVTVSSGGGGSGGGGSGGGGSGGGGSDIQM
TQSPSSLSASLGDRVTITCQASQSISSYLAWYQQKPGQAPNILIYGASRL
KTGVPSRFSGSGSGTSFTLTISGLEAEDAGTYYCQQYASVPVTFGQGTKV
ELK

CD8 hinge/TM (amino acids 275-343 of SEQ ID NO: 69)

(SEQ ID NO: 72)
TTTPAPRPPTPAPTIASQPLSLRPEACRPAAGGAVHTRGLDFACDIYIWA
PLAGTCGVLLLSLVITLYC

4-1 BB ICD (amino acids 344-385 of SEQ ID NO: 69)

(SEQ ID NO: 73)
KRGRKKLLYIFKQPFMRPVQTTQEEDGCSCRFPEEEEGGCEL

CD3ζ (amino acids 386-497 of SEQ ID NO: 69)

(SEQ ID NO: 74)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

Construct 11-CD8 signal sequence-Anti-GARP scFv (L-H)-CD8 hinge/TM-4-1BB ICD-CD3ζ (SEQ ID NO: 75) including CD8 signal sequence (amino acids 1-21 of SEQ ID NO: 75; SEQ ID NO: 76), anti-GARP scFv (L-H) (amino acids 22-274 of SEQ ID NO: 75; SEQ ID NO: 77), CD8 hinge/TM (amino acids 275-343 of SEQ ID NO: 75; SEQ ID NO: 78), 4-1 BB ICD (amino acids 344-385 of SEQ ID NO: 75; SEQ ID NO: 79), CD3ζ (amino acids 386-497 of SEQ ID NO: 75; SEQ ID NO: 80).

(SEQ ID NO: 75)
MALPVTALLLPLALLLHAARPDIQMTQSPSSLSASLGDRVTITCQASQSI
SSYLAWYQQKPGQAPNILIYGASRLKTGVPSRFSGSGSGTSFTLTISGLE
AEDAGTYYCQQYASVPVTFGQGTKVELKGGGGSGGGGSGGGGSGGGGSEV
QLVQPGAELRNSGASVKVSCKASGYRFTSYYIDWVRQAPGQGLEWMGRID
PEDGGTKYAQKFQGRVTFTADTSTSTAYVELSSLRSEDTAVYYCARNEWE
TVVVGDLMYEYEYWGQGTQVTVSSTTTPAPRPPTPAPTIASQPLSLRPEA
CRPAAGGAVHTRGLDFACDIYIWAPLAGTCGVLLLSLVITLYCKRGRKKL
LYIFKQPFMRPVQTTQEEDGCSCRFPEEEEGGCELRVKFSRSADAPAYQQ
GQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR

CD8 signal sequence (amino acids 1-21 of SEQ ID NO: 75)

(SEQ ID NO: 76)
MALPVTALLLPLALLLHAARP

Anti-GARP scFv (L-H) (amino acids 22-274 of SEQ ID NO: 75)

(SEQ ID NO: 77)
DIQMTQSPSSLSASLGDRVTITCQASQSISSYLAWYQQKPGQAPNILIYG
ASRLKTGVPSRFSGSGSGTSFTLTISGLEAEDAGTYYCQQYASVPVTFGQ
GTKVELKGGGGSGGGGSGGGGSGGGGSEVQLVQPGAELRNSGASVKVSCK
ASGYRFTSYYIDWVRQAPGQGLEWMGRIDPEDGGTKYAQKFQGRVTFTAD
TSTSTAYVELSSLRSEDTAVYYCARNEWETVVVGDLMYEYEYWGQGTQVT
VSS

CD8 hinge/TM (amino acids 275-343 of SEQ ID NO: 75)

(SEQ ID NO: 78)
TTTPAPRPPTPAPTIASQPLSLRPEACRPAAGGAVHTRGLDFACDIYIWA
PLAGTCGVLLLSLVITLYC

4-1 BB ICD (amino acids 344-385 of SEQ ID NO: 75)

(SEQ ID NO: 79)
KRGRKKLLYIFKQPFMRPVQTTQEEDGCSCRFPEEEEGGCEL

CD3ζ (amino acids 386-497 of SEQ ID NO: 75)

(SEQ ID NO: 80)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

TABLE 3
Anti-GARP sequences of Construct 10
and Construct 11

| | | |
|---|---|---|
| SEQ ID NO: 81 | CDR-H1 | SYYID |
| SEQ ID NO: 82 | CDR-H2 | RIDPEDGGTKYAQKFQG |
| SEQ ID NO: 83 | CDR-H3 | NEWETVVVGDLMYEYEY |
| SEQ ID NO: 84 | CDR-L1 | QASQSISSYLA |
| SEQ ID NO: 85 | CDR-L2 | GASRLKT |
| SEQ ID NO: 86 | CDR-L3 | QQYASVPVT |
| SEQ ID NO: 87 | VH | EVQLVQPGAELRNSGAS |
| | | VKVSCKASGYRFTSYYI |
| | | DWVRQAPGQGLEWMGRI |
| | | DPEDGGTKYAQKFQGRV |
| | | TFTADTSTSTAYVELSS |
| | | LRSEDTAVYYCARNEWE |
| | | TVVVGDLMYEYEYWGQG |
| | | TQVTVSS |
| SEQ ID NO: 88 | VL | DIQMTQSPSSLSASLGD |
| | | RVTITCQASQSISSYLA |
| | | WYQQKPGQAPNILIYGA |
| | | SRLKTGVPSRFSGSGSG |
| | | TSFTLTISGLEAEDAGT |
| | | YYCQQYASVPVTFGQGT |
| | | KVELK |

TABLE 4
Anti-LAP sequences of Construct 2
and Construct 3

| | | |
|---|---|---|
| SEQ ID NO: 89 | CDR-H1 | RASQDISNYLN |
| SEQ ID NO: 90 | CDR-H2 | YTSRLHS |
| SEQ ID NO: 91 | CDR-H3 | QQGDTLPWT |
| SEQ ID NO: 92 | CDR-L1 | DYYMS |
| SEQ ID NO: 93 | CDR-L2 | FIRNKPNGYTTEYSASV |
| | | KG |
| SEQ ID NO: 94 | CDR-L3 | YTGGGYFDY |
| SEQ ID NO: 95 | VH | MKLWLNWIFLVTLLNDI |
| | | QCEVKLVESGGGLVQPG |
| | | GSLSLSCAASGFTFTDY |
| | | YMSWVRQPPGKALEWLG |
| | | FIRNKPNGYTTEYSASV |
| | | KGRFTISRDNSQSILYL |
| | | QMNVLRAEDSATYYCAR |
| | | YTGGGYFDYWGQGTTLT |
| | | VSS |
| SEQ ID NO: 96 | VL | MMSSAQFLGLLLLCFQG |
| | | TRCDIQMTQTTSSLSAS |
| | | LGDRLTISCRASQDISN |
| | | YLNWYQQKPDGTVKLLI |
| | | YYTSRLHSGVPSRFSGS |
| | | GSGTDYSLTISNLEQAD |
| | | IATYFCQQGDTLPWTFG |
| | | GGTKLEIK |

Construct 12 (Tandem CAR)-IL-13 zetakine-linker-EGFRvIII scFv-CD8 hinge/TM-4-1BB ICD-CD3ζ (SEQ ID NO: 100) including IL-13 zetakine (SEQ ID NO: 101 (amino acids 1-112 of SEQ ID NO: 100)); linker (SEQ ID NO: 102 (amino acids 113-132 of SEQ ID NO: 100)); EGFRvIII scFv (SEQ ID NO: 103 (amino acids 133-378 of SEQ ID NO: 100)); CD8 hinge/TM (SEQ ID NO: 104 (amino acids 379-447 of SEQ ID NO: 100)); 4-1 BB ICD (SEQ ID NO: 105 (amino acids 448-489 of SEQ ID NO: 100)); and CD3 (SEQ ID NO: 106 (amino acids 490-601 of SEQ ID NO: 100))

(SEQ ID NO: 100)
GPVPPSTALRYLIEELVNITQNQKAPLCNGSMVWSINLTAGMYCAALESL
INVSGCSAIEKTQRMLSGFCPHKVSAGQFSSLHVRDTKIEVAQFVKDLLL
HLKKLFREGRFNGGGGSGGGGSGGGGSGGGGSEIQLVQSGAEVKKPGESL
RISCKGSGFNIEDYYIHWVRQMPGKGLEWMGRIDPENDETKYGPIFQGHV
TISADTSINTVYLQWSSLKASDTAMYYCAFRGGVYWGQGTTVTVSSGGGG
SGGGGSGGGGSGGGGSDVVMTQSPDSLAVSLGERATINCKSSQSLLDSDG
KTYLNWLQQKPGQPPKRLISLVSKLDSGVPDRFSGSGSGTDFTLTISSLQ
AEDVAVYYCWQGTHFPGTFGGGTKVEIKTTTPAPRPPTPAPTIASQPLSL
RPEACRPAAGGAVHTRGLDFACDIYIWAPLAGTCGVLLLSLVITLYCKRG
RKKLLYIFKQPFMRPVQTTQEEDGCSCRFPEEEEGGCELRVKFSRSADAP
AYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPP
R

IL-13 zetakine (amino acids 1-112 of SEQ ID NO: 100)

(SEQ ID NO: 101)
GPVPPSTALRYLIEELVNITQNQKAPLCNGSMVWSINLTAGMYCAALESL
INVSGCSAIEKTQRMLSGFCPHKVSAGQFSSLHVRDTKIEVAQFVKDLLL
HLKKLFREGRFN

Linker (amino acids 113-132 of SEQ ID NO: 100)

(SEQ ID NO: 102)
GGGGSGGGGSGGGGSGGGGS

Anti-EGFRvIII scFv (amino acids 133-378 of SEQ ID NO: 100)

(SEQ ID NO: 103)
EIQLVQSGAEVKKPGESLRISCKGSGFNIEDYYIHWVRQMPGKGLEWMGR
IDPENDETKYGPIFQGHVTISADTSINTVYLQWSSLKASDTAMYYCAFRG
GVYWGQGTTVTVSSGGGGSGGGGSGGGGSGGGGSDVVMTQSPDSLAVSLG
ERATINCKSSQSLLDSDGKTYLNWLQQKPGQPPKRLISLVSKLDSGVPDR
FSGSGSGTDFTLTISSLQAEDVAVYYCWQGTHFPGTFGGGTKVEIK

CD8 hinge/TM (amino acids 379-447 of SEQ ID NO: 100)

(SEQ ID NO: 104)
TTTPAPRPPTPAPTIASQPLSLRPEACRPAAGGAVHTRGLDFACDIYIWA
PLAGTCGVLLLSLVITLYC

4-1 BB ICD (amino acids 448-489 of SEQ ID NO: 100)

(SEQ ID NO: 105)
KRGRKKLLYIFKQPFMRPVQTTQEEDGCSCRFPEEEEGGCEL

CD3ζ (amino acids 490-601 of SEQ ID NO: 100)

(SEQ ID NO: 106)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

Further sequences described herein are as follows:

TABLE 5
Sequences

| SEQ | | |
|---|---|---|
| ID | | |
| NO: | Description | Sequence |
| 98 | EGFR BiTE | DILLTQSPVILSVSPGERVSFSCRASQSI |
| | | GTNIHWYQQRTNGSPRLLIKYASESISGI |
| | | PSRFSGSGSGTDFTLSINSVESEDIADYY |
| | | CQQNNNWPTTFGAGTKLELKGGGGSGGGG |
| | | SGGGGSQVQLKQSGPGLVQPSQSLSITCT |
| | | VSGFSLTNYGVHWVRQSPGKGLEWLGVIW |
| | | SGGNTDYNTPFTSRLSINKDNSKSQVFFK |
| | | MNSLQSNDTAIYYCARALTYYDYEFAYWG |
| | | QGTLVTVSAGGGGSDIKLQQSGAELARPG |
| | | ASVKMSCKTSGYTFTRYTMHWVKQRPGQG |
| | | LEWIGYINPSRGYTNYNQKFKDKATLTTD |
| | | KSSSTAYMQLSSLTSEDSAVYYCARYYDD |
| | | HYCLDYWGQGTTLTVSSVEGGSGGSGGSG |
| | | GSGGVDDIQLTQSPAIMSASPGEKVTMTC |
| | | RASSSVSYMNWYQQKSGTSPKRWIYDTSK |
| | | VASGVPYRFSGSGSGTSYSLTISSMEAED |
| | | AATYYCQQWSSNPLTFGAGTKLELK |
| 99 | CD19 BiTE | DIQLTQSPASLAVSLGQRATISCKASQSV |
| | | DYDGDSYLNWYQQIPGQPPKLLIYDASNL |
| | | VSGIPPRFSGSGSGTDFTLNIHPVEKVDA |
| | | ATYHCQQSTEDPWTFGGGTKLEIKGGGGS |
| | | GGGGSGGGGSQVQLQQSGAELVRPGSSVK |
| | | ISCKASGYAFSSYVVMNWVKQRPGQGLEW |
| | | IGQIWPGDGDTNYNGKFKGKATLTADESS |
| | | STAYMQLSSLASEDSAVYFCARRETTTVG |
| | | RYYYAMDYVVGQGTTVTVSSGGGGSDIKL |
| | | QQSGAELARPGASVKMSCKTSGYTFTRYT |
| | | MHWVKQRPGQGLEWIGYINPSRGYTNYNQ |
| | | KFKDKATLTTDKSSSTAYMQLSSLTSEDS |
| | | AVYYCARYYDDHYCLDYVVGQGTTLTVSS |
| | | VEGGSGGSGGSGGSGGVDDIQLTQSPAIM |
| | | SASPGEKVTMTCRASSSVSYMNWYQQKSG |
| | | TSPKRWIYDTSKVASGVPYRFSGSGSGTS |
| | | YSLTISSMEAEDAATYYCQQWSSNPLTFG |
| | | AGTKLELK |
| 111 | Anti-EGFRvIII | EIQLVQSGAEVKKPGESLRISCKGSGFNI |
| | (2173) VH | EDYYIHWVRQMPGKGLEWMGRIDPENDET |
| | | KYGPIFQGHVTISADTSINTVYLQWSSLK |
| | | ASDTAMYYCAFRGGVYWGQGTTVTVSS |
| 112 | Anti-EGFRvIII | DVVMTQSPDSLAVSLGERATINCKSSQSL |
| | (2173) VL | LDSDGKTYLNWLQQKPGQPPKRLISLVSK |
| | | LDSGVPDRFSGSGSGTDFTLTISSLQAED |
| | | VAVYYCWQGTHFPGTFGGGTKVEIK |
| 113 | Anti-EGFRvIII | EIQLQQSGAELVKPGASVKLSCTGSGFNI |
| | (3C10) VH | EDYYIHWVKQRTEQGLEWIGRIDPENDET |
| | | KYGPIFQGRATITADTSSNTVYLQLSSLT |
| | | SEDTAVYYCAFRGGVYWGPGTTLTVSS |
| 114 | Anti-EGFRvIII | HMDVVMTQSPLTLSVAIGQSASISCKSSQ |
| | (3C10) VL | SLLDSDGKTYLNWLLQRPGQSPKRLISLV |
| | | SKLDSGVPDRFTGSGSGTDFTLRISRVEA |
| | | EDLGIYYCWQGTHFPGTFGGGTKLEIK |
| 115 | Construct 5— | EIQLQQSGAELVKPGASVKLSCTGSGFNI |
| | CAR sequence | EDYYIHWVKQRTEQGLEWIGRIDPENDET |
| | only (no BiTE) | KYGPIFQGRATITADTSSNTVYLQLSSLT |
| | | SEDTAVYYCAFRGGVYWGPGTTLTVSSGG |
| | | GGSGGGGSGGGGSHMDVVMTQSPLTLSVA |
| | | IGQSASISCKSSQSLLDSDGKTYLNWLLQ |
| | | RPGQSPKRLISLVSKLDSGVPDRFTGSGS |
| | | GTDFTLRISRVEAEDLGIYYCWQGTHFPG |
| | | TFGGGTKLEIKTTTPAPRPPTPAPTIASQ |
| | | PLSLRPEACRPAAGGAVHTRGLDFACDIY |
| | | IWAPLAGTCGVLLLSLVITLYCKRGRKKL |
| | | LYIFKQPFMRPVQTTQEEDGCSCRFPEEE |
| | | EGGCELRVKFSRSADAPAYQQGQNQLYNE |
| | | LNLGRREEYDVLDKRRGRDPEMGGKPRRK |
| | | NPQEGLYNELQKDKMAEAYSEIGMKGERR |
| | | RGKGHDGLYQGLSTATKDTYDALHMQALP |
| | | PR |
| 116 | Construct 6— | EIQLVQSGAEVKKPGESLRISCKGSGFNI |
| | CAR sequence | EDYYIHWVRQMPGKGLEWMGRIDPENDET |
| | only (no BiTE) | KYGPIFQGHVTISADTSINTVYLQWSSLK |
| | | ASDTAMYYCAFRGGVYWGQGTTVTVSSGG |
| | | GGSGGGGSGGGGSGGGGSDVVMTQSPDSL |
| | | AVSLGERATINCKSSQSLLDSDGKTYLNW |
| | | LQQKPGQPPKRLISLVSKLDSGVPDRFSG |
| | | SGSGTDFTLTISSLQAEDVAVYYCWQGTH |
| | | FPGTFGGGTKVEIKTTTPAPRPPTPAPTI |
| | | ASQPLSLRPEACRPAAGGAVHTRGLDFAC |
| | | DIYIWAPLAGTCGVLLLSLVITLYCKRGR |
| | | KKLLYIFKQPFMRPVQTTQEEDGCSCRFP |
| | | EEEEGGCELRVKFSRSADAPAYQQGQNQL |
| | | YNELNLGRREEYDVLDKRRGRDPEMGGKP |
| | | RRKNPQEGLYNELQKDKMAEAYSEIGMKG |
| | | ERRRGKGHDGLYQGLSTATKDTYDALHMQ |
| | | ALPPR |
| 117 | Construct 4— | QVQLKQSGPGLVQPSQSLSITCTVSGFSL |
| | CAR sequence | TNYGVHWVRQSPGKGLEWLGVIWSGGNTD |
| | only (no | YNTPFTSRLSINKDNSKSQVFFKMNSLQS |
| | camelid) | NDTAIYYCARALTYYDYEFAYVVGQGTLV |
| | | TVSAGGGGSGGGGSGGGGSGGGGSDILLT |
| | | QSPVILSVSPGERVSFSCRASQSIGTNIH |
| | | WYQQRTNGSPRLLIKYASESISGIPSRFS |
| | | GSGSGTDFTLSINSVESEDIADYYCQQNN |
| | | NWPTTFGAGTKLELKTTTPAPRPPTPAPT |
| | | IASQPLSLRPEACRPAAGGAVHTRGLDFA |
| | | CDIYIWAPLAGTCGVLLLSLVITLYCKRG |
| | | RKKLLYIFKQPFMRPVQTTQEEDGCSCRF |
| | | PEEEEGGCELRVKFSRSADAPAYQQGQNQ |
| | | LYNELNLGRREEYDVLDKRRGRDPEMGGK |
| | | PRRKNPQEGLYNELQKDKMAEAYSEIGMK |
| | | GERRRGKGHDGLYQGLSTATKDTYDALHM |
| | | QALPPR |

Some embodiments of the technology described herein can be defined according to any of the following numbered paragraphs:
1. An immune cell engineered to express:
(a) a chimeric antigen receptor (CAR) polypeptide comprising an extracellular domain comprising a first antigen-binding domain that binds to a first antigen and a second antigen-binding domain that binds to a second antigen; and
(b) a bispecific T cell engager (BiTE), wherein the BiTE binds to a target antigen and a T cell antigen.
2. The immune cell of paragraph 1, wherein the CAR polypeptide comprises a transmembrane domain and an intracellular signaling domain.
3. The immune cell of paragraph 1 or 2, wherein the CAR polypeptide further comprises one or more co-stimulatory domains.
4. The immune cell of any one of paragraphs 1-3, wherein the first and second antigens are glioblastoma antigens.
5. The immune cell of any one of paragraphs 1-4, wherein the first and second antigens are independently selected from epidermal growth factor receptor (EGFR), epidermal growth factor receptor variant III (EGFRvIII), CD19, CD79b, CD37, prostate-specific membrane antigen (PSMA), prostate stem cell antigen (PSCA), interleukin-13 receptor alpha 2 (IL-13Rα2), ephrin type-A receptor 1 (EphA1), human epidermal growth factor receptor 2 (HER2), mesothelin, mucin 1, cell surface associated (MUC1), or mucin 16, cell surface associated (MUC16).
6. The immune cell of any one of paragraphs 1-5, wherein the first antigen-binding domain and/or the second antigen-binding domain comprises an antigen-binding fragment of an antibody.
7. The immune cell of paragraph 6, wherein the antigen-binding fragment of the antibody comprises a single domain antibody or a single chain variable fragment (scFv).
8. The immune cell of any one of paragraphs 1-7, wherein the first antigen-binding domain and/or the second antigen-binding domain comprises a ligand of the first and/or second antigen.
9. The immune cell of any one of paragraphs 1-8, wherein the extracellular domain does not comprise a linker between the first antigen-binding domain and the second antigen-binding domain.
10. The immune cell of any one of paragraphs 1-8, wherein the first antigen-binding domain is connected to the second antigen-binding domain by a linker.
11. The immune cell of paragraph 10, wherein the linker comprises an amino acid having at least 90% sequence identity to the linker of SEQ ID NO: 102, 107, 108, 109, or 110.
12. The immune cell of any one of paragraphs 2-11, wherein the transmembrane domain comprises a hinge/transmembrane domain.
13. The immune cell of paragraph 12, wherein the hinge/transmembrane domain comprises the hinge/transmembrane domain of an immunoglobulin-like protein, CD28, CD8, or 4-1 BB.
14. The immune cell of paragraph 12 or 13, wherein the transmembrane domain comprises the hinge/transmembrane domain of CD8, optionally comprising the amino acid sequence of SEQ ID NO: 4, 10, 16, 22, 28, 37, 46, 58, 66, 72, 78, or 104, or an amino acid sequence having at least 90% sequence identity to the amino acid sequence of SEQ ID NO: 4, 10, 16, 22, 28, 37, 46, 58, 66, 72, 78, or 104.
15. The immune cell of any one of paragraphs 2-14, wherein the intracellular signaling domain comprises the intracellular signaling domain of TCFζ, FcRγ, FcRβ, CD3γ, CD3θ, CD3ε, CD3η, CD3ζCD22, CD79a, CD79b, or CD66d.
16. The immune cell of paragraph 15, wherein the intracellular signaling domain comprises the intracellular signaling domain of CDζ, optionally comprising the amino acid sequence of SEQ ID NO: 6, 12, 18, 24, 30, 39, 48, 60, 68, 74, 80, or 106, or an amino acid sequence having at least 90% sequence identity to the amino acid sequence of SEQ ID NO: 6, 12, 18, 24, 30, 39, 48, 60, 68, 74, 80, or 106.
17. The immune cell of any one of paragraphs 3-16, wherein the co-stimulatory domain comprises the co-stimulatory domain of 4-1 BB, CD27, CD28, or OX-40.
18. The immune cell of paragraph 17, wherein the co-stimulatory domain comprises the co-stimulatory domain of 4-1 BB, optionally comprising the amino acid sequence of SEQ ID NO: 5, 11, 17, 23, 29, 38, 47, 59, 67, 73, 79, or 105, or an amino acid sequence having at least 90% sequence identity to the amino acid sequence of SEQ ID NO: 5, 11, 17, 23, 29, 38, 47, 59, 67, 73, 79, or 105.
19. The immune cell of any one of paragraphs 1-18, wherein the first antigen-binding domain comprises an IL-13Rα2-binding domain.
20. The immune cell of any one of paragraphs 1-19, wherein the second antigen-binding domain comprises an EGFRvIII-binding domain.
21. The immune cell of paragraph 19 or 20, wherein the IL-13Rα2-binding domain comprises an anti-IL-13Rα2 scFv or a ligand of IL-13Rα2.
22. The immune cell of paragraph 21, wherein the ligand of IL-13Rα2 comprises IL-13 or IL-13 zetakine, or an antigen-binding fragment thereof.
23. The immune cell of any one of paragraphs 19-22, wherein the IL-13Rα2-binding domain comprises an amino acid sequence having at least 90% sequence identity to the amino acid sequence of SEQ ID NO: 101.
24. The immune cell of paragraph 23, wherein the IL-13Rα2-binding domain comprises the amino acid sequence of SEQ ID NO: 101.
25. The immune cell of any one of paragraphs 20-24, wherein the EGFRvIII-binding domain comprises an antigen-binding fragment of an antibody.
26. The immune cell of any one of paragraphs 20-25, wherein the EGFRvIII-binding domain comprises an anti-EGFRvIII scFv.
27. The immune cell of paragraph 26, wherein the anti-EGFRvIII scFv comprises a heavy chain variable domain (VH) comprising an amino acid sequence having at least 90% sequence identity to the amino acid sequence of SEQ ID NO: 111 or 113 and/or a light chain variable domain (VL) comprising an amino acid sequence having at least 90% sequence identity to the amino acid sequence of SEQ ID NO: 112 or 114.
28. The immune cell of paragraph 27, wherein the VH comprises the amino acid sequence of SEQ ID NO: 111 or 113 and/or the VL comprises the amino acid sequence of SEQ ID NO: 112 or 114.
29. The immune cell of any one of paragraphs 20-28, wherein the EGFRvIII-binding domain comprises an amino acid sequence having at least 90% sequence identity to the amino acid sequence of SEQ ID NO: 103.
30. The immune cell of paragraph 29, wherein the EGFRvIII-binding domain comprises the amino acid sequence of SEQ ID NO: 103.
31. The immune cell of any one of paragraphs 1-30, wherein the CAR polypeptide comprises an amino acid sequence having at least 90% sequence identity to the amino acid sequence of SEQ ID NO: 100.
32. The immune cell of paragraph 31, wherein the CAR polypeptide comprises the amino acid sequence of SEQ ID NO: 100.
33. An immune cell engineered to express:
(i) a CAR polypeptide comprising an amino acid sequence having at least 90% sequence identity to the amino acid sequence of SEQ ID NO: 100; and
(ii) a BiTE, wherein the BiTE binds to a target antigen and a T cell antigen.
34. An immune cell engineered to express:
(i) a CAR polypeptide comprising the amino acid sequence of SEQ ID NO: 100; and
(ii) a BiTE, wherein the BiTE binds to a target antigen and a T cell antigen.
35. The immune cell of any one of paragraphs 1-34, wherein the target antigen is a glioblastoma-associated antigen selected from one of EGFR, EGFRvIII, CD19, CD79b, CD37, PSMA, PSCA, IL-13Rα2, EphA1, HER2, mesothelin, MUC1, or MUC16.
36. The immune cell of any one of paragraphs 1-35, wherein the T cell antigen is CD3.
37. The immune cell of any one of paragraphs 1-36, wherein the target antigen is EGFR and the T cell antigen is CD3.
38. The immune cell of any one of paragraphs 1-37, wherein the BiTE comprises an amino acid sequence having at least 90% sequence identity to the amino acid sequence of SEQ ID NO: 98 or 99.
39. The immune cell of paragraph 38, wherein the BiTE comprises the amino acid sequence of SEQ ID NO: 98 or 99.
40. The immune cell of any one of paragraphs 1-39, wherein the immune cell is a T or natural killer (NK) cell.
41. The immune cell of any one of paragraphs 1-40, wherein the immune cell is a human cell.
42. A polynucleotide encoding the CAR polypeptide and the BiTE of any one of paragraphs 1-41.
43. The polynucleotide of paragraph 42, wherein the polynucleotide comprises a CAR polypeptide encoding sequence and a BiTE encoding sequence, and wherein the CAR polypeptide encoding sequence and the BiTE encoding sequence are separated by a ribosome skipping moiety.
44. The polynucleotide of paragraph 42 or 43, wherein the CAR polypeptide and/or the BiTE is expressed under a constitutive promoter.
45. The polynucleotide of paragraph 44, wherein the constitutive promoter comprises an elongation factor-1 alpha (EF1α) promoter.
46. The polynucleotide of paragraph 42 or 43, wherein the CAR polypeptide and/or the BiTE is expressed under an inducible promoter.
47. The polynucleotide of paragraph 46, wherein the inducible promoter is inducible by T cell receptor (TCR) or CAR signaling.
48. The polynucleotide of paragraph 47, wherein the inducible promoter comprises a nuclear factor of activated T cells (NFAT) response element.
49. The polynucleotide of paragraph 42 or 43, wherein the CAR polypeptide and the BiTE are each expressed under a constitutive promoter.
50. The polynucleotide of paragraph 42 or 43, wherein the CAR polypeptide is expressed under a constitutive promoter and the BiTE is expressed under an inducible promoter.
51. The polynucleotide of any one of paragraphs 42-50, further comprising a suicide gene.
52. The polynucleotide of any one of paragraphs 42-51, further comprising a sequence encoding one or more signal sequences.
53. A vector comprising the polynucleotide of any one of paragraphs 42-52.
54. The vector of paragraph 53, wherein the vector is a lentiviral vector.
55. A pharmaceutical composition comprising the immune cell of any one of paragraphs 1-41, the polynucleotide of any one of paragraphs 42-52, or the vector of paragraph 53 or 54.
56. A method of treating a cancer in a subject in need thereof, the method comprising administering the immune cell of any one of paragraphs 1-41, the polynucleotide of any one of paragraphs 42-52, the vector of paragraph 53 or 54, or the pharmaceutical composition of paragraph 55 to the subject.
57. The method of paragraph 56, wherein the cancer is glioblastoma, lung cancer, pancreatic cancer, lymphoma, or myeloma, optionally wherein the cancer comprises expressing one or more of the group consisting of EGFR, EGFRvIII, CD19, CD79b, CD37, PSMA, PSCA, IL-13Rα2, EphA1, HER2, mesothelin, MUC1, and MUC16.
58. The method of paragraph 57, wherein the glioblastoma comprises cells expressing one or more of the group consisting of IL-13Rα2, EGFRvIII, EGFR, HER2, mesothelin, and EphA1.
59. The method of paragraph 57 or 58, wherein the glioblastoma comprises cells with reduced EGFRvIII expression.
60. An immune cell engineered to express:
(i) a CAR polypeptide comprising an EGFR-binding domain, wherein the CAR polypeptide comprises an amino acid sequence having at least 90% sequence identity to the amino acid sequence of SEQ ID NO: 117; and
(ii) an anti-GARP camelid comprising an amino acid sequence having at least 90% sequence identity to the amino acid sequence of SEQ ID NO: 25.
61. An immune cell engineered to express:
(i) a CAR polypeptide comprising an EGFRvIII-binding domain, wherein the CAR polypeptide comprises an amino acid sequence having at least 90% sequence identity to the amino acid sequence of SEQ ID NO: 115 or 116; and
(ii) a BiTE, wherein the BiTE binds to EGFR and CD3, comprising an amino acid sequence having at least 90% sequence identity to the amino acid sequence of SEQ ID NO: 98 or 99.
62. A polynucleotide encoding the CAR polypeptide and the anti-GARP camelid of paragraph 60.
63. A polynucleotide encoding the CAR polypeptide and the BiTE of paragraph 61.
64. The polynucleotide of paragraph 62 or 63, further comprising a suicide gene.
65. The polynucleotide of any one of paragraphs 62-64, further comprising a sequence encoding one or more signal sequences.
66. A vector comprising the polynucleotide of any one of paragraphs 62-65.
67. The vector of paragraph 66, wherein the vector is a lentiviral vector.
68. A pharmaceutical composition comprising the immune cell of paragraph 60 or 61, the polynucleotide of any one of paragraphs 62-65, or the vector of paragraph 66 or 67.
69. A method of treating glioblastoma having reduced EGFRvIII expression in a subject comprising administering to the subject an immune cell engineered to express: (i) a CAR polypeptide comprising an extracellular EGFRvIII-binding domain; and (ii) a BiTE, wherein the immune cell is optionally selected from the immune cell of any one of paragraphs 1-41, and 61.
70. A method of preventing or reducing immunosuppression in the tumor microenvironment in a subject comprising administering to the subject an immune cell comprising (i) a CAR comprising an extracellular target binding domain; and (ii) a BiTE, wherein the immune cell is optionally selected from the immune cell of any one of paragraphs 1-41, 60, and 61.
71. A method of preventing or reducing T cell exhaustion in the tumor microenvironment in a subject, the method comprising administering to the subject an immune cell comprising (i) a CAR comprising an extracellular target binding domain; and (ii) a BiTE, wherein the immune cell is optionally selected from the immune cell of any one of paragraphs 1-41, 60, and 61.
72. A method of treating a cancer having heterogeneous antigen expression in a subject, the method comprising administering to the subject an immune cell comprising (i) a CAR comprising an extracellular target binding domain; and (ii) a BiTE, wherein the immune cell is optionally selected from the immune cell of any one of paragraphs 1-41, 60, and 61.
73. The method of paragraph 72, wherein the cancer is glioblastoma, prostate cancer, lung cancer, pancreatic cancer, lymphoma, or myeloma.
74. The method of paragraph 72 or 73, wherein the cancer comprises cells expressing one or more of the group consisting of EGFR, EGFRvIII, CD19, PSMA, PSCA, IL-13Rα2, EphA1, Her2, mesothelin, MUC1, and MUC16.
75. A CAR T cell comprising a heterologous nucleic acid molecule, wherein the heterologous nucleic acid molecule comprises:
(a) a first polynucleotide encoding a CAR comprising an extracellular antigen-binding domain, a transmembrane domain, and an intracellular signaling domain; and
(b) a second polynucleotide encoding a therapeutic agent.
76. The CAR T cell of paragraph 75, wherein the therapeutic agent comprises an antibody reagent.
77. The CAR T cell of paragraph 76, wherein the antibody reagent comprises a single chain antibody or a single domain antibody.
78. The CAR T cell of paragraph 76, wherein the antibody reagent comprises a bispecific antibody reagent.
79. The CAR T cell of paragraph 78, wherein the bispecific antibody reagent comprises a BiTE.
80. The CAR T cell of paragraph 77, wherein the single domain antibody comprises a camelid antibody.
81. The CAR T cell of paragraph 75, wherein the therapeutic agent comprises a cytokine.
82. The CAR T cell of any one of paragraphs 75-81, wherein the CAR and the therapeutic agent are produced as separate CAR and therapeutic agent molecules.
83. The CAR T cell of paragraph 82, wherein the CAR T cell comprises a ribosome skipping moiety between the first polynucleotide encoding the CAR and the second polynucleotide encoding the therapeutic agent.
84. The CAR T cell of paragraph 83, wherein the ribosome skipping moiety comprises a 2A peptide.
85. The CAR T cell of paragraph 84, wherein the 2A peptide comprises P2A or T2A.
86. The CAR T cell of any one of paragraphs 75-85, wherein the CAR and the therapeutic agent are each constitutively expressed.
87. The CAR T cell of any one of paragraphs 75-86, wherein expression of the CAR and the therapeutic agent is driven by an EF1α promoter.
88. The CAR T cell of any one of paragraphs 75-85, wherein the therapeutic agent is expressed under the control of an inducible promoter, which is optionally inducible by T cell receptor or CAR signaling.
89. The CAR T cell of paragraph 88, wherein the inducible promoter comprises the NFAT promoter.
90. The CAR T cell of any one of paragraphs 75-89, wherein the CAR is expressed under the control of a constitutive promoter and the therapeutic agent is expressed under the control of an inducible promoter, which is optionally inducible by T cell receptor or CAR signaling.
91. The CAR T cell of any one of paragraphs 75-90, wherein the CAR further comprises one or more co-stimulatory domains.
92. The CAR T cell of any one of paragraphs 75-91, wherein the antigen-binding domain of the CAR comprises an antibody, a single chain antibody, a single domain antibody, or a ligand.
93. The CAR T cell of any one of paragraphs 75-92, wherein the transmembrane domain comprises a hinge/transmembrane domain.
94. The CAR T cell of paragraph 93, wherein the hinge/transmembrane domain comprises the hinge/transmembrane domain of an immunoglobulin-like protein, CD28, CD8, or 4-1 BB.
95. The CAR T cell of any one of paragraphs 75-94, wherein the transmembrane domain of the CAR comprises a CD8 hinge/transmembrane domain, which optionally comprises the sequence of any one of SEQ ID NOs: 4, 10, 16, 22, 28, 37, 46, 58, 66, 72, 78, and 104, or a variant thereof.
96. The CAR T cell of any one of paragraphs 75-95, wherein the intracellular signaling domain comprises the intracellular signaling domain of TCFζ, FcRγ, FcRβ, CD3γ, CD3θ, CD3ε, CD3η, CD3ζ, CD22, CD79a, CD79b, or CD66d.
97. The CAR T cell of any one of paragraphs 75-96, wherein the intracellular signaling domain comprises a CD3ζ intracellular signaling domain, which optionally comprises the sequence of any one of SEQ ID NOs: 6, 12, 18, 24, 30, 39, 48, 60, 68, 74, 80, and 106, or a variant thereof.
98. The CAR T cell of any one of paragraphs 91-97, wherein the co-stimulatory domain comprises the co-stimulatory domain of 4-1 BB, CD27, CD28, or OX-40.
99. The CAR T cell of any one of paragraphs 91-98, wherein the co-stimulatory domain comprises a 4-1 BB co-stimulatory domain, which optionally comprises the sequence of any one of SEQ ID NOs: 5, 11, 17, 23, 29, 38, 47, 59, 67, 73, 79, and 105, or a variant thereof.
100. The CAR T cell of any one of paragraphs 75-99, wherein the CAR antigen-binding domain binds to a tumor-associated antigen or a Treg-associated antigen.
101. The CAR T cell of paragraph 80, wherein the camelid antibody binds to a tumor-associated antigen or a Treg-associated antigen.
102. The CAR T cell of paragraph 79, wherein the BiTE binds to (i) a tumor-associated antigen or a Treg-associated antigen, and (ii) a T cell antigen.
103. The CAR T cell of any one of paragraphs 100-102, wherein the tumor-associated antigen is a solid tumor-associated antigen.
104. The CAR T cell of paragraph 103, wherein the tumor-associated antigen comprises EGFRvIII, EGFR, CD19, PSMA, PSCA, IL-13Rα2, EphA1, Her2, mesothelin, MUC1, or MUC16, and optionally the CAR antigen-binding domain or the therapeutic agent comprises a sequence selected from the group consisting of SEQ ID NO: 21, 27, 33, 36, 42, 45, 51, 55, 57, 63, 65, 103, and variants thereof.
105. The CAR T cell of any one of paragraphs 100-102, wherein the Treg-associated antigen is selected from the group consisting of glycoprotein A repetitions predominant (GARP), latency-associated peptide (LAP), CD25, and cytotoxic T lymphocyte-associated antigen-4 (CTLA-4), and optionally the CAR antigen-binding domain or the therapeutic agent comprises a sequence selected from the group consisting of SEQ ID NO: 3, 9, 15, 25, 71, 77, and variants thereof.
106. A CAR polypeptide comprising an extracellular antigen-binding domain, a transmembrane domain, and an intracellular signaling domain; and the antigen-binding domain binds to a Treg-associated antigen.
107. The CAR polypeptide of paragraph 106, wherein the Treg-associated antigen is selected from the group consisting of GARP, LAP, CD25, and CTLA-4.
108. The CAR polypeptide of paragraph 106 or 107, wherein the CAR further comprises one or more co-stimulatory domains.
109. The CAR polypeptide of any one of paragraphs 106-108, wherein the Treg-associated antigen is GARP or LAP.
110. The CAR polypeptide of any one of paragraphs 106-109, wherein the antigen-binding domain of the CAR comprises:
(a) a heavy chain variable domain (VH) comprising three complementarity determining regions CDR-H1, CDR-H2, and CDR-H3, wherein the CDR-H1 comprises an amino acid sequence of SEQ ID NO: 81, or an amino acid sequence with no more than 1, 2, or 3 amino acid substitutions of SEQ ID NO: 81; the CDR-H2 comprises an amino acid sequence of SEQ ID NO: 82, or an amino acid sequence with no more than 1, 2, or 3 amino acid substitutions of SEQ ID NO: 82; and the CDR-H3 comprises an amino acid sequence of SEQ ID NO: 83, or an amino acid sequence with no more than 1, 2, or 3 amino acid substitutions of SEQ ID NO: 83, and/or
(b) a light chain variable domain (VL) comprising three complementarity determining regions CDR-L1, CDR-L2, and CDR-L3, wherein the CDR-L1 comprises an amino acid sequence of SEQ ID NO: 84, or an amino acid sequence with no more than 1, 2, or 3 amino acid substitutions of SEQ ID NO: 84; the CDR-L2 comprises an amino acid sequence of SEQ ID NO: 85, or an amino acid sequence with no more than 1, 2, or 3 amino acid substitutions of SEQ ID NO: 85; and the CDR-L3 comprises an amino acid sequence of SEQ ID NO: 86, or an amino acid sequence with no more than 1, 2, or 3 amino acid substitutions of SEQ ID NO: 86.
111. The CAR polypeptide of paragraph 110, wherein the VH comprises an amino acid sequence of SEQ ID NO: 87, or an amino acid sequence having at least 90% sequence identity to the amino acid sequence of SEQ ID NO: 87, and/or the VL comprises an amino acid sequence of SEQ ID NO: 88, or an amino acid sequence having at least 90% sequence identity to the amino acid sequence of SEQ ID NO: 88.
112. The CAR polypeptide of any one of paragraphs 106-109, wherein the antigen-binding domain of the CAR comprises:
(a) a heavy chain variable domain (VH) comprising three complementarity determining regions CDR-H1, CDR-H2, and CDR-H3, wherein the CDR-H1 comprises an amino acid sequence of SEQ ID NO: 89, or an amino acid sequence with no more than 1, 2, or 3 amino acid substitutions of SEQ ID NO: 89; the CDR-H2 comprises an amino acid sequence of SEQ ID NO: 90, or an amino acid sequence with no more than 1, 2, or 3 amino acid substitutions of SEQ ID NO: 90; and the CDR-H3 comprises an amino acid sequence of SEQ ID NO: 91, or an amino acid sequence with no more than 1, 2, or 3 amino acid substitutions of SEQ ID NO: 91, and/or
(b) a light chain variable domain (VL) comprising three complementarity determining regions CDR-L1, CDR-L2, and CDR-L3, wherein the CDR-L1 comprises an amino acid sequence of SEQ ID NO: 92, or an amino acid sequence with no more than 1, 2, or 3 amino acid substitutions of SEQ ID NO: 92; the CDR-L2 comprises an amino acid sequence of SEQ ID NO: 93, or an amino acid sequence with no more than 1, 2, or 3 amino acid substitutions of SEQ ID NO: 93; and the CDR-L3 comprises an amino acid sequence of SEQ ID NO: 94, or an amino acid sequence with no more than 1, 2, or 3 amino acid substitutions of SEQ ID NO: 94.
113. The CAR polypeptide of paragraph 112, wherein the VH comprises an amino acid sequence of SEQ ID NO: 95, or an amino acid sequence having at least 90% sequence identity to the amino acid sequence of SEQ ID NO: 95, and/or the VL comprises an amino acid sequence of SEQ ID NO: 96, or an amino acid sequence having at least 90% sequence identity to the amino acid sequence of SEQ ID NO: 96.
114. The CAR polypeptide of any one of paragraphs 110-113, wherein the VH is N-terminal to the VL.
115. The CAR polypeptide of any one of paragraphs 110-113, wherein the VL is N-terminal to the VH.
116. The CAR polypeptide of any one of paragraphs 106-115, wherein the antigen-binding domain of the CAR comprises a scFv or a single domain antibody, which optionally comprises a sequence selected from the group consisting of SEQ ID NO: 3, 9, 15, 25, 71, 77, and variants thereof.
117. The CAR polypeptide of any one of paragraphs 106-116, wherein the transmembrane domain comprises a hinge/transmembrane domain.
118. The CAR polypeptide of paragraph 117, wherein the hinge/transmembrane domain comprises the hinge/transmembrane domain of an immunoglobulin-like protein, CD28, CD8, or 4-1 BB.
119. The CAR polypeptide of any one of paragraphs 106-118, wherein the transmembrane domain of the CAR comprises a CD8 hinge/transmembrane domain, which optionally comprises the sequence of any one of SEQ ID NOs: 4, 10, 16, 22, 28, 37, 46, 58, 66, 72, 78, and 104, or a variant thereof.
120. The CAR polypeptide of any one of paragraphs 106-119, wherein the intracellular signaling domain comprises the intracellular signaling domain of TCFζ, FcRγ, FcRβ, CD3γ, CD3θ, CD3ε, CD3η, CD3ζ, CD22, CD79a, CD79b, or CD66d.
121. The CAR polypeptide of any one of paragraphs 106-120, wherein the intracellular signaling domain comprises a CD3ζ intracellular signaling domain, which optionally comprises the sequence of any one of SEQ ID NOs: 6, 12, 18, 24, 30, 39, 48, 60, 68, 74, 80, and 106, or a variant thereof.
122. The CAR polypeptide of any one of paragraphs 108-121, wherein the co-stimulatory domain comprises the co-stimulatory domain of 4-1 BB, CD27, CD28, or OX-40.
123. The CAR polypeptide of any one of paragraphs 108-122, wherein the co-stimulatory domain comprises a 4-1 BB co-stimulatory domain, which optionally comprises the sequence of any one of SEQ ID NOs: 5, 11, 17, 23, 29, 38, 47, 59, 67, 73, 79, and 105, or a variant thereof.
124. A CAR polypeptide comprising an amino acid sequence having at least 90% sequence identity to the amino acid sequence of any one of SEQ ID NO: 26, SEQ ID NO: 35, SEQ ID NO: 44, SEQ ID NO: 53, SEQ ID NO: 61, SEQ ID NO: 19, SEQ ID NO: 1, SEQ ID NO: 7, SEQ ID NO: 13, SEQ ID NO: 69, SEQ ID NO: 75, and SEQ ID NO: 100.
125. The CAR polypeptide of paragraph 124, comprising the amino acid sequence of any one of SEQ ID NO: 26, SEQ ID NO: 35, SEQ ID NO: 44, SEQ ID NO: 53, SEQ ID NO: 61, SEQ ID NO: 19, SEQ ID NO: 1, SEQ ID NO: 7, SEQ ID NO: 13, SEQ ID NO: 69, SEQ ID NO: 75, and SEQ ID NO: 100.
126. A nucleic acid molecule encoding (i) the CAR polypeptide, or (ii) a polyprotein comprising the CAR polypeptide and the therapeutic agent, of any one of paragraphs 75-125.
127. The nucleic acid molecule of paragraph 126, further comprising a suicide gene. 128. The nucleic acid molecule of paragraph 126 or 127, further comprising a sequence encoding a signal sequence.
129. A vector comprising the nucleic acid molecule of any one of paragraphs 126-128.
130. The vector of paragraph 129, wherein the vector is a lentiviral vector.
131. A polypeptide comprising the CAR polypeptide, or a polyprotein comprising the CAR polypeptide and the therapeutic agent, of any one of paragraphs 75-125.
132. An immune cell comprising the CAR polypeptide of any one of paragraphs 106-125, the nucleic acid molecule of any one of paragraphs 126-128, the vector of paragraph 129 or 130, and/or the polypeptide of paragraph 131.
133. The immune cell of paragraph 132, wherein the immune cell is a T or NK cell.
134. The immune cell of paragraph 132 or 133, wherein the immune cell is a human cell.
135. A pharmaceutical composition comprising one or more CAR T cells, nucleic acid molecules, CAR polypeptides, polyproteins, or immune cells of any one of paragraphs 75-134.
136. A method of treating a patient having cancer, the method comprising administering to the patient the pharmaceutical composition of paragraph 135.
137. The method of paragraph 136, wherein by targeting the tumor microenvironment, systemic toxicity is reduced.
138. The method of paragraph 136 or 137, wherein the cancer is characterized by the presence of one or more solid tumors.
139. The method of any one of paragraphs 136-138, wherein the cancer is characterized by tumor-infiltrating Tregs.
140. The method of any one of paragraphs 136-139, wherein the cancer is a glioblastoma.
141. A method of treating a patient having cancer, the method comprising administering to the patient a CAR T cell product, genetically modified to secrete a tumor-toxic antibody or cytokine, wherein by directing the cancer toxicity locally to the tumor microenvironment, systemic toxicity is reduced.
142. The method of paragraph 141, wherein the CAR T cell is genetically modified to deliver an antibody against CTLA4, CD25, GARP, LAP, IL-15, CSF1R, or EGFR, EGFRvIII, CD19, CD79b, CD37, PSMA, PSCA, IL-13Rα2, EphA1, Her2, mesothelin, MUC1, or MUC16, or a bispecific antibody to the tumor microenvironment.
143. The method of paragraph 142, wherein the bispecific antibody is a BiTE directed against EGFR and CD3.
144. A method of delivering a therapeutic agent to a tissue or organ in a patient to treat a disease or pathology, the method comprising administering to said patient a CAR T cell, genetically modified to secrete a therapeutic antibody, toxin, or agent, wherein the therapeutic antibody, toxin, or agent would, by itself, be unable to enter or penetrate the tissue or organ.
145. The method of paragraph 144, wherein the tissue or organ is in the nervous system.
146. The method of paragraph 145, wherein the nervous system is the central nervous system.
147. The method of paragraph 146, wherein the central nervous system is the brain.
148. The method of any one of paragraphs 144-147, wherein the disease or pathology is a cancer.
149. The method of paragraph 148, wherein the cancer is glioblastoma, prostate cancer, lung cancer, pancreatic cancer, lymphoma, or myeloma.
150. The method of any one of paragraphs 144-149, wherein the therapeutic antibody is anti-EGFR or anti-EGFRvIII.
151. A method of treating glioblastoma having reduced EGFRvIII expression in a subject comprising administering to the subject a CAR T cell engineered to express: (i) a CAR polypeptide comprising an extracellular EGFRvIII-binding domain; and (ii) a BiTE, wherein the CAR T cell is optionally selected from the CAR T cell of any one of paragraphs 75-105.
152. A method of preventing or reducing immunosuppression in the tumor microenvironment in a subject comprising administering to the subject a CAR T cell engineered to express: (i) a CAR polypeptide comprising an extracellular target binding domain; and (ii) a BiTE, wherein the CAR T cell is optionally selected from the CAR T cell of any one of paragraphs 75-105.
153. A method of preventing or reducing T cell exhaustion in the tumor microenvironment in a subject, the method comprising administering to the subject a CAR T cell engineered to express: (i) a CAR polypeptide comprising an extracellular target binding domain; and (ii) a BiTE, wherein the CAR T cell is optionally selected from the CAR T cell of any one of paragraphs 75-105.
154. A method of treating a cancer having heterogeneous antigen expression in a subject, the method comprising administering to the subject a CAR T cell engineered to express: (i) a CAR polypeptide comprising an extracellular target binding domain; and (ii) a BiTE, wherein the CAR T cell is optionally selected from the CAR T cell of any one of paragraphs 75-105.
155. The method of paragraph 154, wherein the cancer is glioblastoma, prostate cancer, lung cancer, pancreatic cancer, lymphoma, or myeloma.
156. The method of paragraph 154 or 155, wherein the cancer comprises cells expressing one or more of EGFR, EGFRvIII, CD19, PSMA, PSCA, IL-13Rα2, EphA1, Her2, mesothelin, MUC1, and MUC16.

Still other embodiments of the technology described herein can be defined according to any of the following additional numbered paragraphs:
1. A chimeric antigen receptor (CAR) T cell comprising a heterologous nucleic acid molecule, wherein the heterologous nucleic acid molecule comprises:
(a) a first polynucleotide encoding a CAR comprising an antigen-binding domain, a transmembrane domain, and an intracellular signaling domain; and
(b) a second polynucleotide encoding a therapeutic agent.
2. The CAR T cell of paragraph 1, wherein the therapeutic agent comprises an antibody reagent.
3. The CAR T cell of paragraph 2, wherein the antibody reagent comprises a single chain antibody or a single domain antibody.
4. The CAR T cell of paragraph 2 or 3, wherein the antibody reagent comprises a bispecific antibody reagent.
5. The CAR T cell of paragraph 4, wherein the bispecific antibody reagent comprises a bispecific T cell engager (BiTE).
6. The CAR T cell of paragraph 3, wherein the single domain antibody comprises a camelid antibody.
7. The CAR T cell of paragraph 1, wherein the therapeutic agent comprises a cytokine.
8. The CAR T cell of any one of paragraphs 1 to 7, wherein the CAR and the therapeutic agent are produced in the form of a polyprotein, which is cleaved to generate separate CAR and therapeutic agent molecules.
9. The CAR T cell of paragraph 8, wherein the polyprotein comprises a cleavable moiety between the CAR and the therapeutic agent.
10. The CAR T cell of paragraph 9, wherein the cleavable moiety comprises a 2A peptide.
11. The CAR T cell of paragraph 10, wherein the 2A peptide comprises P2A or T2A.
12. The CAR T cell of any one of paragraphs 1 to 11, wherein the CAR and the therapeutic agent are each constitutively expressed.
13. The CAR T cell of any one of paragraphs 1 to 12, wherein expression of the CAR and the therapeutic agent is driven by an elongation factor-1 alpha (EF1α) promoter.
14. The CAR T cell of any one of paragraphs 1 to 11, wherein the therapeutic agent is expressed under the control of an inducible promoter, which is optionally inducible by T cell receptor or CAR signaling.
15. The CAR T cell of paragraph 14, wherein the inducible promoter comprises the NFAT promoter.
16. The CAR T cell of any one of paragraphs 1 to 11, wherein the CAR is expressed under the control of a constitutive promoter and the therapeutic agent is expressed under the control of an inducible promoter, which is optionally inducible by T cell receptor or CAR signaling.
17. The CAR T cell of any one of paragraph 1 to 16, wherein the CAR further comprises one or more co-stimulatory domains.
18. The CAR T cell of any one of paragraphs 1 to 17, wherein the antigen-binding domain of the CAR comprises an antibody, a single chain antibody, a single domain antibody, or a ligand.
19. The CAR T cell of any one of paragraphs 1 to 18, wherein the transmembrane domain of the CAR comprises a CD8 hinge/transmembrane domain, which optionally comprises the sequence of any one of SEQ ID NOs: 4, 10, 16, 22, 28, 37, 46, 58, 66, 72, and 78, or a variant thereof.

20. The CAR T cell of any one of paragraphs 1 to 19, wherein the intracellular signaling domain comprises a CD3ζ intracellular signaling domain, which optionally comprises the sequence of any one of SEQ ID NOs: 6, 12, 18, 24, 30, 39, 48, 60, 68, 74, and 80, or a variant thereof.
21. The CAR T cell of any one of paragraphs 1 to 20, comprising a 4-1 BB co-stimulatory domain, which optionally comprises the sequence of any one of SEQ ID NOs: 5, 11, 17, 23, 29, 38, 47, 59, 67, 73, and 79, or a variant thereof.
22. The CAR T cell of any one of paragraphs 1-21, wherein the CAR antigen-binding domain or the therapeutic agent, when the therapeutic agent comprises an antibody reagent, bind to a tumor-associated antigen.
23. The CAR T cell of paragraph 22, wherein the tumor-associated antigen to which the CAR antigen-binding domain or the therapeutic agent binds is a solid tumor-associated antigen.
24. The CAR T cell of paragraph 22 or 23, wherein the tumor-associated antigen to which the CAR antigen-binding domain or the therapeutic agent binds comprises epidermal growth factor receptor variant III (EGFRvIII), epidermal growth factor receptor (EGFR), CD19, prostate-specific membrane antigen (PSMA), or IL-13 receptor alpha 2 (IL-13Rα2), and optionally the CAR antigen-binding domain or the therapeutic agent comprises a sequence selected from the group consisting of SEQ ID NO: 21, 27, 33, 36, 42, 45, 51, 55, 57, 63, 65, and variants thereof.
25. The CAR T cell of any one of paragraphs 1 to 21, wherein the CAR antigen-binding domain or the therapeutic agent, when the therapeutic agent comprises an antibody reagent, binds to a Treg-associated antigen.
26. The CAR T cell of paragraph 25, wherein the Treg-associated antigen to which the CAR antigen-binding domain or the therapeutic agent binds is selected from the group consisting of glycoprotein A repetitions predominant (GARP), latency-associated peptide (LAP), CD25, and cytotoxic T lymphocyte-associated antigen-4 (CTLA-4), and optionally the CAR antigen-binding domain or the therapeutic agent comprises a sequence selected from the group consisting of SEQ ID NO: 3, 9, 15, 25, 71, 77, and variants thereof.
27. A CAR T cell comprising a polynucleotide encoding a CAR, wherein the CAR comprises an antigen-binding domain, a transmembrane domain, and an intracellular signaling domain; and the antigen-binding domain binds to a Treg-associated antigen.
28. The CAR T cell of paragraph 27, wherein the Treg-associated antigen is selected from the group consisting of GARP, LAP, CD25, and CTLA-4.
29. The CAR T cell of paragraph 27 or 28, wherein the CAR further comprises one or more co-stimulatory domains.
30. The CAR T cell of any one of paragraphs 27-29, wherein the Treg-associated antigen is GARP.
31. The CAR T cell of any one of paragraphs 27-30, wherein the antigen-binding domain of the CAR comprises:
(a) a heavy chain variable domain (VH) comprising three complementarity determining regions CDR-H1, CDR-H2, and CDR-H3, wherein the CDR-H1 comprises an amino acid sequence of SEQ ID NO: 81, or an amino acid sequence with no more than 1, 2, or 3 amino acid substitutions of SEQ ID NO: 81; the CDR-H2 comprises an amino acid sequence of SEQ ID NO: 82, or an amino acid sequence with no more than 1, 2, or 3 amino acid substitutions of SEQ ID NO: 82; and the CDR-H3 comprises an amino acid sequence of SEQ ID NO: 83, or an amino acid sequence with no more than 1, 2, or 3 amino acid substitutions of SEQ ID NO: 83, and
(b) a light chain variable domain (VL) comprising three complementarity determining regions CDR-L1, CDR-L2, and CDR-L3, wherein the CDR-L1 comprises an amino acid sequence of SEQ ID NO: 84, or an amino acid sequence with no more than 1, 2, or 3 amino acid substitutions of SEQ ID NO: 84; the CDR-L2 comprises an amino acid sequence of SEQ ID NO: 85, or an amino acid sequence with no more than 1, 2, or 3 amino acid substitutions of SEQ ID NO: 85; and the CDR-L3 comprises an amino acid sequence of SEQ ID NO: 86, or an amino acid sequence with no more than 1, 2, or 3 amino acid substitutions of SEQ ID NO: 86.32. The CAR T cell of paragraph 31, wherein the VH comprises an amino acid sequence of SEQ ID NO: 87, or an amino acid sequence having at least 90% sequence identity to the amino acid sequence of SEQ ID NO: 87, and the VL comprises an amino acid sequence of SEQ ID NO: 88, or an amino acid sequence having at least 90% sequence identity to the amino acid sequence of SEQ ID NO: 88.
33. The CAR T cell of paragraph 31 or 32, wherein the VH is N-terminal to the VL.
34. The CAR T cell of paragraph 31 or 32, wherein the VL is N-terminal to the VH.
35. The CAR T cell of any one of paragraphs 27 to 34, wherein the antigen-binding domain of the CAR comprises a scFv or a single domain antibody, which optionally comprises a sequence selected from the group consisting of SEQ ID NO: 3, 9, 15, 25, 71, 77, and variants thereof.
36. A CAR T cell comprising a heterologous nucleic acid molecule encoding an amino acid sequence having at least 90% sequence identity to the amino acid sequence of any one of SEQ ID NO: 26, SEQ ID NO: 35, SEQ ID NO: 44, SEQ ID NO: 53, SEQ ID NO: 61, SEQ ID NO: 19, SEQ ID NO: 1, SEQ ID NO: 7, SEQ ID NO: 13, SEQ ID NO: 69, and SEQ ID NO: 75.
37. The CAR T cell of paragraph 36, comprising a heterologous nucleic acid molecule encoding an amino acid sequence of any one of SEQ ID NO: 26, SEQ ID NO: 35, SEQ ID NO: 44, SEQ ID NO: 53, SEQ ID NO: 61, SEQ ID NO: 19, SEQ ID NO: 1, SEQ ID NO: 7, SEQ ID NO: 13, SEQ ID NO: 69, and SEQ ID NO: 75.
38. A nucleic acid molecule encoding (i) the CAR polypeptide, or (ii) a polyprotein comprising the CAR polypeptide and the therapeutic agent, of any one of paragraphs 1 to 37.
39. A polypeptide comprising the CAR polypeptide, or polyprotein comprising the CAR polypeptide and the therapeutic agent, of any one of paragraphs 1 to 37.
40. A pharmaceutical composition comprising one or more CAR T cells, nucleic acid molecules, CAR polypeptides, or polyproteins of any one of paragraphs 1 to 39.
41. A method of treating a patient having cancer, the method comprising administering to the patient a pharmaceutical composition comprising one or more CAR T cell of any one of paragraphs 1 to 37 or a pharmaceutical composition of paragraph 40.
42. The method of paragraph 41, wherein by targeting the tumor microenvironment, systemic toxicity is reduced.
43. The method of paragraph 41 or 42, wherein the cancer is characterized by the presence of one or more solid tumors.
44. The method of any one of paragraphs 41 to 43, wherein the cancer is characterized by tumor-infiltrating Tregs.
45. The method of any one of paragraphs 41 to 44, wherein the cancer is a glioblastoma.
46. A method of treating a patient having cancer, the method comprising administering to the patient a CAR T cell product, genetically modified to secrete a tumor-toxic antibody or cytokine, wherein by directing the cancer toxicity locally to the tumor microenvironment, systemic toxicity is reduced.
47. The method of paragraph 46, wherein the CAR T cell is genetically modified to deliver an antibody against CTLA4, CD25, GARP, LAP, IL15, CSF1R, or EGFR, or a bispecific antibody against to the tumor microenvironment.
48. The method of paragraph 47, wherein the bispecific antibody is directed against EGFR and CD3.
49. A method of delivering a therapeutic agent to a tissue or organ in a patient to treat a disease or pathology, the method comprising administering to said patient a CAR T cell, genetically modified to secrete a therapeutic antibody, toxin, or agent, wherein the therapeutic antibody, toxin, or agent would, by itself, be unable to enter or penetrate the tissue or organ.
50. The method of paragraph 49, wherein the tissue or organ is in the nervous system.
51. The method of paragraph 50, wherein the nervous system is the central nervous system.
52. The method of paragraph 51, wherein the central nervous system is the brain.
53. The method of any one of paragraphs 49 to 52, wherein the disease or pathology is glioblastoma.
54. The method of paragraph 49-53, wherein the therapeutic antibody is anti-EGFR or anti-EGFRvIII.
55. A CAR T cell comprising a polynucleotide encoding a CAR, wherein the CAR comprises an extracellular GARP-binding domain, a transmembrane domain, and an intracellular signaling domain, and wherein the GARP-binding domain comprises:
(a) a heavy chain variable domain (VH) comprising three complementarity determining regions CDR-H1, CDR-H2, and CDR-H3, wherein the CDR-H1 comprises an amino acid sequence of SEQ ID NO: 81, or an amino acid sequence with no more than 1, 2, or 3 amino acid substitutions of SEQ ID NO: 81; the CDR-H2 comprises an amino acid sequence of SEQ ID NO: 82, or an amino acid sequence with no more than 1, 2, or 3 amino acid substitutions of SEQ ID NO: 82; and the CDR-H3 comprises an amino acid sequence of SEQ ID NO: 83, or an amino acid sequence with no more than 1, 2, or 3 amino acid substitutions of SEQ ID NO: 83, and
(b) a light chain variable domain (VL) comprising three complementarity determining regions CDR-L1, CDR-L2, and CDR-L3, wherein the CDR-L1 comprises an amino acid sequence of SEQ ID NO: 84, or an amino acid sequence with no more than 1, 2, or 3 amino acid substitutions of SEQ ID NO: 84; the CDR-L2 comprises an amino acid sequence of SEQ ID NO: 85, or an amino acid sequence with no more than 1, 2, or 3 amino acid substitutions of SEQ ID NO: 85; and the CDR-L3 comprises an amino acid sequence of SEQ ID NO: 86, or an amino acid sequence with no more than 1, 2, or 3 amino acid substitutions of SEQ ID NO: 86.
56. The CAR T cell of paragraph 55, wherein the VH comprises an amino acid sequence of SEQ ID NO: 87, or an amino acid sequence having at least 90% sequence identity to the amino acid sequence of SEQ ID NO: 87, and the VL comprises an amino acid sequence of SEQ ID NO: 88, or an amino acid sequence having at least 90% sequence identity to the amino acid sequence of SEQ ID NO: 88.
57. The CAR T cell of paragraph 55 or 56, wherein the VH is N-terminal to the VL.
58. The CAR T cell of paragraph 55 or 56, wherein the VL is N-terminal to the VH.
59. The CAR T cell of any one of paragraphs 55-58, wherein the GARP-binding domain comprises an amino acid sequence SEQ ID NO: 71 or 77, or an amino acid sequence having at least 90% sequence identity to the amino acid sequence of SEQ ID NO: 71 or 77.
60. The CAR T cell of any one of paragraphs 55-59, wherein the CAR further comprises one or more co-stimulatory domains.
61. The CAR T cell of any one of paragraphs 55-60, wherein the transmembrane domain of the CAR comprises a hinge/transmembrane domain.
62. The CAR T cell of any one of paragraphs 61, wherein the hinge/transmembrane domain comprises a CD4, CD28, CD7, or CD8 hinge/transmembrane domain.
63. The CAR T cell of paragraph 62, wherein the hinge/transmembrane domain comprises a CD8 hinge/transmembrane domain of SEQ ID NO: 72 or 78.
64. The CAR T cell of any one of paragraphs 55-63, wherein the intracellular signaling domain comprises an intracellular signaling domain of TCRζ, FcRγ, FcRβ, CD3γ, CD3θ, CD3θ, CD3ε, CD3ζ, CD22, CD79a, CD79b, or CD66d.
65. The CAR T cell of paragraph 64, wherein the intracellular signaling domain comprises a CD3ζ intracellular signaling domain of SEQ ID NO: 74 or 80.
66. The CAR T cell of any one of paragraphs 55-65, wherein the co-stimulatory domain comprises a co-stimulatory domain of CARD11, CD2, CD7, CD27, CD28, CD30, CD40, ICAM, CD83, OX40, 4-1 BB, CD150, CTLA4, LAGS, CD270, PD-L2, PD-L1, ICOS, DAP10, LAT, NKD2C SLP76, TRIM, or ZAP70.
67. The CAR T cell of paragraph 66, wherein the co-stimulatory domain comprises a 4-1 BB co-stimulatory domain of SEQ ID NO: 73 or 79.
68. The CAR T cell of any one of paragraphs 55-67, wherein the polynucleotide encodes a CAR comprising an amino acid sequence having at least 90% sequence identity to the amino acid sequence of SEQ ID NO: 69 or 75, or wherein the polynucleotide encodes a CAR comprising an amino acid sequence having at least 90% sequence identity to the combination of the amino acid sequences of SEQ ID NOs: 71-74 or 77-80.
69. The CAR T cell of paragraph 68, wherein the polynucleotide encodes a CAR comprising an amino acid sequence of SEQ ID NO: 69 or 75, or wherein the polynucleotide encodes a CAR comprising the combination of the amino acid sequences of SEQ ID NOs: 71-74 or 77-80.
70. A pharmaceutical composition comprising the CAR T cell of any one of paragraphs 55-69.
71. A method of treating a patient having cancer, the method comprising administering the CAR T cell of any one of paragraphs 55-69, or the pharmaceutical composition of paragraph 70, to the patient.
72. The method of paragraph 71, wherein by targeting the tumor microenvironment, systemic toxicity is reduced.
73. The method of paragraph 71 or 72, wherein the cancer is characterized by the presence of one or more solid tumors.
74. The method of any one of paragraphs 71-73, wherein the cancer is characterized by tumor-infiltrating Tregs.
75. The method of any one of paragraphs 71-74, wherein the cancer is a glioblastoma. 76. The CAR T cell of any one of paragraphs 1-26, wherein the heterologous nucleic acid molecule further comprises a suicide gene.
77. The CAR T cell of any one of paragraphs 27-35, wherein the polynucleotide further comprises a suicide gene.
78. The CAR T cell of paragraph 36 or 37, wherein the heterologous nucleic acid molecule further comprises a suicide gene.
79. The nucleic acid molecule of paragraph 38, further comprising a suicide gene.
80. The CAR T cell of any one of paragraphs 55-69, wherein the polynucleotide further comprises a suicide gene.
81. A CAR T cell comprising a polynucleotide encoding a CAR, wherein the CAR comprises an extracellular LAP-binding domain, a transmembrane domain, and an intracellular signaling domain, and wherein the LAP-binding domain comprises:
(a) a heavy chain variable domain (VH) comprising three complementarity determining regions CDR-H1, CDR-H2, and CDR-H3, wherein the CDR-H1 comprises an amino acid sequence of SEQ ID NO: 89, or an amino acid sequence with no more than 1, 2, or 3 amino acid substitutions of SEQ ID NO: 89; the CDR-H2 comprises an amino acid sequence of SEQ ID NO: 90, or an amino acid sequence with no more than 1, 2, or 3 amino acid substitutions of SEQ ID NO: 90; and the CDR-H3 comprises an amino acid sequence of SEQ ID NO: 91, or an amino acid sequence with no more than 1, 2, or 3 amino acid substitutions of SEQ ID NO: 91, and
(b) a light chain variable domain (VL) comprising three complementarity determining regions CDR-L1, CDR-L2, and CDR-L3, wherein the CDR-L1 comprises an amino acid sequence of SEQ ID NO: 92, or an amino acid sequence with no more than 1, 2, or 3 amino acid substitutions of SEQ ID NO: 92; the CDR-L2 comprises an amino acid sequence of SEQ ID NO: 93, or an amino acid sequence with no more than 1, 2, or 3 amino acid substitutions of SEQ ID NO: 93; and the CDR-L3 comprises an amino acid sequence of SEQ ID NO: 94, or an amino acid sequence with no more than 1, 2, or 3 amino acid substitutions of SEQ ID NO: 94,
and wherein the CAR does not comprise one or more of SEQ ID NOs: 7, 9, 13, 15, 95, and 96, or wherein the CAR does not comprise the combination of SEQ ID NOs: 9-12 or 15-18.
82. The CAR T cell of paragraph 81, wherein the VH does not comprise SEQ ID NO: 95, and/or the VL does not comprise SEQ ID NO: 96.
83. The CAR T cell of paragraph 81 or 82, wherein the LAP-binding domain does not comprise SEQ ID NO: 9 or 15.
84. The CAR T cell of any one of paragraphs 81-83, wherein the polynucleotide does not encode a CAR of SEQ ID NO: 7 or 13.
85. The CAR T cell of any one of paragraphs 81-84, wherein the CAR does not comprise an amino acid sequence of the combination of SEQ ID NOs: 9-12 or 15-18.
86. The CAR T cell of any one of paragraphs 81-85, wherein the CAR further comprises one or more co-stimulatory domains.
87. The CAR T cell of any one of paragraphs 81-86, wherein the transmembrane domain of the CAR comprises a hinge/transmembrane domain.
88. The CAR T cell of paragraph 87, wherein the hinge/transmembrane domain comprises a CD4, CD28, CD7, or CD8 hinge/transmembrane domain.
89. The CAR T cell of any one of paragraphs 81-88, wherein the intracellular signaling domain comprises an intracellular signaling domain of TCFζ, FcRγ, FcRβ, CD3γ, CD3θ, CD3ε, CD3ε, CD3ζ, CD22, CD79a, CD79b, or CD66d.
90. The CAR T cell of any one of paragraphs 81-89, wherein the co-stimulatory domain comprises a co-stimulatory domain of CARD11, CD2, CD7, CD27, CD28, CD30, CD40, ICAM, CD83, OX40, 4-1 BB, CD150, CTLA4, LAGS, CD270, PD-L2, PD-L1, ICOS, DAP10, LAT, NKD2C SLP76, TRIM, or ZAP70.
91. The CAR T cell of any one of paragraphs 81-90, wherein the polynucleotide further comprises a suicide gene.
92. A pharmaceutical comprising the CAR T cell of any one of paragraphs 81-91.
93. A method of treating a patient having cancer, the method comprising administering the CAR T cell of any one of paragraphs 81-91, or the pharmaceutical composition of paragraph 92, to the patient.
94. The method of paragraph 93, wherein by targeting the tumor microenvironment, systemic toxicity is reduced.
95. The method of paragraph 93 or 94, wherein the cancer is characterized by the presence of
one or more solid tumors.

96. The method of any one of paragraphs 93-95, wherein the cancer is characterized by tumor-infiltrating Tregs.
97. The method of any one of paragraphs 93-96, wherein the cancer is a glioblastoma.

The following claims are meant to be representative only and not to limit the scope of the disclosed invention. In at least one aspect, we claim:

What is claimed is:

1. An immune cell engineered to express:
(a) a chimeric antigen receptor (CAR) polypeptide comprising an extracellular domain comprising a first antigen-binding domain that binds to a first antigen and a second antigen-binding domain that binds to a second antigen; and
(b) a bispecific T cell engager (BiTE), wherein the BiTE binds to a target antigen and a T cell antigen.

2. The immune cell of claim 1, wherein the CAR polypeptide comprises a transmembrane domain and an intracellular signaling domain.

3. The immune cell of claim 1, wherein the CAR polypeptide further comprises one or more co-stimulatory domains.

4. The immune cell of claim 1, wherein the first and second antigens are glioblastoma antigens.

5. The immune cell of claim 1, wherein the first and second antigens are independently selected from epidermal growth factor receptor (EGFR), epidermal growth factor receptor variant III (EGFRvIII), CD19, CD79b, CD37, prostate-specific membrane antigen (PSMA), prostate stem cell antigen (PSCA), interleukin-13 receptor alpha 2 (IL-13Rα2), ephrin type-A receptor 1 (EphA1), human epidermal growth factor receptor 2 (HER2), mesothelin, mucin 1, cell surface associated (MUC1), or mucin 16, cell surface associated (MUC16).

6. The immune cell of claim 1, wherein the first antigen-binding domain and/or the second antigen-binding domain comprises an antigen-binding fragment of an antibody.

7. The immune cell of claim 6, wherein the antigen-binding fragment of the antibody comprises a single domain antibody or a single chain variable fragment (scFv).

8. The immune cell of claim 1, wherein the first antigen-binding domain and/or the second antigen-binding domain comprises a ligand of the first and/or second antigen.

9. The immune cell of claim 1, wherein the extracellular domain does not comprise a linker between the first antigen-binding domain and the second antigen-binding domain.

10. The immune cell of claim 1, wherein the first antigen-binding domain is connected to the second antigen-binding domain by a linker.

11. The immune cell of claim 10, wherein the linker comprises an amino acid having at least 90% sequence identity to the linker of SEQ ID NO: 102, 107, 108, 109, or 110.

12. The immune cell of claim 2, wherein the transmembrane domain comprises a hinge/transmembrane domain.

13. The immune cell of claim 12, wherein the hinge/transmembrane domain comprises the hinge/transmembrane domain of an immunoglobulin-like protein, CD28, CD8, or 4-1 BB.

14. The immune cell of claim 12, wherein the transmembrane domain comprises the hinge/transmembrane domain of CD8, optionally comprising the amino acid sequence of SEQ ID NO: 4, 10, 16, 22, 28, 37, 46, 58, 66, 72, 78, or 104, or an amino acid sequence having at least 90% sequence identity to the amino acid sequence of SEQ ID NO: 4, 10, 16, 22, 28, 37, 46, 58, 66, 72, 78, or 104.

15. The immune cell of claim 2, wherein the intracellular signaling domain comprises the intracellular signaling domain of TCRζ, FcRγ, FcRβ, CD3γ, CD3θ, CD3ε, CD3η, CD3ζ, CD22, CD79a, CD79b, or CD66d.

16. The immune cell of claim 15, wherein the intracellular signaling domain comprises the intracellular signaling domain of CD3ζ, optionally comprising the amino acid sequence of SEQ ID NO: 6, 12, 18, 24, 30, 39, 48, 60, 68, 74, 80, or 106, or an amino acid sequence having at least 90% sequence identity to the amino acid sequence of SEQ ID NO: 6, 12, 18, 24, 30, 39, 48, 60, 68, 74, 80, or 106.

17. The immune cell of claim 3, wherein the co-stimulatory domain comprises the co-stimulatory domain of 4-1 BB, CD27, CD28, or OX-40.

18. The immune cell of claim 17, wherein the co-stimulatory domain comprises the co-stimulatory domain of 4-1 BB, optionally comprising the amino acid sequence of SEQ ID NO: 5, 11, 17, 23, 29, 38, 47, 59, 67, 73, 79, or 105, or an amino acid sequence having at least 90% sequence identity to the amino acid sequence of SEQ ID NO: 5, 11, 17, 23, 29, 38, 47, 59, 67, 73, 79, or 105.

19. The immune cell of claim 1, wherein the first antigen-binding domain comprises an IL-13Rα2-binding domain.

20. The immune cell of claim 1, wherein the second antigen-binding domain comprises an EGFRvIII-binding domain.

21. The immune cell of claim 19, wherein the IL-13Rα2-binding domain comprises an anti-IL-13Rα2 scFv or a ligand of IL-13Rα2.

22. The immune cell of claim 21, wherein the ligand of IL-13Rα2 comprises IL-13 or IL-13 zetakine, or an antigen-binding fragment thereof.

23. The immune cell of claim 19, wherein the IL-13Rα2-binding domain comprises an amino acid sequence having at least 90% sequence identity to the amino acid sequence of SEQ ID NO: 101.

24. The immune cell of claim 23, wherein the IL-13Rα2-binding domain comprises the amino acid sequence of SEQ ID NO: 101.

25. The immune cell of claim 20, wherein the EGFRvIII-binding domain comprises an antigen-binding fragment of an antibody.

26. The immune cell of claim 20, wherein the EGFRvIII-binding domain comprises an anti-EGFRvIII scFv.

27. The immune cell of claim 26, wherein the anti-EGFRvIII scFv comprises a heavy chain variable domain (VH) comprising an amino acid sequence having at least 90% sequence identity to the amino acid sequence of SEQ ID NO: 111 or 113 and/or a light chain variable domain (VL) comprising an amino acid sequence having at least 90% sequence identity to the amino acid sequence of SEQ ID NO: 112 or 114.

28. The immune cell of claim 27, wherein the VH comprises the amino acid sequence of SEQ ID NO: 111 or 113 and/or the VL comprises the amino acid sequence of SEQ ID NO: 112 or 114.

29. The immune cell of claim 20, wherein the EGFRvIII-binding domain comprises an amino acid sequence having at least 90% sequence identity to the amino acid sequence of SEQ ID NO: 103.

30. The immune cell of claim 29, wherein the EGFRvIII-binding domain comprises the amino acid sequence of SEQ ID NO: 103.

31. The immune cell of claim 1, wherein the CAR polypeptide comprises an amino acid sequence having at least 90% sequence identity to the amino acid sequence of SEQ ID NO: 100.

32. The immune cell of claim 31, wherein the CAR polypeptide comprises the amino acid sequence of SEQ ID NO: 100.

33. An immune cell engineered to express:
(i) a CAR polypeptide comprising an amino acid sequence having at least 90% sequence identity to the amino acid sequence of SEQ ID NO: 100; and
(ii) a BiTE, wherein the BiTE binds to a target antigen and a T cell antigen.

34. An immune cell engineered to express:
(i) a CAR polypeptide comprising the amino acid sequence of SEQ ID NO: 100; and
(ii) a BiTE, wherein the BiTE binds to a target antigen and a T cell antigen.

35. The immune cell of claim 1, 33, or 34, wherein the target antigen is a glioblastoma-associated antigen selected from one of EGFR, EGFRvIII, CD19, CD79b, CD37, PSMA, PSCA, IL-13Rα2, EphA1, HER2, mesothelin, MUC1, or MUC16.

36. The immune cell of claim 1, 33, or 34, wherein the T cell antigen is CD3.

37. The immune cell of claim 1, 33, or 34, wherein the target antigen is EGFR and the T cell antigen is CD3.

38. The immune cell of claim 1, 33, or 34, wherein the BiTE comprises an amino acid sequence having at least 90% sequence identity to the amino acid sequence of SEQ ID NO: 98 or 99.

39. The immune cell of claim 38, wherein the BiTE comprises the amino acid sequence of SEQ ID NO: 98 or 99.

40. The immune cell of claim 1, 33, or 34, wherein the immune cell is a T or natural killer (NK) cell.

41. The immune cell of claim 1, 33, or 34, wherein the immune cell is a human cell.

42. A polynucleotide encoding the CAR polypeptide and the BiTE of claim 1, 33, or 34.

43. The polynucleotide of claim 42, wherein the polynucleotide comprises a CAR polypeptide encoding sequence and a BiTE encoding sequence, and wherein the CAR polypeptide encoding sequence and the BiTE encoding sequence are separated by a ribosome skipping moiety.

44. The polynucleotide of claim 42, wherein the CAR polypeptide and/or the BiTE is expressed under a constitutive promoter.

45. The polynucleotide of claim 44, wherein the constitutive promoter comprises an elongation factor-1 alpha (EF1α) promoter.

46. The polynucleotide of claim 42, wherein the CAR polypeptide and/or the BiTE is expressed under an inducible promoter.

47. The polynucleotide of claim 46, wherein the inducible promoter is inducible by T cell receptor (TCR) or CAR signaling.

48. The polynucleotide of claim 47, wherein the inducible promoter comprises a nuclear factor of activated T cells (NFAT) response element.

49. The polynucleotide of claim 42, wherein the CAR polypeptide and the BiTE are each expressed under a constitutive promoter.

50. The polynucleotide of claim 42, wherein the CAR polypeptide is expressed under a constitutive promoter and the BiTE is expressed under an inducible promoter.

51. The polynucleotide of claim 42, further comprising a suicide gene.

52. The polynucleotide of claim 42, further comprising a sequence encoding one or more signal sequences.

53. A vector comprising the polynucleotide of claim 42.

54. The vector of claim 53, wherein the vector is a lentiviral vector.

55. A pharmaceutical composition comprising the immune cell of claim 1, 33, or 34.

56. A method of treating a cancer in a subject in need thereof, the method comprising administering the immune cell of claim 1, 33, or 34, a pharmaceutical composition thereof, to the subject.

57. The method of claim 56, wherein the cancer is glioblastoma, lung cancer, pancreatic cancer, lymphoma, or myeloma, optionally wherein the cancer comprises expressing one or more of the group consisting of EGFR, EGFRvIII, CD19, CD79b, CD37, PSMA, PSCA, IL-13Rα2, EphA1, HER2, mesothelin, MUC1, and MUC16.

58. The method of claim 57, wherein the glioblastoma comprises cells expressing one or more of the group consisting of IL-13Rα2, EGFRvIII, EGFR, HER2, mesothelin, and EphA1.

59. The method of claim 57, wherein the glioblastoma comprises cells with reduced EGFRvIII expression.

60. An immune cell engineered to express:
(i) a CAR polypeptide comprising an EGFR-binding domain, wherein the CAR polypeptide comprises an amino acid sequence having at least 90% sequence identity to the amino acid sequence of SEQ ID NO: 117; and
(ii) an anti-GARP camelid comprising an amino acid sequence having at least 90% sequence identity to the amino acid sequence of SEQ ID NO: 25.

61. An immune cell engineered to express:
(i) a CAR polypeptide comprising an EGFRvIII-binding domain, wherein the CAR polypeptide comprises an amino acid sequence having at least 90% sequence identity to the amino acid sequence of SEQ ID NO: 115 or 116; and
(ii) a BiTE, wherein the BiTE binds to EGFR and CD3, comprising an amino acid sequence having at least 90% sequence identity to the amino acid sequence of SEQ ID NO: 98 or 99.

62. A polynucleotide encoding the CAR polypeptide and the anti-GARP camelid of claim 60.

63. A polynucleotide encoding the CAR polypeptide and the BiTE of claim 61.

64. The polynucleotide of claim 62 or 63, further comprising a suicide gene.

65. The polynucleotide of claim 62 or 63, further comprising a sequence encoding one or more signal sequences.

66. A vector comprising the polynucleotide of claim 62 or 63.

67. The vector of claim 66, wherein the vector is a lentiviral vector.

68. A pharmaceutical composition comprising the immune cell of claim 60 or 61.

69. A method of treating glioblastoma having reduced EGFRvIII expression in a subject comprising administering to the subject an immune cell engineered to express: (i) a CAR polypeptide comprising an extracellular EGFRvIII-binding domain; and (ii) a BiTE, wherein the immune cell is optionally selected from the immune cell of any one of claims 1, 33, 34, 60, and 61.

70. A method of preventing or reducing immunosuppression in the tumor microenvironment in a subject comprising administering to the subject an immune cell comprising (i) a CAR comprising an extracellular target binding domain; and (ii) a BiTE, wherein the immune cell is optionally selected from the immune cell of any one of claims 1, 33, 34, 60, and 61.

71. A method of preventing or reducing T cell exhaustion in the tumor microenvironment in a subject, the method comprising administering to the subject an immune cell comprising (i) a CAR comprising an extracellular target binding domain; and (ii) a BiTE, wherein the immune cell is optionally selected from the immune cell of any one of claims 1, 33, 34, 60, and 61.

72. A method of treating a cancer having heterogeneous antigen expression in a subject, the method comprising administering to the subject an immune cell comprising (i) a CAR comprising an extracellular target binding domain; and (ii) a BiTE, wherein the immune cell is optionally selected from the immune cell of any one of claims 1, 33, 34, 60, and 61.

73. The method of claim 72, wherein the cancer is glioblastoma, prostate cancer, lung cancer, pancreatic cancer, lymphoma, or myeloma.

74. The method of claim 72, wherein the cancer comprises cells expressing one or more of the group consisting of EGFR, EGFRvIII, CD19, PSMA, PSCA, IL-13Rα2, EphA1, Her2, mesothelin, MUC1, and MUC16.

75. A CAR T cell comprising a heterologous nucleic acid molecule, wherein the heterologous nucleic acid molecule comprises:
(a) a first polynucleotide encoding a CAR comprising an extracellular antigen-binding domain, a transmembrane domain, and an intracellular signaling domain; and
(b) a second polynucleotide encoding a therapeutic agent.

76. The CAR T cell of claim 75, wherein the therapeutic agent comprises an antibody reagent.

77. The CAR T cell of claim 76, wherein the antibody reagent comprises a single chain antibody or a single domain antibody.

78. The CAR T cell of claim 76, wherein the antibody reagent comprises a bispecific antibody reagent.

79. The CAR T cell of claim 78, wherein the bispecific antibody reagent comprises a BiTE.

80. The CAR T cell of claim 77, wherein the single domain antibody comprises a camelid antibody.

81. The CAR T cell of claim 75, wherein the therapeutic agent comprises a cytokine.

82. The CAR T cell of claim 75, wherein the CAR and the therapeutic agent are produced as separate CAR and therapeutic agent molecules.

83. The CAR T cell of claim 82, wherein the CAR T cell comprises a ribosome skipping moiety between the first polynucleotide encoding the CAR and the second polynucleotide encoding the therapeutic agent.

84. The CAR T cell of claim 83, wherein the ribosome skipping moiety comprises a 2A peptide.

85. The CAR T cell of claim 84, wherein the 2A peptide comprises P2A or T2A.

86. The CAR T cell of claim 75, wherein the CAR and the therapeutic agent are each constitutively expressed.

87. The CAR T cell of claim 75, wherein expression of the CAR and the therapeutic agent is driven by an EF1α promoter.

88. The CAR T cell of claim 75, wherein the therapeutic agent is expressed under the control of an inducible promoter, which is optionally inducible by T cell receptor or CAR signaling.

89. The CAR T cell of claim 88, wherein the inducible promoter comprises the NFAT promoter.

90. The CAR T cell of claim 75, wherein the CAR is expressed under the control of a constitutive promoter and the therapeutic agent is expressed under the control of an inducible promoter, which is optionally inducible by T cell receptor or CAR signaling.

91. The CAR T cell of claim 75, wherein the CAR further comprises one or more co-stimulatory domains.

92. The CAR T cell of claim 75, wherein the antigen-binding domain of the CAR comprises an antibody, a single chain antibody, a single domain antibody, or a ligand.

93. The CAR T cell of claim 75, wherein the transmembrane domain comprises a hinge/transmembrane domain.

94. The CAR T cell of claim 93, wherein the hinge/transmembrane domain comprises the hinge/transmembrane domain of an immunoglobulin-like protein, CD28, CD8, or 4-1 BB.

95. The CAR T cell of claim 75, wherein the transmembrane domain of the CAR comprises a CD8 hinge/transmembrane domain, which optionally comprises the sequence of any one of SEQ ID NOs:
4, 10, 16, 22, 28, 37, 46, 58, 66, 72, 78, and 104, or a variant thereof.

96. The CAR T cell of claim 75, wherein the intracellular signaling domain comprises the intracellular signaling domain of TCRζ, FcRγ, FcRβ, CD3γ, CD3θ, CD3ε, CD3η, CD3ζ, CD22, CD79a, CD79b, or CD66d.

97. The CAR T cell of claim 75, wherein the intracellular signaling domain comprises a CD3 intracellular signaling domain, which optionally comprises the sequence of any one of SEQ ID NOs: 6, 12, 18, 24, 30, 39, 48, 60, 68, 74, 80, and 106, or a variant thereof.

98. The CAR T cell of claim 91, wherein the co-stimulatory domain comprises the co-stimulatory domain of 4-1 BB, CD27, CD28, or OX-40.

99. The CAR T cell claim 91, wherein the co-stimulatory domain comprises a 4-1 BB co-stimulatory domain, which optionally comprises the sequence of any one of SEQ ID NOs: 5, 11, 17, 23, 29, 38, 47, 59, 67, 73, 79, and 105, or a variant thereof.

100. The CAR T cell of claim 75, wherein the CAR antigen-binding domain binds to a tumor-associated antigen or a Treg-associated antigen.

101. The CAR T cell of claim 80, wherein the camelid antibody binds to a tumor-associated antigen or a Treg-associated antigen.

102. The CAR T cell of claim 79, wherein the BiTE binds to (i) a tumor-associated antigen or a Treg-associated antigen, and (ii) a T cell antigen.

103. The CAR T cell of any one of claims 100-102, wherein the tumor-associated antigen is a solid tumor-associated antigen.

104. The CAR T cell of claim 103, wherein the tumor-associated antigen comprises EGFRvIII, EGFR, CD19, PSMA, PSCA, IL-13Rα2, EphA1, Her2, mesothelin, MUC1, or MUC16, and optionally the CAR antigen-binding domain or the therapeutic agent comprises a sequence selected from the group consisting of SEQ ID NO: 21, 27, 33, 36, 42, 45, 51, 55, 57, 63, 65, 103, and variants thereof.

105. The CAR T cell of any one of claims 100-102, wherein the Treg-associated antigen is selected from the group consisting of glycoprotein A repetitions predominant (GARP), latency-associated peptide (LAP), CD25, and cytotoxic T lymphocyte-associated antigen-4 (CTLA-4), and optionally the CAR antigen-binding domain or the therapeutic agent comprises a sequence selected from the group consisting of SEQ ID NO: 3, 9, 15, 25, 71, 77, and variants thereof.

106. A CAR polypeptide comprising an extracellular antigen-binding domain, a transmembrane domain, and an intracellular signaling domain; and the antigen-binding domain binds to a Treg-associated antigen.

107. The CAR polypeptide of claim 106, wherein the Treg-associated antigen is selected from the group consisting of GARP, LAP, CD25, and CTLA-4.

108. The CAR polypeptide of claim 106, wherein the CAR further comprises one or more co-stimulatory domains.

109. The CAR polypeptide of claim 106, wherein the Treg-associated antigen is GARP or LAP.

110. The CAR polypeptide of claim 106, wherein the antigen-binding domain of the CAR comprises:
(a) a heavy chain variable domain (VH) comprising three complementarity determining regions CDR-H1, CDR-H2, and CDR-H3, wherein the CDR-H1 comprises an amino acid sequence of SEQ ID NO: 81, or an amino acid sequence with no more than 1, 2, or 3 amino acid substitutions of SEQ ID NO:
81; the CDR-H2 comprises an amino acid sequence of SEQ ID NO: 82, or an amino acid sequence with no more than 1, 2, or 3 amino acid substitutions of SEQ ID NO: 82; and the CDR-H3 comprises an amino acid sequence of SEQ ID NO: 83, or an amino acid sequence with no more than 1, 2, or 3 amino acid substitutions of SEQ ID NO: 83, and/or
(b) a light chain variable domain (VL) comprising three complementarity determining regions CDR-L1, CDR-L2, and CDR-L3, wherein the CDR-L1 comprises an amino acid sequence of SEQ ID NO: 84, or an amino acid sequence with no more than 1, 2, or 3 amino acid substitutions of SEQ ID NO: 84;
the CDR-L2 comprises an amino acid sequence of SEQ ID NO: 85, or an amino acid sequence with no more than 1, 2, or 3 amino acid substitutions of SEQ ID NO: 85; and the CDR-L3 comprises an amino acid sequence of SEQ ID NO: 86, or an amino acid sequence with no more than 1, 2, or 3 amino acid substitutions of SEQ ID NO: 86.

111. The CAR polypeptide of claim 110, wherein the VH comprises an amino acid sequence of SEQ ID NO: 87, or an amino acid sequence having at least 90% sequence identity to the amino acid sequence of SEQ ID NO: 87, and/or the VL comprises an amino acid sequence of SEQ ID NO: 88, or an amino acid sequence having at least 90% sequence identity to the amino acid sequence of SEQ ID NO: 88.

112. The CAR polypeptide of claim 106, wherein the antigen-binding domain of the CAR comprises:
(a) a heavy chain variable domain (VH) comprising three complementarity determining regions CDR-H1, CDR-H2, and CDR-H3, wherein the CDR-H1 comprises an amino acid sequence of SEQ ID NO: 89, or an amino acid sequence with no more than 1, 2, or 3 amino acid substitutions of SEQ ID NO: 89; the CDR-H2 comprises an amino acid sequence of SEQ ID NO: 90, or an amino acid sequence with no more than 1, 2, or 3 amino acid substitutions of SEQ ID NO: 90; and the CDR-H3 comprises an amino acid sequence of SEQ ID NO: 91, or an amino acid sequence with no more than 1, 2, or 3 amino acid substitutions of SEQ ID NO: 91, and/or
(b) a light chain variable domain (VL) comprising three complementarity determining regions CDR-L1, CDR-L2, and CDR-L3, wherein the CDR-L1 comprises an amino acid sequence of SEQ ID NO: 92, or an amino acid sequence with no more than 1, 2, or 3 amino acid substitutions of SEQ ID NO: 92; the CDR-L2 comprises an amino acid sequence of SEQ ID NO: 93, or an amino acid sequence with no more than 1, 2, or 3 amino acid substitutions of SEQ ID NO: 93; and the CDR-L3 comprises an amino acid sequence of SEQ ID NO: 94, or an amino acid sequence with no more than 1, 2, or 3 amino acid substitutions of SEQ ID NO: 94.

113. The CAR polypeptide of claim 112, wherein the VH comprises an amino acid sequence of SEQ ID NO: 95, or an amino acid sequence having at least 90% sequence identity to the amino acid sequence of SEQ ID NO: 95, and/or the VL comprises an amino acid sequence of SEQ ID NO: 96, or an amino acid sequence having at least 90% sequence identity to the amino acid sequence of SEQ ID NO: 96.

114. The CAR polypeptide of claim 110 or 112, wherein the VH is N-terminal to the VL.

115. The CAR polypeptide of claim 110 or 112, wherein the VL is N-terminal to the VH.

116. The CAR polypeptide of claim 106, wherein the antigen-binding domain of the CAR comprises a scFv or a single domain antibody, which optionally comprises a sequence selected from the group consisting of SEQ ID NO: 3, 9, 15, 25, 71, 77, and variants thereof.

117. The CAR polypeptide of claim 106, wherein the transmembrane domain comprises a hinge/transmembrane domain.

118. The CAR polypeptide of claim 117, wherein the hinge/transmembrane domain comprises the hinge/transmembrane domain of an immunoglobulin-like protein, CD28, CD8, or 4-1 BB.

119. The CAR polypeptide of claim 106, wherein the transmembrane domain of the CAR comprises a CD8 hinge/transmembrane domain, which optionally comprises the sequence of any one of SEQ ID NOs: 4, 10, 16, 22, 28, 37, 46, 58, 66, 72, 78, and 104, or a variant thereof.

120. The CAR polypeptide of claim 106, wherein the intracellular signaling domain comprises the intracellular signaling domain of TCFζ, FcRγ, FcRβ, CD3γ, CD3θ, CD3ε, CD3η, CD3ζ, CD22, CD79a, CD79b, or CD66d.

121. The CAR polypeptide of claim 106, wherein the intracellular signaling domain comprises a CD3ζ intracellular signaling domain, which optionally comprises the sequence of any one of SEQ ID NOs: 6, 12, 18, 24, 30, 39, 48, 60, 68, 74, 80, and 106, or a variant thereof.

122. The CAR polypeptide of claim 108, wherein the co-stimulatory domain comprises the co-stimulatory domain of 4-1 BB, CD27, CD28, or OX-40.

123. The CAR polypeptide of claim 108, wherein the co-stimulatory domain comprises a 4-1 BB co-stimulatory domain, which optionally comprises the sequence of any one of SEQ ID NOs: 5, 11, 17, 23, 29, 38, 47, 59, 67, 73, 79, and 105, or a variant thereof.

124. A CAR polypeptide comprising an amino acid sequence having at least 90% sequence identity to the amino acid sequence of any one of SEQ ID NO: 26, SEQ ID NO: 35, SEQ ID NO: 44, SEQ ID NO: 53, SEQ ID NO: 61, SEQ ID NO: 19, SEQ ID NO: 1, SEQ ID NO: 7, SEQ ID NO: 13, SEQ ID NO: 69, SEQ ID NO: 75, and SEQ ID NO: 100.

125. The CAR polypeptide of claim 124, comprising the amino acid sequence of any one of SEQ ID NO: 26, SEQ ID NO: 35, SEQ ID NO: 44, SEQ ID NO: 53, SEQ ID NO: 61, SEQ ID NO: 19, SEQ ID NO: 1, SEQ ID NO: 7, SEQ ID NO: 13, SEQ ID NO: 69, SEQ ID NO: 75, and SEQ ID NO: 100.

126. A nucleic acid molecule encoding (i) the CAR polypeptide, or (ii) a polyprotein comprising the CAR polypeptide and the therapeutic agent, of claim 75 or 106.

127. The nucleic acid molecule of claim 126, further comprising a suicide gene.

128. The nucleic acid molecule of claim 126, further comprising a sequence encoding a signal sequence.

129. A vector comprising the nucleic acid molecule of claim 126.

130. The vector of claim 129, wherein the vector is a lentiviral vector.

131. A polypeptide comprising the CAR polypeptide, or a polyprotein comprising the CAR polypeptide and the therapeutic agent, of claim 75 or 106.

132. An immune cell comprising the CAR polypeptide of claim 106.

133. The immune cell of claim 132, wherein the immune cell is a T or NK cell.

134. The immune cell of claim 132, wherein the immune cell is a human cell.

135. A pharmaceutical composition comprising one or more CAR T cells, nucleic acid molecules, CAR polypeptides, polyproteins, or immune cells of claim 75 or 106.

136. A method of treating a patient having cancer, the method comprising administering to the patient the pharmaceutical composition of claim 135.

137. The method of claim 136, wherein by targeting the tumor microenvironment, systemic toxicity is reduced.

138. The method of claim 136, wherein the cancer is characterized by the presence of one or more solid tumors.

139. The method of claim 136, wherein the cancer is characterized by tumor-infiltrating Tregs.

140. The method of claim 136, wherein the cancer is a glioblastoma.

141. A method of treating a patient having cancer, the method comprising administering to the patient a CAR T cell product, genetically modified to secrete a tumor-toxic antibody or cytokine, wherein by directing the cancer toxicity locally to the tumor microenvironment, systemic toxicity is reduced.

142. The method of claim 141, wherein the CAR T cell is genetically modified to deliver an antibody against CTLA4, CD25, GARP, LAP, IL-15, CSF1R, or EGFR, EGFRvIII, CD19, CD79b, CD37, PSMA, PSCA, IL-13Rα2, EphA1, Her2, mesothelin, MUC1, or MUC16, or a bispecific antibody to the tumor microenvironment.

143. The method of claim 142, wherein the bispecific antibody is a BiTE directed against EGFR and CD3.

144. A method of delivering a therapeutic agent to a tissue or organ in a patient to treat a disease or pathology, the method comprising administering to said patient a CAR T cell, genetically modified to secrete a therapeutic antibody, toxin, or agent, wherein the therapeutic antibody, toxin, or agent would, by itself, be unable to enter or penetrate the tissue or organ.

145. The method of claim 144, wherein the tissue or organ is in the nervous system.

146. The method of claim 145, wherein the nervous system is the central nervous system.

147. The method of claim 146, wherein the central nervous system is the brain.

148. The method of claim 144, wherein the disease or pathology is a cancer.

149. The method of claim 148, wherein the cancer is glioblastoma, prostate cancer, lung cancer, pancreatic cancer, lymphoma, or myeloma.

150. The method of claim 144, wherein the therapeutic antibody is anti-EGFR or anti-EGFRvIII.

151. A method of treating glioblastoma having reduced EGFRvIII expression in a subject comprising administering to the subject a CAR T cell engineered to express: (i) a CAR polypeptide comprising an extracellular EGFRvIII-binding domain; and (ii) a BiTE, wherein the CAR T cell is optionally the CAR T cell of claim 75.

152. A method of preventing or reducing immunosuppression in the tumor microenvironment in a subject comprising administering to the subject a CAR T cell engineered to express: (i) a CAR polypeptide comprising an extracellular target binding domain; and (ii) a BiTE, wherein the CAR T cell is optionally the CAR T cell of claim 75.

153. A method of preventing or reducing T cell exhaustion in the tumor microenvironment in a subject, the method comprising administering to the subject a CAR T cell engineered to express: (i) a CAR polypeptide comprising an extracellular target binding domain; and (ii) a BiTE, wherein the CAR T cell is optionally the CAR T cell of claim 75.

154. A method of treating a cancer having heterogeneous antigen expression in a subject, the method comprising administering to the subject a CAR T cell engineered to express: (i) a CAR polypeptide comprising an extracellular target binding domain; and (ii) a BiTE, wherein the CAR T cell is optionally the CAR T cell of claim 75.

155. The method of claim 154, wherein the cancer is glioblastoma, prostate cancer, lung cancer, pancreatic cancer, lymphoma, or myeloma.

156. The method of claim 154, wherein the cancer comprises cells expressing one or more of EGFR, EGFRvIII, CD19, PSMA, PSCA, IL-13Rα2, EphA1, Her2, mesothelin, MUC1, and MUC16.


SEQUENCE LISTING (117 sequences)

SEQ ID NO: 1 - protein, 351 aa, Artificial Sequence
  Synthetic Construct
MALPVTALLLPLALLLHAARPDIQMTQSPSSLSASLGDRVTITCQASQSISSYLAWYQQKPGQAPNILIYGASRLKTGVPSRFSGSGSGTSFTLTISGLEAEDAGTYYCQQYASVPVTFGQGTKVELKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 21 aa, Artificial Sequence
  Synthetic Construct
MALPVTALLLPLALLLHAARP

SEQ ID NO: 3 - protein, 107 aa, Artificial Sequence
  Synthetic Construct
DIQMTQSPSSLSASLGDRVTITCQASQSISSYLAWYQQKPGQAPNILIYGASRLKTGVPSRFSGSGSGTSFTLTISGLEAEDAGTYYCQQYASVPVTFGQGTKVELK

SEQ ID NO: 4 - protein, 69 aa, Artificial Sequence
  Synthetic Construct
TTTPAPRPPTPAPTIASQPLSLRPEACRPAAGGAVHTRGLDFACDIYIWAPLAGTCGVLLLSLVITLYC

SEQ ID NO: 5 - protein, 42 aa, Artificial Sequence
  Synthetic Construct
KRGRKKLLYIFKQPFMRPVQTTQEEDGCSCRFPEEEEGGCEL

SEQ ID NO: 6 - protein, 112 aa, Artificial Sequence
  Synthetic Construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 530 aa, Artificial Sequence
  Synthetic Construct
MALPVTALLLPLALLLHAARPMKLWLNWIFLVTLLNDIQCEVKLVESGGGLVQPGGSLSLSCAASGFTFTDYYMSWVRQPPGKALEWLGFIRNKPNGYTTEYSASVKGRFTISRDNSQSILYLQMNVLRAEDSATYYCARYTGGGYFDYWGQGTTLTVSSGGGGSGGGGSGGGGSGGGGSMMSSAQFLGLLLLCFQGTRCDIQMTQTTSSLSASLGDRLTISCRASQDISNYLNWYQQKPDGTVKLLIYYTSRLHSGVPSRFSGSGSGTDYSLTISNLEQADIATYFCQQGDTLPWTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 21 aa, Artificial Sequence
  Synthetic Construct
MALPVTALLLPLALLLHAARP

SEQ ID NO: 9 - protein, 286 aa, Artificial Sequence
  Synthetic Construct
MKLWLNWIFLVTLLNDIQCEVKLVESGGGLVQPGGSLSLSCAASGFTFTDYYMSWVRQPPGKALEWLGFIRNKPNGYTTEYSASVKGRFTISRDNSQSILYLQMNVLRAEDSATYYCARYTGGGYFDYWGQGTTLTVSSGGGGSGGGGSGGGGSGGGGSMMSSAQFLGLLLLCFQGTRCDIQMTQTTSSLSASLGDRLTISCRASQDISNYLNWYQQKPDGTVKLLIYYTSRLHSGVPSRFSGSGSGTDYSLTISNLEQADIATYFCQQGDTLPWTFGGGTKLEIK

SEQ ID NO: 10 - protein, 69 aa, Artificial Sequence; identical to SEQ ID NO: 4
  Synthetic Construct

SEQ ID NO: 11 - protein, 42 aa, Artificial Sequence; identical to SEQ ID NO: 5
  Synthetic Construct

SEQ ID NO: 12 - protein, 112 aa, Artificial Sequence; identical to SEQ ID NO: 6
  Synthetic Construct

SEQ ID NO: 13 - protein, 530 aa, Artificial Sequence
  Synthetic Construct
MALPVTALLLPLALLLHAARPMMSSAQFLGLLLLCFQGTRCDIQMTQTTSSLSASLGDRLTISCRASQDISNYLNWYQQKPDGTVKLLIYYTSRLHSGVPSRFSGSGSGTDYSLTISNLEQADIATYFCQQGDTLPWTFGGGTKLEIKGGGGSGGGGSGGGGSGGGGSMKLWLNWIFLVTLLNDIQCEVKLVESGGGLVQPGGSLSLSCAASGFTFTDYYMSWVRQPPGKALEWLGFIRNKPNGYTTEYSASVKGRFTISRDNSQSILYLQMNVLRAEDSATYYCARYTGGGYFDYWGQGTTLTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 21 aa, Artificial Sequence
  Synthetic Construct
MALPVTALLLPLALLLHAARP

SEQ ID NO: 15 - protein, 286 aa, Artificial Sequence
  Synthetic Construct
MMSSAQFLGLLLLCFQGTRCDIQMTQTTSSLSASLGDRLTISCRASQDISNYLNWYQQKPDGTVKLLIYYTSRLHSGVPSRFSGSGSGTDYSLTISNLEQADIATYFCQQGDTLPWTFGGGTKLEIKGGGGSGGGGSGGGGSGGGGSMKLWLNWIFLVTLLNDIQCEVKLVESGGGLVQPGGSLSLSCAASGFTFTDYYMSWVRQPPGKALEWLGFIRNKPNGYTTEYSASVKGRFTISRDNSQSILYLQMNVLRAEDSATYYCARYTGGGYFDYWGQGTTLTVSS

SEQ ID NO: 16 - protein, 69 aa, Artificial Sequence; identical to SEQ ID NO: 4
  Synthetic Construct

SEQ ID NO: 17 - protein, 42 aa, Artificial Sequence; identical to SEQ ID NO: 5
  Synthetic Construct

SEQ ID NO: 18 - protein, 112 aa, Artificial Sequence; identical to SEQ ID NO: 6
  Synthetic Construct

SEQ ID NO: 19 - protein, 654 aa, Artificial Sequence
  Synthetic Construct
MALPVTALLLPLALLLHAARPQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRPGSGSGATNFSLLKQAGDVEENPGPRTAMETDTLLLWVLLLWVPGSTGDDIQMTQSPSSLSASLGDRVTITCQASQSISSYLAWYQQKPGQAPNILIYGASRLKTGVPSRFSGSGSGTSFTLTISGLEAEDAGTYYCQQYASVPVTFGQGTKVELKHHHHHHSG

SEQ ID NO: 20 - protein, 21 aa, Artificial Sequence
  Synthetic Construct
MALPVTALLLPLALLLHAARP

SEQ ID NO: 21 - protein, 246 aa, Artificial Sequence
  Synthetic Construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 22 - protein, 69 aa, Artificial Sequence; identical to SEQ ID NO: 4
  Synthetic Construct

SEQ ID NO: 23 - protein, 42 aa, Artificial Sequence; identical to SEQ ID NO: 5
  Synthetic Construct

SEQ ID NO: 24 - protein, 112 aa, Artificial Sequence; identical to SEQ ID NO: 6
  Synthetic Construct

SEQ ID NO: 25 - protein, 107 aa, Artificial Sequence; identical to SEQ ID NO: 3
  Synthetic Construct

SEQ ID NO: 26 - protein, 1006 aa, Artificial Sequence
  Synthetic Construct
EIQLQQSGAELVKPGASVKLSCTGSGFNIEDYYIHWVKQRTEQGLEWIGRIDPENDETKYGPIFQGRATITADTSSNTVYLQLSSLTSEDTAVYYCAFRGGVYWGPGTTLTVSSGGGGSGGGGSGGGGSHMDVVMTQSPLTLSVAIGQSASISCKSSQSLLDSDGKTYLNWLLQRPGQSPKRLISLVSKLDSGVPDRFTGSGSGTDFTLRISRVEAEDLGIYYCWQGTHFPGTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPPRMETDTLLLWVLLLWVPGSTGDDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 27 - protein, 243 aa, Artificial Sequence
  Synthetic Construct
EIQLQQSGAELVKPGASVKLSCTGSGFNIEDYYIHWVKQRTEQGLEWIGRIDPENDETKYGPIFQGRATITADTSSNTVYLQLSSLTSEDTAVYYCAFRGGVYWGPGTTLTVSSGGGGSGGGGSGGGGSHMDVVMTQSPLTLSVAIGQSASISCKSSQSLLDSDGKTYLNWLLQRPGQSPKRLISLVSKLDSGVPDRFTGSGSGTDFTLRISRVEAEDLGIYYCWQGTHFPGTFGGGTKLEIK

SEQ ID NO: 28 - protein, 69 aa, Artificial Sequence; identical to SEQ ID NO: 4
  Synthetic Construct

SEQ ID NO: 29 - protein, 42 aa, Artificial Sequence; identical to SEQ ID NO: 5
  Synthetic Construct

SEQ ID NO: 30 - protein, 112 aa, Artificial Sequence; identical to SEQ ID NO: 6
  Synthetic Construct

SEQ ID NO: 31 - protein, 22 aa, Artificial Sequence
  Synthetic Construct
GSGATNFSLLKQAGDVEENPGP

SEQ ID NO: 32 - protein, 21 aa, Artificial Sequence
  Synthetic Construct
METDTLLLWVLLLWVPGSTGD

SEQ ID NO: 33 - protein, 241 aa, Artificial Sequence
  Synthetic Construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 34 - protein, 243 aa, Artificial Sequence
  Synthetic Construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 35 - protein, 1009 aa, Artificial Sequence
  Synthetic Construct
EIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPPRMETDTLLLWVLLLWVPGSTGDDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 36 - protein, 246 aa, Artificial Sequence
  Synthetic Construct
EIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIK

SEQ ID NO: 37 - protein, 69 aa, Artificial Sequence; identical to SEQ ID NO: 4
  Synthetic Construct

SEQ ID NO: 38 - protein, 42 aa, Artificial Sequence; identical to SEQ ID NO: 5
  Synthetic Construct

SEQ ID NO: 39 - protein, 112 aa, Artificial Sequence; identical to SEQ ID NO: 6
  Synthetic Construct

SEQ ID NO: 40 - protein, 22 aa, Artificial Sequence
  Synthetic Construct
GSGATNFSLLKQAGDVEENPGP

SEQ ID NO: 41 - protein, 21 aa, Artificial Sequence
  Synthetic Construct
METDTLLLWVLLLWVPGSTGD

SEQ ID NO: 42 - protein, 241 aa, Artificial Sequence; identical to SEQ ID NO: 33
  Synthetic Construct

SEQ ID NO: 43 - protein, 243 aa, Artificial Sequence; identical to SEQ ID NO: 34
  Synthetic Construct

SEQ ID NO: 44 - protein, 1018 aa, Artificial Sequence
  Synthetic Construct
EIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPPRMETDTLLLWVLLLWVPGSTGDDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 45 - protein, 246 aa, Artificial Sequence; identical to SEQ ID NO: 36
  Synthetic Construct

SEQ ID NO: 46 - protein, 69 aa, Artificial Sequence; identical to SEQ ID NO: 4
  Synthetic Construct

SEQ ID NO: 47 - protein, 42 aa, Artificial Sequence; identical to SEQ ID NO: 5
  Synthetic Construct

SEQ ID NO: 48 - protein, 112 aa, Artificial Sequence; identical to SEQ ID NO: 6
  Synthetic Construct

SEQ ID NO: 49 - protein, 22 aa, Artificial Sequence
  Synthetic Construct
GSGATNFSLLKQAGDVEENPGP

SEQ ID NO: 50 - protein, 21 aa, Artificial Sequence
  Synthetic Construct
METDTLLLWVLLLWVPGSTGD

SEQ ID NO: 51 - protein, 250 aa, Artificial Sequence
  Synthetic Construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 52 - protein, 243 aa, Artificial Sequence; identical to SEQ ID NO: 34
  Synthetic Construct

SEQ ID NO: 53 - protein, 985 aa, Artificial Sequence
  Synthetic Construct
METDTLLLWVLLLWVPGSTGDDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHHEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 21 aa, Artificial Sequence
  Synthetic Construct
METDTLLLWVLLLWVPGSTGD

SEQ ID NO: 55 - protein, 241 aa, Artificial Sequence; identical to SEQ ID NO: 33
  Synthetic Construct

SEQ ID NO: 56 - protein, 243 aa, Artificial Sequence; identical to SEQ ID NO: 34
  Synthetic Construct

SEQ ID NO: 57 - protein, 246 aa, Artificial Sequence; identical to SEQ ID NO: 36
  Synthetic Construct

SEQ ID NO: 58 - protein, 69 aa, Artificial Sequence; identical to SEQ ID NO: 4
  Synthetic Construct

SEQ ID NO: 59 - protein, 42 aa, Artificial Sequence; identical to SEQ ID NO: 5
  Synthetic Construct

SEQ ID NO: 60 - protein, 112 aa, Artificial Sequence; identical to SEQ ID NO: 6
  Synthetic Construct

SEQ ID NO: 61 - protein, 994 aa, Artificial Sequence
  Synthetic Construct
METDTLLLWVLLLWVPGSTGDDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHHEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 21 aa, Artificial Sequence
  Synthetic Construct
METDTLLLWVLLLWVPGSTGD

SEQ ID NO: 63 - protein, 250 aa, Artificial Sequence; identical to SEQ ID NO: 51
  Synthetic Construct

SEQ ID NO: 64 - protein, 243 aa, Artificial Sequence; identical to SEQ ID NO: 34
  Synthetic Construct

SEQ ID NO: 65 - protein, 246 aa, Artificial Sequence; identical to SEQ ID NO: 36
  Synthetic Construct

SEQ ID NO: 66 - protein, 69 aa, Artificial Sequence; identical to SEQ ID NO: 4
  Synthetic Construct

SEQ ID NO: 67 - protein, 42 aa, Artificial Sequence; identical to SEQ ID NO: 5
  Synthetic Construct

SEQ ID NO: 68 - protein, 112 aa, Artificial Sequence; identical to SEQ ID NO: 6
  Synthetic Construct

SEQ ID NO: 69 - protein, 497 aa, Artificial Sequence
  Synthetic Construct
MALPVTALLLPLALLLHAARPEVQLVQPGAELRNSGASVKVSCKASGYRFTSYYIDWVRQAPGQGLEWMGRIDPEDGGTKYAQKFQGRVTFTADTSTSTAYVELSSLRSEDTAVYYCARNEWETVVVGDLMYEYEYWGQGTQVTVSSGGGGSGGGGSGGGGSGGGGSDIQMTQSPSSLSASLGDRVTITCQASQSISSYLAWYQQKPGQAPNILIYGASRLKTGVPSRFSGSGSGTSFTLTISGLEAEDAGTYYCQQYASVPVTFGQGTKVELKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - protein, 21 aa, Artificial Sequence
  Synthetic Construct
MALPVTALLLPLALLLHAARP

SEQ ID NO: 71 - protein, 253 aa, Artificial Sequence
  Synthetic Construct
EVQLVQPGAELRNSGASVKVSCKASGYRFTSYYIDWVRQAPGQGLEWMGRIDPEDGGTKYAQKFQGRVTFTADTSTSTAYVELSSLRSEDTAVYYCARNEWETVVVGDLMYEYEYWGQGTQVTVSSGGGGSGGGGSGGGGSGGGGSDIQMTQSPSSLSASLGDRVTITCQASQSISSYLAWYQQKPGQAPNILIYGASRLKTGVPSRFSGSGSGTSFTLTISGLEAEDAGTYYCQQYASVPVTFGQGTKVELK

SEQ ID NO: 72 - protein, 69 aa, Artificial Sequence; identical to SEQ ID NO: 4
  Synthetic Construct

SEQ ID NO: 73 - protein, 42 aa, Artificial Sequence; identical to SEQ ID NO: 5
  Synthetic Construct

SEQ ID NO: 74 - protein, 112 aa, Artificial Sequence; identical to SEQ ID NO: 6
  Synthetic Construct

SEQ ID NO: 75 - protein, 497 aa, Artificial Sequence
  Synthetic Construct
MALPVTALLLPLALLLHAARPDIQMTQSPSSLSASLGDRVTITCQASQSISSYLAWYQQKPGQAPNILIYGASRLKTGVPSRFSGSGSGTSFTLTISGLEAEDAGTYYCQQYASVPVTFGQGTKVELKGGGGSGGGGSGGGGSGGGGSEVQLVQPGAELRNSGASVKVSCKASGYRFTSYYIDWVRQAPGQGLEWMGRIDPEDGGTKYAQKFQGRVTFTADTSTSTAYVELSSLRSEDTAVYYCARNEWETVVVGDLMYEYEYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 21 aa, Artificial Sequence
  Synthetic Construct
MALPVTALLLPLALLLHAARP

SEQ ID NO: 77 - protein, 253 aa, Artificial Sequence
  Synthetic Construct
DIQMTQSPSSLSASLGDRVTITCQASQSISSYLAWYQQKPGQAPNILIYGASRLKTGVPSRFSGSGSGTSFTLTISGLEAEDAGTYYCQQYASVPVTFGQGTKVELKGGGGSGGGGSGGGGSGGGGSEVQLVQPGAELRNSGASVKVSCKASGYRFTSYYIDWVRQAPGQGLEWMGRIDPEDGGTKYAQKFQGRVTFTADTSTSTAYVELSSLRSEDTAVYYCARNEWETVVVGDLMYEYEYWGQGTQVTVSS

SEQ ID NO: 78 - protein, 69 aa, Artificial Sequence; identical to SEQ ID NO: 4
  Synthetic Construct

SEQ ID NO: 79 - protein, 42 aa, Artificial Sequence; identical to SEQ ID NO: 5
  Synthetic Construct

SEQ ID NO: 80 - protein, 112 aa, Artificial Sequence; identical to SEQ ID NO: 6
  Synthetic Construct

SEQ ID NO: 81 - protein, 5 aa, Artificial Sequence
  Synthetic Construct
SYYID

SEQ ID NO: 82 - protein, 17 aa, Artificial Sequence
  Synthetic Construct
RIDPEDGGTKYAQKFQG

SEQ ID NO: 83 - protein, 17 aa, Artificial Sequence
  Synthetic Construct
NEWETVVVGDLMYEYEY

SEQ ID NO: 84 - protein, 11 aa, Artificial Sequence
  Synthetic Construct
QASQSISSYLA

SEQ ID NO: 85 - protein, 7 aa, Artificial Sequence
  Synthetic Construct
GASRLKT

SEQ ID NO: 86 - protein, 9 aa, Artificial Sequence
  Synthetic Construct
QQYASVPVT

SEQ ID NO: 87 - protein, 126 aa, Artificial Sequence
  Synthetic Construct
EVQLVQPGAELRNSGASVKVSCKASGYRFTSYYIDWVRQAPGQGLEWMGRIDPEDGGTKYAQKFQGRVTFTADTSTSTAYVELSSLRSEDTAVYYCARNEWETVVVGDLMYEYEYWGQGTQVTVSS

SEQ ID NO: 88 - protein, 107 aa, Artificial Sequence; identical to SEQ ID NO: 3
  Synthetic Construct

SEQ ID NO: 89 - protein, 11 aa, Artificial Sequence
  Synthetic Construct
RASQDISNYLN

SEQ ID NO: 90 - protein, 7 aa, Artificial Sequence
  Synthetic Construct
YTSRLHS

SEQ ID NO: 91 - protein, 9 aa, Artificial Sequence
  Synthetic Construct
QQGDTLPWT

SEQ ID NO: 92 - protein, 5 aa, Artificial Sequence
  Synthetic Construct
DYYMS

SEQ ID NO: 93 - protein, 19 aa, Artificial Sequence
  Synthetic Construct
FIRNKPNGYTTEYSASVKG

SEQ ID NO: 94 - protein, 9 aa, Artificial Sequence
  Synthetic Construct
YTGGGYFDY

SEQ ID NO: 95 - protein, 139 aa, Artificial Sequence
  Synthetic Construct
MKLWLNWIFLVTLLNDIQCEVKLVESGGGLVQPGGSLSLSCAASGFTFTDYYMSWVRQPPGKALEWLGFIRNKPNGYTTEYSASVKGRFTISRDNSQSILYLQMNVLRAEDSATYYCARYTGGGYFDYWGQGTTLTVSS

SEQ ID NO: 96 - protein, 127 aa, Artificial Sequence
  Synthetic Construct
MMSSAQFLGLLLLCFQGTRCDIQMTQTTSSLSASLGDRLTISCRASQDISNYLNWYQQKPDGTVKLLIYYTSRLHSGVPSRFSGSGSGTDYSLTISNLEQADIATYFCQQGDTLPWTFGGGTKLEIK

SEQ ID NO: 97 - protein, 6 aa, Artificial Sequence
  Synthetic Construct
HHHHHH

SEQ ID NO: 98 - protein, 489 aa, Artificial Sequence
  Synthetic Construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 99 - protein, 498 aa, Artificial Sequence
  Synthetic Construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 100 - protein, 601 aa, Artificial Sequence
  Synthetic Construct
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 101 - protein, 112 aa, Artificial Sequence
  Synthetic Construct
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 102 - protein, 20 aa, Artificial Sequence
  Synthetic Construct
GGGGSGGGGSGGGGSGGGGS

SEQ ID NO: 103 - protein, 246 aa, Artificial Sequence; identical to SEQ ID NO: 36
  Synthetic Construct

SEQ ID NO: 104 - protein, 69 aa, Artificial Sequence; identical to SEQ ID NO: 4
  Synthetic Construct

SEQ ID NO: 105 - protein, 42 aa, Artificial Sequence; identical to SEQ ID NO: 5
  Synthetic Construct

SEQ ID NO: 106 - protein, 112 aa, Artificial Sequence; identical to SEQ ID NO: 6
  Synthetic Construct

SEQ ID NO: 107 - protein, 12 aa, Artificial Sequence
  Synthetic Construct
GGGSGGGSGGGS

SEQ ID NO: 108 - protein, 15 aa, Artificial Sequence
  Synthetic Construct
GGGGSGGGGSGGGGS

SEQ ID NO: 109 - protein, 18 aa, Artificial Sequence
  Synthetic Construct
GSTSGSGKPGSGEGSTKG

SEQ ID NO: 110 - protein, 18 aa, Artificial Sequence
  Synthetic Construct
GGSSRSSSSGGGGSGGGG

SEQ ID NO: 111 - protein, 114 aa, Artificial Sequence
  Synthetic Construct
EIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSS

SEQ ID NO: 112 - protein, 112 aa, Artificial Sequence
  Synthetic Construct
DVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIK

SEQ ID NO: 113 - protein, 114 aa, Artificial Sequence
  Synthetic Construct
EIQLQQSGAELVKPGASVKLSCTGSGFNIEDYYIHWVKQRTEQGLEWIGRIDPENDETKYGPIFQGRATITADTSSNTVYLQLSSLTSEDTAVYYCAFRGGVYWGPGTTLTVSS

SEQ ID NO: 114 - protein, 114 aa, Artificial Sequence
  Synthetic Construct
HMDVVMTQSPLTLSVAIGQSASISCKSSQSLLDSDGKTYLNWLLQRPGQSPKRLISLVSKLDSGVPDRFTGSGSGTDFTLRISRVEAEDLGIYYCWQGTHFPGTFGGGTKLEIK

SEQ ID NO: 115 - protein, 466 aa, Artificial Sequence
  Synthetic Construct
EIQLQQSGAELVKPGASVKLSCTGSGFNIEDYYIHWVKQRTEQGLEWIGRIDPENDETKYGPIFQGRATITADTSSNTVYLQLSSLTSEDTAVYYCAFRGGVYWGPGTTLTVSSGGGGSGGGGSGGGGSHMDVVMTQSPLTLSVAIGQSASISCKSSQSLLDSDGKTYLNWLLQRPGQSPKRLISLVSKLDSGVPDRFTGSGSGTDFTLRISRVEAEDLGIYYCWQGTHFPGTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - protein, 469 aa, Artificial Sequence
  Synthetic Construct
EIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 117 - protein, 469 aa, Artificial Sequence
  Synthetic Construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR